SEQUENCE LISTING (136 sequences)

SEQ ID NO: 1 - protein, 519 aa, Thamnostylum piriforme
MDNKAILRQAVYSYRYHIGIAAAVAIVCQQIYSRVFRVPKNLRHIPAISYWSQMKALLKKEAITPRTKRLVYPLLSKANGLYLNRMPFDWTVYVANPMIAKTVLFKPEFATKMSLIGSFSDDSAVLQFVGKDNIAVSNGHQWKKQRKIMNPAFHRSMPVALFASLMPKVFCMIDETQENGDSVGAIQLMQALTLEALGKAVFGFDFGGLDDKNSVWVKTYNEVFKAFTDFTVFLPRLNAFLRRVSPPHRSRHREMLKLIGLLDEMVDKKRQALLDAKNKSVEKVPEHEKDLLTLMLEAEMDGEGVWEKEELRHNIGILFVAGHDTTANSLAFAVYNLAVNKDVQDKARKEIIDLLGDEPKDVAPTLDDCKHMDYIDMIIKETLRMNAPANDLLARIASEDLELGGVLIPKGTMVSVDFHALHLHPDLWEDSERFNPQRFQDNGEHSKHEGVTWVPFSGGSRQCIGINFSMMEQRVALSTLLRKYEWELPADSIHRNGLVMDQPFSFAPSSLKIKFKKRY

SEQ ID NO: 2 - DNA, 1560 bp, Thamnostylum piriforme
  source 1..1560: mol_type = other DNA
atggacaacaaagcaattttacgacaagccgtttactcgtatcgttaccacatcgggatcgcggcggctgtagctatcgtttgccagcaaatatacagtcgcgtctttcgtgttcccaaaaatttgcgtcatattccagcaattagctattggagtcaaatgaaagcacttttgaaaaaggaggcaatcactccacgtacgaagaggctagtttacccgctgctttcaaaagcgaatggactctacctgaatcgaatgccatttgactggacagtttatgtcgcgaatccaatgattgcgaagactgtcttgtttaagccagagtttgcaaccaaaatgtcattaataggctcattcagcgatgatagtgcagttttgcaatttgttggaaaggataatattgcggtatcaaacggtcaccagtggaaaaagcaaagaaagattatgaaccctgctttccatcgatcaatgccggtagctctctttgcaagtctcatgccaaaggttttttgtatgattgatgaaacacaagagaacggtgattctgttggtgccatacaacttatgcaggcccttactcttgaagcgcttggaaaagcagttttcggttttgattttggaggattggacgataaaaattctgtttgggtcaagacatacaacgaagtattcaaggcatttacagacttcacggtattccttccacgacttaatgcttttcttcggcgggtaagcccaccgcatcgaagtcgtcatagagaaatgctcaagttgatcggccttctggatgaaatggtggacaaaaagagacaagcgctcttggatgccaagaataaatccgtcgagaaagtgcctgagcatgaaaaggatctacttactctcatgctcgaggctgaaatggatggtgaaggtgtttgggaaaaagaagaattacggcacaatatcgggatattattcgtggcagggcatgatacaaccgccaattcattggcatttgcggtttacaatctagccgtcaacaaagacgtacaagataaggcccgcaaagaaattatcgacttactaggagacgagcctaaggatgttgcgcccaccttggatgactgcaaacatatggattatatcgatatgattatcaaagagacgctccgcatgaatgctcctgcaaatgacttgcttgctcgaatagcatcggaagacttggaactaggaggggttttaatacccaaggggacaatggtctcggtcgactttcacgcactgcaccttcaccctgatttatgggaggattcagagcgtttcaacccgcagcgatttcaagataatggagagcatagcaaacacgaaggagtaacatgggttccttttagtggaggctcaagacaatgcattggaatcaattttagtatgatggagcaacgggtggccctatcgacgctattgagaaaatatgaatgggagctacctgctgattctatccaccgtaatggacttgtgatggatcaaccttttagttttgcaccaagctctctcaaaattaagtttaaaaaacgttattga

SEQ ID NO: 3 - DNA, 1560 bp, Thamnostylum piriforme
  source 1..1560: mol_type = other DNA
atggataataaagctattttgagacaagctgtttattcttatagataccatattggtattgctgctgctgttgctattgtttgtcaacaaatttattctagagtttttagagttccaaaaaatttgagacatattccagctatttcttattggtctcaaatgaaagcattgttgaaaaaagaagctattactccaagaactaaaagattggtttatccattgttgtctaaagctaatggtttgtatttgaatagaatgccatttgattggactgtttatgttgctaatccaatgattgctaaaactgttttgtttaaaccagaatttgctactaaaatgtctttgattggttctttttctgatgattctgctgttttgcaatttgttggtaaagataatattgctgtttctaatggtcatcaatggaaaaaacaaagaaaaattatgaatccagcttttcatagatctatgccagttgctttgtttgcttctttgatgccaaaagttttttgtatgattgatgaaactcaagaaaatggtgattctgttggtgctattcaattgatgcaagcattgactttggaagcattgggtaaagctgtttttggttttgattttggtggtttggatgataaaaattctgtttgggttaaaacttataatgaagtttttaaagcatttactgattttactgtttttttgccaagattgaatgcttttttgagaagagtttctccaccacatagatctagacatagagaaatgttgaaattgattggtttgttggatgaaatggttgataaaaaaagacaagcattgttggatgctaaaaataaatctgttgaaaaagttccagaacatgaaaaagatttgttgactttgatgttggaagctgaaatggatggtgaaggtgtttgggaaaaagaagaattgagacataatattggtattttgtttgttgctggtcatgatactactgctaattctttggcttttgctgtttataatttggctgttaataaagatgttcaagataaagctagaaaagaaattattgatttgttgggtgatgaaccaaaagatgttgctccaactttggatgattgtaaacatatggattatattgatatgattattaaagaaactttgagaatgaatgctccagctaatgatttgttggctagaattgcttctgaagatttggaattgggtggtgttttgattccaaaaggtactatggtttctgttgattttcatgctttgcatttgcatccagatttgtgggaagattctgaaagatttaatccacaaagatttcaagataatggtgaacattctaaacatgaaggtgttacttgggttccattttctggtggttctagacaatgtattggtattaatttttctatgatggaacaaagagttgctttgtctactttgttgagaaaatatgaatgggaattgccagctgattctattcatagaaatggtttggttatggatcaaccattttcttttgctccatcttctttgaaaattaaatttaaaaaaagatattga

SEQ ID NO: 4 - protein, 509 aa, Thamnostylum piriforme
MAILQNLVLQSDQRHLGVVTAAVFLSAYALYRHESKKELGNECPTVPYTNPMFGSTDEYRKNPAAFVEKWSAALGPVFRVHIFGRMQTVVSGRYVREVLFNDNFSFVEGIRTRFDLRLLTGIPNSHLNDKDVREVVVKSLTAQMKKYTPRAVHYLSVGLQEALGDLKDPRTLDNLFLTVQNMVAKASASIFVGEELCNNIELVDTFKHVTSDIGSEIRLDNTWLEFFKTINQIRMWYVGKHSPRVTKHRRQLIQAMAPEIDRRLQGLAGNDPSWTRPEDILQEIMENYPSPSTVPADIYTYYANWMIVLIFASVHTTTEHATIVLYRLLQQPELIDELLQEQKEALGQGTVFTGEVIRKLVKLDSVCRESLRIKNEYFGLPHRNMSHENISLSNGVVIKPGDNVMLNVWTNHHDSDLQKDVHGNHDKFEPFRYVNADRPATKIGDDYLIFGEGKHACPGRWFALQEIKTIVSVLIRDYTLTPCGPIVFPSGTSTGIPSGEVTIQRKSEV

SEQ ID NO: 5 - DNA, 1530 bp, Thamnostylum piriforme
  source 1..1530: mol_type = other DNA
atggcaattcttcaaaaccttgtcctacaatctgatcagcgacaccttggtgtcgttacagctgctgtttttttgtcagcgtacgcgctctatcgacatgagtcgaaaaaggaactggggaatgagtgtcctactgtaccctataccaatccaatgttcggttccactgacgaatatcgcaagaatccagccgcatttgttgaaaaatggagcgctgcacttggcccagtatttcgcgttcatatctttggcagaatgcagacggtagtttcgggccgttatgtccgcgaagttttgttcaacgacaactttagttttgtggaaggaatccggacacggtttgaccttcgattgctcaccggtatacctaacagtcacctcaatgacaaagatgttcgagaggttgttgtcaagtcgttgactgcgcaaatgaagaagtatacaccgcgcgcagtacactacctcagcgttggcttgcaagaggcactaggagatttgaaagatcctcgaactcttgacaacctattcttgactgttcaaaacatggtcgccaaagctagcgcctccatcttcgtcggtgaagaactttgtaacaatattgagttggttgacacattcaagcacgttacaagcgacattggttcggagatacgtctcgacaacacctggctggaattctttaaaacaataaaccaaattcgaatgtggtacgttggcaagcattctcccagagtgacaaagcacagacgacagctcatacaggcgatggcacctgaaatcgacagacggctgcaaggtcttgcagggaatgatccttcttggaccagaccagaagacattttgcaagagattatggaaaactatccttccccgtccactgtaccagcagatatttacacctattatgccaattggatgattgtgttgatatttgcatccgttcacacgaccactgagcatgcaacaattgtcctttaccgtttactccaacagcccgagcttatcgacgagcttcttcaagaacaaaaggaggcccttggccaaggcactgtatttacaggcgaggtcattcgcaaactggtgaaactggatagcgtgtgtcgtgagtctttgcggatcaaaaacgagtactttggtcttccacacaggaatatgtctcatgaaaatatttctttgagcaatggagttgttatcaaaccaggcgacaatgtcatgctgaatgtttggacaaaccatcatgacagtgacttgcaaaaggatgtacacggcaaccatgacaagtttgaacctttccgttacgttaatgctgatcgccctgcaacgaagattggtgatgattacttgatatttggggagggaaagcacgcttgccccggtcgatggtttgctttacaagaaattaagacgattgtatcagtactaattcgagattacacgctaacaccatgtggtccaattgtgtttccatccggcacaagtactggtattccaagtggagaagtgacaattcaacgaaagtcagaagtgtaa

SEQ ID NO: 6 - DNA, 1530 bp, Thamnostylum piriforme
  source 1..1530: mol_type = other DNA
atggctattttgcaaaatttggttttgcaatctgatcaaagacatttgggtgttgttactgctgctgtttttttgtctgcttatgctttgtatagacatgaatctaaaaaagaattgggtaatgaatgtccaactgttccatatactaatccaatgtttggttctactgatgaatatagaaaaaatccagcagcttttgttgaaaaatggtctgctgctttgggtccagtttttagagttcatatttttggtagaatgcaaactgttgtttctggtagatatgttagagaagttttgtttaatgataatttttcttttgttgaaggtattagaactagatttgatttgagattgttgactggtattccaaattctcatttgaatgataaagatgttagagaagttgttgttaaatctttgactgctcaaatgaaaaaatatactccaagagctgttcattatttgtctgttggtttgcaagaagcattgggtgatttgaaagatccaagaactttggataatttgtttttgactgttcaaaatatggttgctaaagcatctgcttctatttttgttggtgaagaattgtgtaataatattgaattggttgatacttttaaacatgttacttctgatattggttctgaaattagattggataatacttggttggaattttttaaaactattaatcaaattagaatgtggtatgttggtaaacattctccaagagttactaaacatagaagacaattgattcaagctatggctccagaaattgatagaagattgcaaggtttggctggtaatgatccatcttggactagaccagaagatattttgcaagaaattatggaaaattatccatctccatctactgttccagcagatatttatacttattatgctaattggatgattgttttgatttttgcttctgttcatactactactgaacatgctactattgttttgtatagattgttgcaacaaccagaattgattgatgaattgttgcaagaacaaaaagaagcattgggtcaaggtactgtttttactggtgaagttattagaaaattggttaaattggattctgtttgtagagaatctttgagaattaaaaatgaatattttggtttgccacatagaaatatgtctcatgaaaatatttctttgtctaatggtgttgttattaaaccaggtgataatgttatgttgaatgtttggactaatcatcatgattctgatttgcaaaaagatgttcatggtaatcatgataaatttgaaccatttagatatgttaatgctgatagaccagctactaaaattggtgatgattatttgatttttggtgaaggtaaacatgcttgtccaggtagatggtttgctttgcaagaaattaaaactattgtttctgttttgattagagattatactttgactccatgtggtccaattgtttttccatctggtacttctactggtattccatctggtgaagttactattcaaagaaaatctgaagtttaa

SEQ ID NO: 7 - protein, 514 aa, Lichtheimia ramosa
MTEIKEHIYRYRHYIGVAAAVALVCQQVYYRIFRIPKNLRHIPAIPYGQQLKALRSDEALTSRTKRLVFPLLSKCNGVYLNRMPFKWTIYVADPEIARAILFKPEFGHKTRSVTDSLDKNSSLFEFVGDDNIAIVNGHEWKEQRKIMNPAFHRATPVGMFGSLMPKVFRLVEEQPTLPALDLMQKLTLDALGKSVFGFEFGALDDPDSVWVKTYRQVFDSFTDVFSLVFPRLDPIYRYFSAKHREQYNAVYKLIDLLDGMADKKRSMLQDASNSDIKDVPDHEKDLLQLMLEAELRGEGSWTKRELRHNMAIFFVAGQDTTSHALTFCLYLLAKNQDIQKKAREEILNVFGDEPKDVFPTLEDCKKLNYLDMVIKESMRIYPPANDVLARDVNEDLNVKGVFIPKGSMVSVDIHALHHRPDLWHEPDKFNPDRFLPGGEHDSHVGVTYAPFSSGSRQCIALKFATMQQRVVLSMLLRKYEWELPKDSKHKDSIQFQIPFNIAPKDLELTFHKRY

SEQ ID NO: 8 - DNA, 1545 bp, Lichtheimia ramosa
  source 1..1545: mol_type = other DNA
atgactgaaattaaagaacatatttatagatatagacattatattggtgttgctgctgctgttgctttggtttgtcaacaagtttattatagaatttttagaataccaaaaaatttgagacatattccagctattccatatggtcaacaattgaaagcattgagatctgatgaagcattgacttctagaactaaaagattggtttttccattgttgtctaaatgtaatggtgtttatttgaatagaatgccatttaaatggactatttatgttgctgatccagaaattgctagagctattttgtttaaaccagaatttggtcataaaactagatctgttactgattctttggataaaaattcttctttgtttgaatttgttggtgatgataatattgctattgttaatggtcatgaatggaaagaacaaagaaaaattatgaatccagcttttcatagagctactccagttggtatgtttggttctttgatgccaaaagtttttagattggttgaagaacaaccaactttgccagctttggatttgatgcaaaaattgactttggatgctttgggtaaatctgtttttggttttgaatttggtgctttggatgatccagattctgtttgggttaaaacttatagacaagtttttgattcttttactgatgttttttctttggtttttccaagattggacccaatttatagatatttttctgctaaacatagagaacaatataatgctgtttataaattgattgatttgttggatggtatggctgataaaaaaagatctatgttgcaagatgcttctaattctgatattaaagatgttccagatcatgaaaaagatttgttgcaattgatgttggaagctgaattgagaggtgaaggttcttggactaaaagagaattgagacataatatggctattttttttgttgctggtcaagatactacttctcatgctttgactttttgtttgtatttgttggctaaaaatcaagatattcaaaaaaaagctagagaagaaattttgaatgtttttggtgatgaaccaaaagatgtttttccaactttggaagattgtaaaaaattgaattatttggatatggttattaaagaatctatgagaatttatccaccagctaatgatgttttggctagagatgttaatgaagatttgaatgttaaaggtgtttttattccaaaaggttctatggtttctgttgatattcatgctttgcatcatagaccagatttgtggcatgaaccagataaatttaatccagatagatttttgccaggtggtgaacatgattctcatgttggtgttacttatgctccattttcttctggttctagacaatgtattgctttgaaatttgctactatgcaacaaagagttgttttgtctatgttgttgagaaaatatgaatgggaattgccaaaagattctaaacataaagattctattcaatttcaaattccatttaatattgctccaaaagatttggaattgacttttcataaaagatattaa

SEQ ID NO: 9 - protein, 701 aa, Thamnostylum piriforme
MAQSSPALLDSLDIVFLGTIGLGTIAWFARRQIAERIFGSKDDANKNVGNGNAPTAPKRERNFVKVMQEQGRKVIFFYGSQTGTAEDYASRLAKECSQKYGVSCMTADIELYDLTYLDTVPEDFLVFFIMATYGEGEPTDNAVDFWEQLTEEEPQFSEGDTLGNLRYVVFGLGNKTYEHYNEVARRMDKLLTKLGAKRIGERGEGDDDASLEEDFLAWQDSMWPAFCDALGVDESNGSSGPRQAMYAVEELEGQEAVYLGELGEKPKEGVKVVYDAKRPYNAPLVSQDLFKNTDRHCLHIDIDVSDSNLSYQTGDHIAIWPTNSDDEVARLASLLGLTDKLDTSVMVKAIDSTASKQYPFPVPATYRSIFRHYLDICAPASRQTLMSLVEYAPTEASKEALRLLSKDKDEYRLKVGEAVRNLGEVLELAAGADARPGLFSTVPFDLIVESVSRLQPRYYSISSSAKESPKVIAVTAVTLTYNPDPTPERTVYGVNTNYLWQIHAAKHNVNDGQRYPTYDLAGPRNALQGAKVPVHIRRSQFKLPRNPTVPVIMVGPGTGVAPFRGFVRERAAQKTDGKPVGPTLLFFGCRNSQQDFLYKDEWPELFATLGDESRIVTAFSRETPQKVYVQHRLQENGEELWNLLQKGAYIYVCGDAKNMARDVNQTFVNFAIEFGGQTEEKAHDYVKNLRNSGRYQEDVWS

SEQ ID NO: 10 - DNA, 2106 bp, Thamnostylum piriforme
  source 1..2106: mol_type = other DNA
atggcgcaatcgtcacctgctcttctcgactctttggacattgtgttcctcggaacgattggtcttggcaccattgcctggtttgcacgtcgacagattgcagaacgaatctttggatccaaggatgatgcaaacaaaaacgtcggcaatggcaacgctcctactgcgccgaagcgagagcgcaactttgtcaaagtgatgcaagaacagggtcgcaaagtcatcttcttttacggttcgcagaccggcacggctgaagactacgcttctcggctagccaaggaatgctcgcaaaagtacggtgtgagctgtatgacagccgatatcgagctgtacgatctgacctatctggacactgtgcctgaagacttcttggtgttcttcatcatggcgacgtacggcgagggtgagcccaccgacaacgccgtcgacttctgggagcaattgacggaggaggagccccagttctcggaaggcgacactttgggtaacttgcgctatgtggtgtttggcctgggcaacaagacgtacgagcattacaacgaggtggctcgtcggatggacaagctgctgacgaagctgggtgccaagcgcattggcgagcggggcgaaggcgacgacgatgcttcgttggaagaggactttttggcgtggcaagatagcatgtggcccgcgttctgcgacgctctgggcgtggacgagagcaacggatccagcgggccccgccaggccatgtacgcggtggaggagcttgagggccaagaggccgtgtatctcggcgagctcggagagaagcccaaggagggcgttaaggttgtgtacgacgccaagcgcccctacaacgcgccgctcgtctcgcaggacctcttcaaaaacacagaccgccattgcctgcacatcgacatcgacgtctcggactccaacctgtcgtaccagaccggcgaccacatcgcgatctggcccaccaacagcgatgacgaagtggcgcgtctcgcttccctgctcggcctgaccgacaagctcgacaccagcgtcatggtcaaggccatcgactccaccgcctccaagcagtacccattccccgtgcccgccacctaccggtccattttccgccactacctcgacatctgcgcgcccgcgtcgcgccagacgctcatgtcgctggtcgagtacgcgcccacggaggcgtccaaagaggccttgcgtctgctgtccaaggacaaggacgagtaccgcctcaaggtcggcgaggctgtccgcaacctgggcgaggtgcttgagctggctgccggagcggatgcccgaccgggcctgttctccaccgtgccctttgacctgatcgtcgagagcgtgtcgcgcctgcagccccgctactactccatctcctcatcggccaaggagtcgcccaaggtcattgccgtcaccgccgtcaccttgacatacaacccagaccccacgcccgagcgcaccgtgtatggcgtcaacaccaactatttgtggcaaatccacgccgccaagcacaacgtcaacgacggccagcgctaccccacctacgacctggctggcccgcgcaacgccctgcaaggcgccaaggtccccgtccacatccgccgctcgcagttcaagctgccgcgcaaccccaccgtccctgtcatcatggtcggcccaggcaccggtgtcgcgcccttccgtggctttgttcgcgagcgtgccgcccaaaagaccgacggcaagcccgtcggccccaccctgctcttcttcggctgccgcaactcgcaacaggacttcttgtacaaggacgagtggcccgagctgttcgccacccttggcgacgagtcgcgtatcgtgactgcattctcgcgcgagactccccagaaggtctacgtccagcaccggctccaggagaacggcgaggagctgtggaatctgctgcaaaagggagcctacatttacgtgtgcggtgacgcaaagaacatggcacgcgatgtcaaccagaccttcgtcaactttgcgatcgagtttggcggccagaccgaagagaaggcgcatgactacgtcaagaacctcagaaacagcggtcgataccaggaggatgtgtggagctaa

SEQ ID NO: 11 - DNA, 2106 bp, Thamnostylum piriforme
  source 1..2106: mol_type = other DNA
atggctcaatcttctccagctttgttggattctttggatattgtttttttgggtactattggtttgggtactattgcttggtttgctagaagacaaattgctgaaagaatttttggttctaaagatgatgctaataaaaatgttggtaatggtaatgctccaactgctccaaaaagagaaagaaattttgttaaagttatgcaagaacaaggtagaaaagttatttttttttatggttctcaaactggtactgctgaagattatgcttctagattggctaaagaatgttctcaaaaatatggtgtttcttgtatgactgctgatattgaattgtatgatttgacttatttggatactgttccagaagattttttggttttttttattatggctacttatggtgaaggtgaaccaactgataatgctgttgatttttgggaacaattgactgaagaagaaccacaattttctgaaggtgatactttgggtaatttgagatatgttgtttttggtttgggtaataaaacttatgaacattataatgaagttgctagaagaatggataaattgttgactaaattgggtgctaaaagaattggtgaaagaggtgaaggtgatgatgatgcttctttggaagaagattttttggcttggcaagattctatgtggccagctttttgtgatgctttgggtgttgatgaatctaatggttcttctggtccaagacaagctatgtatgctgttgaagaattggaaggtcaagaagctgtttatttgggtgaattgggtgaaaaaccaaaagaaggtgttaaagttgtttatgatgctaaaagaccatataatgctccattggtttctcaagatttgtttaaaaatactgatagacattgtttgcatattgatattgatgtttctgattctaatttgtcttatcaaactggtgatcatattgctatttggccaactaattctgatgatgaagttgctagattggcttctttgttgggtttgactgataaattggatacttctgttatggttaaagctattgattctactgcttctaaacaatatccatttccagttccagctacttatagatctatttttagacattatttggatatttgtgctccagcttctagacaaactttgatgtctttggttgaatatgctccaactgaagcatctaaagaagcattgagattgttgtctaaagataaagatgaatatagattgaaagttggtgaagctgttagaaatttgggtgaagttttggaattggctgctggtgctgatgctagaccaggtttgttttctactgttccatttgatttgattgttgaatctgtttctagattgcaaccaagatattattctatttcttcttctgctaaagaatctccaaaagttattgctgttactgctgttactttgacttataatccagatccaactccagaaagaactgtttatggtgttaatactaattatttgtggcaaattcatgctgctaaacataatgttaatgatggtcaaagatacccaacttatgatttggctggtccaagaaatgctttgcaaggtgctaaagttccagttcatattagaagatctcaatttaaattgccaagaaatccaactgttccagttattatggttggtccaggtactggtgttgctccatttagaggttttgttagagaaagagctgctcaaaaaactgatggtaaaccagttggtccaactttgttgttttttggttgtagaaattctcaacaagattttttgtataaagatgaatggccagaattgtttgctactttgggtgatgaatctagaattgttactgctttttctagagaaactccacaaaaagtttatgttcaacatagattgcaagaaaatggtgaagaattgtggaatttgttgcaaaaaggtgcttatatttatgtttgtggtgatgctaaaaatatggctagagatgttaatcaaacttttgttaattttgctattgaatttggtggtcaaactgaagaaaaagctcatgattatgttaaaaatttgagaaattctggtagataccaagaagatgtttggtcttag

SEQ ID NO: 12 - protein, 707 aa, Thamnostylum piriforme
MSKKPTTFAISPLDLLLLGTLGIGALLYITRKLRAAPPPAAGDAARPSDKAPSNKPERNFVKLMEQQKRRVIFFYGSQTGTAEDYASRLAKESSQKYGVSSMAADIELYDLSYLDTVPEDKLVVFVMATYGEGEPTDNAVDFWETLTDEAPMFSLGEELAPLRNLNYIVFGLGNKTYEHYNEVARVLDKRLAALGATRHGVRGEGDDDASLEEDFLAWQEDMWPAFCAALNVDESNAQAGPRQAMYGVKELEGHDENGLFLGELGEWLQPEQLQKQTAVHDAKHPYLAPIAASRDLFSHAADRHCLHLEVDIASSNLTYQTGDHIAIWPTNNDVEVLRLATVLGLADKLDTAIMVKALDSAASKKYPFPVPTTYRAAFKHYLDICSPASRQTLISLVDYAPTASSKDALHKLATDKDAYKTQVSEAARNLAEVLELCQDDEGTTAAPGFFSTVPFDLIVESVSRLQPRYYSISSSSLTSPKSVAVTAVTLSYQPSTTKSRTVYGVNTNYLWRIHAPHDASVPAYDLDGPRNTLQGKKVPVHIRRSTFKLPRNTKLPVIMVGPGTGVAPFRGFIHDRVHQKQSGKEVGPTVLFYGCRHSKQDFLYAEEWPALFEALGEGSQLITAFSRESAQKVYVQHRLKEHGEQMWKYIEQGAYIYVCGDAKNMARDVQQTFIEFAQALGNKTESQAQDYVKNLRNTGRYQEDVWS

SEQ ID NO: 13 - DNA, 2124 bp, Thamnostylum piriforme
  source 1..2124: mol_type = other DNA
atgtcaaaaaagcctactacgtttgccatcagtccgcttgacctgctgctgctgggcacgctggggattggcgccctcctctacataacgagaaagctcagggcagcgcctccacccgcggctggcgacgcagcacgcccatccgataaagcgccgagcaacaagcctgaacggaacttcgtcaagctgatggagcagcagaaacggcgcgtcattttcttttacggatctcagacaggcactgcagaggattacgcctcacggctggccaaggaaagctcccagaaatatggcgtgagcagcatggcggccgacattgagctgtacgacctcagctacctggacaccgtacccgaggacaagctggtggtgtttgtcatggccacctacggcgagggcgagccgaccgacaacgccgtcgacttctgggaaacgctgaccgacgaagcgcccatgttctcgctgggagaagagttagcgccgctgcgaaacctgaactacattgtgtttggactgggcaacaagacctacgagcattacaacgaagtggcccgtgtccttgacaagcgccttgcggctctgggcgccacgcggcacggcgtgcggggcgaaggcgacgacgacgcttctctggaagaggactttctggcatggcaggaggatatgtggcctgcgttctgcgctgcgttgaatgtcgacgagagcaatgcgcaggcgggtccgcggcaggcgatgtacggcgtgaaggagctcgagggccacgacgaaaacggtctgtttttgggagagctgggcgagtggctgcagcctgaacagctgcagaagcagacagccgtgcatgacgccaaacatccgtacctggcccccatcgctgcctcgcgtgatctgttcagccacgccgccgaccgccactgcctgcacctggaagtcgacatcgccagcagcaacctcacctaccagaccggcgaccacatcgccatctggcccaccaacaacgacgtcgaggtgctgcgcctggccaccgtgctgggtctggcggacaagctggacaccgccatcatggtcaaggctttggacagcgccgcctccaaaaagtaccccttccctgtccccaccacgtaccgcgccgcattcaaacactacctcgatatctgctctcctgcatctcgccagacgctgatctcgttggtcgactacgcgcccaccgcgtcgtccaaggacgcgctgcacaaactggcaaccgacaaggacgcctataaaacgcaggtcagcgaggccgcgcgcaacctggccgaggtgctggagctgtgccaggacgacgagggcacgacagccgcgcctggcttcttttccaccgtgcccttcgacctgattgtcgaaagcgtgtcgcgtctccagccacggtactactccatctcgtcatcctcgctcacctctccgaaatccgtcgctgtcaccgccgtcacgctgtcctaccagccatccaccaccaaaagccgcaccgtctacggcgtcaacaccaactacctgtggcgcatccatgcaccgcacgacgcgtccgtcccagcctacgatctcgacggtccgcgcaacactctccaaggcaaaaaggtgccggtgcatatccgccgatcgaccttcaaactgccgcgcaacaccaagcttcccgtcatcatggtggggcctggcacgggcgtcgcgccgttccgcgggttcatccatgatcgcgtgcatcagaagcaaagtggcaaggaggtggggcccacggtgctgttctatggatgccgccattccaaacaggactttttgtatgcggaagaatggccggcgctctttgaggcgttgggcgaaggatcgcagttgatcaccgcgttttcgcgagaatcagcgcaaaaggtctatgttcagcaccggttaaaggagcacggcgagcagatgtggaagtatatcgagcagggagcgtatatctacgtttgcggtgacgccaaaaacatggccagggacgtgcagcagacgttcattgagtttgcacaggctctgggaaacaaaacggaaagccaggcacaggactacgtcaagaatctgcgaaacaccggacgctaccaggaggacgtgtggtcgtag

SEQ ID NO: 14 - DNA, 2124 bp, Thamnostylum piriforme
  source 1..2124: mol_type = other DNA
atgtctaaaaaaccaactacttttgctatttctccattggatttgttgttgttgggtactttgggtattggtgctttgttgtatattactagaaaattgagagctgctccaccaccagctgctggtgatgctgctagaccatctgataaagctccatctaataaaccagaaagaaattttgttaaattgatggaacaacaaaaaagaagagttatttttttttatggttctcaaactggtactgctgaagattatgcttctagattggctaaagaatcttctcaaaaatatggtgtttcttctatggctgctgatattgaattgtatgatttgtcttatttggatactgttccagaagataaattggttgtttttgttatggctacttatggtgaaggtgaaccaactgataatgctgttgatttttgggaaactttgactgatgaagctccaatgttttctttgggtgaagaattggctccattgagaaatttgaattatattgtttttggtttgggtaataaaacttatgaacattataatgaagttgctagagttttggataaaagattggctgctttgggtgctactagacatggtgttagaggtgaaggtgatgatgatgcttctttggaagaagattttttggcttggcaagaagatatgtggccagctttttgtgctgctttgaatgttgatgaatctaatgctcaagctggtccaagacaagctatgtatggtgttaaagaattggaaggtcatgatgaaaatggtttgtttttgggtgaattgggtgaatggttgcaaccagaacaattgcaaaaacaaactgctgttcatgatgctaaacatccatatttggctccaattgctgcttctagagatttgttttctcatgctgctgatagacattgtttgcatttggaagttgatattgcttcttctaatttgacttatcaaactggtgatcatattgctatttggccaactaataatgatgttgaagttttgagattggctactgttttgggtttggctgataaattggatactgctattatggttaaagcattggattctgctgcttctaaaaaatatccatttccagttccaactacttatagagctgcttttaaacattatttggatatttgttctccagcttctagacaaactttgatttctttggttgattatgctccaactgcttcttctaaagatgctttgcataaattggctactgataaagatgcttataaaactcaagtttctgaagctgctagaaatttggctgaagttttggaattgtgtcaagatgatgaaggtactactgctgctccaggttttttttctactgttccatttgatttgattgttgaatctgtttctagattgcaaccaagatattattctatttcttcttcttctttgacttctccaaaatctgttgctgttactgctgttactttgtcttatcaaccatctactactaaatctagaactgtttatggtgttaatactaattatttgtggagaatccatgctccacatgatgcttctgttccagcttatgatttggatggtccaagaaatactttgcaaggtaaaaaagttccagttcatattagaagatctacttttaaattgccaagaaatactaaattgccagttattatggttggtccaggtactggtgttgctccatttagaggttttattcatgatagagttcatcaaaaacaatctggtaaagaagttggtccaactgttttgttttatggttgtagacattctaaacaagattttttgtatgctgaagaatggccagctttgtttgaagcattgggtgaaggttctcaattgattactgctttttctagagaatctgctcaaaaagtttatgttcaacatagattgaaagaacatggtgaacaaatgtggaaatatattgaacaaggtgcttatatttatgtttgtggtgatgctaaaaatatggctagagatgttcaacaaacttttattgaatttgctcaagcattgggtaataaaactgaatctcaagctcaagattatgttaaaaatttgagaaatactggtagataccaagaagatgtttggtcttag

SEQ ID NO: 15 - DNA, 2085 bp, Thamnostylum piriforme
  source 1..2085: mol_type = other DNA
atggcaaaaaatactacattaaacaagcaagtggtgcttctcatcagtgccctgggcctgagcgctgttgcctacatggccagccgatacttttttggaaacagcgcaagcacccctgaacaatcgaaaaagcccattcaaccagaagaaaaagaagacgaatcaaactttgtgcaaaccatgaagaagcagaatcgtaaagtagtgatcttttatggttcgcaaacaggcacggctgaagattgtgcgcagcgacttggcaagcagtgcaaaaagcaattcggcgttcctgctttggttcttgatatcgaacaatgcaacatgtcgtacttggatcagattcccgaggactgtgttgcgatatttgtcatggcaacttatggcgaaggtgaacccactgacaacgccactgacttttgggaaatgcttgaagcccatcctgaattttcccaaagcgacaacctcaagaatctccgatacttcatcttcggacttggcaacagctcctatacctattacaactgggtctccaaaactgtcgacaagaaactcactgaattaggcgcgacacgacttggcaaacttggcttgggcgacgacgaaaagtcgctcgaagacgactttgaagcatggcaggaaggcatgtggcctatttttggagaagctgtggaaacggacgccgccagcaatatcagcggcggacacgagcccacataccatgtggtggagctaaccgaacatgacggccacgtgtattctggtgaattgggagacaaatcgcaagctctctatagcggcaagaagccttacccagcacctattaccacgcgcgatttgttgaacggatcggatcgccactgtctacacctggaagtcgatatcagtggatcaggcatgagctataccactggcgatcatattgccatctggcccacaaacagcgaagacgaggtcttgagcttggcacgcgccattggtctcgaaaacaagctggataccgtcatttgcgttactgctgcggatgagacgtccgccaagcaatcaccgttccctcagccaacaacataccgtgccatgctgcgacactacttggatatttgccaaatgccatcacggcagacactggagatgctcgtaccttttgcaccgtcgcccgaagctgcagagactctagaaaagctggcaaaggacaaggatgagcatcgtcgagtggttctgggaccagttcgtaacttggctgctgtgctgagttatgcgtcgtcgggcgctgcatacaagatcccatccgatgtgctgcttgagtgccttggtcgactacagccccgatattactcgatttcctcctctgccttggagagcccaaacagtgtcagtgtcactgccgttacactcaaattcaaccccgaaccaacgcctgagcgcactgtctttggcgtcaataccaactacctgtgggccgtccataccgcgctcaacgatgacgacgaggcaatccaagttgaaaccgagtattccatcggtggacctaacggtcaatactttgacgagaagctcaaaactgcaagattgcctgttcacatccgccaatcaaacttcaagctgccagcagacacaactaagccagtgatcatgattggccccggcaccggtgtcgctcctttccgagccttcgttcgcgagcgtgcctatcaaaagaagcaccttggcaaatccatcggtccaactattctgtactttggctgccgacgttccaacgaggactttttgtatcaggaagagtggccagagctctttaacgaattgggcggctcctctcgattgatcactgcattctcccgggaaacggatcgaaaagtatacgtccaagacaagctcaacgagaacggcccagagacttgggatttgattaataacaagggcgcctatgtctatgtttgtggcgacgctaagcgtatggccaaagacgtccagtccgcacttgttagttttgcaaagcagtatggcagctatgacgacgctggtgcagctgactacatcagcaagttaagagatgccggacgatatcaagaagacgtctgggcataa

SEQ ID NO: 16 - protein, 710 aa, Cunninghamella elegans
MAQQTPAVIDTLDLILLGSIGLGTIAWFTRRQLSERLFGTGQSNATPKPTTPQAPKRERNFVKVMEQQGRKVIFFYGSQTGTAEDFASRLAKQCSQKYGVSCMTADIEMYDLSYLDTLSEDSLVCFVMATYGEGEPTDNAVDFWEQFITDESPVFSEGGETLENLRYLMFGLGNKTYEHYNAVARILDKKLTGLGAKRIGERGEGDDDGSLEEDFLAWQESMWPTFCDALGVDENNAQQGPRQASYSVDELKEEYKNDDVYFGELGTKSKDSSRVVYDAKRPYNAPITTRELFNSSERHCLHVDIDISGTNLSYQTGDHVAMWPTNNEDEVLRLANILGLQDKLDNVISVKAIDPAAPKQHPFPVPTTYRAIFRHYIDICAPASRQSLMSFVEFAPTDSAKDLLKLLATDKDEYRLKVGEAVRNLGEVLELVSGNDKDTQPGSFTSVPFDLIVETIPRLQPRYYSISSSSKENSSIISATCVTLAYQPDPTPDRTVYGVNTNFLYRIHTQSSDNDIQGLPKYDLAGPRKAFLNEQGQSHKLPIHIRRSQFKLPRNTSCPVIMIGPGTGVAPFRGFVRERALQKKEGKSVGPTVLFFGNRHSEHDFLYSDEWPELFNTLGDDSKLITAFSRETEHKVYVQHRLEENGKDIWQLLEKGAYIYVCGDARNMARDVNQTFVNLAMEYGEKTEQKALDYVKSLRNTGRYQEDVWS

SEQ ID NO: 17 - DNA, 2133 bp, Cunninghamella elegans
  source 1..2133: mol_type = other DNA
atggctcaacaaactccagctgttattgatactttggatttgattttgttgggttctattggtttgggtactattgcttggtttactagaagacaattgtctgaaagattgtttggtactggtcaatctaatgctactccaaaaccaactactccacaagctccaaaaagagaaagaaattttgttaaagttatggaacaacaaggtagaaaagttatttttttttatggttctcaaactggtactgctgaagattttgcttctagattggctaaacaatgttctcaaaaatatggtgtttcttgtatgactgctgatattgaaatgtatgatttgtcttatttggatactttgtctgaagattctttggtttgttttgttatggctacttatggtgaaggtgaaccaactgataatgctgttgatttttgggaacaatttattactgatgaatctccagttttttctgaaggtggtgaaactttggaaaatttgagatatttgatgtttggtttgggtaataaaacttatgaacattataatgctgttgctagaattttggataaaaaattgactggtttgggtgctaaaagaattggtgaaagaggtgaaggtgatgatgatggttctttggaagaagattttttggcttggcaagaatctatgtggccaactttttgtgatgctttgggtgttgatgaaaataatgctcaacaaggtccaagacaagcatcttattctgttgatgaattgaaagaagaatataaaaatgatgatgtttattttggtgaattgggtactaaatctaaagattcttctagagttgtttatgatgctaaaagaccatataatgctccaattactactagagaattgtttaattcttctgaaagacattgtttgcatgttgatattgatatttctggtactaatttgtcttatcaaactggtgatcatgttgctatgtggccaactaataatgaagatgaagttttgagattggctaatattttgggtttgcaagataaattggataatgttatttctgttaaagctattgatccagctgctccaaaacaacatccatttccagttccaactacttatagagctatttttagacattatattgatatttgtgctccagcttctagacaatctttgatgtcttttgttgaatttgctccaactgattctgctaaagatttgttgaaattgttggctactgataaagatgaatatagattgaaagttggtgaagctgttagaaatttgggtgaagttttggaattggtttctggtaatgataaagatactcaaccaggttcttttacttctgttccatttgatttgattgttgaaactattccaagattgcaaccaagatattattctatttcttcttcttctaaagaaaattcttctattatttctgctacttgtgttactttggcttatcaaccagatccaactccagatagaactgtttatggtgttaatactaattttttgtatagaatccatactcaatcttctgataatgatattcaaggtttgccaaaatatgatttggctggtccaagaaaagcatttttgaatgaacaaggtcaatctcataaattgccaattcatattagaagatctcaatttaaattgccaagaaatacttcttgtccagttattatgattggtccaggtactggtgttgctccatttagaggttttgttagagaaagagctttgcaaaaaaaagaaggtaaatctgttggtccaactgttttgttttttggtaatagacattctgaacatgattttttgtattctgatgaatggccagaattgtttaatactttgggtgatgattctaaattgattactgctttttctagagaaactgaacataaagtttatgttcaacatagattggaagaaaatggtaaagatatttggcaattgttggaaaaaggtgcttatatttatgtttgtggtgatgctagaaatatggctagagatgttaatcaaacttttgttaatttggctatggaatatggtgaaaaaactgaacaaaaagcattggattatgttaaatctttgagaaatactggtagataccaagaagatgtttggtcttag

SEQ ID NO: 18 - protein, 713 aa, Gibberella fujikuroi
MAELDTLDIVVLGVIFLGTVAYFTKGKLWGVTKDPYANGFAAGGASKPGRTRNIVEAMEESGKNCVVFYGSQTGTAEDYASRLAKEGKSRFGLNTMIADLEDYDFDNLDTVPSDNIVMFVLATYGEGEPTDNAVDFYEFITGEDASFNEGNDPPLGNLNYVAFGLGNNTYEHYNSMVRNVNKALEKLGAHRIGEAGEGDDGAGTMEEDFLAWKDPMWEALAKKMGLEEREAVYEPIFAINERDDLTPEANEVYLGEPNKLHLEGTAKGPFNSHNPYIAPIAESYELFSAKDRNCLHMEIDISGSNLKYETGDHIAIWPTNPGEEVNKFLDILDLSGKQHSVVTVKALEPTAKVPFPNPTTYDAILRYHLEICAPVSRQFVSTLAAFAPNDDIKAEMNRLGSDKDYFHEKTGPHYYNIARFLASVSKGEKWTKIPFSAFIEGLTKLQPRYYSISSSSLVQPKKISITAVVESQQIPGRDDPFRGVATNYLFALKQKQNGDPNPAPFGQSYELTGPRNKYDGIHVPVHVRHSNFKLPSDPGKPIIMIGPGTGVAPFRGFVQERAKQARDGVEVGKTLLFFGCRKSTEDFMYQKEWQEYKEALGDKFEMITAFSREGSKKVYVQHRLKERSKEVSDLLSQKAYFYVCGDAAHMAREVNTVLAQIIAEGRGVSEAKGEEIVKNMRSANQYQVCSDFVTLHCKETTYANSELQEDVWS

SEQ ID NO: 19 - DNA, 2142 bp, Gibberella fujikuroi
  source 1..2142: mol_type = other DNA
atggcagaattagatacacttgatatagtagtattaggtgttatctttttgggtactgtggcatactttactaagggtaaattgtggggtgttaccaaggatccatacgctaacggattcgctgcaggtggtgcttccaagcctggcagaactagaaacatcgtcgaagctatggaggaatcaggtaaaaactgtgttgttttctacggcagtcaaacaggtacagcggaggattacgcatcaagacttgcaaaggaaggaaagtccagattcggtttgaacactatgatcgccgatctagaagattatgacttcgataacttagacactgttccatctgataacatcgttatgtttgtattggctacttacggtgaaggcgaaccaacagataacgccgtggatttctatgagttcattactggcgaagatgcctctttcaatgagggcaacgatcctccactaggtaacttgaattacgttgcgttcggtctgggcaacaatacctacgaacactacaactcaatggtcaggaacgttaacaaggctctagaaaagttaggagctcatagaattggagaagcaggtgagggtgacgacggagctggaactatggaagaggactttttagcttggaaagatccaatgtgggaagccttggctaaaaagatgggcttggaggaaagagaagctgtatatgaacctattttcgctatcaatgagagagatgatttgacccctgaagcgaatgaggtatacttgggagaacctaataagctacacttggaaggtacagcgaaaggtccattcaactcccacaacccatatatcgcaccaattgcagaatcatacgaacttttctcagctaaggatagaaattgtctgcatatggaaattgatatttctggtagtaatctaaagtatgaaacaggcgaccatatcgcgatctggcctaccaacccaggtgaagaggtcaacaaatttcttgacattctagatctgtctggtaagcaacattccgtcgtaacagtgaaagccttagaacctacagccaaagttccttttccaaatccaactacctacgatgctatattgagataccatctggaaatatgcgctccagtttctagacagtttgtctcaactttagcagcattcgcccctaatgatgatatcaaagctgagatgaaccgtttgggatcagacaaagattacttccacgaaaagacaggaccacattactacaatatcgctagatttttggcctcagtctctaaaggtgaaaaatggacaaagataccattttctgctttcatagaaggccttacaaaactacaaccaagatactattctatctcttcctctagtttagttcagcctaaaaagattagtattactgctgttgtcgaatctcagcaaattccaggtagagatgacccattcagaggtgtagcgactaactacttgttcgctttgaagcagaaacaaaacggtgatccaaatccagctccttttggccaatcatacgagttgacaggaccaaggaataagtatgatggtatacatgttccagtccatgtaagacattctaactttaagctaccatctgatccaggcaaacctattatcatgatcggtccaggtaccggtgttgccccttttagaggcttcgtccaagagagggcaaaacaagccagagatggtgtagaagttggtaaaacactgctgttctttggatgtagaaagagtacagaagatttcatgtatcaaaaagagtggcaagagtacaaggaagctcttggcgacaaattcgaaatgattacagctttttcaagagaaggatctaaaaaggtttatgttcaacacagactgaaggaaagatcaaaggaagtttctgatcttctatcccaaaaagcatacttctacgtttgcggagacgccgcacatatggcacgtgaagtgaacactgtgttagcacagatcatagcagaaggccgtggtgtatcagaagccaagggtgaggaaattgtcaaaaacatgagatcagcaaatcaataccaagtgtgttctgatttcgtaactttacactgtaaagagacaacatacgcgaattcagaattgcaagaggatgtctggagttaa

SEQ ID NO: 20 - protein, 691 aa, Saccharomyces cerevisiae
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 21 - DNA, 2076 bp, Saccharomyces cerevisiae
  source 1..2076: mol_type = other DNA
atgccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgctgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcatcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaccgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccagttgttggtgttacgactaacttgttaagaaacattcaattggctcaaaacaatgttaacattgccgaaactaacctacctgttcactacgatttaaatggcccacgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggtag

SEQ ID NO: 22 - protein, 503 aa, Homo sapiens
MALIPDLAMETWLLLAVSLVLLYLYGTHSHGLFKKLGIPGPTPLPFLGNILSYHKGFCMFDMECHKKYGKVWGFYDGQQPVLAITDPDMIKTVLVKECYSVFTNRRPFGPVGFMKSAISIAEDEEWKRLRSLLSPTFTSGKLKEMVPIIAQYGDVLVRNLRREAETGKPVTLKDVFGAYSMDVITSTSFGVNIDSLNNPQDPFVENTKKLLRFDFLDPFFLSITVFPFLIPILEVLNICVFPREVTNFLRKSVKRMKESRLEDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGMFIPKGVVVMIPSYALHRDPKYWTEPEKFLPERFSKKNKDNIDPYIYTPFGSGPRNCIGMRFALMNMKLALIRVLQNFSFKPCKETQIPLKLSLGGLLQPEKPVVLKVESRDGTVSGA

SEQ ID NO: 23 - DNA, 1533 bp, Homo sapiens
  source 1..1533: mol_type = other DNA
aagcttaaaatggcactgattcccgatttagcaatggagacttggttgttgcttgccgtaagtctcgtcttactgtatttgtacggaacgcactcgcacggattatttaagaaattgggcattcccgggcccactccactaccattcctaggtaatatacttagttatcacaagggtttctgcatgttcgacatggaatgtcacaagaagtatggtaaggtatggggattctacgacggccaacaacctgtgttggcgatcaccgacccagacatgatcaaaactgtcctcgtaaaagaatgttactctgttttcactaatagaagaccatttggacctgtaggattcatgaagagtgctatatccattgcagaggacgaagaatggaagaggcttagaagccttctgtcacctacattcacaagcggcaagttgaaggagatggtccctatcatcgcccagtatggcgatgtcttagtgagaaatttacgtagggaagctgaaactggtaagcctgtgacgctgaaggatgtctttggagcttatagcatggacgtgatcacttcgacttcctttggcgtaaatatcgattcgcttaacaacccacaggatccctttgttgagaacacgaagaaactgttgagatttgactttctcgatccgttcttcctttccattaccgtcttccctttcctcatacccattctggaagtcctgaatatatgcgtattcccaagagaggtgacgaacttcttaaggaaatcggtaaagaggatgaaggaatcccgtcttgaggacacccagaagcacagggtcgactttctacagttaatgatagattcacagaattcgaaagagacggagagccataaggctttaagtgatcttgagcttgtagcacagagtatcatcttcattttcgccggctacgagacgacgagcagtgtgttgtcgttcatcatgtacgagttggcgacacatcccgacgtgcagcagaagttacaggaagaaatcgatgctgttttaccaaataaagccccacccacttatgatacagttttgcagatggagtacttagacatggtggttaacgaaacactaagactgtttccgattgctatgcgattggaacgagtgtgtaagaaggacgtagaaattaacggcatgtttataccaaagggcgtagttgtcatgataccttcttatgctttgcatcgagaccctaagtactggaccgaacctgaaaagtttctcccagagcgctttagcaagaagaataaggataacatagacccctacatttatacgccattcggatccggtccacgtaactgtataggcatgcgtttcgctctgatgaacatgaagctggcgttaatcagggtattacagaacttttcattcaaaccatgcaaagagacacagataccgttgaagctctctttaggtggacttctgcagcccgagaagccagtggttctcaaagttgagagcagggacggtacagtttcaggcgcttagaagactccgcgg

SEQ ID NO: 24 - protein, 503 aa, Pongo abelii
MDLIPDLAMETWILLAVSLVLLYLYGTHSHGLFKKLGIPGPTPLPLLGNVFSYRKGFWRFDMECHKKYGKVWGFYDGRQPVLAITDPDMIKTVLVKECYSVFTNRRPFGPVGFMKSAISIAEDEEWKRIRSLLSPTFTSGKLKEMVPIIARYGDVLVRNLRREAETGKPVTLKDVFGAYSMDVITSTSFGVNIDSLNNPQDPFVENTKKLLRFDFLDPFFLSIIVFPFLTPILEILNISVFPREVTSFLRKSVKRMKESRLKDTQKHRVDFLQLMIDSQNSKETESHKALSDLELVAQSIIFIFAGYETTSSVLSFIMYELATHPDVQRKLQEEIDAVLPNKAPPTYDTVLQMEYLDMVVNETLRLFPIAMRLERVCKKDVEINGMFIPKGVVVMIPSYALHHDPKYWTEPGKFLPERFSKKNKDNIEPYVYTPFGTGPRNCIGMRFALMNMKLAVIRVLQNFSFKPCKETQIPLKLRLGGLLQTEKPIVLKVESRDGTLSGA

SEQ ID NO: 25 - DNA, 1533 bp, Pongo abelii
  source 1..1533: mol_type = other DNA
aagcttaaaatggaccttatccctgacctcgctatggagacttggatcttacttgcggtttctctagtgctgttatacttgtatggaacacactcccatggattgtttaagaaattaggtatcccaggaccaacaccgttgcctttgctaggaaacgtcttcagttatcgaaagggcttctggagatttgatatggaatgtcacaagaagtacggcaaggtgtggggcttctacgatggcaggcagcctgtcctcgcaataacagatccggatatgataaagacggttttagtaaaagaatgctactctgtatttactaacaggcgccctttcgggccagtggggttcatgaagagcgccatctctatcgccgaagatgaggagtggaagcgtatccgatccttattatctcctacgtttacaagcgggaagttaaaagagatggtgccaataatagccaggtatggagacgttctggttaggaatttgagaagagaggctgagacggggaagcctgttaccttaaaggacgtcttcggcgcctacagcatggacgtaataaccagtacttcatttggcgtcaacatcgacagtctaaacaaccctcaggatccatttgtcgagaataccaagaaactcttaaggttcgactttctagacccattcttcctgagcattattgtgttccctttccttacccctattctcgagatactaaatatctctgttttcccaagagaggtaacgtcgttcttacgcaaatctgtaaagagaatgaaagagagtagattgaaggatacacaaaagcacagagtagacttccttcagctaatgattgattctcagaattcaaaggagaccgagagccacaaggccctttctgacctggaattagtagcacagtcaattatcttcattttcgccggatacgaaacaactagttctgtgttatcatttattatgtacgagttagcaactcacccggacgtacagaggaaattacaggaagagatagatgcagttcttccaaacaaggccccaccaacttatgacaccgtcctacaaatggagtatttggatatggttgtgaacgagacactgagattgtttcctatagcaatgagacttgagcgtgtgtgtaagaaagatgtcgagataaatggaatgttcatccctaagggcgtagtcgtgatgattccgtcctacgcactccaccacgaccctaagtattggacggagccgggtaagttcttacctgagaggttcagtaagaagaacaaagacaacatagaaccttatgtatacacgccctttggaaccgggccacgtaactgcattgggatgcgtttcgcactcatgaacatgaaacttgcggtcatacgagtgcttcaaaacttttcctttaagccatgcaaggagacacaaataccgctaaagttgagattgggtggattgttgcagacagagaagccgattgtccttaaggtcgaatcgcgcgacggcacattatctggcgcctagaagactccgcgg

SEQ ID NO: 26 - protein, 503 aa, Papio anubis
MDLIPDLAVETWLLLAVTLVLLYLYGTHSHGLFKKLGIPGPTPLPLLGNVLSYRKGFWTFDMECYKKYGKVWGFYDGRQPVLAITDPNMIKTVLVKECYSVFTNRRPFGPVGFMKSAISIAEDEEWKRIRSLLSPTFTSGKLKEMVPIIAKYGDVLVRNLRRETETGKPVTLKDVFGAYSMDVITSTSFGVNIDSLNNPQDPFVENTKKLLRFDFLDPFFLSITIFPFLIPILEVLNISIFPREVTSFLRKSVKRIKESRLKDTQKHRVDFLQLMIDSQNSKETESHKALSDLELVAQSIIFIFAGYETTSSVLSFIMYELATHPDVQQKLQEEIDAVLPNKAPPTYDTVLQMEYLDMVVNETLRLFPIAMRLERVCKKDVEINGIFIPKGVVVMIPTYALHHDSKYWTEPEKFLPERFSKKNKDNIDPYIYTPFGSGPRNCIGMRFALMNMKLALIRVLQNFSFKPCKETQIPLKLRLGGLLQTEKPIVLKIESRDGTVSGA

SEQ ID NO: 27 - DNA, 1533 bp, Papio anubis
  source 1..1533: mol_type = other DNA
aagcttaaaatggatcttattcctgacctcgctgttgagacatggctgcttttggcagttacactagtactactttacctgtacggaacccactcccacggcctatttaagaagttgggaatcccaggacctactccgctacctctactagggaatgtattatcataccgtaaaggtttctggactttcgacatggagtgttataagaagtacggaaaggtctggggcttttatgatggacgccaaccggtcctagccattacagatccaaacatgataaagaccgttttggtaaaagagtgctactctgttttcacgaatagaagaccctttggaccagttggcttcatgaagagtgcgatctctatcgccgaggatgaggagtggaagagaatcagatccctgttatcgccaaccttcacttcaggcaagctcaaagagatggtgccaataattgcgaaatacggtgacgtacttgtccgtaatctgcgtagagagacagagaccggaaagccagttacattaaaggacgtctttggtgcatactctatggacgtgatcacaagtacttctttcggtgtgaatattgattctttgaataacccacaggatccttttgtggagaacactaagaaattgctgaggtttgacttcttggatcccttcttcctgagcataaccatctttccttttctaattccaatcttggaagttttaaatatcagtatctttcccagggaagtcacttctttcctacgaaagtcggtgaagcgtataaaagagagcagattgaaggatacgcaaaagcatcgcgtcgactttctccaactcatgatagacagccagaattcgaaagaaacagaatcacacaaggcgctctctgacttggagttggtggcccaatcgataatattcatattcgcaggatatgagacaacatcgtccgtgttgagctttattatgtacgagttggccacccaccccgacgtacagcagaagttgcaggaagagatcgacgccgttttgccgaacaaagctccgcccacttatgataccgtgctacagatggaatacctagacatggtagttaatgagacgttgaggttattcccaatagcaatgaggttggaaagagtttgtaagaaggatgtagagataaatggtatctttattccaaaaggggtcgtagtgatgatccccacatacgccttacaccacgattctaaatattggacagagccggagaaattcttgccggaacgcttctcaaagaagaacaaagataacatcgacccgtacatctacactccatttggatcgggcccgaggaactgtattggtatgaggttcgcgcttatgaacatgaagctagccttgattagggtattacagaacttcagctttaagccatgtaaagagacccagataccgctgaaactgaggttgggtgggctgttgcagaccgagaagcccatagttttgaagattgaatcgagggacgggaccgtatcaggtgcgtagaagactccgcgg

SEQ ID NO: 28 - protein, 504 aa, Gorilla gorilla
MALIPDLAMETWLLLAVSLVLLYLYGTHSHGLFKKLGIPGPTPLPFLGNIWSYRKGFCMFDMECHKKYGKVWGFYDGRQPVLAITDPDMIKTVLVKECYSVFTNRRPFGPVGFMKSAISIAEDEEWKRLRSLLSPTFTSGKLKEMVPLIAQYGDVLVRNLSREAETGKPVTMKDLFNTFSTTMEISTTDAGQNIDSLNNPQDPFVENTKKLLRFDFLDPFFLSIIVFPFLTPILEVLNISVFPRAVTSFLRKSVKRMKESRLEDTQKHRVDFLQLMIDSQNSKETESHKALSDLELVAQSIIFIFAGYETTSSVLSFITYELATHPDVQQKLQEEIDAVLPNKAPPTYDTVLQMEYLDMVVNETLRLFPIAMRLERVCKKDVEINGMFIPKGVVVMIPSYALHHDPKYWTEPEKFLPERFSKKNKDNIDPYIYTPFGSGPRNCIGMRFALMNMKLALIRVLQNFSFKPCKETQIPLKLRLGGLLQPEKPIVLKVESRDGTVSGA

SEQ ID NO: 29 - DNA, 1536 bp, Gorilla gorilla
  source 1..1536: mol_type = other DNA
aagcttaaaatggctttaattcccgacttggccatggagacctggttgttgttagctgttagcctagtcttgttgtacttgtacgggacccattctcacggcttatttaagaaactcggtataccagggcctactccattaccattcttagggaacatttggtcatatagaaagggattttgcatgttcgacatggagtgtcacaagaaatacgggaaggtatggggattctatgacggtcgtcaacccgtgttggccataaccgaccctgatatgattaagactgtcttagtcaaggagtgttattctgttttcacgaacaggcgtcctttcggtccagtcggcttcatgaaatcagcaatatccattgcagaggacgaggagtggaaacgattacgttctttgttgtctcctacttttacatcagggaaacttaaagagatggtcccgttaatcgctcagtacggcgacgtgttagttaggaacctgtccagagaggcagaaactggaaaacctgtcaccatgaaagacttattcaacaccttctcgaccacgatggagattagtaccactgatgcaggccagaacatcgattccctcaacaatccgcaagaccctttcgtagagaacaccaagaagttacttagatttgatttcctagacccgttctttctttcgataatcgtgttcccttttctaacaccaatcctagaggtattgaatatatcggtcttcccccgcgctgtaacatctttccttagaaaatcagtaaagcgtatgaaagaatctagattggaagatacacaaaagcaccgtgtcgacttcttacaattaatgatcgacagtcaaaactcgaaagagacggaatcacacaaagcactttcggacctggagttagttgcgcaaagcataattttcatctttgcggggtacgagactacatcctcagttttgtcattcataacctacgagctggccactcatccggatgtgcagcaaaagctgcaggaagaaattgacgcagtcttacccaacaaggctcccccaacctacgacactgttcttcagatggagtacctcgacatggtagtgaatgagacattgagattgtttcccatcgccatgaggttagaaagggtatgcaagaaggacgtcgagataaacggcatgttcatccctaagggcgtggttgtaatgatcccctcttacgcgctccaccatgatccaaaatactggacagagcccgaaaagttccttccggagagattcagcaagaagaacaaggacaacatagacccttacatatataccccattcggtagtggaccacgtaactgcatcggcatgaggtttgctttaatgaacatgaaacttgcattgatcagagttctgcaaaacttttccttcaagccctgtaaagagacgcagattccgttaaagctaaggttaggtggcctactgcaaccagagaagccgattgtactcaaagtcgagagtagggatggtactgtctctggcgcttagaagactccgcgg

SEQ ID NO: 30 - protein, 503 aa, Canis lupus
MDLIPSFSMETWLLLATSLVLLYLYGTYTHGVFKKLGIPGPTPLPFVGTALGYRKGFSVFDENCFRKYGRMWGFYDGRQPVLAITDPDMIKTVLVKECYSVFTNRRSFGPVGFMKSAISLSEDEEWKRIRTLLSPTFTSGKLKEMFPIIGQYGDVLVRNLRKEAEKGKSINLKDIFGAYSMDVITSTSFGVNIDSLNNPQDPFVENIKKLLKFDFLDPFFFSILLFPFLTPVFEVLNIWLFPKSVTDFFTKSVKRMKENRLKDKQKHRVDFLQLMINSQNSKETDTHKALSDLELVAQSIIFIFAGYETTSTSLSFLMYELATHPDVQQKLQEEIDATFPNKALPTYDALVQMEYLDMVLNETLRLYPIAGRLERVCKKDVEISGVFIPKGTVVMVPTFTLHRDQSLWPEPEEFRPERFSRKNKDSINPYTYLPFGTGPRNCIGMRFAIMNMKLALVRVLQNFSFKPCKETQIPLKLNAQGIIQPEKPIVLKVEPRDGSVNGA

SEQ ID NO: 31 - DNA, 1533 bp, Canis lupus
  source 1..1533: mol_type = other DNA
aagcttaaaatggacttgatccctagcttcagtatggaaacatggctactgctcgcgacatcccttgtattgctttatctctacggcacatacactcatggcgtgtttaagaagttgggtatcccaggacctacacctcttcctttcgtcggcactgcgttaggatataggaagggcttctcagtctttgacgagaattgcttccgtaagtacggaagaatgtggggtttctatgacggaaggcagcctgtgttggcgatcacggatccagacatgattaagaccgtattagtcaaagagtgctatagcgtattcacgaacagacgttcctttgggcccgtcggtttcatgaagtcagccatctcgttgagcgaagacgaggagtggaaaagaattaggacacttctgtctccaacatttacttcaggcaaactcaaggaaatgtttcctatcataggacagtacggggacgtccttgttcgtaacctcaggaaggaagccgagaaagggaagtccataaacttaaaggacatcttcggagcttattctatggacgtcatcacttccacgtccttcggcgttaatattgatagtttaaacaacccccaagacccgttcgtggagaacatcaagaaattattgaagttcgatttcctggaccctttcttcttctcaattctactgttccccttcttaacacctgttttcgaggttttgaatatatggttgtttccgaagagcgttacggatttctttacaaagtcggtaaagcgtatgaaagagaaccgcctaaaagacaagcagaaacatagagtagacttcttgcagcttatgataaactcacaaaattctaaagagactgacacccacaaagccctgtccgacttggagttggttgctcagtcgataatcttcatattcgccggctatgagacgaccagtacttcgctgtccttcttaatgtatgaattagcgacccaccccgatgtccagcagaagttacaagaggaaattgacgccacgttcccaaataaagcgttacccacttacgatgctctggtgcagatggagtacttggatatggttttaaacgaaactctccgtttatacccaatcgccgggagattggagagagtatgcaagaaagacgttgagattagtggagtattcatccccaagggaactgtcgtgatggttccaactttcaccctacatcgtgatcagagcttgtggccggagcctgaggaatttagacccgagaggttctcaagaaagaataaggattccatcaacccatatacttaccttccgttcggtactggacctagaaactgtattggaatgcgcttcgccataatgaacatgaagctagctctagttcgcgtattgcagaacttctcatttaagccgtgtaaagagacacaaatcccattaaagctgaatgctcagggtatcatacagccagagaagccaattgttttaaaggttgagcctagggacggtagtgtgaacggggcttagaagactccgcgg

SEQ ID NO: 32 - protein, 490 aa, Homo sapiens
MEPFVVLVLCLSFMLLFSLWRQSCRRRKLPPGPTPLPIIGNMLQIDVKDICKSFTNFSKVYGPVFTVYFGMNPIVVFHGYEAVKEALIDNGEEFSGRGNSPISQRITKGLGIISSNGKRWKEIRRFSLTTLRNFGMGKRSIEDRVQEEAHCLVEELRKTKASPCDPTFILGCAPCNVICSVVFQKRFDYKDQNFLTLMKRFNENFRILNSPWIQVCNNFPLLIDCFPGTHNKVLKNVALTRSYIREKVKEHQASLDVNNPRDFIDCFLIKMEQEKDNQKSEFNIENLVGTVADLFVAGTETTSTTLRYGLLLLLKHPEVTAKVQEEIDHVIGRHRSPCMQDRSHMPYTDAVVHEIQRYSDLVPTGVPHAVTTDTKFRNYLIPKGTTIMALLTSVLHDDKEFPNPNIFDPGHFLDKNGNFKKSDYFMPFSAGKRICAGEGLARMELFLFLTTILQNFNLKSVDDLKNLNTTAVTKGIVSLPPSYQICFIPV

SEQ ID NO: 33 - DNA, 1494 bp, Homo sapiens
  source 1..1494: mol_type = other DNA
aagcttaaaatggagccctttgtcgtgttggtactttgcttgagcttcatgttgttgttttccttatggagacagtcgtgtcgtaggagaaaactaccaccgggtcctactccactcccaataattggtaacatgctgcagatcgatgttaaagacatctgtaaatcattcacaaacttctcgaaggtatatggtcccgtctttactgtctacttcggtatgaacccgatcgtggtcttccacggttacgaggccgtgaaggaagcattgatagataatggggaagaattctcagggcgcggcaacagtcccattagtcagaggatcaccaaggggttgggaataatctcttcgaatggtaagagatggaaggagatccgaagattcagccttaccactctcagaaatttcggcatgggaaaacgcagtatagaggaccgtgtccaagaggaagctcactgcttagtagaggagttgaggaagacgaaagcatctccatgtgatcctaccttcatcttaggttgcgctccatgcaatgttatatgctcagtggtattccaaaagagattcgactataaagaccagaacttcctaactttgatgaagcgctttaatgagaatttccgtatccttaactcaccgtggatccaggtctgcaacaactttccacttctgattgattgtttcccgggcactcacaacaaagtgcttaagaacgttgctctcactcgtagttacatccgtgaaaaggttaaagagcaccaagctagtctggacgttaacaacccacgtgacttcatagactgcttcctaatcaagatggaacaagaaaaggataaccagaaatctgagtttaacatagaaaacctggtaggtacggtggctgatctgttcgtcgctggcactgagacaactagcactaccttgaggtacgggctacttctgttgttgaagcacccagaggtaactgctaaagtacaagaggaaattgatcatgtaatcggtcgtcaccgtagtccgtgcatgcaggatagatcccacatgccctacacagacgcagtggtccacgaaatccagagatactctgacctagtgccaactggcgtcccacacgcagtcacaaccgacacgaagtttagaaattaccttattccgaaaggtacgactatcatggctttgctaacatcagtgctacatgatgacaaggagttccccaaccctaatatctttgatcctggccattttcttgataagaacggcaacttcaagaaatcggactactttatgccgttttcggctgggaagaggatctgcgctggagaaggattagctagaatggagttgttcttatttctgacgactattctacagaacttcaatttaaagtcggttgacgatttgaagaacctgaacacaactgcagttacaaagggcatagttagtctaccgccatcttatcaaatatgctttatcccggtatagaagactccgcgg

SEQ ID NO: 34 - protein, 490 aa, Pan troglodytes
MEPFVVLVLCLSFMLLFSLWRQSSGRRKLPPGPTPLPIIGNMLQIDVKDICKSFSNFSKVYGPVFTVYFGMNPIVVLHGYEAVKEALIDNGEEFSGRGSSPISQRITKGLGIISSNGKRWKEIRRFSLTTLRNFGMGKRSIEDRVQEEAHCLVEELRKTKASPCDPTFILGCAPCNVICSVVFQKRFDYKDQNFLTLMKRFNENFRILNSPWIQVCNNFPLLIDCFPGTHNKVLTNVALTQSYIREKVKEHQASLDVNNPRDFIDCFLIKMEQEKDNQKSEFNIENLVGTVADLFVAGTETTSTTLRYGLLLLLMHPEVTAKVQEEIDHVIGRHRTPCMQDRSHMPYTDAVVHEIQRYSDLVPTGVPHAVTTDTKFRNYLIPKGTTIMTLLTSVLHDDKEFPNPNIFDPGHFLDKNGNFKKSDYFMPFSAGKRICAGEGLARMELFLFLTTILQNFNLKSVDDLKNLNTTAVTKGIVSLPPSYQICFIPV

SEQ ID NO: 35 - DNA, 1494 bp, Pan troglodytes
  source 1..1494: mol_type = other DNA
aagcttaaaatggagcccttcgtcgtattagttctctgcctttcttttatgttgctattctcattgtggcgccagagttccggacgacgtaaattaccacctggaccaacaccactcccaataataggtaacatgctacagatagacgtaaaagacatatgcaagtccttctcaaacttttctaaggtatatggacctgtattcacggtatattttgggatgaatcccatcgtggttctgcacggatacgaagccgttaaagaggctcttattgataatggcgaggaatttagtggcaggggctcgtcgccgatatctcagaggataactaaaggactaggaattattagctccaatggaaagcgctggaaggagatcagaaggttcagcctgacgacactacgtaacttcggaatgggtaagcgatcaatagaggatagggttcaggaagaggcacactgcctggtagaagaattacgcaagactaaagcatccccatgtgatccgacattcatcctcggctgcgcaccatgcaacgtaatctgctcggtcgttttccagaagcgatttgattacaaggaccagaactttcttacgttgatgaagcgctttaatgagaatttcaggattttgaatagcccttggatccaggtctgcaataacttcccgcttctaatagactgcttccctggtacccataataaggtgttaaccaacgtcgcactgacccaatcctacattcgagagaaagtcaaagagcatcaagcatcacttgacgtcaataaccctagagacttcattgactgcttcttgatcaaaatggagcaggagaaggacaatcagaagtcagaattcaacatcgaaaacttagtcggtactgtggctgatctgtttgtcgcagggacggaaactacttccacgaccttaagatacggattattgctgttgcttatgcaccccgaggttacagcgaaagtccaagaggagatcgaccatgtaattggccgtcaccgtacgccctgtatgcaagaccgttcgcacatgccctacacagatgcggttgtacacgagatccagcgatactcagacctggtgccaactggggtgccccacgctgtaactactgacacgaagtttcgtaattacttaattccaaaaggcacgaccattatgacgctattaacgtctgtcctgcacgacgacaaggagttccctaaccctaacatattcgacccaggccattttctagataagaacgggaacttcaagaaatcggattactttatgcctttctcagctgggaagaggatctgcgcaggtgagggcttggccaggatggagttattcttgttcttgactactatattacagaacttcaacttgaagtcagttgatgacttgaagaaccttaacactacagcagtcactaagggtatagtctcattaccccctagttatcaaatctgcttcattcctgtttagaagactccgcgg

SEQ ID NO: 36 - protein, 490 aa, Pongo abelii
MEPFVVLVLCLSFMLLFSLWRQSSGRRKLPPGPTPLPIIGNMLQIDIKDICKSFSNFSKVYGPVFTVYFGMNPMVVLHGYEAVKEALIDNGEEFSGRGSSPISQRITKGLGIISSNGNRWKEIRRFSLTTLRNFGMGKRSIEDRVQEEARCLVEELRKTKASPCDPTFILGCAPCNVICSVVFQKRFDYKDQNFLTLMKRFNENFRILNSPWIQVCNNFPLLIDCFPGTHNKLLKNVALTRSYIREKVKEHQTSLDVTSPRDFIDCFLIKMEQEKDNQKSEFNIENLVGTVADLFVAGTETTSTTLRYGLLLLLKHPEVTAKVQEEIDHVIGRHRSPCMQDRSHMPYTDAVVHEIQRYIDLVPTGVPHAVTTDIQFRNYLIPKGTTIMTLLTSVLHDDKEFPNPKIFDPGHFLDKNGNFKKSDYFMPFSAGKRICAGEGLARMELFLFLTTILQNFNLKSVDDLKNLNTTAVTKGIVSLPPSYQICFIPV

SEQ ID NO: 37 - DNA, 1494 bp, Pongo abelii
  source 1..1494: mol_type = other DNA
aagcttaaaatggagccatttgtggtacttgtactgtgtcttagcttcatgcttctgttctccctatggagacagtcatctggacgacgtaagttgccgccagggccaacccctttacccatcattgggaacatgcttcaaatagatattaaggacatatgcaagagcttttcgaacttctcaaaggtttatgggcctgtgtttaccgtatatttcggaatgaacccaatggtcgtgttgcatggttacgaggctgttaaggaagcactgatagacaacggcgaggagttctcaggccgtgggtcatctcctatctcacaaaggattaccaagggattgggaataatatcgtcaaacggtaaccgctggaaggagatcagaagattcagcctcactaccttgcgtaactttggtatggggaaaagaagtatcgaggaccgtgtacaggaagaggcaaggtgtcttgtcgaggagctacgtaagaccaaggcgtccccgtgcgacccaacctttatcttgggctgcgctccgtgtaacgtaatctgctcagtcgtgttccagaagaggtttgattacaaggaccagaacttcttgactttaatgaagcgttttaatgagaactttaggattctaaactccccgtggattcaggtttgtaacaatttcccgttacttatagactgttttccaggtactcacaataagctactcaagaatgtggcactaacccgtagctatattcgcgagaaagtcaaagagcaccagacgtccctagatgtgacttcacccagggacttcatagactgcttcttaataaagatggagcaggagaaggataaccagaagagtgaattcaatattgagaatttagtcggtacggttgccgatctattcgttgccggaacggagactacatcgactacactgcgttatggtttgttattgttgttaaagcaccctgaggtcacagctaaagtgcaggaagagattgatcacgtcatcggtagacacagaagtccatgcatgcaagacagaagccacatgccatatactgacgcagttgtccacgagatacagcgttacatcgatttggtcccaacaggggtgccacacgctgttaccacggatattcagtttagaaactatcttataccaaaaggaacgacaatcatgacccttctgacctctgttctacatgacgacaaagagttcccaaatcctaagatcttcgacccaggtcacttcttggacaagaatggaaacttcaagaagtccgattacttcatgcctttcagtgccggtaagagaatctgtgctggtgaaggactagcgaggatggagcttttcttattcctaacaaccatattgcagaactttaatcttaaatcagttgacgacctaaagaacctaaatacaactgccgtgacgaaaggcatagttagtctcccaccgtcctaccagatatgtttcattccagtatagaagactccgcgg

SEQ ID NO: 38 - protein, 490 aa, Chlorocebus aethiops
MEPFVVLVLCLSFVLLFSLWRQSSGRRNLPPGPTPFPIIGNMLQIDVKDICKSFSNFSKVYGPVFTVYLGMNPVVVLHGYEAVKEALIDNAEEFSGRGILPISERITKGLGIISSNGKRWKETRRFSLTTLRNFGMGKRSIEDRVQEEARCLVEELRKTKASPCDPTFILGCAPCNVICSVVFQKRFDYKDENFLTLMKRFTENFRILTSPWIQVCNNFPLLIDCFPGTHNKLLKNVALTKSYIREKVKEHQATLDINNPRDFIDCFLIKMEKEKDNQQSEFTIENLVGTVADLFVAGTETTSTTLRYGLLLLLKHPEVTAKVQEEIDHVIGRHRSPCMQDRSHMPYTDAVVHEIQRYIDLVPTGVPHAVTTDIKFRNYLIPKGTIIMTLLTSVLHDDKEFPNPKIFDPGHFLDENGNFKKSDYFMPFSAGKRICAGEGLARMELFLFLTTILQNFNLKSVADLKNLNTTSATRGIISLPPSYQICFIPV

SEQ ID NO: 39 - DNA, 1494 bp, Chlorocebus aethiops
  source 1..1494: mol_type = other DNA
aagcttaaaatggagccattcgttgttttagtcctgtgcttatcttttgttttactgttctctttatggcgacagtcgagcggtcgacgaaacctaccaccaggccccactccgttcccgatcataggtaatatgcttcaaatcgacgtaaaggatatctgcaagtccttctccaatttcagtaaagtctacggcccggtgtttacagtatatttgggtatgaacccggtagtcgtactacacggatacgaggcggttaaagaagcattaatcgataacgcagaggaattctcagggcgcgggatattacccatatctgagcgcatcacaaaggggcttggcattatttccagcaatggtaaaagatggaaggagacacgaagattttcactaacaactttgcgtaacttcggaatgggaaaacgttcgatcgaggaccgcgtacaagaggaagcacgatgtcttgtggaagagttgagaaagaccaaggcttcgccatgtgatcctacattcattcttggatgtgcaccctgcaacgtaatctgcagtgtggtattccagaagcgatttgactataaggacgagaacttcttaactttaatgaaaagattcacggagaactttaggatcttgacttcaccttggatccaggtatgcaataactttcctcttctaatcgactgcttcccaggaacacataacaagttgttgaagaacgtagcactaactaagtcatacatacgtgagaaggtgaaagaacaccaagcaacacttgatattaataatcctcgtgactttattgactgctttctaatcaagatggagaaagagaaggacaaccagcagtctgagttcactatagagaacctagttgggactgtagctgacttgtttgtcgccggtaccgagacaacatcaacaaccttgaggtatggtctattattgttactgaagcatcccgaagtgacggcaaaggtccaggaagagatagaccacgtaatcggccgacaccgttcaccgtgcatgcaggatagatcacatatgccatataccgatgcggtcgtacatgagatccaaaggtacattgacttggttcctacaggcgttccccacgccgtaacaactgacattaaattccgcaactatctaatacccaagggcactatcatcatgacgctactgacgtccgtcctgcatgatgacaaggagtttccaaaccctaagatcttcgacccgggacacttcctagacgagaatggtaacttcaagaaatcagattacttcatgcccttctcagctggtaaacgaatctgcgccggagaaggcctcgctcgtatggagcttttcttgttcttaacgaccatattgcagaacttcaacctaaagtcagttgcagaccttaagaatttaaatacaaccagcgccactcgtggcatcatctctttgccgccgtcatatcagatatgctttattcctgtatagaagactccgcgg

SEQ ID NO: 40 - protein, 502 aa, Homo sapiens
MDLIPNLAVETWLLLAVSLVLLYLYGTRTHGLFKRLGIPGPTPLPLLGNVLSYRQGLWKFDTECYKKYGKMWGTYEGQLPVLAITDPDVIRTVLVKECYSVFTNRRSLGPVGFMKSAISLAEDEEWKRIRSLLSPTFTSGKLKEMFPIIAQYGDVLVRNLRREAEKGKPVTLKDIFGAYSMDVITGTSFGVNIDSLNNPQDPFVESTKKFLKFGFLDPLFLSIILFPFLTPVFEALNVSLFPKDTINFLSKSVNRMKKSRLNDKQKHRLDFLQLMIDSQNSKETESHKALSDLELAAQSIIFIFAGYETTSSVLSFTLYELATHPDVQQKLQKEIDAVLPNKAPPTYDAVVQMEYLDMVVNETLRLFPVAIRLERTCKKDVEINGVFIPKGSMVVIPTYALHHDPKYWTEPEEFRPERFSKKKDSIDPYIYTPFGTGPRNCIGMRFALMNMKLALIRVLQNFSFKPCKETQIPLKLDTQGLLQPEKPIVLKVDSRDGTLSGE

SEQ ID NO: 41 - DNA, 1530 bp, Homo sapiens
  source 1..1530: mol_type = other DNA
aagcttaaaatggacttgatcccaaacttggctgttgaaacttggttgttattggccgtttctttggtcttgttgtacttgtatggtactagaacccatggtttgttcaagagattgggtattccaggtccaactccattgccattattgggtaatgttttgtcctacagacaaggcttgtggaagtttgatactgagtgctataagaaatacggtaagatgtggggtacttacgaaggtcaattgccagttttggctattactgatccagatgttatcagaaccgtcttggtcaaagaatgctactctgttttcaccaacagaagatctttgggtccagttggttttatgaagtccgctatttctttggccgaagatgaagaatggaagagaatcagatctttgttgtctccaactttcacctccggtaagttgaaagaaatgttcccaattattgcccaatacggtgatgttttggtcagaaacttgagaagagaagctgaaaaaggtaagccagttaccttgaaggatattttcggtgcttactccatggatgttattaccggtacttctttcggtgtcaacatcgattctttgaacaatccacaagatcccttcgttgaatctaccaagaagtttttgaagttcggtttcttggaccccttgttcttgtccattattttgttcccatttctgaccccagttttcgaagccttgaatgtttctttgtttccaaaggacaccatcaatttcttgagcaagtccgttaacaggatgaagaagtctagattgaacgacaagcaaaagcacaggttggatttcttgcaactgatgatcgattcccagaactctaaagaaactgaatcccataaggctttgtccgatttggaattggctgcccaatctatcattttcattttcgctggttacgaaaccacctcctctgttttgtcttttaccttgtatgaattggccactcatccagacgttcaacaaaagttgcaaaaagaaatcgatgccgtcttgccaaacaaagctccaccaacttatgatgctgttgtccaaatggaatacttggatatggttgtcaacgaaaccttgaggttgtttccagttgctatcagattggaaagaacctgcaaaaaggatgtcgaaatcaacggtgttttcatcccaaaaggttccatggttgtgattccaacttacgcattgcatcatgatccaaagtattggactgaaccagaagaattcagaccagaaagattctccaagaagaaggattccattgatccttacatctacactccatttggtactggtcctagaaactgtattggtatgagattcgctctgatgaacatgaagttggccttgattagagtgttgcagaacttctctttcaaaccctgtaaagaaacgcagatcccattgaagttggatacacaaggtttattgcaaccagaaaagcctatcgttttgaaggtcgattctagagatggtactttgtctggtgagtgaaagactccgcgg

SEQ ID NO: 42 - protein, 502 aa, Pan troglodytes
MDLIPNLAVETWLLLAVSLVLLYLYGTRTHGLFKRLGIPGPTPLPLLGNVLSYRQGLWKFDTECYKKYGKMWGMYDGQLPVLAITDPDMIRTVLVKECYSVFTNRRSLGPVGFMKSAISLAEDEEWKRIRSLLSPTFTSGKLKEMFPIIAQYGDVLVRNLRREAEKGKPVTLKDIFGAYSMDVITGTSFGVNIDSLNNPQDPFVESTKKFLKFGFLDPLFLSIILFPFLTPVFEALNVSLFPKDTINFLSKSVNRMKKSRLNDKQKHRLDFLQLMIDSQNSKETESHKALSDLELAAQSIIFIFAGYETTSSVLSFTLYELATHPDVQQKLQKEIDAVLPNKAPPTYDAVVQMEYLDMVVNETLRLFPVAIRLERTCKKDVEINGVFIPKGSMVVIPTYALHHDPKYWTEPEEFRPERFSKKKDSIDPYIYTPFGTGPRNCIGMRFALMNMKLALIRVLQNFSFKPCKETQIPLKLDTQGLLQPEKPIVLKVDSRDGTLSGE

SEQ ID NO: 43 - DNA, 1530 bp, Pan troglodytes
  source 1..1530: mol_type = other DNA
aagcttaaaatggacttgatcccaaacttggctgttgaaacttggttgttattggccgtttctttggtcttgttgtacttgtatggtactagaacccatggtttgttcaagagattgggtattccaggtccaactccattgccattattgggtaatgttttgtcctacagacaaggcttgtggaagtttgatactgagtgctataagaaatacggtaagatgtggggtatgtacgatggtcaattgccagttttggctattactgatccagatatgatcagaaccgtcttggtcaaagaatgctactctgttttcaccaacagaagatctttgggtccagttggttttatgaagtccgctatttctttggccgaagatgaagaatggaagagaatcagatctttgttgtctccaactttcacctccggtaagttgaaagaaatgttcccaattattgcccaatacggtgatgttttggtcagaaacttgagaagagaagctgaaaaaggtaagccagttaccttgaaggatattttcggtgcttactccatggatgttattaccggtacttctttcggtgtcaacatcgattctttgaacaatccacaagatcccttcgttgaatctaccaagaagtttttgaagttcggtttcttggaccccttgttcttgtccattattttgttcccatttctgaccccagttttcgaagccttgaatgtttctttgtttccaaaggacaccatcaatttcttgagcaagtccgttaacaggatgaagaagtctagattgaacgacaagcaaaagcacaggttggatttcttgcaactgatgatcgattcccagaactctaaagaaactgaatcccataaggctttgtccgatttggaattggctgcccaatctatcattttcattttcgctggttacgaaaccacctcctctgttttgtcttttaccttgtatgaattggccactcatccagatgttcaacagaagttgcaaaaagaaatcgatgccgttttgccaaacaaagctccaccaacttatgatgctgttgtccaaatggaatacttggatatggttgtcaacgaaaccttgaggttgtttccagttgctatcagattggaaagaacctgcaaaaaggatgtcgaaatcaacggtgttttcatcccaaaaggttccatggttgtgattccaacttacgcattgcatcatgatccaaagtattggactgaaccagaagaattcagaccagaaagattctccaagaagaaggattccattgatccttacatctacactccatttggtactggtcctagaaactgtattggtatgagattcgctctgatgaacatgaagttggccttgattagagtgttgcagaacttctctttcaaaccctgtaaagaaacgcagatcccattgaagttggatacacaaggtttattgcaaccagaaaagcctatcgttttgaaggtcgattctagagatggtactttgtctggtgagtgaaagactccgcgg

SEQ ID NO: 44 - protein, 503 aa, Macaca fascicularis
MDLIPNLAMETWLLLAVSLVLLYLYGTRSYGLFKRQGIPGPTPLPFLGNILSYRQGLWKFDTECYKKYGKMWRTQDGQLPVLTITDPDMIKTVLVKECYSVFTNRRPLGPVGLMKSAISIAEDEEWKRIRSLLSPTFTSGKLKEMFPIIAQYGDMLVRNLRREAEKGKPVTLKDIFGAYSMDVITSTSFGVNIDSLNNPKDPFVESVKKFLKFDFLDPLFLLTILFPFLIPAFEALNVSLFPKDAINFLNKSVNSMKKSRLNDKQKHRVDFLQLMIDSQNSKETESHKALSDQELVAQSIIFIFAGYETTSSVLSFIIYELATHPDVQQKLQKEIDAVLPNKAPATYDAMVQMEYLDMVVNETLRLFPIAIRLERACKKDVEINGVFIPKGAMVVIPTYALHHDPKYWTEPEEFRPERFSKKNKDSIDPYIYTPFGSGPRNCIGMRFALMNMKLAIIKVLQNFSFKPCKETQIPLKLGNQGLLQSEKPIVLKVESRDGTLSGE

SEQ ID NO: 45 - DNA, 1533 bp, Macaca fascicularis
  source 1..1533: mol_type = other DNA
aagcttaaaatggacttgatcccaaacttggctatggaaacttggttgttgttggctgtttctttggtcttgttgtacttgtacggtactagatcttacggtttgttcaagagacaaggtattccaggtccaactccattgccatttttgggtaacattttgtcctacagacaaggcttgtggaagttcgatactgaatgctataagaaatacggcaagatgtggcgtactcaagatggtcaattgccagttttgactattaccgatccagatatgatcaagaccgtcttggtcaaagaatgctactctgttttcactaacagaaggccattgggtccagttggtttgatgaagtctgctatttctattgccgaagatgaagaatggaagaggatcagatctttgttgtctccaactttcacttccggcaaattgaaagaaatgttcccaattattgcccagtacggtgatatgttggttagaaacttgagaagagaagccgaaaaaggtaagccagttaccttgaaggatattttcggtgcttactccatggacgttattacttctacttctttcggtgtcaacatcgacagtttgaacaatccaaaggatccattcgttgagagcgttaagaagtttttgaagttcgactttttggaccccttgttcttgttgactattctgttcccatttttgattccagctttcgaagccttgaacgtttctttgtttccaaaggacgctatcaacttcctgaacaagtctgttaactccatgaagaagtctaggttgaacgataagcaaaagcacagggttgatttcttgcagttgatgatcgattcccagaactctaaagaaaccgaatctcataaggctttgtccgatcaagaattggttgcccaatccatcattttcattttcgctggttacgaaaccacctcctctgttttgtctttcatcatctatgaattggctacccatccagatgtccaacaaaagttgcaaaaagaaatcgatgccgtcttgccaaacaaagctccagctacttatgatgctatggtccaaatggaatacttggatatggttgtcaacgaaaccttgaggttgttccctattgctatcagattggaaagggcttgcaaaaaagatgtcgaaatcaacggtgttttcattccaaaaggtgccatggttgttattccaacttacgcattgcatcacgatccaaagtattggactgaaccagaagaattcagaccagaaagattctccaagaaaaacaaggattccatcgatccttacatctacactccatttggttctggtccaagaaactgtatcggtatgagatttgctctgatgaacatgaagttggccattattaaggtcctgcagaacttctctttcaaaccatgcaaagaaacccagattccattgaagttgggtaatcaaggtctgttgcaatctgaaaagccaatcgttttgaaggttgaatccagagatggtactttgtctggtgagtaaaagactccgcgg

SEQ ID NO: 46 - protein, 503 aa, Callithrix jacchus
MDLIPNLAVETWLLLALSLVLLYLYGTRSHGIFKKLGIPGPAPLPFVGNILSYRQGIWKFDSECHKKYGKMWGSYDGQLPVLAITDPDIIKAVLVKECYSIFTNRRPLGPVGFMKSAITVAQDDEWKRIRSLLSPTFTSGKLKEMFPIIAQYGDVLVRNLRREAGKGKPVTMKDIFGAYSMDVITGTSFGVNIDSLNNPKDPFVESVKKFLKFDFLDPLFLSTIFFPFLTPVFEALNFSLFPKDAINFLKQSVNRMKKSRLNDKQKHRVDFLQLMIDSQNSNETASHKALSDLELLAQSIIFIFAGYETTSSVLSFTIYELATNPDVQQKLQEEIDVVLPNKAPATYDAVVQMEYLDMVVNETLRLYPIAVRLERVCKKDVEINGVFIPKGALVVIPTYALHHDPKYWTEPKEFRPERFSKKNKDSIDPYIYTPFGTGPRNCIGMRFALMNMKLALIRVLQNFSFKPCKETQIPLKLGVQGLLQAEKPIILKVESRDGTLSGE

SEQ ID NO: 47 - DNA, 1514 bp, Callithrix jacchus
  source 1..1514: mol_type = other DNA
aagcttaaaatggacttgatcccaaacttggctgttgaaacttggttgttattggccttgtctttggtcttgttgtacttgtatggtactagatcccatggcatctttaagaagttgggtattccaggtccagctccattgccatttgttggtaacattttgtcttacaggcaaggcatttggaagttcgattctgaatgccataagaaatacggtaagatgtggggttcttacgatggtcaattgccagttttggctattactgatccagatattatcaaggccgtcttggtcaaagaatgctactctgtttttaccaacagaaggccatttggtccagttggtttgatgaagtctgctatttctttggcccaagatgatgaatggaagagaatcagatctttgttgtctccaactttcacctccggtaagttgaaagaaatgttcccaattattgcccaatacggtgatgttttggtcagaaacttgagaagagaagctggtaaaggtaagccagttaccatgaaggatattttcggtgcttactccatggatgttattaccggtacttctttcggtgtcaacatcgattctttgaacaatccaaaggatcccttcgttgaatctgtcaagaagtttttgaagtttgacttcttggaccccttgttcttgtctactattttctttccattcttgacgccagttttcgaagccttgaattttagcttgttcccaaaggatgctatcaacttcctgaagcaatccgttaacaggatgaagaagtctagattgaacgacaagcaaaagcacagggttgatttcttgcaactgatgatcgattcccagaactctaacgaaactgcttctcataaggctttgtctgatttggaattgctggcccaatccattattttcattttcgctggttacgaaaccacctcctctgttttgtcttttaccatctatgaattggccaccaatccagatgttcaacagaaattgcaagaagaaatcgacgttgtcttgccaaacaaagctccagctacttatgatgctgttgtccaaatggaatacttggatatggttgtcaacgaaaccttgaggttgtatccaattgctgtcagattggaaagggtctgcaaaaaggatgttgaaatcaacggtgttttcattccaaagggtgctttagttgttattccaacttacgccttgcatcacgatcctaaatattggactgaacccaaagaattcagaccagaaagattctccaagaaaaacaaggattccatcgatccttacatctacactccatttggtactggtcctagaaactgtattggtatgagattcgctctgatgaacatgaagttggccttgattagagtgttgcagaacttctctttcaaaccctgtaaagaaacccagattccattgaaattgggtgtccaaggtttgttgcaagctgaaaaacctatcatcttgaaggttgaatccagagatggtactttgtctgg

SEQ ID NO: 48 - protein, 134 aa, Homo sapiens
MAEQSDEAVKYYTLEEIQKHNHSKSTWLILHHKVYDLTKFLEEHPGGEEVLREQAGGDATENFEDVGHSTDAREMSKTFIIGELHPDDRPKLNKPPETLITTIDSSSSWWTNWVIPAISAVAVALMYRLYMAED

SEQ ID NO: 49 - DNA, 419 bp, Homo sapiens
  source 1..419: mol_type = other DNA
aagcttaaaatggccgagcaatctgatgaagcagtgaagtactacacattagaagagatacaaaaacacaaccattcaaaaagcacttggctcattctacaccataaagtttatgatttgactaagtttttggaggaacatccaggaggtgaagaggtccttagagaacaggcaggcggtgacgctactgaaaactttgaagatgttgggcattcaaccgacgctagagaaatgagtaaaactttcattattggtgaacttcacccagatgacagaccaaaactgaataagcctcctgaaacactaataacgacaatcgattcttcctcttcatggtggacaaattgggttatcccagcgatctctgccgtcgctgtagcattaatgtataggttgtacatggctgaagattaaccgcg

SEQ ID NO: 50 - protein, 677 aa, homo sapiens
MGDSHVDTSSTVSEAVAEEVSLFSMTDMILFSLIVGLLTYWFLFRKKKEEVPEFTKIQTLTSSVRESSFVEKMKKTGRNIIVFYGSQTGTAEEFANRLSKDAHRYGMRGMSADPEEYDLADLSSLPEIDNALVVFCMATYGEGDPTDNAQDFYDWLQETDVDLSGVKFAVFGLGNKTYEHFNAMGKYVDKRLEQLGAQRIFELGLGDDDGNLEEDFITWREQFWPAVCEHFGVEATGEESSIRQYELVVHTDIDAAKVYMGEMGRLKSYENQKPPFDAKNPFLAAVTTNRKLNQGTERHLMHLELDISDSKIRYESGDHVAVYPANDSALVNQLGKILGADLDVVMSLNNLDEESNKKHPFPCPTSYRTALTYYLDITNPPRTNVLYELAQYASEPSEQELLRKMASSSGEGKELYLSWVVEARRHILAILQDCPSLRPPIDHLCELLPRLQARYYSIASSSKVHPNSVHICAVVVEYETKAGRINKGVATNWLRAKEPAGENGGRALVPMFVRKSQFRLPFKATTPVIMVGPGTGVAPFIGFIQERAWLRQQGKEVGETLLYYGCRRSDEDYLYREELAQFHRDGALTQLNVAFSREQSHKVYVQHLLKQDREHLWKLIEGGAHIYVCGDARNMARDVQNTFYDIVAELGAMEHAQAVDYIKKLMTKGRYSLDVWS

SEQ ID NO: 51 - DNA, 2040 bp, homo sapiens
  source 1..2040: mol_type = other DNA
taaaatgggtgattcacatgttgatacatcttctacagtttcagaagctgttgctgaagaagtttctttattttcaatgactgatatgattttgttttcattaattgttggtttgttgacatattggttcttgtttagaaagaagaaggaagaagttccagaatttactaagattcaaaccttgacatcttcagttagagaatcatcttttgttgaaaagatgaagaagactggtagaaatattattgttttctacggttctcaaactggtactgctgaagaatttgctaacagattgtcaaaggatgcacatagatatggtatgagaggtatgtctgctgatcctgaagaatatgatttagcagatttatcttcattgcctgaaattgataatgctttagttgttttctgtatggctacttatggtgaaggtgatcctactgataacgctcaagatttttatgattggttgcaagaaactgatgttgatttatctggtgttaagttcgctgttttcggtttgggtaataagacttatgaacatttcaacgctatgggtaaatacgttgataaaagattggaacaattgggtgctcaaagaatttttgaattaggtttgggtgatgatgatggtaacttagaagaagattttattacatggagagaacaattttggcctgcagtttgtgaacatttcggtgttgaagctaccggtgaagaatcatctattagacaatatgaattggttgttcatacagatattgatgctgctaaagtttacatgggtgaaatgggtagattgaaatcatatgaaaatcaaaagccacctttcgatgctaagaatcctttcttagctgcagttacaacaaacagaaagttgaatcaaggtacagaaagacatttgatgcatttggaattggatatttctgattcaaaaattagatacgaatctggtgatcatgttgctgtttatccagctaatgattctgctttagttaaccaattaggtaaaattttgggtgctgatttagatgttgttatgtctttaaataacttagatgaagaatctaacaagaaacatccattcccatgtcctacttcatacagaacagctttaacttattatttagatattacaaaccctccaagaactaatgttttgtatgaattggctcaatatgcatctgaaccttctgaacaagaattgttaagaaaaatggcttcttcatctggtgaaggtaaagaattatacttgtcatgggttgttgaagctagaagacatattttagcaattttacaagattgtccatcattgagaccaccaattgatcatttgtgtgaattgttgcctagattacaagctagatattattctattgcatcatcttctaaagttcatccaaattcagttcatatttgtgctgttgttgttgaatatgaaactaaggctggtagaattaacaaaggtgttgcaactaattggttgagagctaaggaacctgctggtgaaaatggtggtagagcattagttccaatgttcgttagaaagtctcaatttagattgccatttaaagctactacaccagttattatggttggtcctggtacaggtgttgctccttttattggtttcattcaagaaagagcatggttaagacaacaaggtaaggaagttggtgaaacattattatattacggttgtagaagatcagatgaagattatttgtacagagaagaattagcacaattccatagagatggtgctttgacacaattgaatgttgctttttcaagagaacaatctcataaagtttatgttcaacatttgttgaaacaagatagagaacatttatggaagttgattgaaggtggtgcacatatttatgtttgtggtgatgctagaaacatggcaagagatgttcaaaatacattctatgatattgttgctgaattaggtgctatggaacatgcacaagcagttgattacattaagaaattgatgacaaaaggtagatattctttggatgtttggtcttaacc

SEQ ID NO: 52 - DNA, 1572 bp, Mucor circinelloides
  source 1..1572: mol_type = other DNA
atgactttgattgcttcttataatcaaactttgttggaaagagttatttctgttgttagaaaaaataaaacttcttatattggtatggctattttgtgtgttgttttgcaacaagtttatgcttcttttactgttccaccaaaatatttgagaagatacccaactgtttctttttttgaaatgatgaaatctttttatagaaaagaatctgttttgaatagaaataaaagattggttactccattgactaatgctggtcatggtttttatgtttgtagaataccattggattggactatttatgttactgatccaattgctgctaaaactttgttgttgaaaactgataattttccaaaatctcatgctatttttggtgctttgggtgaatcttctccagcagttaaatttatgggttctgaaaatgttgctgtttctaatggtgaaatgtggaaaaaacaaagaaaaattatgaatccagcttttcatagatctcaaccagttaaattgtttggtggtgttatgccagatttgtttgctttgattgatcaagatccagaacatgtttttgttgctccaagaatgaaatcttttgctttggatgctttgggtttgtctgcttttggttttgattttcaatctttgaaaggtgatccagaaggttggacttctaaatataatactgttatttctactttgtttaatccatttattaatttgtttgctaaatatgattttttgattaaatatatttctccagaaagaagaagagttattaaagctactgatgaatttaatgttatgttgtctaatttggctgataaaagaagacaagaaattttggatggtgaaaaaaaagatatggctgaaaatgaaaaagatttgttgactttgatgattgaagctgatattagagaaggtgttgaaactactactactgaattgagacataatatggctattttttttttggctggtcatgatactactgctaatactttggctttgtgtttgtatcaattggctaaacataaacatgttcaaaaaaaagctagacaagaagttttggatattttgggtgatgatccatgtgatgttactccaactttggaagatttgaaaaaattgaattatgttaatatggttattaaagaaaatttgagaagaaattctccagttgataatttgatggctagagatactcatcaagatattgatttgaatggtacttttattccaaaaggttctaaagttactgttaatgttgcttctattcatttgaatccaaaaatttggcatgatccagaatcttttattccagaaagatttgaaccaggtggtgaatatgatggtcatgatggttttacttggattccattttctaatggttctagacaatgtttgggtttgaatttttctttgactgaacaaagagttgttttgtgtatgttgttgaaaagatatgaaattgaaattccaaaagattctattcattataatgaaattgtttttgataaaccatttacttttgctccacaatctttggaattgtcttttaaaaaaagatattag

SEQ ID NO: 53 - protein, 523 aa, Mucor Circinellloides
MTLIASYNQTLLERVISVVRKNKTSYIGMAILCVVLQQVYASFTVPPKYLRRYPTVSFFEMMKSFYRKESVLNRNKRLVTPLTNAGHGFYVCRIPLDWTIYVTDPIAAKTLLLKTDNFPKSHAIFGALGESSPAVKFMGSENVAVSNGEMWKKQRKIMNPAFHRSQPVKLFGGVMPDLFALIDQDPEHVFVAPRMKSFALDALGLSAFGFDFQSLKGDPEGWTSKYNTVISTLFNPFINLFAKYDFLIKYISPERRRVIKATDEFNVMLSNLADKRRQEILDGEKKDMAENEKDLLTLMIEADIREGVETTTTELRHNMAIFFLAGHDTTANTLALCLYQLAKHKHVQKKARQEVLDILGDDPCDVTPTLEDLKKLNYVNMVIKENLRRNSPVDNLMARDTHQDIDLNGTFIPKGSKVTVNVASIHLNPKIWHDPESFIPERFEPGGEYDGHDGFTWIPFSNGSRQCLGLNFSLTEQRVVLCMLLKRYEIEIPKDSIHYNEIVFDKPFTFAPQSLELSFKKRY

SEQ ID NO: 54 - DNA, 1542 bp, Lichtheimia corymbifera
  source 1..1542: mol_type = other DNA
atgactgaaattaaagatcatatttatagatatagacattatattggtgttgctgctgctgttttgttggtttgtcaacaagtttatcatagaatttttagaataccaaaaaatttgagacatttgccagctattccatacggtaaacaattgaaagcattgcaatctcaagaaaatttgatttctagaactcaaagattggtttttccattgttgccaaaagctcatggtgtttatttgaatagaatgccatttcaatggactatttatgttgctgatccagaaattgctagaattgttttttttaaaccagaatttgctaataaaacttcttctgttttggattctattgatcaaaatactactttgattgaatttgttggtaatgataatgtttctattgttaatggtcatcattggaaagatcaaagaaaaattatgaatccagcttttcatagagctactccagttggtatgtttggttctttgatgccaaaagtttttagattggttgaagaacaaccaactgttccagttttggaattgatgcaaaaattgactttggatgctttgggtaaatctgtttttggttttgattttggtggtttggatgatccagattctgtttgggttaaaacttatagattgttgtttgatggttttactaatgttattccattggtttttccaagattggatggtttgtatagatatttttctgctaaaagaagagcacaacatgatgctgtttataaattgattgatttgttggatggtgttgctgataaaaaaagatctatgttgcaagatgattctaattctcataatgatgttccagaacatgaaaaagatttgttgcaattgatgttggaagctgaattgagaggtgaaggtaattggactaaaaaagaattgagacataatatggctattttttttgttgctggtcatgatactacttctcatgctttgactttttgtttgtatttgttggctatgaatcaagatattcaaaaaaaagctagagaagaaattttgagagttttgggtgatgaaccaaaagatgtttttccaactttggaagattgtaaaaaattggattatttggatatggttattaaagaatctatgagaatttatccaccagctaatgatattttggctagagatgttcatgaagatttgaatgttaaaggtatttttattccaaaaggtgctatggtttctgttgatattcaagcattgcatcatagaccagatttgtggcatgaaccagaaaaatttaatccagatagatttttgccaggtggtgaacatgattctcatgaaggtattgcttatgctccattttcttctggtgctagacaatgtattgctttgaaattttctattatgcaacaaagagttgttttggctatgttgttgagaaaatttgaatgggaattgccaaaagaatctaaacataaagatggtattcaatttgaaattccatttaatttggctccaaaagatttggaattgatttttcataaaagatattag

SEQ ID NO: 55 - protein, 513 aa, Lichtheimia corymbifera
MTEIKDHIYRYRHYIGVAAAVLLVCQQVYHRIFRIPKNLRHLPAIPYGKQLKALQSQENLISRTQRLVFPLLPKAHGVYLNRMPFQWTIYVADPEIARIVFFKPEFANKTSSVLDSIDQNTTLIEFVGNDNVSIVNGHHWKDQRKIMNPAFHRATPVGMFGSLMPKVFRLVEEQPTVPVLELMQKLTLDALGKSVFGFDFGGLDDPDSVWVKTYRLLFDGFTNVIPLVFPRLDGLYRYFSAKRRAQHDAVYKLIDLLDGVADKKRSMLQDDSNSHNDVPEHEKDLLQLMLEAELRGEGNWTKKELRHNMAIFFVAGHDTTSHALTFCLYLLAMNQDIQKKAREEILRVLGDEPKDVFPTLEDCKKLDYLDMVIKESMRIYPPANDILARDVHEDLNVKGIFIPKGAMVSVDIQALHHRPDLWHEPEKFNPDRFLPGGEHDSHEGIAYAPFSSGARQCIALKFSIMQQRVVLAMLLRKFEWELPKESKHKDGIQFEIPFNLAPKDLELIFHKRY

SEQ ID NO: 56 - DNA, 1593 bp, Lichtheimia corymbifera
  source 1..1593: mol_type = other DNA
atgtcttctttgaatattttgtctttgttggataataaacatattaatgctttttttgataatccatctagagcacaatctggtattattgctggttctgttgctattgtttctttgtgtgctgcttattctttgggttctagattgagaggttctaataatggtgttccattggttccatatacttttccaattattggttctactaaagaatatcaagctgatccacaagcatttattgaaaaatggactgctaaattgggtccagtttttagagttcatttgtttggtagaatacatactgttgtttctgatagatatgttagagaagtttttttgaataatgattttgattttttgaaaggtactggtaaaagatttgatactttgttgttgactgatactactttggaagatgttgatattgaagtttttagaactgttgttatgaaacatttgactaaagaaatgaaacattatactccaagagttgttgaacatttgactgctggtggtgatgaaaaattgggtgatgctactgaaccaaaagaattggttcatttgtttccattgttgcaacacatggttgctaaagcatctgcttctatttttgttggtactgaattggctgctgatgatgctgttgttgaaacttgtaaaaatattgctattgatattggttctgaattgggtccatcttcttatattatggatgcttttccatctttggctagattgagaatgtggtatattggtaaatatggtaaagctattaataaacatagacaacatttgttgcacgctttgggtccagttattgataaaagattggctgctgctgaaaaaggtggtgattgggatagaccacaagatattttgcaagatattattgaaactattaatttgactttggataatccaaaaagacatattttgccagttaaatggttgttggctttgttttttgcttctattcatactacttctgaaaattctactattgttttgtatagaattatgcaaaatccagaaattattgatgttttgttggaagaacaaaatgaaattttggaaaaacattatggtactaatattgattattctgatactactaaattgtttactggtgaagttattaaagaaatggttaaattggattctgtttgtagagaagctatgagatctagaaattcttatttggaattgccacatacttatgttggtaaatctagaattactttgtcttgtggtgctgttattgaaccaggtcatgatgttttgattaatatgtggggtaatcatagagatgctaaaattcaaagagatactattggtgatcatcatgattttaaaccatttagatttgttggtttggatagacaatctactaaaattggtgatgattttttgatgtttggtcaaggtagacatgcttgtccaggtagatggtttgctattcaagaaattaaaactattgtttctgttttgattagatattataaattgactccaaaaggtccaattacttttccaactcatccaagaatgccaatgccaactggtgaagttattattcaaagaagacaagaatag

SEQ ID NO: 57 - protein, 530 aa, Lichtheimia corymbifera
MSSLNILSLLDNKHINAFFDNPSRAQSGIIAGSVAIVSLCAAYSLGSRLRGSNNGVPLVPYTFPIIGSTKEYQADPQAFIEKWTAKLGPVFRVHLFGRIHTVVSDRYVREVFLNNDFDFLKGTGKRFDTLLLTDTTLEDVDIEVFRTVVMKHLTKEMKHYTPRVVEHLTAGGDEKLGDATEPKELVHLFPLLQHMVAKASASIFVGTELAADDAVVETCKNIAIDIGSELGPSSYIMDAFPSLARLRMWYIGKYGKAINKHRQHLLHALGPVIDKRLAAAEKGGDWDRPQDILQDIIETINLTLDNPKRHILPVKWLLALFFASIHTTSENSTIVLYRIMQNPEIIDVLLEEQNEILEKHYGTNIDYSDTTKLFTGEVIKEMVKLDSVCREAMRSRNSYLELPHTYVGKSRITLSCGAVIEPGHDVLINMWGNHRDAKIQRDTIGDHHDFKPFRFVGLDRQSTKIGDDFLMFGQGRHACPGRWFAIQEIKTIVSVLIRYYKLTPKGPITFPTHPRMPMPTGEVIIQRRQE

SEQ ID NO: 58 - DNA, 1548 bp, Absidia repens
  source 1..1548: mol_type = other DNA
atgaataaattgtttccagttggtacttctaataaaaaaaaagctgttttgttgtctttgggtgctactgttgctttgatttgtagagctatttatagaatttattatgttccaagatctttgagacatattccatctgttggttattttagattgattagatctattttgagaagagaagatgctactactagagctagaactgtttatggtccagctattaaaaaaggtaatggtatttatttggctaattttccaattacttggtctgtttttgtttcttctccaattgctgctaaatctgttttgatgaaaactgataattttccaaaatctatggaaatttttcatgctttgggtaaagaaaatccagttgttagattttttggtattgataatgttgctattgttaatggtgaaaaatggaaaaaacaaagaaaagttatgaatccagcttttcatagagctatgccagttcaaatgtttggtagattgatgttgaaaggttttaaaaatattgaaaaacaagattatcaagttccaattttggatttttttcaaagattgactttggatgctttgggtattgctggttttggttttgattttcattctgttgatgatccagaatctatttggactaaaacttatgaaaatgttagattgggtttgagatctccattttcttttttgtttccatctatggattggttgttgaaatatattattccaggtagaaaagaattggataaatctgttgataaattgaatgctttgttgatgtctatggctcaagaaagaagattgcaagttagagcttctattgatgataatgttccagattctgaaaaagatttgttgactttgatgttggaagctgaattgagaggtgaaggtactgcttctgatgaacaattgagatctaatttggctgttttttttttggctggtcatgaaactactgctaatactatgtctttttgtttgtataatttggctatgaataaagaagttcaatctaaagctagacaagaagttttgactgttttgggtgatgaaccagttgatattttgccacaaattgatgaattgagacaaatgccatatattgatatggttttgaaagaaaatttgagaagatttggtccagcttctatgttgattgctagaaaatctcaagaagattttgatttgaatggtgtttttattccaaaaaatactccagttgttgttgaaactcatgctttgcatcataatccagatgtttggaaaaatccagaacaatttgatccagaaagatttgctccaggtggtgaacatgaaacttgtcatgaaggtatggcttggttgccattttcttctggttctagaggttgtttgggtatgaatttttctttggctgaacaaagaacttttttggttatgttgttgagaaaatatgaatgggaattgccagaagattctattcatagaaatggtgttaaaattcaaaattttcaaaatgctgctccagaatctttggaaattaaattttcttctagatattag

SEQ ID NO: 59 - protein, 515 aa, Absidia repens
MNKLFPVGTSNKKKAVLLSLGATVALICRAIYRIYYVPRSLRHIPSVGYFRLIRSILRREDATTRARTVYGPAIKKGNGIYLANFPITWSVFVSSPIAAKSVLMKTDNFPKSMEIFHALGKENPVVRFFGIDNVAIVNGEKWKKQRKVMNPAFHRAMPVQMFGRLMLKGFKNIEKQDYQVPILDFFQRLTLDALGIAGFGFDFHSVDDPESIWTKTYENVRLGLRSPFSFLFPSMDWLLKYIIPGRKELDKSVDKLNALLMSMAQERRLQVRASIDDNVPDSEKDLLTLMLEAELRGEGTASDEQLRSNLAVFFLAGHETTANTMSFCLYNLAMNKEVQSKARQEVLTVLGDEPVDILPQIDELRQMPYIDMVLKENLRRFGPASMLIARKSQEDFDLNGVFIPKNTPVVVETHALHHNPDVWKNPEQFDPERFAPGGEHETCHEGMAWLPFSSGSRGCLGMNFSLAEQRTFLVMLLRKYEWELPEDSIHRNGVKIQNFQNAAPESLEIKFSSRY

SEQ ID NO: 60 - DNA, 1560 bp, Thamnostylum piriforme
  source 1..1560: mol_type = other DNA
atgatttcttatccagttgttagagatagattgtctagatataaatattatattgttattggtgctttgttttctattgcttggcaacaaatttattatagattttttagagttccaagaaatttgagacatattccatctattgcttatgctcaacaattgttggctttgttgagaggtgaaccaattactttgagaactcaaagattggtttttccattgttgtctaaagctaatggtatttatttgaatagagcaccatttttgtggactatttttgttgctaatccagttttggctaaagaagttttgtttaaatctgaattggctaataaaaatactattttggatgcttttccaaaagattctgcttttgttgaatttgttggtaaagataatgttgctttttctaatggtgaaaaatggaaaagacaaagaaaaattatgaatccaatttttcatagagctactactgctgctgcttttgcttctgttactttgaaattgtttagagttattgatgatactcaagctgctggttctcatgttgctgttccaattttgatgaaaagattggctttggaagcattgggtagaactatgtttggttttgattttggtggtttggaagatgaaggttctgaatgggttgctgcttgtaatgaagtttttacttctttttctgattttacttctttgtctgctagagctgatgctatgttgagagttttttctagatctagacaagaaaaatataaagctagatttagattgattaatatgtttgatgatatgattgaacaaagaagagttactttgttggatactaaagaaacttcttctactaatgttccagataatgaaaaagatttgttgactttgttgttggaagctgaaatgagaggtgaaggtacttggagaaaaaatgaattgagatataatattgctactttgttttttgctggtcatgatactactgcttctactttgtcttttgctttgtattctttggctgttaataaagaagttcaatctaaagctagagaagaagttaatgatttgttgggtaataatcataatgatgttgctccaactttggaacaatgtaaacaattgccatatattgatatgattattaaagaaactttgagaatgtattctccatctgatcatttgtttgctagagttgcttctgatgatttggaattgggtggtgttttgattccaaaaggtgctaaagtttctgttgattttcatgctttgcattatcatccagatttgtgggaagctccagaaagatttattccagaaagattttctgattctggtgaacattctaaacatgaaggtattacttgggctccattttctggtggttctagacaatgtattggtttgaatttttctatgactgaacaaagagttgctttgtctatgttgttgagaaaatatgaatgggaattgccagcaggttctattcatgaaaaaggtttggttatggatcaaccatataattttgctccaatgtctttggaattgagatttaaaaaaatttattag

SEQ ID NO: 61 - protein, 519 aa, Thamnostylum piriforme
MISYPVVRDRLSRYKYYIVIGALFSIAWQQIYYRFFRVPRNLRHIPSIAYAQQLLALLRGEPITLRTQRLVFPLLSKANGIYLNRAPFLWTIFVANPVLAKEVLFKSELANKNTILDAFPKDSAFVEFVGKDNVAFSNGEKWKRQRKIMNPIFHRATTAAAFASVTLKLFRVIDDTQAAGSHVAVPILMKRLALEALGRTMFGFDFGGLEDEGSEWVAACNEVFTSFSDFTSLSARADAMLRVFSRSRQEKYKARFRLINMFDDMIEQRRVTLLDTKETSSTNVPDNEKDLLTLLLEAEMRGEGTWRKNELRYNIATLFFAGHDTTASTLSFALYSLAVNKEVQSKAREEVNDLLGNNHNDVAPTLEQCKQLPYIDMIIKETLRMYSPSDHLFARVASDDLELGGVLIPKGAKVSVDFHALHYHPDLWEAPERFIPERFSDSGEHSKHEGITWAPFSGGSRQCIGLNFSMTEQRVALSMLLRKYEWELPAGSIHEKGLVMDQPYNFAPMSLELRFKKIY

SEQ ID NO: 62 - DNA, 1650 bp, Cunninghamella echinulata
  source 1..1650: mol_type = other DNA
atgtctcaatttatttctactatttctcaatattgggatgttaataattttattgatcaacaagcatggtctagattttatgaaacttatttgttgaaatatactgattctaaagctaaaagattgactattggtgtttctgtttctttgttgttgttgggttatggtattaaaagattgttgactccaccaaaacatttgagacatattaaacatgcttcttttttgagatttacttataattttgttattaaaggtgttactccaaaagaaatgcatactactgtttctaaaaaaattgttgaagaaaataatggtttttatttgcaattggaaagaactggttgggttgtttatgttgctaatccagaagctgttaaacaagttttgttgaaatctgatatttttccaaaaatggatttttctaaattggcttctgaagaaggttcttattttgaaaaatttattggttttaaaaatattttgatgactactggttctgaatggttgaaacatagaaaattgactaatccagcttttcatagatctttgccagctaaattgtttggtgaaactgctcaaggtttgtttaaaatttgggataatgaatataatgataaaccatttgaagttaatattcataatatgaatgaaagaattactttggatattattggtaaagctggttttgcttttgattttaatgctgttgctgatgaaaaatctccttggaaaaaaacttatgatattattaatactgctgcttctgatccattgttttttatgtttccaattttggaaaataaattgttgtggttgtttccaaaaagacaagaatcttttaaaaaattggatgaatggaaagctatgttgacttctgttattgaaaataaaagaagattgttgaaagataatattgatcaaggtgttgaagaagctgaaaaagatttgttgactttgatgattgaatctgaatttagaggtgaaggtgttttgactaaagaagaattgattggtaatttgactgttttttttttggctggtcatgatactacttcttttgctttgacttctgctatttattatatggctagatacccagaaattcaagaaaaagctagacaagaagttaattctattttgtgtccaaatggtgaaccaaatgaaggtattttgccaactattgaagatactaaaaaattggtttatttgaatcaaattatgaaagaatctatgagaattggtaatccagttttgtttttgttgtctccaagacaagctactaaagattttaatttgaatggtacttttattccaaaaggtactcaagttaatattaatattcatgatttgcatcattcttctaatgtttgggatgatccagatacttttaatccagatagatttgctccaggtggtgaagctgataaaaaaactggtattgcttgggttccattttctaatggttctagacaatgtttgggtatgaatttttctttgttggaacaaagagttattttgtcttgtttgttgagaaaatatgaatggactttgccagaaaattctattcataaaaatgatatgatttctaaatttgatttgattccatctccaattgatatgaaaattagatttgaaagaagatattaa

SEQ ID NO: 63 - protein, 549 aa, Cunninghamella echinulata
MSQFISTISQYWDVNNFIDQQAWSRFYETYLLKYTDSKAKRLTIGVSVSLLLLGYGIKRLLTPPKHLRHIKHASFLRFTYNFVIKGVTPKEMHTTVSKKIVEENNGFYLQLERTGWVVYVANPEAVKQVLLKSDIFPKMDFSKLASEEGSYFEKFIGFKNILMTTGSEWLKHRKLTNPAFHRSLPAKLFGETAQGLFKIWDNEYNDKPFEVNIHNMNERITLDIIGKAGFAFDFNAVADEKSPWKKTYDIINTAASDPLFFMFPILENKLLWLFPKRQESFKKLDEWKAMLTSVIENKRRLLKDNIDQGVEEAEKDLLTLMIESEFRGEGVLTKEELIGNLTVFFLAGHDTTSFALTSAIYYMARYPEIQEKARQEVNSILCPNGEPNEGILPTIEDTKKLVYLNQIMKESMRIGNPVLFLLSPRQATKDFNLNGTFIPKGTQVNINIHDLHHSSNVWDDPDTFNPDRFAPGGEADKKTGIAWVPFSNGSRQCLGMNFSLLEQRVILSCLLRKYEWTLPENSIHKNDMISKFDLIPSPIDMKIRFERRY

SEQ ID NO: 64 - DNA, 1392 bp, Rhizopus delemar
  source 1..1392: mol_type = other DNA
atggtcaagtccttcttgaaggctgaatctccaactgatagatacaagagaatcattcatccagtcgcctctaaaggtaatggtttctacgtttctaaggtcccatttttgtgggctgtttacgttactaatccagttgttgctaagcaggtcttgttgaagtctgatattttcccaaagaaccatgccgttttccatcaaattggtaaggattctccattcactcagttcttgggtttagataacgttgctttgtctaacggtgatgtctggaaaaagcaaagaaaggttatgaacccagccttccatagatctttgccaatcaaaactatggcctctgttgttaacaccttgttctccgttattgataactctgatggtccagttttgatcacttctgctatgcaaaacttcaccttggatgttttaggtttggccattttcggtttcgaattcaaggctttacaaggtgatccagataactggactaagacttacaagaccgttatcgaatctttgttcgatccagtcatgaacgttttcgctactttcgatactttgttgacatgggtttaccccaagagaagagaatgttctgttgccatttctaagatgaacttgaagttcgatgaactggccaaacaaaaaagagccgaagttaagtctggtgcttatgctaatgttccagatcacgataaggatctgttgaccttgatgttggaagctatggaaaaaggtgaagccttgacttctcaagacgaattgagacataacattgccgtttttttcttggctggtcatggtactactgctcatactttgtctttctgcttttaccatctggctaagaacaagcacatccagagaaaattgagggaagaaatcatctccgttttgggtgatgaaccagttgatatagttccatccttggaacaaatgaagcacatgaagtatatgaacctggtcatcaaagaaaacctgagaatgaatactccagccgataccttgtttactagagatactgttgaggatattaacttggccggtcatatcattccaaaggataccgctatttccatcgatattaacgccattcatcacgatccaaagtactggcataatccagaacatttcatcccagaaagatttgctgaaggtggtgaacaagaatctcatgaaggtttgacttggttgccattctcaaatggttctagacaatgtatcggcatgaacttctcattggctgaacagagattggttttggctatgttggttaggaagtacgaaatcgatatcccaaaggattccatccactacgaaagaatcttgtttgacagaccaggtaacattgctccattgtctttgggtttgaccttcaccaagagatattaa

SEQ ID NO: 65 - protein, 463 aa, Rhizopus delemar
MVKSFLKAESPTDRYKRIIHPVASKGNGFYVSKVPFLWAVYVTNPVVAKQVLLKSDIFPKNHAVFHQIGKDSPFTQFLGLDNVALSNGDVWKKQRKVMNPAFHRSLPIKTMASVVNTLFSVIDNSDGPVLITSAMQNFTLDVLGLAIFGFEFKALQGDPDNWTKTYKTVIESLFDPVMNVFATFDTLLTWVYPKRRECSVAISKMNLKFDELAKQKRAEVKSGAYANVPDHDKDLLTLMLEAMEKGEALTSQDELRHNIAVFFLAGHGTTAHTLSFCFYHLAKNKHIQRKLREEIISVLGDEPVDIVPSLEQMKHMKYMNLVIKENLRMNTPADTLFTRDTVEDINLAGHIIPKDTAISIDINAIHHDPKYWHNPEHFIPERFAEGGEQESHEGLTWLPFSNGSRQCIGMNFSLAEQRLVLAMLVRKYEIDIPKDSIHYERILFDRPGNIAPLSLGLTFTKRY

SEQ ID NO: 66 - DNA, 1584 bp, Rhizopus microsporus
  source 1..1584: mol_type = other DNA
atggaattgaccgaaatcgccatcgaaacttatcatagagctttggataagctggtcccaatcttgcaaaaaagatccaagagatcctacattggtgttgctgttgctttggttgttttggaaagaatctacagcttcttcagagtcccaaagtccattagacatattccagctgttccatactttgctatggctaagtctttcttgacttctgaagctccatcttccagacatcaaagaatagttttgccagtcatcgagaaaggtaatggtatctacgttaacaagttgccattggaatggactgttaatgttgctactccaacctctgctaaacacgttttgttgaagtctgaaatctaccccaagtccgaatcctttttgaaattattgggtccacaatctccagccgttttgtttttaggtggtaagaatgttggtttcgttaacggtgatgcttggagaaagcaaagaaagattatgaacccagccttccatagatccatgccaattcaaactatggcttctgttatgccagatttcttctcagccattgataagtatggtgatgaaggtattccaatctccaccatcatgagagatttcaccttggatgttttgggtcatactgcttttggtttcgatttcaaggctttgaaaggtgatccagataactggactagaacctaccatatcattaacaacgctttgttcaacccaaccgctaatatgttgacttctttgaaccctatcctgtccattatctctccagaaagaagaagaattttggaggccatcaaaaagttgaacggtatgttggaagccatgatcaagcaaaagagacaagaagttcaatccaacgctcaagctcatgctccagaaaacgaaaaagatttgttgaccttgatgttggaggctcaacaaagaggtgaaggtttggctactgatgaagaattgaagcacaatgtctctggttttttcttggctggtcatgatacaacctctgaaactttgtctttctacttctacaacattgccaagaacaaggacgtccaaagaaagttgagagaagagttgaatgctgtcttgggtgataagccagttgatgttattccaaccttggagcaattgaagtccatggaatatttgaactgcaccatcaaagaaaacttgaggttgaatggtccagccgataatgttttgccaagagttgctactgaagatatggttgttgatggtactccaattccaaagggtactgttgtgaacgttgatattcatgccattcatcacgataccagatactggaaagatccatacaagtttgtcccagaaagatttttgccaggtggtgaacatgattctcattctggtatgacttggttgccttttggtaatggtgctagacaatgtttgggtatgaatttctcattggccgaacagagattggttattgctatgactgttaggaagtacgatatcgaagttccaaaggattccatccattacgatcatccaatcctggaatcttctaaaacaaaagctccagcctccttgaagttgatcttcagaaagagatactga

SEQ ID NO: 67 - protein, 527 aa, Rhizopus microsporus
MELTEIAIETYHRALDKLVPILQKRSKRSYIGVAVALVVLERIYSFFRVPKSIRHIPAVPYFAMAKSFLTSEAPSSRHQRIVLPVIEKGNGIYVNKLPLEWTVNVATPTSAKHVLLKSEIYPKSESFLKLLGPQSPAVLFLGGKNVGFVNGDAWRKQRKIMNPAFHRSMPIQTMASVMPDFFSAIDKYGDEGIPISTIMRDFTLDVLGHTAFGFDFKALKGDPDNWTRTYHIINNALFNPTANMLTSLNPILSIISPERRRILEAIKKLNGMLEAMIKQKRQEVQSNAQAHAPENEKDLLTLMLEAQQRGEGLATDEELKHNVSGFFLAGHDTTSETLSFYFYNIAKNKDVQRKLREELNAVLGDKPVDVIPTLEQLKSMEYLNCTIKENLRLNGPADNVLPRVATEDMVVDGTPIPKGTVVNVDIHAIHHDTRYWKDPYKFVPERFLPGGEHDSHSGMTWLPFGNGARQCLGMNFSLAEQRLVIAMTVRKYDIEVPKDSIHYDHPILESSKTKAPASLKLIFRKRY

SEQ ID NO: 68 - DNA, 1488 bp, Parasitella parasitica
  source 1..1488: mol_type = other DNA
atggccgttttgtgcttggtcttggaaagaatctatgcttcttatgctgttccaccatcctacttgagacattacccaaaggtttctttgttcgacatgctgaagtccttctacatcaaagaatctgttgcctccagaaacaagagattgattgctccattgactaatgctggtcatggtttttacgtctgcagaattccattgaactggactatctatgttaccgatccattggctgctaagactttgttgttgaagtctgaaaacttcccaaagaacaaggctattttcactgctttgggtgattcttcaccagtcattaagttcatgggtaaagaaaacgttgccatgtctaatggtgaagagtggaaaaagcaacgtaagattatgaacccagccttccatagatctcaaccagttaagacttttggtaacgttatgccagatttgttcgccttgattgatcaagatccagaaagagttttcatcacgccaaagatgaagtcttttgctttggatgctttaggtttgtctgctttcggtttcgatttccaatctttgaaaggtgatccagaaggttggactagaaagtacaatgttgttatctccaccttgttcaaccccttcgttaatatctttgcctccttggatttcctggtcaagtatatttcaccagaaagaagaagagttatcaaggccactgatgagttcaattctatgttgggtgaattggcagataagagaaggcaagaaattttggacggtgagaaaaagaacatcccagaaaacgaaaaggacctgttgaccttgatgattgaagccgatattagagacaacattaagactactaccaccgaattgagacataacatggccattttctttttggccggtcatgattctacagctaacactttgtctctgtgcttgtacaatttggctaaacacaagcacgttcaaaagaaggctagacaagaagttttggctatcttgggtgatgaaccattggatgttattccaactgctgaggacttgaagaagttggattacgttaacatggtcatcaaagaaaacctgagaagaaatggtccagccgataatttgatgtctagagatactcaacaggacatgaacttgaacggtactattattccaaagggctccaaggtttctgttaacgttgctgctattcatctgaacccaaagatttggcatgacccagaaaatttcattccagaacgttttgagcaaggtggtgaattcgaacaacatgatggttttacttggatcccattctctaacggttctagacaatgtttgggtctgaacttctcattgactgaacaaagggttgttctgtgcatgatcttgaagagatacgaaatcgatatccccaaggattccatccattacaacgaaatcgttttcgatggtgcttttactttcgctccacaatctttggaactgtccttcaagagaagatactga

SEQ ID NO: 69 - protein, 495 aa, Parasitella parasitica
MAVLCLVLERIYASYAVPPSYLRHYPKVSLFDMLKSFYIKESVASRNKRLIAPLTNAGHGFYVCRIPLNWTIYVTDPLAAKTLLLKSENFPKNKAIFTALGDSSPVIKFMGKENVAMSNGEEWKKQRKIMNPAFHRSQPVKTFGNVMPDLFALIDQDPERVFITPKMKSFALDALGLSAFGFDFQSLKGDPEGWTRKYNVVISTLFNPFVNIFASLDFLVKYISPERRRVIKATDEFNSMLGELADKRRQEILDGEKKNIPENEKDLLTLMIEADIRDNIKTTTTELRHNMAIFFLAGHDSTANTLSLCLYNLAKHKHVQKKARQEVLAILGDEPLDVIPTAEDLKKLDYVNMVIKENLRRNGPADNLMSRDTQQDMNLNGTIIPKGSKVSVNVAAIHLNPKIWHDPENFIPERFEQGGEFEQHDGFTWIPFSNGSRQCLGLNFSLTEQRVVLCMILKRYEIDIPKDSIHYNEIVFDGAFTFAPQSLELSFKRRY

SEQ ID NO: 70 - DNA, 1545 bp, Rhizopus delemar
  source 1..1545: mol_type = other DNA
atggctttggaaggtgctttgcctttgttccaaaagagatccaaatcctcttatatcgttgccgccattttgttgattaccgtcaaacaaatctacagcttcttcagagttcctaccaacttgagacatttgccatgtgtttcttttttcgccatggccaaatctttgttgacttgtgaaccaccatacaacagattcaagagaattactttcccagccatccaagaaggtaacggtttttacgtttctaagattccaactggttggaccgtttatgttgctaatccagttgctgctaaacagctgctattgaagtctaacaatttccccaaatctcactacggtttggataccattggtgaaaaatctaccgctgttcaattcgttggtagagataacgttgttttgtccaacggtgaaatctggaaaaagcagagaaagatcatgaacccagttttccatagatccatgccaatcaaaactgttgcttctttggttcccttgttgttctctgcaattgaagaagcaaacggcagaattatgattacgccaaagatgaaggatttcactttggatgctttgggtttgaccatcttcgattttgattttaaagccttgcagggtgatccagataattggacttctatctacagattgatcaccaggtctatcttcgatccaatctcttacgttttctgtgctttggaacctttgttggtttacgtttacccaaagcgtagaagatctgttgatgctgttgctaagattaacgccaagttcgatcaagtcatctccaaaaaaagggaagaattgcagaacggcatcttctctaacaaaccagataacgaaaaggacttggtcaccttgatgttggaagctggtatgcaagaagatgtttctatcaccaacgaagaattgagacataacatggccgttttgtttttggctggtcatgattctacttccaacactttgtctttctgcttgtaccatttggctaaaaacaagagagcccaacagaagttgagagaggaaattatcaacatcttgggtgatgatgatatcgacatcgttccatccttggaagagttgaaacaaatgaagtacatgaacatggtcatcaaagaaaccttgagattgggtatgccattggatttgttgaccccaagaaaaacagttgaagatacctttgtcgccgatacctttattccaaaggataccgttattgctgttgacgctggtgcattgcatagagatcctagatcttggaaagatccagacgaatttgtcccagaaagattcgaagatgatggtgaacaaaactcccatgaaggtttgacttgggttccattttctaacggtactagacaatgtatcggcatgaacttctcattgatggaacagagattgaccctgactatgttgttgagaaagtacgaagttgatctgccaaaggattccatccattacgatcatatcatctacgaacaaccatcctacgtttgtccagaatctttggaattgatcttcaccaagaggtactaa

SEQ ID NO: 71 - protein, 514 aa, Rhizopus delemar
MALEGALPLFQKRSKSSYIVAAILLITVKQIYSFFRVPTNLRHLPCVSFFAMAKSLLTCEPPYNRFKRITFPAIQEGNGFYVSKIPTGWTVYVANPVAAKQLLLKSNNFPKSHYGLDTIGEKSTAVQFVGRDNVVLSNGEIWKKQRKIMNPVFHRSMPIKTVASLVPLLFSAIEEANGRIMITPKMKDFTLDALGLTIFDFDFKALQGDPDNWTSIYRLITRSIFDPISYVFCALEPLLVYVYPKRRRSVDAVAKINAKFDQVISKKREELQNGIFSNKPDNEKDLVTLMLEAGMQEDVSITNEELRHNMAVLFLAGHDSTSNTLSFCLYHLAKNKRAQQKLREEIINILGDDDIDIVPSLEELKQMKYMNMVIKETLRLGMPLDLLTPRKTVEDTFVADTFIPKDTVIAVDAGALHRDPRSWKDPDEFVPERFEDDGEQNSHEGLTWVPFSNGTRQCIGMNFSLMEQRLTLTMLLRKYEVDLPKDSIHYDHIIYEQPSYVCPESLELIFTKRY

SEQ ID NO: 72 - DNA, 1569 bp, Rhizopus microsporus
  source 1..1569: mol_type = other DNA
atggaccacttgatccaggtctacaactcttctactcaagttttgattccagtcttgcagaagagatctaaggcttcttatattaccgctgccattgctttgattattgcccaaagactgtactcctacttcagagttccaaaacatttgagaggtttcccaaagttgccatactttggtattgctaagtcattcttcgccaaagaatctccaagagaaagagtcaagaagtacatcttgccaatcatcaacgaaagagatggcttctacattagcaacattccatttggttggatgctgtacgttaccaatccaattgctgctaagcaaatcctgttgaagtctaacggttttccaaaaaaccacggtttgctagaagatatgggtgagaatttgttcatcgagttcatcggtaaggataacgttgttttgactaacggtgatacctggaagagacaaagaaaggttatgaatccagccttccatcattccttgcctattaagactatgtccaacgtcgttttctccttgatctccgttattgatcaagctaatggtactgttccagttgcttctactatgcaaaacttcaccttggatactttgggtttagccattttcggttttgactttaaggcattgcaaggtgatggtgatgaatggactaagacttacagattggtttccgattgcttgttcgacccaattatcaacgttttcagctcctactccttcatcttcgatagaatctacccaagacgtagaagaggtgctatggctactagaaaattgggtgaaaagttcttggaaatcgcccagcaaaaaaggatggaaatcaaatctggtgctttcgctgatgttccagataacgaaaaagatctgttgaccttgatgttggaagctgaagaaaaaggtgatgtctggacttctgaagatgaattgagacataacattgccgttttgttcttggctggtcatgatacaactgctcatgctttgtctttctgcttttaccatttggctaagaacaaggacatccagcagaagttgagaaaagaagttttggatttgttgggtgatgaaccagttgatgttgttccaactgttgaacaattgaaggacatgcagtacttgaacatggtcatcaaagaaaacctgaggatgaattctccagccgatatgttgttttccagagatgttcaagaggatatcgttttggctaacacctttattccaaagggtactgtcatctccattaacattgaagccttgcattgcaatccaaagttgtggcataatccagatcaattcgatccagaaagatttgctccaggtggtgaacatgaacaacatgaaggtatgacttggttgccattttctaacggtactagacaatgtctgggtatgaacttcagcttgtttgaacagagattggttatcgccatgatcttgaagaagtacgaaatctccattccagaggattccatccatagaaaccacatcattaacgacatgccattcaatgttgctcccaagtctttggaattgactttcactaagaggtactaa

SEQ ID NO: 73 - protein, 522 aa, Rhizopus microsporus
MDHLIQVYNSSTQVLIPVLQKRSKASYITAAIALIIAQRLYSYFRVPKHLRGFPKLPYFGIAKSFFAKESPRERVKKYILPIINERDGFYISNIPFGWMLYVTNPIAAKQILLKSNGFPKNHGLLEDMGENLFIEFIGKDNVVLTNGDTWKRQRKVMNPAFHHSLPIKTMSNVVFSLISVIDQANGTVPVASTMQNFTLDTLGLAIFGFDFKALQGDGDEWTKTYRLVSDCLFDPIINVFSSYSFIFDRIYPRRRRGAMATRKLGEKFLEIAQQKRMEIKSGAFADVPDNEKDLLTLMLEAEEKGDVWTSEDELRHNIAVLFLAGHDTTAHALSFCFYHLAKNKDIQQKLRKEVLDLLGDEPVDVVPTVEQLKDMQYLNMVIKENLRMNSPADMLFSRDVQEDIVLANTFIPKGTVISINIEALHCNPKLWHNPDQFDPERFAPGGEHEQHEGMTWLPFSNGTRQCLGMNFSLFEQRLVIAMILKKYEISIPEDSIHRNHIINDMPFNVAPKSLELTFTKRY

SEQ ID NO: 74 - DNA, 1452 bp, Syncephalastrum racemosum
  source 1..1452: mol_type = other DNA
atgttcagaccaccagcaggtttgccaaaattgccaattatcaattacttcaggttgctgtgggccttgtacagaaaagaatctccaactagaagatcccagagattgattttgccagccttgcaaaaacataacgctaaagcttacttggctaaggttccatatacttggactgtttacttggttgatccagttgctgttaagaccttcttgatgagaaatggtactttcccaaagaccatggaatccattgatgctttcgatcaatctcacccaaatgttcaattttggggtagagaaaacttgggtttctctgttggtgattcttggaagagacaacgtaagattatctttccagcttttagaagggctatgccagttcaattattcggtgaattggttccaaagatgttcgacttgatcgacaaagaacattccaccattgccatcgatttgatgcaaagattcaccttggatgctttgggtttagctgctttttctttcgatttccatgccttggataaccacaacaatgaatgggaagttgcctacgagatgatcagaaaagagttggtttctccactgactaacatgttggctagatacgattacatcctgaagtacattattccaggtagagctgaaaaacaagctgccgtttctaagatcaaccacttgttgtctgatattgccaacgagagaagaaagatgattaagaggaatcccgatatgttgaaggttccagattctgaaaaggacttgttgaccttgatgttggaatccgatttggaaaactctgatgatccagcttccgaagaattgattagagctaatttggctaccttcttcttggctggtcatgatacaactgctaacactttgtctttctggttgtaccatttggccatgtacaaggatattcaaaagaaggctagagaagaggtcattgaaactatgggtggtgctccagaagatatagttccaactgctgaacagctgaagaaaatgaagtacatggactgcatcatcaaagaaaacctgagattcatgggtccagctttggaattatttccaaggattgccaaagaggactacaacttgaacggtattttcatcccaaagggtactagagtttctgttgacttgcataccttacatcatcatccagatgtttggaaagaaccagagagatttgatccattgagattcgttgaaaacggcgaacattctaaacacgaaggtttgtcttggattgctttttcatctggtgctagagtatgcatcggtcaaaatttctcattggttgaacaaagggtcgtcatgtctatgttgttgagaagatacgaatgggacatcccagaagattccattcatagagaaggcttgcaattgaaggacaccaacaatcaagctccaagatcattggaaatcgtgttcaagaagaggtactaa

SEQ ID NO: 75 - protein, 483 aa, Syncephalastrum racemosum
MFRPPAGLPKLPIINYFRLLWALYRKESPTRRSQRLILPALQKHNAKAYLAKVPYTWTVYLVDPVAVKTFLMRNGTFPKTMESIDAFDQSHPNVQFWGRENLGFSVGDSWKRQRKIIFPAFRRAMPVQLFGELVPKMFDLIDKEHSTIAIDLMQRFTLDALGLAAFSFDFHALDNHNNEWEVAYEMIRKELVSPLTNMLARYDYILKYIIPGRAEKQAAVSKINHLLSDIANERRKMIKRNPDMLKVPDSEKDLLTLMLESDLENSDDPASEELIRANLATFFLAGHDTTANTLSFWLYHLAMYKDIQKKAREEVIETMGGAPEDIVPTAEQLKKMKYMDCIIKENLRFMGPALELFPRIAKEDYNLNGIFIPKGTRVSVDLHTLHHHPDVWKEPERFDPLRFVENGEHSKHEGLSWIAFSSGARVCIGQNFSLVEQRVVMSMLLRRYEWDIPEDSIHREGLQLKDTNNQAPRSLEIVFKKRY

SEQ ID NO: 76 - DNA, 1485 bp, Parasitella parasitica
  source 1..1485: mol_type = other DNA
atggccatcgaaaacttcatgcagtcttacaacagagccattgaaaacgtcttgccaatcttgagaaaaaggtctaaggcttcctatattggtatggccattatgtgcatcgttatcaagcaagtttactccgcttactctgttccaaaacacttgcaaagattccccaaggtttcattcttgtccatgatcagatcctacttgatcaaagaagccgttgtcgaaagaactaagagattggttactccattgactgatgctggtcatggtttttacgtctgtaaaattccattgacctggaccgtttttgttaccgatccaattgctgctaagaccttgttgttgaaaactgagtttttcccaaagtctcacgctttctttgatgctttgggtgataattctccagccgttcaatttttgggtagagaaaatgttgctgcctccaatggtgaaatctggaaaaagcaacgtaaattcatgaaccccgctttcttgagatcttctccagttaagactttctcctctgttacccacaacttgattaaggttatcgaaacccaatctgatgctgttccaattgcaaactgtatgaaggctttcactattgacgctttaggtttgtctgctttcggtttcgatttccaatctttgaatggtgatccagaaggttggactgaaacttacaatattgctattgccggtttgttcgacccattcattaacatctttgttaaggtcgacttcatgatgaactacatctcttctaagaggaagagaatcaacaaggccattaccagattcaactccatgttggaagatttggctaacaagagaaggcaagaaatcttgaacggtgaaactttgggtgttccagaaaacgaaaaggatttgttgaccttgatgatcgaagccgatattagagaaggttccagaactacttctaccgaattgagacataacattgccttgtttttcttggccggtcatgatacaactgctcatactttggctttttgcttgtacaacctggctaaaaacaaacacgttcaagctaaagctagagccgaagttttggatattttaggtgatgaacctaaggatgttgacccaactatggaagatctgaagagaatggattacctgaacatggttatcaaagaaaacttgagaagatgcggtccagttgacaagttgttgtctagagatactgccgaagatatcgatttgaacggtactttgattccaaagggccaaaagatctccatcgatttcaactctatccacatgaatccaaagttgtggcataacccagaagaatttgtcccagaaagatttgaaccaggtggtgaatttgatcaacatactggttttacttggctgccattttctcatggttctagacaatgtatcggcatgaacttttcattgaccgagcaaaaagtcctgttgtctatgatctgtaagagagccatcgaagagatctga

SEQ ID NO: 77 - protein, 494 aa, Parasitella parasitica
MAIENFMQSYNRAIENVLPILRKRSKASYIGMAIMCIVIKQVYSAYSVPKHLQRFPKVSFLSMIRSYLIKEAVVERTKRLVTPLTDAGHGFYVCKIPLTWTVFVTDPIAAKTLLLKTEFFPKSHAFFDALGDNSPAVQFLGRENVAASNGEIWKKQRKFMNPAFLRSSPVKTFSSVTHNLIKVIETQSDAVPIANCMKAFTIDALGLSAFGFDFQSLNGDPEGWTETYNIAIAGLFDPFINIFVKVDFMMNYISSKRKRINKAITRFNSMLEDLANKRRQEILNGETLGVPENEKDLLTLMIEADIREGSRTTSTELRHNIALFFLAGHDTTAHTLAFCLYNLAKNKHVQAKARAEVLDILGDEPKDVDPTMEDLKRMDYLNMVIKENLRRCGPVDKLLSRDTAEDIDLNGTLIPKGQKISIDFNSIHMNPKLWHNPEEFVPERFEPGGEFDQHTGFTWLPFSHGSRQCIGMNFSLTEQKVLLSMICKRAIEEI

SEQ ID NO: 78 - DNA, 1626 bp, Lichtheimia corymbifera
  source 1..1626: mol_type = other DNA
atggctgttgcttctccacagatcaagaacatggttactatggatagattgcaggacatctactctaccgtttctcaacacgttgttgctcatgcttcagctgttacttctagacaaagaaagatttccatttccgctgctgttgcattggttgctttttacactgtttacaaggttgttactccaccagctaacttgagacatattccatctatgggtttcttctcttacttgaacgctttcttgagaggtaagcaattgcacgatatctccaagaatgttgttttgccacatgccgttaatgttgataacggtgtttacttgagattcgacattttaggttggaccgttcatattgctagaccagaagctgctaagagattcttgttgaagtctgatattttcccaaaggccgacatgatttctgaaagaggtaatactttgttcggcaagttcgtgttcaacagaaacatcgttatgttgaacggtgatgattggaaggctcaaagaaaagttgctaatccagctttccatagagctatgccagttgaattattcggtagattgactcaaaagaccctgaaaaagatggaagaagaaatggacggcggtactttgaacttccatgatattacagaaaggtacaccttggaagttattggtttggctggttttgaatttgaattcggtgctatcgaaaacccaaagtctgaatgggttgatagataccagagattgattgaagctactttcaacccatggttcattgcttttccaaacttggacatgagatacaggtttttgttcccatctaggaagagattgcacagagaaatggatgctttcttggacaagatgtcccaagttattactcacaagagagagttgttgaaccaccaaaaaactaccgttccagaatccgaaagagatatcttgaccttgatgatcgaagctgaaaagaatggtgaaggttctatgaccaatgaagagttgcagaacaacctgctggtttttttcattgctggtcatgatacaacatccttggctttgtcttatgctgcttattacttggctgttaacccagatgttcaaagaaaggctagagaagaagccattagagttttgggtgatgctcctgaagatgttatgccaacagttgaacaaactagacagttgacctacatcaacatggtcatcaaagaaaccttgagaatgtctccaccattggctactattccagttagagaagctagtgaagataccgaattgtgtggtacttttatcccaaagggtacaagaaccttcttggatatctacgaaatccaacataacccaaccgtttggaaagatccagaaacttttaagcccgaaagattcaaaccaggtggtgaagctgaagaattagctggttctggtatgtcttggttgccattttctaatggttccagacaatgtatcggcctgaatttctcattggtagaacaaagggttttcctgccaatgttgctgagaaagtatgaatggcatttgccagaagattccatccacaaagaacgtattcaaacaatgggtttagccgtcattaagccaaaggatttgaagttgactttcaagaagaggtactaa

SEQ ID NO: 79 - protein, 541 aa, Lichtheimia corymbifera
MAVASPQIKNMVTMDRLQDIYSTVSQHVVAHASAVTSRQRKISISAAVALVAFYTVYKVVTPPANLRHIPSMGFFSYLNAFLRGKQLHDISKNVVLPHAVNVDNGVYLRFDILGWTVHIARPEAAKRFLLKSDIFPKADMISERGNTLFGKFVFNRNIVMLNGDDWKAQRKVANPAFHRAMPVELFGRLTQKTLKKMEEEMDGGTLNFHDITERYTLEVIGLAGFEFEFGAIENPKSEWVDRYQRLIEATFNPWFIAFPNLDMRYRFLFPSRKRLHREMDAFLDKMSQVITHKRELLNHQKTTVPESERDILTLMIEAEKNGEGSMTNEELQNNLLVFFIAGHDTTSLALSYAAYYLAVNPDVQRKAREEAIRVLGDAPEDVMPTVEQTRQLTYINMVIKETLRMSPPLATIPVREASEDTELCGTFIPKGTRTFLDIYEIQHNPTVWKDPETFKPERFKPGGEAEELAGSGMSWLPFSNGSRQCIGLNFSLVEQRVFLPMLLRKYEWHLPEDSIHKERIQTMGLAVIKPKDLKLTFKKRY

SEQ ID NO: 80 - DNA, 1578 bp, Choanephora cucurbitarum
  source 1..1578: mol_type = other DNA
atgttcactgaaaccgccttgcaaatctaccatcaaatggctgaaaaggtcttgccaattttgaagagacagccaaagtcatcttatattggtgctgctttggttttcttgttggttgctgaaattagaaggcaactgtccgttccaaaacacttgaaaaagttcccaaccattggtgtgttcaccttgatgaagtctttcatgagaaacgactccgttatcgaaagacacaatagattggttgctccaatcgttaagcaaggtcataagttttacgctgccaagattccatttgactggtctttgtctatagtcgatccagaaattgccaagatcatgttgatgaagagtgatgcttttccaaagtctcaaggtttcactagaaagttgggtgactcttcattgatcgttagattcactggtagagacaacgtgtctatttctaatggtcatgtctggaagaggcagagaaagtttatgaatccagctttcagaagaagcactccaatcaagacttttggtgaattgaccttgaagttcttcgattgcgttgatgaacaaccacaagattttccagctgccattaagttgaagaactacactttggatgctttgggtattgctgcttttgatttcgacttcaagtctttgtcaggtgatccagaaggttggactgaaatctacaacgttattatgaagggtatgttcgacccttgggtttttttgtttggtaagatggaattcatcctgcagtacatcattccatctaagagagaatgcattaagtccgtcgttaagttcaacaagatgttggttgaaatggccgataagagaaggcaagaaattcaaaacggtaagaagttgaacaccccaaactctgaaaaggatctgttgactttgatgatcgaagctgaaatggaagagggtattatgactaccaacgaagaactgagagaaaacattgccttgtttttcttggctggtcaagattctacaggcaactctttgtctttttgcttgtaccatttggccaagaacaagcacgttcaagataagttgagaagggaaatcatgtccgttatgggtgatagagatttggatgcaattccaaccgtcgaagattttaaggatatgccctacttgaacatggtcatcaaagaaaacttgaggttgtctggtccagctgatagatttttggatagagttgttgccgaagatatcgtcttaggtggtgaactaattccaaagggtactttgattaccgttgatgttgcctccattcattacaatccagaatattggcatgaccccgaagttttcattccagaaagatttgaacctaacggtgaattcgatcaacatgctggtgttgcttggttgccattttcaaatggtgctagacaatgtatcggcatgaatttctcattggctgaacaaagggtctttctgactatgttgttgagaagatacgaagtcggtatctccaaggattctatccattacgattccatcgtctacgaacaatcttttactttcgctccatctagcctgactttgaactttactaagctgaactaa

SEQ ID NO: 81 - protein, 525 aa, Choanephora cucurbitarum
MFTETALQIYHQMAEKVLPILKRQPKSSYIGAALVFLLVAEIRRQLSVPKHLKKFPTIGVFTLMKSFMRNDSVIERHNRLVAPIVKQGHKFYAAKIPFDWSLSIVDPEIAKIMLMKSDAFPKSQGFTRKLGDSSLIVRFTGRDNVSISNGHVWKRQRKFMNPAFRRSTPIKTFGELTLKFFDCVDEQPQDFPAAIKLKNYTLDALGIAAFDFDFKSLSGDPEGWTEIYNVIMKGMFDPWVFLFGKMEFILQYIIPSKRECIKSVVKFNKMLVEMADKRRQEIQNGKKLNTPNSEKDLLTLMIEAEMEEGIMTTNEELRENIALFFLAGQDSTGNSLSFCLYHLAKNKHVQDKLRREIMSVMGDRDLDAIPTVEDFKDMPYLNMVIKENLRLSGPADRFLDRVVAEDIVLGGELIPKGTLITVDVASIHYNPEYWHDPEVFIPERFEPNGEFDQHAGVAWLPFSNGARQCIGMNFSLAEQRVFLTMLLRRYEVGISKDSIHYDSIVYEQSFTFAPSSLTLNFTKLN

SEQ ID NO: 82 - DNA, 1530 bp, Absidia repens
  source 1..1530: mol_type = other DNA
atgggcacctcttacaaaagaaaggctgcttatgttactattggtgctgctgttgttttgctgttcagatctatctacggtttctactacgttccaaagtccttgagacatttgccatgtgttggttatagagaattggcctactctatcatgaagagggaagatgctgctactagagctagatctgttttgggtcaagctatcaaagaaaagaacggtgcttttgttgctaagttcccaatggaatggactgtttttttgacttccccaagaaccattcaagccgttttgttgaagtctgacaagtttccaaagacctacgatatgtttcatgccttgggtgaatcttctccattcgttaagtttttgggtatcaagaacgtcggtttctctaatggtgatgattggaagagacaacgtaaggttatgaatccagccttccaaagatctgttccagttgaaatgttcggtaagttgatgcaaaagggtattttggccattgaaaagcaaggttacgaagttagagctttggacttcttcgaaagaatcaccttggattccatcggtattttcttgttctctttcgacttcggttctttggataacccaaattctgtttggtctttgacctacgacactattagaaggggtattaagaatccagctttgaccgtttttccacaattggattgcttgttgaaatacgttactccaggtagaaggcatttggatcaatgtgttactaagctgaacaacctgttgatggaagtcgctaaagaaaaaagaaggcaagtccaatctccatccgataagttgattccagattccgaaaaggatctgttgaccttgattttggaagctgaattgagaggtgatggttctgcttctgatgaagaactaagaggtaatttggccttgttttttttggctggtcacgaaactactgcttccgctatgtgtttttgcttgtacaatttggccatgaacaaggacattcaagagaaggctagaaaagaagtgttggaagttttgggtgatgaaccagttgatgttagaccagatatccaagacttgaagcaattcaggtacatggatatgatcttgcacgaaaacttgagaagatttggtccagctgctatgttgttgccaagaaaatctgaagaggatttcgttgccgatggtattttgattccaaagaataccccagttgtcatcgatttgaacaccatgcatcataatccacaagtttggaaggatccagaaatctttgacccagaaagatttgctcctggtggtgaatatgaagctaacggtgaaaaaatggcttggttgccattttcttcaggttctagagtctgcatcggtaagtctttttctatggctgaacaaagggtgttcctgtcaatgttgttgaaaaagtacgaatgggatttcccagatgactccattcatagagatggcattaagatggaaaacttcgaaaacaacgctcccgaatctttgtcttttagatttcatccaaggtactaa

SEQ ID NO: 83 - protein, 509 aa, Absidia repens
MGTSYKRKAAYVTIGAAVVLLFRSIYGFYYVPKSLRHLPCVGYRELAYSIMKREDAATRARSVLGQAIKEKNGAFVAKFPMEWTVFLTSPRTIQAVLLKSDKFPKTYDMFHALGESSPFVKFLGIKNVGFSNGDDWKRQRKVMNPAFQRSVPVEMFGKLMQKGILAIEKQGYEVRALDFFERITLDSIGIFLFSFDFGSLDNPNSVWSLTYDTIRRGIKNPALTVFPQLDCLLKYVTPGRRHLDQCVTKLNNLLMEVAKEKRRQVQSPSDKLIPDSEKDLLTLILEAELRGDGSASDEELRGNLALFFLAGHETTASAMCFCLYNLAMNKDIQEKARKEVLEVLGDEPVDVRPDIQDLKQFRYMDMILHENLRRFGPAAMLLPRKSEEDFVADGILIPKNTPVVIDLNTMHHNPQVWKDPEIFDPERFAPGGEYEANGEKMAWLPFSSGSRVCIGKSFSMAEQRVFLSMLLKKYEWDFPDDSIHRDGIKMENFENNAPESLSFRFHPRY

SEQ ID NO: 84 - DNA, 1620 bp, Absidia glauca
  source 1..1620: mol_type = other DNA
atgccaatcgttaacgccttgcaaacctattcttatgaagaacctatcaacttcttgaaggacgtttggttgaacagattatttccagctggtacgtctaacagaaaaaaggctgctttgttgactttgggtgctactattgctttggtatgtagaactgtttacaggttgtacagagtcccaagatccttgagacatattccagctgttggttatgttgccatgttgagatctattttgaagagggaagatgctactactagagccatgactatttttcaaccagccatgaagaaaggtaacggtgtttttttggttaacttcccattggagtggtctatctatgttgctgaaccattggctgctaaagctgttttgatgaagtctgacaatttccccaagtccatcgatttcattcatgccttgggtaaagaaaacccagttgttaagtttttcggtactgataacgttgccttggttaatggtgaacaatggaagagacaacgtaaggttatgaatccagctttccatagagctatgccagttcaaatgttcggtaggttgttgcaaaagggtttcaagaacattgaagaacaaggtcatcaagttgctgccttggattttttccaaagattgacattggatgctttgggtcatgctgtttttggttttgattttggtgccttgaacgatagagaagctgtttggactgttacctacgaatccattagattgaacttgagaaacccattggctttcgcttttccatctatggattggttgttgaagtacgttattccaggtagattgcaaatggctgcttcagttgataagttgaacggtttgatgatggacatgatcaaagagaagaggatcaagttgttggaatcccaagctcaagatgatactccagaaaacgaaaaggatctgttgaccttgatgttggaagctgaacaaagaggtgaaggtactaccaatgatgaagaattgagatccaatatggccgtttttttcttggctggtcacgaaactactgctaacactatgtctttttgcttgtacaacttggccatggataagtccgttcaagaaaaagctagacaagaggttatcaaggttttgggtgatgaaccagaagatgttttgccaagaaacgaagagttgagacaaatcccatacctggatatgatcctgaaagaaaacttgcgtagatttggtccagcttctatgttgaatgctagaaagactgttgaggacttcgatatgaacggtacttttatcccaaagaacacctccgttatcgttgaaactaatgccttgcatcataacccagaagtttggaagaatccagaacaattcgatccagaaagatttgctccaggtggtgaacacgaaacttctcatgaaggtatggcttggttgccattttcttcaggttctagaggttgtttgggcatgaatttttcaatggcagaacagagggttttcctggctatgttgttgagaaagtatgaatgggacttgccaaaggattccatccataagaacggtattcaaatcggtaacttccaaaacgcttgtccagattctttgaagattcaattcaccccaaggtactaa

SEQ ID NO: 85 - protein, 539 aa, Absidia glauca
MPIVNALQTYSYEEPINFLKDVWLNRLFPAGTSNRKKAALLTLGATIALVCRTVYRLYRVPRSLRHIPAVGYVAMLRSILKREDATTRAMTIFQPAMKKGNGVFLVNFPLEWSIYVAEPLAAKAVLMKSDNFPKSIDFIHALGKENPVVKFFGTDNVALVNGEQWKRQRKVMNPAFHRAMPVQMFGRLLQKGFKNIEEQGHQVAALDFFQRLTLDALGHAVFGFDFGALNDREAVWTVTYESIRLNLRNPLAFAFPSMDWLLKYVIPGRLQMAASVDKLNGLMMDMIKEKRIKLLESQAQDDTPENEKDLLTLMLEAEQRGEGTTNDEELRSNMAVFFLAGHETTANTMSFCLYNLAMDKSVQEKARQEVIKVLGDEPEDVLPRNEELRQIPYLDMILKENLRRFGPASMLNARKTVEDFDMNGTFIPKNTSVIVETNALHHNPEVWKNPEQFDPERFAPGGEHETSHEGMAWLPFSSGSRGCLGMNFSMAEQRVFLAMLLRKYEWDLPKDSIHKNGIQIGNFQNACPDSLKIQFTPRY

SEQ ID NO: 86 - DNA, 1584 bp, Rhizopus microsporus
  source 1..1584: mol_type = other DNA
atggccttgaccgaaattgccattgaaacttatcatagagccttggataagctggttccaatcttgcaaaagagatctaagcactcctatattggtgttgctgttgttttggttgtcttggaaagaatctacagcttcttcagagttccaaagtccattagacatattccagccgtttcttactttgctatggctaagtctttgttgactgctgaagctccatcttctagatacaagagaatagttttgcccgtcgtcaaaaaaggtaatggcttgtacgtttctaagttgccattggaatggactgttcatgttgctactccaatgtctgctaaacacgttttgttgaagtctgagatctacccaaagtccgaatctttcttgaaattgctaggtccacaatctccagctgttttgtttttgggtgcttctaatgttggtttcgttaacggtcatatttggaagaaccagcgtaagattatgaacccagcttttcatagatccatgccaattcaaactatggcctctgttatgccagatttcttttccgctattgacaaacatggtactgatggtacaccaatttctgccgttatgagagatttcaccttggatgttttgggtcatactgcttttggtttcgatttcaaggctttgaaaggtgatccagatcattggactagaacctaccatattatcaacaacgctttgttcaacccaaccgctaatatgttgacttctttgaacccaatcctgtccattatctctccagaaagaagaagaattttggaggccatcaaaaagttgaacggtatgttggaagccatgatcaaacagaaaagacaagaagttcaaaacaacgcccaagccaatattccagaaaacgaaaaagatctgctgaccttgatgttagaagcccaacaaagaggtgaaggtttggctactgatgaagaattgaagcacaatgtctctggttttttcttggctggtcatgatacaacctctgaaactttgtctttctacttctacaacattgccaagaacaaggatgcccaaagaaagttgagagaagaattgtctactatcttgggtgataagccagttgatgttattccaaccttggagcaattgaagtccatggaatatttgaactgcaccatcaaagaaaacttgagattgaatggtccagccgataacattttgcctagaattgctactgaagatatggttgttgacggtactccaattccaaaaggtactgttgttaacgttgatatccacgccattcatcatgataccagatattggcaagatccctacaagtttgtccctgaaagatttttgccaggtggtgaacatgaatctcattctggtatgacttggttgccttttggtaatggtgctagacaatgtttgggtatgaatttctcattggccgaacagagattggttattgctatgactgttaggaagtacgacatcgaaattccaaaggattccatccattacgatcacccaattctggaatcttcatctacaaaagctccagcctctttgaagttggtttttagaaagaggtactaa

SEQ ID NO: 87 - protein, 527 aa, Rhizopus microsporus
MALTEIAIETYHRALDKLVPILQKRSKHSYIGVAVVLVVLERIYSFFRVPKSIRHIPAVSYFAMAKSLLTAEAPSSRYKRIVLPVVKKGNGLYVSKLPLEWTVHVATPMSAKHVLLKSEIYPKSESFLKLLGPQSPAVLFLGASNVGFVNGHIWKNQRKIMNPAFHRSMPIQTMASVMPDFFSAIDKHGTDGTPISAVMRDFTLDVLGHTAFGFDFKALKGDPDHWTRTYHIINNALFNPTANMLTSLNPILSIISPERRRILEAIKKLNGMLEAMIKQKRQEVQNNAQANIPENEKDLLTLMLEAQQRGEGLATDEELKHNVSGFFLAGHDTTSETLSFYFYNIAKNKDAQRKLREELSTILGDKPVDVIPTLEQLKSMEYLNCTIKENLRLNGPADNILPRIATEDMVVDGTPIPKGTVVNVDIHAIHHDTRYWQDPYKFVPERFLPGGEHESHSGMTWLPFGNGARQCLGMNFSLAEQRLVIAMTVRKYDIEIPKDSIHYDHPILESSSTKAPASLKLVFRKRY

SEQ ID NO: 88 - DNA, 1590 bp, Syncephalastrum racemosum
  source 1..1590: mol_type = other DNA
atgacccaattgactaccccagataccttaggtaagttgttggctatctatagacaaactccagcttcaagaagggcttcttggattggttgggctgttgctgctactgttattgcttctcaactgtacaaaactttggtcccaccatctaatttgccaaacttgccaactattaactacttcaggctgctgtcatctttcttgagaagagattctccaaccatcagatcccaagaaattttgattccagctttggaaaagcacgaacaaaagaaagcttacttggctaagttgccattgacttggactgtttttttggttgatccaattgccgccaagatcttcttgatgaagatggatattttccccaagatcaaagaaaccatcgatgccttggattctcaacatccaatcgttgaatttttcggtagagaaaacgttgccatctctgttggtgaagcttggagaaatcagagaaaggttatgaatccagctttcagacgtgctatgccaatttctattttcggcaacttgatgttcaaggtgttcaagaacatcgaagaaaacaagccagttatcgccatcgatttgatgcaagcttttactttggatgctttgggtagagctgctttctcttttgattttcacgctttggatgaccaacactccatttgggttgaaacctacgaaatcatcagacagtctttgagaaatccagctaatgctgttttggccagatacaacttcatcacgaaatatttgatgccaggtagagctaaacaaaaggctgctactcataagctgaacagcttgttgttggaaatggcttctagaaagagagccgaattggaaaaacatccagaattgagagaattgccagactccgaaaaagatttgttgaccttgatggttgaagccgatatggaatctggtgatgatccaacttctgccgaattattgagagctaacttgtccgtttttttcttggctggtcatgataccactgctaacactttgtcattttggttgtaccatatggccgttaacaaagatgtacaagctaaggctagagaagaggtcatcaagattttaggtgatggtcctgaagatgttatgccaactgctgatcaatgtaagcaaatgacttacctggactgcatcatcaaagagaatttgagattattgggtccagcctctcaattgattccaagaatggctaccgaagatgttgatttgaacggtattttcatcccaaagggtactagagttactgttgatatgcataccatgcactactctccaatgttgtggaaagatccaggtgtttttaagccagatagattcttggatgaaggtgaacactctcaacatagaggtttggcttggttgccattttcttcaggtggtagacaatgtttgggtatgaacttctcattgaccgaacaaagggttgtcatgtctatgatgttaagaaagtacacctgggaattgcctgccgattctattcatagagatggtgttaagttgaacgaagttaacactgttgctccaagaaccttgtccattatcttccaacaaagataccactaa

SEQ ID NO: 89 - protein, 529 aa, Syncephalastrum racemosum
MTQLTTPDTLGKLLAIYRQTPASRRASWIGWAVAATVIASQLYKTLVPPSNLPNLPTINYFRLLSSFLRRDSPTIRSQEILIPALEKHEQKKAYLAKLPLTWTVFLVDPIAAKIFLMKMDIFPKIKETIDALDSQHPIVEFFGRENVAISVGEAWRNQRKVMNPAFRRAMPISIFGNLMFKVFKNIEENKPVIAIDLMQAFTLDALGRAAFSFDFHALDDQHSIWVETYEIIRQSLRNPANAVLARYNFITKYLMPGRAKQKAATHKLNSLLLEMASRKRAELEKHPELRELPDSEKDLLTLMVEADMESGDDPTSAELLRANLSVFFLAGHDTTANTLSFWLYHMAVNKDVQAKAREEVIKILGDGPEDVMPTADQCKQMTYLDCIIKENLRLLGPASQLIPRMATEDVDLNGIFIPKGTRVTVDMHTMHYSPMLWKDPGVFKPDRFLDEGEHSQHRGLAWLPFSSGGRQCLGMNFSLTEQRVVMSMMLRKYTWELPADSIHRDGVKLNEVNTVAPRTLSIIFQQRYH

SEQ ID NO: 90 - DNA, 1569 bp, Rhizopus microsporus
  source 1..1569: mol_type = other DNA
atggaccacttgatccaggtctacaactcttctactcaagttttgattccagtcttgcagaagagatctaaggcttcttatattaccgctgccattgctttgattattgcccaaagactgtactcctacttcagagttccaaaacatttgagaggtttcccaaagttgccatactttggtattgctaagtctttgttgaccagagaatctccaagagaaagagttaagaagtacgtcttgccaatcatcgacgaaaatgatggtttctacatcagcaagattccattcggttggatgttgtacattgctaatccagttgctgctaagcagttgttgttgaaatcttctggttttccaaagaaccacggtttgttggataatatgggtgaaaacttgttcgtcgagttcatcggtaaagataacgttgctttgtctaacggtgatacctggaaaagacaaagaaaggttatgaacccagccttccatcattctttgcctattaagactatgtccaaggtcgtgttctctttgatttccgctattgaacaagccaacggtactattccagttgcatctgctatgcaaaacttcaccttggatactttgggtttagccattttcggttttgactttaaggcattgcaaggtgatccagatgaatggactaagacttacagattcgtttccgattgcatcttcgatccagttatcaacgttttctcctcctactccttcatcatcgatagaatctatccaagacgtagaagaggtgctattgctaccaaaaagttgtccgaaaagttcttgaagatcgcccaacaaaagcgtaaagaaatcaaatctggtgcttacgctgatgttccagataacgaaaaagacttgttgaccttgatgttggaagctgaagaaaaaggtgatacttggacctcagaagatgaattgagacataacattgccatcttgttcgttgctggtcatgatacaactgctcatgctttgtccttttgcttttaccatttggccaagaacaaggatgtgcaacagaagttgagaaaagaggtcttgtctatcttgggtgataagccagttgatgttgttccaactgttgagcaattgaagaacatgcagtacttgaacatggtcatcaaagaaaacctgaggattaactctccagccgatatgttgttttccagggatgtaaaagaagataccatcttggctaacaccttgattccaaaaggtactgttgttaccattaacatcgaagccttgcattacaatccaagattgtggcataatccagatcaattcgacccagaaagatttgctccaggtggtgaacacgaaaaacatgaaggtatgacttggttgccattctctaatggtactagacaatgtctgggtatgaacttcagcttgtttgaacagagattggttatcgccatgatcctgaaaaagtacgaaatctccattccagaggattccatccatagagatcatatcgtttctgacattccattcaatggtgctccaaagtctttgaagttgactttcactaagaggcactaa

SEQ ID NO: 91 - protein, 522 aa, Rhizopus microsporus
MDHLIQVYNSSTQVLIPVLQKRSKASYITAAIALIIAQRLYSYFRVPKHLRGFPKLPYFGIAKSLLTRESPRERVKKYVLPIIDENDGFYISKIPFGWMLYIANPVAAKQLLLKSSGFPKNHGLLDNMGENLFVEFIGKDNVALSNGDTWKRQRKVMNPAFHHSLPIKTMSKVVFSLISAIEQANGTIPVASAMQNFTLDTLGLAIFGFDFKALQGDPDEWTKTYRFVSDCIFDPVINVFSSYSFIIDRIYPRRRRGAIATKKLSEKFLKIAQQKRKEIKSGAYADVPDNEKDLLTLMLEAEEKGDTWTSEDELRHNIAILFVAGHDTTAHALSFCFYHLAKNKDVQQKLRKEVLSILGDKPVDVVPTVEQLKNMQYLNMVIKENLRINSPADMLFSRDVKEDTILANTLIPKGTVVTINIEALHYNPRLWHNPDQFDPERFAPGGEHEKHEGMTWLPFSNGTRQCLGMNFSLFEQRLVIAMILKKYEISIPEDSIHRDHIVSDIPFNGAPKSLKLTFTKRH

SEQ ID NO: 92 - DNA, 1569 bp, Mucor ambiguus
  source 1..1569: mol_type = other DNA
atgtactccaccgcctcttacaatcaaaccttggaaagagttgtttccgtcgtcagaaaaaacaagacctcttatattggtatggccatcttgtgtgttgtcttgcaacaaatctactcctcttttgctgttccaccaaaacacttgagaagattcccaaaggtttccttcatggaactgatgaagtccttctacaagaaagaatccgtcatgaacagaaacaagagattggttactccattgactaatgctggtcatggtttctacatttccagaattccattggattggaccatctacgttactgatccaattgctgctaagaccttgttgttgaaaactgacaatttcccaaagtctcatgctatttttggtgccttgggtgaatcttcaccagttgttaagtttatgggtaacgaaaacgttgccgtttccaacgatattctttggagaaagcaacgtaagattatgaacccagctttccatagatctcaaccagttaaggtttttggtggtgttatgccagatttgttcgccttgattgatcaagatccagaacatgttttcatcacgccaaagattaagtcctttgctttggatgctttgggtttgtctgcttttggtttcgatttccagtctttgaagaatgatccagaaggttggacttctaagtacaataccgttgtctcttctctgttcaacccattcgttaacttgttcgctaagtgcgacttcctgattaagtacatttctgccgaaagaagaagagtcatcaaggttactgatgaattcaacgccatgttgtctaacttggctgataagagaaggcaagaaatcatgaacggtgagaaaaagaacattgccgaaaacgaaaaggacctgttgactttgatgattgaagctgatacaagagaaggtgttgaaactactactaccgaattgagacataacatggccatttttttcttggccggtcatgatacaactgctaatactttggctttgtgcttgtaccaattggccaaacataagcacgttcaaaagaaggctagacaagaagttttggatatcttgggtgatgatccatgggatgttgctccatctttggaagatttgaagaagctgaactacctgaacatggtcatcaaagaaaacctgagaagaaacggtccagttgacaatttgatgtctagagatacccaacaggacatcaatttgaacggtacttttatcccaaagggttctaaggttgttatcaacgttgcctccattcatttgaacccaaagatttggcataaccccgaatctttcattccagaaaggtttgaacaaggtggtgaattcgattctcatgatggttttacttggctgccattttctaacggttctagacaatgtttgggcctgaatttctcattgactgaacaaagggttgctctgtgcatgttattgaagagatacgaaattgacatccccaaggattctatccattacgacgaaatcgttttcgataaggcttttactttcgccccacaatcattggaattgtccttcaaaaagaggtactaa

SEQ ID NO: 93 - protein, 522 aa, Mucor ambiguus
MYSTASYNQTLERVVSVVRKNKTSYIGMAILCVVLQQIYSSFAVPPKHLRRFPKVSFMELMKSFYKKESVMNRNKRLVTPLTNAGHGFYISRIPLDWTIYVTDPIAAKTLLLKTDNFPKSHAIFGALGESSPVVKFMGNENVAVSNDILWRKQRKIMNPAFHRSQPVKVFGGVMPDLFALIDQDPEHVFITPKIKSFALDALGLSAFGFDFQSLKNDPEGWTSKYNTVVSSLFNPFVNLFAKCDFLIKYISAERRRVIKVTDEFNAMLSNLADKRRQEIMNGEKKNIAENEKDLLTLMIEADTREGVETTTTELRHNMAIFFLAGHDTTANTLALCLYQLAKHKHVQKKARQEVLDILGDDPWDVAPSLEDLKKLNYLNMVIKENLRRNGPVDNLMSRDTQQDINLNGTFIPKGSKVVINVASIHLNPKIWHNPESFIPERFEQGGEFDSHDGFTWLPFSNGSRQCLGLNFSLTEQRVALCMLLKRYEIDIPKDSIHYDEIVFDKAFTFAPQSLELSFKKRY

SEQ ID NO: 94 - DNA, 1572 bp, Phycomyces biakesleeanus
  source 1..1572: mol_type = other DNA
atggacaccatcaaccacatctctccatctttcgaatcttacattaccgttttcgctaagatcgttccaaagggtttttatgttgttgctgctgttgctttgttcctgtgtaaaaaggtttacgattttaccgctgctccatacaagttgagacattttccaaaggtttctttcttcgccttctccaaatctattttctccgctgaatctgttgaacacagaactaagagattgatctctccactgttgcataagtacaagggtttctacattgctaagttcccattatactggactgttttcgttactgaaccacatgctgttcaatacgttttgatgaagggtgaaatcttcccaaaaaagaccagattcatgaacaccttgaacaaggactcattgatgattaggttgttcggctcctctaatattgcttttgcttctggtgaaatttggaagcaccaacgtaagattatgaacccagcttttcatagaactgccccaattgaattattcggtagaatgatcccagacatgttcagattgatcgataagtccaacggtaacattatgttggccgatttgttgcatagaattacctttgatgctatgggcaaagccttgtttggtttcgatttcaaaactgccagagaagaaaactctgaatggacttctgcttacaacgatgctatgtctggtatttctgctccaattttgaacatcaccccatccttggaacatatcactagatacttgtatccagattacgctaaggccaaaaagggtattgataagttgactgaattgaccttggaaatggtcaacgagaggaaagaaaagattcaagaagctatcggtcaaccagatgatggtagagaaaaggatttcttgaccttgattatcgaagccgaaatgaaggaagataaggcttctggttctggtggtttgagagaaaatttgaaggcttttttggttgctggtcatgcttctactgctagctctatttctttctgcatctaccatttggccatgaacaaagatgtccaaaacaaggcaagaaaagaagccttggatatcttgggtgatgatgctaatatttctaccccaaccgttaacgaatgtaagcacattacctacatcaacatgatcatcaaagaaacgttgagattgaacgctccattcggtactttgttcgaaagaattgctactgaagatgtcaccttgtccggtgtttttattccaaaaggtactatcatctccgtcgacattgaaaccattcataagaatccagccatttggaagtctccaactgtttttgatccagagagattttctaaaggcggtgaacatgaccaacatgaaggtattacttgggctccattttctgatggtaacagaaaatgcttgggcatcaatttctctatggctgaacaacaagtcatcctgctgatgttgttgaaacattacgaatgggacttgtccgaaaactccattcacaaaaatggtatcgtttacgacggcatctctttgtttactccaagatccttgtacatcaagttcaacaagagacactaa

SEQ ID NO: 95 - protein, 523 aa, Phycomyces biakesleeanus
MDTINHISPSFESYITVFAKIVPKGFYVVAAVALFLCKKVYDFTAAPYKLRHFPKVSFFAFSKSIFSAESVEHRTKRLISPLLHKYKGFYIAKFPLYWTVFVTEPHAVQYVLMKGEIFPKKTRFMNTLNKDSLMIRLFGSSNIAFASGEIWKHQRKIMNPAFHRTAPIELFGRMIPDMFRLIDKSNGNIMLADLLHRITFDAMGKALFGFDFKTAREENSEWTSAYNDAMSGISAPILNITPSLEHITRYLYPDYAKAKKGIDKLTELTLEMVNERKEKIQEAIGQPDDGREKDFLTLIIEAEMKEDKASGSGGLRENLKAFLVAGHASTASSISFCIYHLAMNKDVQNKARKEALDILGDDANISTPTVNECKHITYINMIIKETLRLNAPFGTLFERIATEDVTLSGVFIPKGTIISVDIETIHKNPAIWKSPTVFDPERFSKGGEHDQHEGITWAPFSDGNRKCLGINFSMAEQQVILLMLLKHYEWDLSENSIHKNGIVYDGISLFTPRSLYIKFNKRH

SEQ ID NO: 96 - DNA, 1515 bp, Phycomyces biakesleeanus
  source 1..1515: mol_type = other DNA
atgtgcctgttcttctgcaagaaggtttacgattttactgccgttccatacgagttgaagaagtttccaaatatccccttcttctacttcgtcaaatccatcttgtctgttgaatccgtcgaaaacagaactaagagattggttttgcctctgttgcataagaacaacggtttctacgtttctaggtttccattctactggactgttttcactactgatccagatgctgttcagtacttgttgatgaagggtgaaattttcccaaaggataccagattcacttccgctgtttctaaggattccttgattattaggctgttcggttcttctaacgttgcctttatttctggtgaaccattgaagcaccaatgcagaactatgaatccagctttcagaagaactgccccagttaacattttcggtagattgattccagacatgttcaggttgatcgataagtccgatggtgatatcttgatcgtcaacttgttgcaaagaatgacctttgatgctttgggtaaagctttgttcggtttcgatttcaagaccatgaaggaagataattctgcttggatgactgcttactctgatgctatgtctggtgttactgctccagttttgaacattattccatccttggagtacatcctgagatacttctatccaaactacaccaaggctaagaacggtgttgataagttgaacagattgatcttggagctggtcaacaagaagaagcaagaattggaagaatccatgccacaatcttgctctaacaacaacaatggtgatactgatggtgatgcttacgaccaagaaaaggatttgttgaccttgattttggaagccgaaatgaaggacaacaagtcatctggttctgatgatttgagagctaacatggctactttcattatggctggtcacgaaactactgcttcatctgtttctttctgcatctaccacatggttatcaacaaggatgttagagataaggctagaagagaagccttggatattttgggttacgatgatttcatttccccaccaactttcgatgaatgcaagagagttaactacatcaacatggttgtcaaagaggctttgagattgtgtactccaggtggtttgttgttcgaaagaattgctactgaggacgttttcttgtccggtgtttttattccaaagggcaccagaatttccgttgatattgaagccttgcacaagaatccagcaatttggaaaaacccatccgttttcgatccagagagattttctaaaggcggtgaacatgaacaacatagaggtattacttgggctccattttctgatggtaacagaaaatgcttgggcatcaacttctctatgactgaacaacaggtcatcctgctgatgttgttgaaacattatgaatgggacctgtccgaaaactccatccataacaatggtatggtttacgacaacgttttctccttcgctccaaaaaccttgtctatcaagttccataagaggcactga

SEQ ID NO: 97 - protein, 504 aa, Phycomyces biakesleeanus
MCLFFCKKVYDFTAVPYELKKFPNIPFFYFVKSILSVESVENRTKRLVLPLLHKNNGFYVSRFPFYWTVFTTDPDAVQYLLMKGEIFPKDTRFTSAVSKDSLIIRLFGSSNVAFISGEPLKHQCRTMNPAFRRTAPVNIFGRLIPDMFRLIDKSDGDILIVNLLQRMTFDALGKALFGFDFKTMKEDNSAWMTAYSDAMSGVTAPVLNIIPSLEYILRYFYPNYTKAKNGVDKLNRLILELVNKKKQELEESMPQSCSNNNNGDTDGDAYDQEKDLLTLILEAEMKDNKSSGSDDLRANMATFIMAGHETTASSVSFCIYHMVINKDVRDKARREALDILGYDDFISPPTFDECKRVNYINMVVKEALRLCTPGGLLFERIATEDVFLSGVFIPKGTRISVDIEALHKNPAIWKNPSVFDPERFSKGGEHEQHRGITWAPFSDGNRKCLGINFSMTEQQVILLMLLKHYEWDLSENSIHNNGMVYDNVFSFAPKTLSIKFHKRH

SEQ ID NO: 98 - DNA, 1536 bp, Phycomyces biakesleeanus
  source 1..1536: mol_type = other DNA
atgctggccaaaaaggttaatggttacgctggttttaccttggatgctattcaaaccatctacttcaacagagccatcaagttggttagaccaccaaaagctttgtcccatattccacatgttccatacttcagctacatgtcctcattgatcaagaaagagaacaccatctccagaaacaagagattcgctaacaagttgttcgaagatccagaatctaacggcttgtacttaagaccaaatgtcaaaggttgggaagttgttgttactagaccagaagatgccaagaagctgttgttcaagtctgatatttttccaaaggccgatttcacctctggtattgatggtactattctgtccaagttttctaggggtccaaacttgttgtttactactggtgctcattggaaggctcaaagaatggttgctaatccagcttttcatagatctgctccagttaagttgtttggtgaattgacccaaaagctgttcagagttatggataacagagctaacaagaccgttgatatcaccagattgatggaagcttgggctttagatgctattggtttggctggttttgatttcgatttcaacgctatcgaaaaccccaattcttcttgggttagaatctacgaaagagtcgataaggctttggttcatccattctactcatttttcccaaaagcagacaagtacctgttgtgggttttgccaaatagaaagcaagctcataccgatttggacatcttcttgaagatgatcgacaacgttattatcgccaagaaaaaagccttgcacgagaacaagtctaacaagcacttggaaaactccgaaaaggatttgttgaccttgatgttggaatctgaagctaagggttctactttgtccgctaaagaattgagatctaacatgtgcgtttttttcgctgctggtcacgaaactactgctaattctttggcttacgccatctatttcatggctgttaatccagatgttcaatgcaaggctagagaagaagcttttaaggttttgggtgatgcccaagaggatattatgccaactattgaacagaccaagagcatggattacattaacgccgttatcaaagaaaccttgagatcacattctccagctttgggtacttttgctagaaaagctactaaggacactgaattaggtggtgttttgattccaaaggacaccatgatttccatggatatcttcaacttgcatcacaatccaaacgtctggaacaacccagatgaatttgatccatctagatttttgccaggtggtgaagctgaaaagcaaatcaacaatggtttctcctggattccatttggtacaggtgctagacaatgtatcggcatgaatttcagcttgatcgaacaaagggttatgctgtctatgatgttgagaaagtttgcttggtctttgccagaagattccatccataaggattacttgcataccaccaacttggttatttcttccgctaaggatctgaacatcaacttcgaaaagctgtactaa

SEQ ID NO: 99 - protein, 511 aa, Phycomyces blakesleeanus
MLAKKVNGYAGFTLDAIQTIYFNRAIKLVRPPKALSHIPHVPYFSYMSSLIKKENTISRNKRFANKLFEDPESNGLYLRPNVKGWEVVVTRPEDAKKLLFKSDIFPKADFTSGIDGTILSKFSRGPNLLFTTGAHWKAQRMVANPAFHRSAPVKLFGELTQKLFRVMDNRANKTVDITRLMEAWALDAIGLAGFDFDFNAIENPNSSWVRIYERVDKALVHPFYSFFPKADKYLLWVLPNRKQAHTDLDIFLKMIDNVIIAKKKALHENKSNKHLENSEKDLLTLMLESEAKGSTLSAKELRSNMCVFFAAGHETTANSLAYAIYFMAVNPDVQCKAREEAFKVLGDAQEDIMPTIEQTKSMDYINAVIKETLRSHSPALGTFARKATKDTELGGVLIPKDTMISMDIFNLHHNPNVWNNPDEFDPSRFLPGGEAEKQINNGFSWIPFGTGARQCIGMNFSLIEQRVMLSMMLRKFAWSLPEDSIHKDYLHTTNLVISSAKDLNINFEKLY

SEQ ID NO: 100 - DNA, 1380 bp, Rhizopus microsporus
  source 1..1380: mol_type = other DNA
atggccatctcgttcttgtatagagaaggtccagctgaaagattgaagaggttgaaattgccagctatgagaaaaggtaacggcttctatttgtctagagttccattttgttggaccgtttacgttgctaatccaattgctgctaaacagttgttgttgaaggctgaaaatttccccaagtctcactttttgatggttggtaaggattccccattcgttcaatttttgggtccagataacgttgtcaactctaatggtgaaaactggaaaaagcagcgtaaggttatgaatccagctttccatagatcaatgccagttaagactattgccggtgttgttttgactttgttcgccgttattgataagtacaaaggtaaggttccagtcacctctactatgcaagaattcaccttggatgttttgggtttagccatcttcaatttcgacttcggttctttgaaaggtgatgctaaaaaatggcgtgctgagtacaaattggttatgttgtttgatccagtgaccaacgttttcactggtttcgattttttgctgaggtacatctaccctaagagaatcaaaggtgctaacgctgttaacaacctgaacaagttgttcgatcaattggtcaagcagaagagattggaagttcaatctggtgttcatgctaacaagccacaaaacgaaaaggatttgttgaccttgatgttggaagctgaacaaaggggtgaagctatgactactgatatggaaatgagacataacgtcgccgtttttttcttggctggtcatgaatctactgcccatgttttgtctttcaccttgtactttttggccaagaacaagtacgtgcaacagaagttgagagaagaggtttctagagttatgggtagaaagtctgttgatgttgctccaactttggaagaattgagacagatggaatacttgtacgccgtcatcaaagaatccttgagattgtgctctatcttcgacgttttgattttgagagatgccgtggaagatatgtacttggatgatacttttattcccaagggcaccagaattaccattgatgtttctgccattcagagagatccaaaggtttggaacaatccagatgatttcatcccagaaagattcatggaaggtggtgaagccgaaggtcatgaaggtatgacttggttgccatttggtggtggtgctagacaatgtattggtatgaatttctccctgaccgaacaaaaagttgctttggctatgttggttcagagatacgatattgatgttcccaaggactccatccattacgaagatattgcttacgaaaggccattttacttggctccacaatctttggaattgaccttcactaagttgcactaa

SEQ ID NO: 101 - protein, 459 aa, Rhizopus microsporus
MAISFLYREGPAERLKRLKLPAMRKGNGFYLSRVPFCWTVYVANPIAAKQLLLKAENFPKSHFLMVGKDSPFVQFLGPDNVVNSNGENWKKQRKVMNPAFHRSMPVKTIAGVVLTLFAVIDKYKGKVPVTSTMQEFTLDVLGLAIFNFDFGSLKGDAKKWRAEYKLVMLFDPVTNVFTGFDFLLRYIYPKRIKGANAVNNLNKLFDQLVKQKRLEVQSGVHANKPQNEKDLLTLMLEAEQRGEAMTTDMEMRHNVAVFFLAGHESTAHVLSFTLYFLAKNKYVQQKLREEVSRVMGRKSVDVAPTLEELRQMEYLYAVIKESLRLCSIFDVLILRDAVEDMYLDDTFIPKGTRITIDVSAIQRDPKVWNNPDDFIPERFMEGGEAEGHEGMTWLPFGGGARQCIGMNFSLTEQKVALAMLVQRYDIDVPKDSIHYEDIAYERPFYLAPQSLELTFTKLH

SEQ ID NO: 102 - DNA, 1581 bp, Phycomyces blakesleenanus
  source 1..1581: mol_type = other DNA
atggccaactcctctttcgaaaacctgcaatctttgttcgttgatttcgtccaaccacaattggtccaaagaaaaaaacaagccggtgttattttctccgccgttgttttggttttgtgttacaacaccatcaacaagatcatctacccaccaaagtccttgagacatattccacacgttaactacattgcctacaccagatcacaattgagaaaacaaccagcttctgaacaagctagagaactgttgttgccattattggctgaaaacaatggtttctacgccattccatttctaggtaagtggtctgttcaagttgctaacccaattgccattaagaccatcctgttgaagtttgacatgttcccaaaggctaactccttgtctaaatctcaaggtacattggcccatagattcattggtggtccaaatgtttttttgttggagggtaaaaagtggcgtaagcagagaatgatttctaacccagctttctctagatccatgccagttgatatgtttggtaggattaccatcaacctgtttaagtacttggataacatcgatccaaccatcgatgttttggataccatgaagaaatggaccttggatactttgggttctgctgtttttgatttcgacttcgaatctttgaccaagccaaacaatgaatggacctctatctactacgagattaacgcctctttgtttgtcccaatcttcaacattttgccagtcttggaaaagtccttcttgtggatgtttcctaagagaaaaagggttcacgataagatgaccaagttgaaggatatgatgagacaagtcatcatccaaaagcaggctaggttgaaagaaaacaaacccaatccaaacttgaaggacaccgaaaaggatttgttgaccttgttgttggaatccgaaaatgaaggtcatgagccaatgtctgaagatgagttgatgtctaatttgtgcgcttttttcttcgctggtcatgatacaactgctaacgctttatcttctgccttgtatcatttggctgttcaacaagacgttcaaaagaaagcaagagaagaagtcatcaacgttttgggtgatgaaccagaagatgtcattccatctattgaagataccagacagttggactacctgaacttgatcatcaaagaaaacatgaggatcaacccaccagttggtggaccattagatagattggttactgaagatatcgttttggacggtgttttgttgccaaaaggtacttctgttaaggttgccgtttactccttgcatagaaatccattattgtgggattccccagaagaattcagaccagaaagatttttgccaggtggtgaagctgataagattgaaggtatgggttacatcccattttctgatggtggtagacaatgtatcggtatgaacttctcattggttgaacaaagggttttgttggctatgatgttgagaaagtacacctggaagttgtctgagaacactattaacaaggacgaattgcaagtttacgcctttaacattatggccccattcgatttgaagatcactttggaaaagaggtactaa

SEQ ID NO: 103 - protein, 526 aa, Phycomyces blakesleeanus
MANSSFENLQSLFVDFVQPQLVQRKKQAGVIFSAVVLVLCYNTINKIIYPPKSLRHIPHVNYIAYTRSQLRKQPASEQARELLLPLLAENNGFYAIPFLGKWSVQVANPIAIKTILLKFDMFPKANSLSKSQGTLAHRFIGGPNVFLLEGKKWRKQRMISNPAFSRSMPVDMFGRITINLFKYLDNIDPTIDVLDTMKKWTLDTLGSAVFDFDFESLTKPNNEWTSIYYEINASLFVPIFNILPVLEKSFLWMFPKRKRVHDKMTKLKDMMRQVIIQKQARLKENKPNPNLKDTEKDLLTLLLESENEGHEPMSEDELMSNLCAFFFAGHDTTANALSSALYHLAVQQDVQKKAREEVINVLGDEPEDVIPSIEDTRQLDYLNLIIKENMRINPPVGGPLDRLVTEDIVLDGVLLPKGTSVKVAVYSLHRNPLLWDSPEEFRPERFLPGGEADKIEGMGYIPFSDGGRQCIGMNFSLVEQRVLLAMMLRKYTWKLSENTINKDELQVYAFNIMAPFDLKITLEKRY

SEQ ID NO: 104 - DNA, 1341 bp, Lichtheimia ramosa
  source 1..1341: mol_type = other DNA
atggataaccagacctggtctagtgttccaggtgcttcattttggaaaggcaaattggtttctggtcactacgttagggaagtgtttttgaacaacgatttcgacttcttgaaaggcactggtaagagattcgatactttgttgttgactgataccaccactcaagatgttgacatcgaagttttcagaaccgttgttatgaagcacttgaccaaagaaatgaaggcttacactccaagagttgttcaacatttgactgctggtggtgacgaaaaattgggtgatgctaaagaaccacaagaattggttcatctgttccctttgttacaacatatggttgctaaagcctccgcctctatttttgttggtactgaattggcttccgatgatgatgttgttgaaacctgtaagaacattgccatcgacattggttctgaattaggtccaggttcctatatcatggatgcttttccatctttggccagattgagaatgtggtacattggtaaatttggcaaggccattaacaagcacagacaacatttgttgagagctttgggtccagttattgataagagattggctgctgctgaaaaaggtggtgattgggatagaccacaagatatattgcaagacatcatcgaaaccatcaacttgactttggataacccaaagagacatatcttgccagttaagtggttgttggctttgttctttgcttctattcataccacctccgaaaactctactatcgtcttgtacagaatcatgcagaacccagaaatcatcgatgtcttgttggaagaacaaaaccaggtcttggaaaaacactacggttccaacattgattactccgataccactaagttgttcactggtgaagtcatcaaagagttggttaagttggattccttgtgcagagaagctatgagagctagaaattcctatttggaattgccacatacttacgtcggtaagtccagaattactttgtcatgcggtgctattattgaaccaggtcatgatgttttgatcaacatgtggggtaatcatagagatgccaaaattcaaagggataccatcggtgatcatcacgattttaagccattcagattcgttggtttggacagacaatctaccaagattggtgatgactttttgatgttcggtcaaggtagacatgcttgtccaggtagatggtttgctattcaagaaatcaagaccatcgtgtccgttttgattaggtactacaagttgactccaaacggtccaattacttttccaactcatccaagaatgccaatgcctatgggtcaagttataattcagagaaggcagtaa

SEQ ID NO: 105 - protein, 446 aa, Lichtheimia ramosa
MDNQTWSSVPGASFWKGKLVSGHYVREVFLNNDFDFLKGTGKRFDTLLLTDTTTQDVDIEVFRTVVMKHLTKEMKAYTPRVVQHLTAGGDEKLGDAKEPQELVHLFPLLQHMVAKASASIFVGTELASDDDVVETCKNIAIDIGSELGPGSYIMDAFPSLARLRMWYIGKFGKAINKHRQHLLRALGPVIDKRLAAAEKGGDWDRPQDILQDIIETINLTLDNPKRHILPVKWLLALFFASIHTTSENSTIVLYRIMQNPEIIDVLLEEQNQVLEKHYGSNIDYSDTTKLFTGEVIKELVKLDSLCREAMRARNSYLELPHTYVGKSRITLSCGAIIEPGHDVLINMWGNHRDAKIQRDTIGDHHDFKPFRFVGLDRQSTKIGDDFLMFGQGRHACPGRWFAIQEIKTIVSVLIRYYKLTPNGPITFPTHPRMPMPMGQVIIQRRQ

SEQ ID NO: 106 - DNA, 1593 bp, Hesseltinella vesiculosa
  source 1..1593: mol_type = other DNA
atgctgttccagtacatccatcatttggccgaaagattcgatcacaaaaagaccttggatcaattgcaaactttcgcttctactccagaaggtgctgttggtattactgctgctgttgctttggttgctggtgcttcttatttgaagaacaagaacaacgatagaggttgcccaaaagttccaggttcttctgtttggggtacttctactgaagagtatagagctgatccaaaggcttttatcgttaagtggcaaaatgaattgggtccagtttaccatgctgagttgtttggtcatactgctacagttgtttctggttcttacgtcagagaaatcttcttgaacgataagttcgatttcatggctggcttgcatagaactttcgataacatgttgttgaccaactgtggtccatacgaagatttgtctgctgctcatacttctgaagttgtcaagaagtttttgtcgccacatttgaaacatttcaccccaagagttatcgaacacttgcaacaaggtttgaaagctcaaaccggtgaaatttctgctgaaggtaaacaattcccacacgtttatggtttggttcaacatccagttgctttagcttcagcttctgtttttgttggtccagagttgtctaagaacgagttgttgattgactccttcaagaacatggttattgatgtcggttccgagttgttaccaaatccatggttggaaccatttccaagattgaacagattgagaatgtggtgggttggtaagacttcttctactgttaagagacacagaggtcaattggctactgctttgaaaccagaattggatagaagattgaaggctatggcttccaatgattctaattgggaaagaccagatgacatcttgcagaacttgttggaacattacactccaccaaaaggtatggataccttgaactacatggttaattggatgactcaattgactttcgctgctattcatacaacctctgaaggttctacttgggtcttgtacagattattacaaactccaggtttgtgggaagagttgtaccaagaacaaaacgaagttttggaagcctccggtattgattcttcagctggtgctgaagttttcaccagagaaattttgaacaagttcgttaagatggactccgtcattagagaaactatgagagctagaactgcctacattactttgccacatatcaacaagtccaacgaagtcgttactttgtctaatggtgctaaaatctacccaggtgaatccgcttatatcaacgtttggtctaatcacaatgacccctcattgcaaaattccatgaaggacttacaacagttcaagccattgagatttttggacgctgaaaagaactctaccaagatcggtgaagatttcctgtttttcggtatgggtaaacatgcttgtccaggtagatggtttgctgttcaagaaatcaaaaccatcgttgccttgttgttgagagagtacaaattggaagctgttggcgatttgtttttcccagaagttgaatccattccttttccaatgggtgaattcaagatctacccaagaaagcaagttgcctga

SEQ ID NO: 107 - protein, 530 aa, Hesseltinella vesiculosa
MLFQYIHHLAERFDHKKTLDQLQTFASTPEGAVGITAAVALVAGASYLKNKNNDRGCPKVPGSSVWGTSTEEYRADPKAFIVKWQNELGPVYHAELFGHTATVVSGSYVREIFLNDKFDFMAGLHRTFDNMLLTNCGPYEDLSAAHTSEVVKKFLSPHLKHFTPRVIEHLQQGLKAQTGEISAEGKQFPHVYGLVQHPVALASASVFVGPELSKNELLIDSFKNMVIDVGSELLPNPWLEPFPRLNRLRMWWVGKTSSTVKRHRGQLATALKPELDRRLKAMASNDSNWERPDDILQNLLEHYTPPKGMDTLNYMVNWMTQLTFAAIHTTSEGSTWVLYRLLQTPGLWEELYQEQNEVLEASGIDSSAGAEVFTREILNKFVKMDSVIRETMRARTAYITLPHINKSNEVVTLSNGAKIYPGESAYINVWSNHNDPSLQNSMKDLQQFKPLRFLDAEKNSTKIGEDFLFFGMGKHACPGRWFAVQEIKTIVALLLREYKLEAVGDLFFPEVESIPFPMGEFKIYPRKQVA

SEQ ID NO: 108 - DNA, 1572 bp, Phycomyces blakesleeanus
  source 1..1572: mol_type = other DNA
atgatcgagtacctgaaagaattcgtcgacaggattgattctaagaccttggaaaacgttaagtccttggccttttctaaagaaggtgccattggtatttctaccgccattattttgtcctccgcttacaattatcacaagtggtcctctagaaccatttctaatggttgtccaagagttccacataccttgccatttgttggtttgactagagtttaccgtaaggactctaaggctttttgtgaagaatggcatgctaaattgggtccagtttttagagcacacttgttcggtaaagaagttaccgttgtttctggtcactacgtcagagaagtttttttgaacaagcacttcgatttcgttaagggtgttgctaaggtttttgataccaggttgttgactgattccggttccagagaagattttactccagaagatttgagggaaatcatcacgaaatacttgaccccaaagttgaacttctacaccaagagagttatcaagagattgaagcaaggtgtcgaatcttctttgggtgataaggattccatcgaattggataacttgtacccattcgttcaacacttggttgttaatgcctctgcctctatttttgtcggtgaagaattgtcccaaaacaagttgttgatcgactccttcaagaacatggttagagatgtgggtaaagagatcaagcaaaatccatggtttgaaccctttccaaccatcaacaagtttaggatgtggttgattggtaagacctctccagttattaagaaccacaaagagcaactgttgaacgccattaagccagaagttgagtatagattgtctcaggctagatctaatccagactggaaaaaacctaccgatatgttgcaagacttgttggaaaattctaagccaccagctcatttggatttgatggatcatttggttcacatcatcacgttcttgatttttgttgccttgcataccacctctgaaaacactactgttttgttgtacaggatgttggagaatccagctatcgttgatgaattggtcatcgaacaacaagaagtcttggaacaagaaggtttggacgctaattgtggttccgaagttttcaccagagatatcttgaagaagttcgtcaagttggattctgtctgtagagaaaccttcagaatgaagaaccagtacatctctttgccacatgaatacgatggtaaggttccattgactttgtctaatggtgctgttattaacccaggtgaagatgttttgattgatgtttggactaaccaccagtacactgaagatgctaatgatgttgaagatgccgatcaattcaaggcctttagatttgttgatcaggacaagcaatctaccaaggttggtgaagattacttgttttttggtatgggtagacgtgcttgtccaggtagatggtttgctattcaagaagtccaaaccattttggccatgttggttcgtgagtataagtttatgccaaagggtccaatcgttttcccaactgaagaaagatctccaattccaaccggtaagtgtattatccagagaaagtga

SEQ ID NO: 109 - protein, 523 aa, Phycomyces blakesleeanus
MIEYLKEFVDRIDSKTLENVKSLAFSKEGAIGISTAIILSSAYNYHKWSSRTISNGCPRVPHTLPFVGLTRVYRKDSKAFCEEWHAKLGPVFRAHLFGKEVTVVSGHYVREVFLNKHFDFVKGVAKVFDTRLLTDSGSREDFTPEDLREIITKYLTPKLNFYTKRVIKRLKQGVESSLGDKDSIELDNLYPFVQHLVVNASASIFVGEELSQNKLLIDSFKNMVRDVGKEIKQNPWFEPFPTINKFRMWLIGKTSPVIKNHKEQLLNAIKPEVEYRLSQARSNPDWKKPTDMLQDLLENSKPPAHLDLMDHLVHIITFLIFVALHTTSENTTVLLYRMLENPAIVDELVIEQQEVLEQEGLDANCGSEVFTRDILKKFVKLDSVCRETFRMKNQYISLPHEYDGKVPLTLSNGAVINPGEDVLIDVWTNHQYTEDANDVEDADQFKAFRFVDQDKQSTKVGEDYLFFGMGRRACPGRWFAIQEVQTILAMLVREYKFMPKGPIVFPTEERSPIPTGKCIIQRK

SEQ ID NO: 110 - DNA, 1587 bp, Licthheimia ramosa
  source 1..1587: mol_type = other DNA
atgccaagcttgattccaaagaacgcttccgatgctattgaacagtacatctctagactgaaaaccatggatagaaggcacatgggtattattgctggttctgttgctgttgtttccttgtacactttctacagacatttgaccagatccagagatgatattccattggttccatatacctggccattgattggttctactccatcttttaacagagatccagttgcctttgttgagaagtggtcacaagaatatggtccagtttttagagcacacttgcaaggtagaatccaaactattattgccgccgaatacgttagagacatcttcatgaattccgacttcgattttttgttcgccgtcaacaaaagattcgatccacatttgttggccgatatcgatgataacactttcactaccgaaatgttgagaaaggtcgttatgaagttgactacccagttgaaatcttacactccaagagctgttgaattcttggatgttggtagaaacgaattcttgtctcattttccagctggtcctgttcatttgccacacttgtatccattgatccaacatatggttggtaaagcctctgctgctatttttgctggtacaaagttggcttctaacccagaagttgttgagtccttcaagaacattactttggaagttggtgctgaaatcgccgttgattctgtttttttggaaagatactggggcttgaacagattcagaatgtggttgatgggtaaattctccaagtccatgaagagacatcacagagttttgaaagatgccctaagaccagaaatcaaggatagaattgaagcctcttctgacccatctagagaaagaccagatgatatgttgcagatcatcatcgagtcttactacactgaaagaagaggtgaatccgttgatgatttgactgaagatttggtcaagtggctgatctctttgattttcgctgctattcataccacctctgaaaactctactgttgtcttgtacaggatcttgtctaagcctgatgttgttgaagagttgttggaagaacaaagagaagttttggttaggcatggtatttctccagacgaaaaagatccatctaagatgtttactggtgccgttattaaggacttggttaagttggattctgcttgcagagaaggtatgagaatgagaaacgattacttgactttgggtcatacctacttgggtaaaaagccaattacattgtcttgcggtgctgttatcaaaccaggtgaagatgttattatcaacacctggtacaaccaccgtaacaacaagatccaaaaaatccaaggtgacgactactctaactacaacccattcagatttgttggttccgatagacaagctgctagaattggtgatgatttcttgatcttcggtgaaggtaaacatgcttgtccaggtagatggtttgctctgcaagaaatgaagaccatcatctcgtttttgatcagggattacaaaatggctccagaaggtccaattactttcccaaagaatcctaagatgactttgccaatgggtcaagtgattttggaatccagacattaa

SEQ ID NO: 111 - protein, 528 aa, Lichtheimia ramosa
MPSLIPKNASDAIEQYISRLKTMDRRHMGIIAGSVAVVSLYTFYRHLTRSRDDIPLVPYTWPLIGSTPSFNRDPVAFVEKWSQEYGPVFRAHLQGRIQTIIAAEYVRDIFMNSDFDFLFAVNKRFDPHLLADIDDNTFTTEMLRKVVMKLTTQLKSYTPRAVEFLDVGRNEFLSHFPAGPVHLPHLYPLIQHMVGKASAAIFAGTKLASNPEVVESFKNITLEVGAEIAVDSVFLERYWGLNRFRMWLMGKFSKSMKRHHRVLKDALRPEIKDRIEASSDPSRERPDDMLQIIIESYYTERRGESVDDLTEDLVKWLISLIFAAIHTTSENSTVVLYRILSKPDVVEELLEEQREVLVRHGISPDEKDPSKMFTGAVIKDLVKLDSACREGMRMRNDYLTLGHTYLGKKPITLSCGAVIKPGEDVIINTWYNHRNNKIQKIQGDDYSNYNPFRFVGSDRQAARIGDDFLIFGEGKHACPGRWFALQEMKTIISFLIRDYKMAPEGPITFPKNPKMTLPMGQVILESRH

SEQ ID NO: 112 - DNA, 1584 bp, Absidia repens
  source 1..1584: mol_type = other DNA
atgctgacccagtacatccaccaattcttcaacaatttcgaccagaaaaagaccatggaccaattgcaatctgttgcctcttctaaggatggtgttattggtattgctaccgccttgattttgatttctggtgttgctgcttacaagtcctctactgttgaaagaggttgtccacaagttccatgtggtactttttctttcggttctacttctgagtacagagaaaatccagttgcctttgttaagaagtgggaagaaaaattgggtccagttttcggtgctcaattatttggtcaatacgctactatagtctctggtcctcaagttagagaaatcttctccaacgaaaacttctctttcatggccggtattcaaagagatttcgatacttacttgttggctaacggtggttccattcatgatttgccaccacatgttgtttctggtggtattaagaagaacctgtctccaaagttgccattctacacctctagagttatcgaacatttgaagatcggcttgtacgaacaatgtggtgttgttccagatgagggtaaagaattcgatcatgtttacccattcgttcaacatatggttgctaaagcttccgcttctgtttttgttggtccagaattggctaaggatttgaacttggttgactccttcaagaacatggttttggaagttggttctgatatgggtccaaagccatacttggaacattttccacatttgatgagactgaggatgtggtacattggtaagacttctacaaacgtcaagagacacagagatcaattattcgctgctttggttccacaaatcgactctagattgaaagccatgaaggaaaaggattccaattgggatagaccaaacgatttcttgcaggatattttggaaaccgatgattgtccacctcacatggatatctattcttactgtgttgattggatgacccaaattatctttgctgccttgcataccacatctgaaaatggtactattgtcctgtaccgtttgttggataatccaaaagtcttggaagagttgtacgaagaacaaaacgctgtattggaagaagctggttacgacgatactgttggtcctgaagttttcaccagagagattttgaacaagttcgtcaagatggactccgtcattagagaatcttgtagattgagaaacgactacattggtttgccacataccaatgttggtaagaaaaccatcgttttgtctggtggtgctatgattagaccaggtgaaaatgctttcgtcaacttctactcaaaccatagggatgagaagttgcaaaagtctggtatgaatgccaacaacttcgaaccatacagattcgttgatcagggtaagaactctaccaagattggtgatgatttcatgttcttcggtatgttcaaacatgcttgtccaggtagatggtttgctatccaagaaatcaagaccattttggccatgctgatcagatcttacaagatgtctgctatcgattccgttgttttcccaacttctgattacactagaattccaaccggtagattcaaaatcgtcccaagaaagtga

SEQ ID NO: 113 - protein, 527 aa, Absidia repens
MLTQYIHQFFNNFDQKKTMDQLQSVASSKDGVIGIATALILISGVAAYKSSTVERGCPQVPCGTFSFGSTSEYRENPVAFVKKWEEKLGPVFGAQLFGQYATIVSGPQVREIFSNENFSFMAGIQRDFDTYLLANGGSIHDLPPHVVSGGIKKNLSPKLPFYTSRVIEHLKIGLYEQCGVVPDEGKEFDHVYPFVQHMVAKASASVFVGPELAKDLNLVDSFKNMVLEVGSDMGPKPYLEHFPHLMRLRMWYIGKTSTNVKRHRDQLFAALVPQIDSRLKAMKEKDSNWDRPNDFLQDILETDDCPPHMDIYSYCVDWMTQIIFAALHTTSENGTIVLYRLLDNPKVLEELYEEQNAVLEEAGYDDTVGPEVFTREILNKFVKMDSVIRESCRLRNDYIGLPHTNVGKKTIVLSGGAMIRPGENAFVNFYSNHRDEKLQKSGMNANNFEPYRFVDQGKNSTKIGDDFMFFGMFKHACPGRWFAIQEIKTILAMLIRSYKMSAIDSVVFPTSDYTRIPTGRFKIVPRK

SEQ ID NO: 114 - DNA, 1605 bp, Lichtheimia corymbifera
  source 1..1605: mol_type = other DNA
atgtacaccctggtgtctttcttgaaggaccaaaacattattgccgttttgaagaacgctattcacgctcaacaacaaggtactactgcttcctctattaccattttgtctttggctgttgctattaccactttcgccgttcatagaattaggctgtattccgtaaaagaacacggtgttccattggttccatatgttttgcctttcattggttcttcaccagagtacagaaaagatccaaaggcttttttggaaaagtggactgctaaattcggtccagtttttagagcccatattttcggtagagtttacaccattatctccggtcattacgtcagagaagttttcttgaacgaggacttctcattcgaagttggtatgggtaaaactttcgatggttggttgattaccgataccaaaaagtctgagttgttctctacaccattggtcagatctatggttatgaagcacttagccgttgatgttaagaactatactccaagagctgttgagcatttgactattgctgctagagaaatgttgggtgatatcggtgaatctaaagaattgccacacttgtacccattgatccaacatatggtttctaccgctattgcctctattttggttggtttgaaaatctgcaaggacaaggatttgttggagacttttaagaacgttgccgtcgatattggttctgaattgaatccagattcctacttgtacgaagctttcccaactatctctagattgagacaatggtacttgggtaaatacggtaaggctattaacaagcaccaagagcatatgttgagagttttgggtccagaaattgacgaaagattggctgctatggaacgtggtgatggtggatgggaaagaccagaagatattttacaaggtattctggaaaccgccaagttgacttctgatcatccacaaagatatatggtcccaatcaagtggttcctgattttggtttttgcttccattcataccacctctcaaaacactacagttgctatgtataggttgttgcagcatccagaagttattgacgaactgttggaagaacaaaaccaggtctttgaaaaacaccacggttctaactacgatgatcatgatattaccaagttgttgaccggtgaagttattaaggatttggtcaagttggattccgtctgtagagaagctatgagaatctcttctttctacgctgaattgcctcatacttacatcggtaaatctccattgactatgtccaacggtactattatcaatccaggtgatgatgtcttgatcaacggttacactaaccatcatgatccagatatccaaattgatggtggtggtgattatgctgaattcaagccattcagattcgtcgaaaaaggtagacaatctaccagaatcggtgatgactacttgattttcggtcaaggtaaacatgcttgtccaggtagatggtttgctatgcaagaaatgaagaccaccatctcgtttttgatcagacagtacattattaccgccaagggtgacattatgtttttgaagggtcatagacaaaagatccctatgggtcaagttatcttccagaagagatga

SEQ ID NO: 115 - protein, 534 aa, Lichtheimia corymbifera
MYTLVSFLKDQNIIAVLKNAIHAQQQGTTASSITILSLAVAITTFAVHRIRLYSVKEHGVPLVPYVLPFIGSSPEYRKDPKAFLEKWTAKFGPVFRAHIFGRVYTIISGHYVREVFLNEDFSFEVGMGKTFDGWLITDTKKSELFSTPLVRSMVMKHLAVDVKNYTPRAVEHLTIAAREMLGDIGESKELPHLYPLIQHMVSTAIASILVGLKICKDKDLLETFKNVAVDIGSELNPDSYLYEAFPTISRLRQWYLGKYGKAINKHQEHMLRVLGPEIDERLAAMERGDGGWERPEDILQGILETAKLTSDHPQRYMVPIKWFLILVFASIHTTSQNTTVAMYRLLQHPEVIDELLEEQNQVFEKHHGSNYDDHDITKLLTGEVIKDLVKLDSVCREAMRISSFYAELPHTYIGKSPLTMSNGTIINPGDDVLINGYTNHHDPDIQIDGGGDYAEFKPFRFVEKGRQSTRIGDDYLIFGQGKHACPGRWFAMQEMKTTISFLIRQYIITAKGDIMFLKGHRQKIPMGQVIFQKR

SEQ ID NO: 116 - DNA, 1563 bp, Syncephalastrum racemosum
  source 1..1563: mol_type = other DNA
atgaacgccttgttgcaaagacaggataagatgagagatttcctgaacactagagaaggtgtttacggtattactgctgctgttgctgttgttttgatttccgcttattctttgagaagaaccgtctacacctctagaagaaaagacgaaattgctaccgttccatacaagttgccattgaaaggttctactgaagagtatagagctgatccaagagctttcgttgaaaagtacgctaaaatgtacggtccagtttacagagcttacttgtttggtgaaatgatgaccatcgtttccgattcttacgtcagagaaatcttcttgaacgacaacttcaacttcttccaagccgttaaggatagattcgatatggttaagttgtgcaactgcgctaatgatgaaggtcatggtattgctgatatcgtcaagagatgtttgaacccaagattggatatgtacaccgaaagagtcaacgaacaattagaggaagcctccaaagaaatcttgactgaagttgataccaagggctctcaagaattgatgcacatgtacattttggtccaacatatggttgctagagcttccgctactgtttttgttggtccagaattggctaagaactccgaattgattgactccttcaagaacatggttatccaagtcggttctcaactaagaccaaatccatggttggaaccatttccaagattgaactcattgaggatgtggtacattggtaagacttctccagttgttagaaagcacagattgcaattgagatctgctgttaagccatccatcgatgatagattggctagacaaaagtctcaaggtaacgcttttcaaagaccagacgatttgttgcaggatatcattgaaagacatccagaagccaagaccaagtctgatatctatgattacgttgttgataccttgaccgctttgatttttgctgcattgcataccacctctgaaaactctactgttgtcttgtataggttgttgcagcatcctgaattgatggaagaattggttgctgaacaagatgccgttttgttggaacacggtttgccaaaagattcttctgctagagttatgaccagacagatgatcaagaagttcgaaaagttggattctgtctgcagggaatctttcagattgagaaatgattacttgggtttgccacatacctacgaaggtaaaaaggatatcgttttgtctaacggtgccattattaagccaggtgaaaaggctattatcaacttgtggggtaatcatcattctggtaacactccacaatctacctctcaagattatttcggtttcgacccatacagattcgttaagcaagataagcaagctaccaagatctctgaggattttttgtttttcggtttgggtaaacatgcttgcccaggtagattttttgctgtccaagaagctaaggttctgatctctgttttgttgaggaactacaagttgtcaccagttgatccaccatactttgctactgatgataccatgaagattccagccggtagaattagaatcgaaagaaggtaa

SEQ ID NO: 117 - protein, 520 aa, Syncephalastrum racemosum
MNALLQRQDKMRDFLNTREGVYGITAAVAVVLISAYSLRRTVYTSRRKDEIATVPYKLPLKGSTEEYRADPRAFVEKYAKMYGPVYRAYLFGEMMTIVSDSYVREIFLNDNFNFFQAVKDRFDMVKLCNCANDEGHGIADIVKRCLNPRLDMYTERVNEQLEEASKEILTEVDTKGSQELMHMYILVQHMVARASATVFVGPELAKNSELIDSFKNMVIQVGSQLRPNPWLEPFPRLNSLRMWYIGKTSPVVRKHRLQLRSAVKPSIDDRLARQKSQGNAFQRPDDLLQDIIERHPEAKTKSDIYDYVVDTLTALIFAALHTTSENSTVVLYRLLQHPELMEELVAEQDAVLLEHGLPKDSSARVMTRQMIKKFEKLDSVCRESFRLRNDYLGLPHTYEGKKDIVLSNGAIIKPGEKAIINLWGNHHSGNTPQSTSQDYFGFDPYRFVKQDKQATKISEDFLFFGLGKHACPGRFFAVQEAKVLISVLLRNYKLSPVDPPYFATDDTMKIPAGRIRIERR

SEQ ID NO: 118 - DNA, 1584 bp, Absidia caerulea
  source 1..1584: mol_type = other DNA
atgctgaccgagtacatccatcacttcatcaacaatttcgaccagaaaaagaccatggaccaattgcaaactatggtgtcatctaaagaaggtatgattggtttggctactgctgctgttttgatgtctggtgctgcagtttacaagtctaccagaattgaaagaggttgtccacaagtcccaaatcagtcttactttatgggttctaccaaagagtacagaaacaatccagctgcctttatcgaaaagtgggaaaaagaattgggtccagtttatggtgcttacttgtttggtcagtacactactgttgtttctggtcctcaagttagggaagttttcttgaacgatgacttcgatttcattgccggtatcagaagagatttcgataccaacttgttgtctaacggtggtgatttgagagatttgccagttcataagtttgccggttccattaagaagaacttgtctccaaaattgcccttctacacctccagagttattgaacatttgaagatcggcctgaaagaattctgtggtgttgttccagatgagggtaaagaattcgatcatgtttacccattggttcaacatatggttgctaaagcttccgcttctgtttttgttggtccagaattggctaagaacgaacaattgattgactccttcaagaacatggttttggaagtcggttctgaattggctccaaaaccttacttggaattcttcccaaatttgatgagactgaggatgtggttcattggtaagacttctcaaaaggtcaagagacacagagatcaattgagagctgctttggctccacaagttgagtatagattgaaagccatgaaggaaaacgattccaattgggatagaccaaacgatttcttgcaggacattttggaatctggtgatattccagctcatgttgatgttactgatcattgctgtgattggatgacccaaattatctttgctgccttgcataccacatctgaaaatggtactttgtccttctacaggttgttggataatccaaaggtcttggaagatttgttggaagaacaaaaccaggtgttagaagatgctggttacgattcttctgttggtcctgaagttttcaccagagaaatcttgaacaagttcgtcaagatggactccgttattagagaaacctctagattgagaaacgactacattggtttgccacacaagaacatttcttctaagaccattactttgtctggtggtgctatgattagaccaggtgaaagagcttacgttaacgcttattctaaccacagagatggtactatccaaaaggttaccgataacttgaagtccttcgaaccatacagattcgttaaccaggatagaaactctaccaagatcggtgaagatttcatctttttcggtatgggtaagcacgcttgtccaggtagatggtttgctattcaagaaatcaagaccatcattgccatgatgatcagatcctatcaattgtctgctttgggtcctgttactttcccaactgatgattactctagaattcccatgggtagattcaaaatcgtgccaagaaagtaa

SEQ ID NO: 119 - protein, 527 aa, Absidia caerulea
MLTEYIHHFINNFDQKKTMDQLQTMVSSKEGMIGLATAAVLMSGAAVYKSTRIERGCPQVPNQSYFMGSTKEYRNNPAAFIEKWEKELGPVYGAYLFGQYTTVVSGPQVREVFLNDDFDFIAGIRRDFDTNLLSNGGDLRDLPVHKFAGSIKKNLSPKLPFYTSRVIEHLKIGLKEFCGVVPDEGKEFDHVYPLVQHMVAKASASVFVGPELAKNEQLIDSFKNMVLEVGSELAPKPYLEFFPNLMRLRMWFIGKTSQKVKRHRDQLRAALAPQVEYRLKAMKENDSNWDRPNDFLQDILESGDIPAHVDVTDHCCDWMTQIIFAALHTTSENGTLSFYRLLDNPKVLEDLLEEQNQVLEDAGYDSSVGPEVFTREILNKFVKMDSVIRETSRLRNDYIGLPHKNISSKTITLSGGAMIRPGERAYVNAYSNHRDGTIQKVTDNLKSFEPYRFVNQDRNSTKIGEDFIFFGMGKHACPGRWFAIQEIKTIIAMMIRSYQLSALGPVTFPTDDYSRIPMGRFKIVPRK

SEQ ID NO: 120 - DNA, 1584 bp, Absidia caerulea
  source 1..1584: mol_type = other DNA
atgttcacccaatacctgcatcagttgttcactaacttcgaccaaaaaaagaccatggaccacttgcattctctggtttcttctaaagaaggtgctattggtttggctactgctgctgttttgatgtctggtgctgcagtttacagatcttctatgatggatagaggttgtccagttgttccatctggtttgtttactttgtcatctactgctgagtacagacaagatccagcttctttcattaagaagtggcagaaagaattgggtccagtttatggtgcttacttgtttggtcaatacgttaccgttgtttccggttctcaagttagagaaattttcctgaacgagaacttctccttcatcgatggtatttccagagatttcgatacctacttgttggctaatgctggtacttatcatgatttgccaacttccactattgccgacatgattaagaagaacttgtctccaaagttgcagttctacaccggtagagttattgaacatttgaagatggccttgcatgaacaatgtggtgttgttccagctgaaggtaaagaattcaatcacgtttacccattcgttcaacatatggttgctaaagcttccgcttctgtttttgttggtgttgaattggctaagaacgaagccttggttgattctttcactaacatggttttagaagttggtggtgctttgggtccaaaaccttatatggaatacttccccaacttgatgaagttgcatatgtggtacattggcaagacttccaagaacgttaagagacaccaagaccaattgagatctgctttgaaaccagaaatcgacactagattgaaggccatgaaggaaaaagattcctcttgggttagaccaaacgatttcttacaagacttgttggaaaccgatgaatgcccagatcatattgacatctactccagagttatctactggatcacccaaattatctttgctgccttgcataccacatctgaaaatggtactttggccttgtataggttgttggataatccagaattattcgaggacttgtacgaagaacaaaaccaggtcttggaacaagctggttatgatagatctgttggtccagaagttttcaccagagaaatcttgaacaagttcgtcaagatggactccttgattagagaaacctctagattgaggaacgagttcatttctttgccacatatgaacacctccaacaagactattactttatcaggtggtgccatgattagaccaggtgaaaatgttttcattaacttctacgccaaccaccacgacgaaaaattgcaaaaagttgctgacaatttgggcaagttcgaaccatacagattcgttaatcaagacaagaactctaccaaggtcggtgaagattttgtttttttcggtatgggtaagcacgcttgtccaggtagatggtttgctattcaagaaatcaagaccatcatctccatgttgatcagagactacaaaatctctccattgggtcctgttgttttcccagtttctgattacactagaattccaaccggtagattcaaaatcgtcccaagaaagtga

SEQ ID NO: 121 - protein, 527 aa, Absidia caerulea
MFTQYLHQLFTNFDQKKTMDHLHSLVSSKEGAIGLATAAVLMSGAAVYRSSMMDRGCPVVPSGLFTLSSTAEYRQDPASFIKKWQKELGPVYGAYLFGQYVTVVSGSQVREIFLNENFSFIDGISRDFDTYLLANAGTYHDLPTSTIADMIKKNLSPKLQFYTGRVIEHLKMALHEQCGVVPAEGKEFNHVYPFVQHMVAKASASVFVGVELAKNEALVDSFTNMVLEVGGALGPKPYMEYFPNLMKLHMWYIGKTSKNVKRHQDQLRSALKPEIDTRLKAMKEKDSSWVRPNDFLQDLLETDECPDHIDIYSRVIYWITQIIFAALHTTSENGTLALYRLLDNPELFEDLYEEQNQVLEQAGYDRSVGPEVFTREILNKFVKMDSLIRETSRLRNEFISLPHMNTSNKTITLSGGAMIRPGENVFINFYANHHDEKLQKVADNLGKFEPYRFVNQDKNSTKVGEDFVFFGMGKHACPGRWFAIQEIKTIISMLIRDYKISPLGPVVFPVSDYTRIPTGRFKIVPRK

SEQ ID NO: 122 - DNA, 1461 bp, Phycomyces blakesleeanus
  source 1..1461: mol_type = other DNA
atggccatcatcttgtcctctgtttacaactatcataagtggtcctccagaaccatttctaatggttgtccaagagttccacataccttgccattttttggtttgaccaaggtttaccgtaaggattctaaggctttttgcgaagaatggcatgctaaattgggtccagtttttagagcacacttgttcggtaaagaagttaccgttgtttctggtcactacgtcagagaagtttttttgaacaagcacttcgacttcatcaagggtatcgttaaggtttttgataccaggttgttgaccgataacggttctagagaagattttccaccagaagatttgagggaaatcatcactaagtacttgaccccaaagttgaacatctacactagaagattgatcaagcagttgaagcaagacgtcgaaaacattttgggtttgatcgagttcgataacttgtacccattcgttcaacacttgatcgttaatgcttccgcctctattttcttgggtgaagaaaattcccagaacaagttgttgatcgacagcatcaagaacatggttagattggttggttccgaagttaagcaaaacccatggattgaaccattcagcccaatcaaaaagatcagaatgtgggttattggtaagacctctccagttatcaggtcctacaaagaacaattgattaacgccatcaagccagttgttgagtacagattgtctgaagctagaagaaatccagactggaaaaaacctactgatgtgttgcaagacttgttggaaaattctaaaccaccagctcacatggatttgatggattacttggtctgcattatcaccatcttgattttcgttgccttgcattccactattgaaaacactaccgttctgttgtacaggatcttggaaaacccagaaatcatggatgaattggacttggaacaaagggaagtcattgaacaagaaggtttggataccaattgtggctctgaattattcaccagagacatcttgaagaagttcaccaaattggattctgtctgcagagaaaccttcagaatgaagaaccagtacatcactttgccacatgaatacgatggtaaggttccattgactttgtctaatggtgctgttattaacccaggtgatgatgttttgattgatgtttggaccaaccacaggtacaagaaagaaactacttctgttaaggatgccgacgaattcagaccattcagatttgttaatcagaacaagcagtctaccaaggtcggtgaagattacttgttttttggtatgggtagacatgcctgtccaggtagatggtttgctatgcaagaaattcaagctattaccgccatcttggttagagaatgcaagtttattccaaagggcccaattatctttccaaccgctgaaagatctccaattccaactggtagatgtatcatccagagaaagtga

SEQ ID NO: 123 - protein, 486 aa, Phycomyces blakesleeanus
MAIILSSVYNYHKWSSRTISNGCPRVPHTLPFFGLTKVYRKDSKAFCEEWHAKLGPVFRAHLFGKEVTVVSGHYVREVFLNKHFDFIKGIVKVFDTRLLTDNGSREDFPPEDLREIITKYLTPKLNIYTRRLIKQLKQDVENILGLIEFDNLYPFVQHLIVNASASIFLGEENSQNKLLIDSIKNMVRLVGSEVKQNPWIEPFSPIKKIRMWVIGKTSPVIRSYKEQLINAIKPVVEYRLSEARRNPDWKKPTDVLQDLLENSKPPAHMDLMDYLVCIITILIFVALHSTIENTTVLLYRILENPEIMDELDLEQREVIEQEGLDTNCGSELFTRDILKKFTKLDSVCRETFRMKNQYITLPHEYDGKVPLTLSNGAVINPGDDVLIDVWTNHRYKKETTSVKDADEFRPFRFVNQNKQSTKVGEDYLFFGMGRHACPGRWFAMQEIQAITAILVRECKFIPKGPIIFPTAERSPIPTGRCIIQRK

SEQ ID NO: 124 - DNA, 1608 bp, Lichtheimia corymbifera
  source 1..1608: mol_type = other DNA
atgtacaaggccttcttggacaacggtatcgtttcctctattatctctaccttggataacaaggacatctccgtttttttgaacgatccaaaccatgctaagaccagatctgttgctttggtttctttctgtactgttattgctgcttacgccttgtctagatcaagatctcattctaaggataaggataccccaatggttccatatacttggccattgattggttcttctagagaataccgtaaagatcctgaagcctttatcaagaagtggtcatctgaattgggtgatgtttacaaggttcacttgttcggtagaatccaaactgttgtttccggtaaacacgtttactgcttgcaaaaggatttcgactttcaacagggtatgtctaagaccttcgatatctggttgttgttggatgctccattaggtggtagattcactttggataagattagacatgccaccatcaagttcaccagaactaagatgttgactaacactccatgcgttgtcaagcaattgattgctgctgaacacgaaatgattggtgatgctcaaactccatctgaaattgctaacttgtacccattgatggaacacttggttgctattgcttccgctactaattttgttggtccaggtttgaccaaagataaggatttggttgaaacctacaagcacttggctgttgatgttggttcagaattaggtgatggtaacgaattcttggaagctttcccatggatttccagattgagaatgtggtacttgggtaaatacggtaactctgttgataagcacagaaagcgtttgttgagagctatgaagccaattatcgacgaaagattggctgctgcagaaaacggtattgaaaatccacaagatttcatccaggacatcatcgaagaatccgaaattacttctggtgatccagacaagtacattttggcagttagatggatcttggttatgattgcttctgctggtcataccactactgaaaacactaccattatcctgtacagaatcttgcaacacccagaagtcattgacgaactattggaagaacaaagacaggtcttggaaaaacatcatggtccagatgttaaggataacgaagatttggctactttgttcactggtgaagttattaaggacttggtcaagttggattctgtctgtagagaaaccatgagattgaggtccttctacattgatttgccacatacctacgttggtaaatctccattggctttgactaatacctgtaccattaagccaggtgaagatgtcttgttgaatatgtggttgaaccataacagaaccgccatgcaatatgatggtttgggtgattacaatgagttcaagccattcagatttgtcggtttggatagatcctctactaagttaggtggtgactttttgttgttcggtttgggtactcatgcttgtccaggtagatggtttgctatgcatcaaactaagaccatcctgtccatgttgttgagaagataccaaattactccccaagaaaccatcgttttcccaattggtaatagatcccatgttccatctggtaaggttacctttcaaaagagacagtaa

SEQ ID NO: 125 - protein, 535 aa, Lichtheimia corymbifera
MYKAFLDNGIVSSIISTLDNKDISVFLNDPNHAKTRSVALVSFCTVIAAYALSRSRSHSKDKDTPMVPYTWPLIGSSREYRKDPEAFIKKWSSELGDVYKVHLFGRIQTVVSGKHVYCLQKDFDFQQGMSKTFDIWLLLDAPLGGRFTLDKIRHATIKFTRTKMLTNTPCVVKQLIAAEHEMIGDAQTPSEIANLYPLMEHLVAIASATNFVGPGLTKDKDLVETYKHLAVDVGSELGDGNEFLEAFPWISRLRMWYLGKYGNSVDKHRKRLLRAMKPIIDERLAAAENGIENPQDFIQDIIEESEITSGDPDKYILAVRWILVMIASAGHTTTENTTIILYRILQHPEVIDELLEEQRQVLEKHHGPDVKDNEDLATLFTGEVIKDLVKLDSVCRETMRLRSFYIDLPHTYVGKSPLALTNTCTIKPGEDVLLNMWLNHNRTAMQYDGLGDYNEFKPFRFVGLDRSSTKLGGDFLLFGLGTHACPGRWFAMHQTKTILSMLLRRYQITPQETIVFPIGNRSHVPSGKVTFQKRQ

SEQ ID NO: 126 - DNA, 1596 bp, Hesseltinella vesiculosa
  source 1..1596: mol_type = other DNA
atgctgacccaatacttgcaattcgctgctgatagattgggtcaaaaaaagaccttggatcaattgcaagctgttgtcacttctaagcaaggtgttgttggtattactactgctgttgctttgattgctttggccaatcattttagatccccaaagattgatagaggttgcccacaagttgaaggtaaaggttggtttggttatgctaccgaagaattcagagaaaacccaggtaagtttttgtctgaatggcacgaaaaattgggtccagtttacggtgttaagatctttggtcattacgctactgttgtttctggtccatatgtcagagaagttttcttggatgacaggttctctttcattgctgctattaccaagttgttcgacccaaacttgatgactgattctggtcattcttctgaacaaactgctaagaatgctgccgattccattaagagatttctgtctccaaatctgaagcactacaccccaagagttattgaacatttgaacttgggtatcgaagattggtgtggtgaagttccagctgaaggtattgaaattgaaaacgctttcccattcttgcaacatttggttgctagagcttcagcttctgttttcgttggtattgaattggccaagaacgaagaattggttgactctttccaaaacatggtgtccaacatttcttctggtttgaaacctaaaccttggttggaatactttccctccttgaccaaattgggtatgtacatgattggtaagactaacccagctgttaagagacatagaactcaaatggctaacgccttaagaccagaagttgatagaagattgaaagccatggcctctaatgataccaattgggatagaccagatgatatgttgcagcacattttggaatcttatccagctcctgaaggtttggatgttattacctatttgatcaactggatgacgcagttgatttttgctgcattgcataccacatctgaaaacggtaaagttgtcttgtacagattgctacaacacccagaagtcatggaagaattatacgctgaacaaaacgaagttttggctgctgctggttatgatgaatctgctggtccagaagtttttgacagagagatgttgaacaagttcgtcaagttggattctgctgttagagaagcttgtaggttgaagaatgaattcgttggtttgccacacgaaaacactactgataagactttgactttgtccaacggtgctgttattttgccaggtgaatttgtttacatcaaccagttcgttaaccacagggatccagaattacaagctgctattgatgatgtccatcagttcaaaccattcagattcgtaggtactgatcataacgctgctaaagtttccgaaggttttgtcttttttggtatgggtagacatgcttgtccaggtagatggtttgctattcaagaaatcaagaccatcgtgtccttgttgttgagaaagtacaaggttgaacctatcgacccaatcgtgttctctaatcaagaaagagatgcctttccaattggtccatgcagaattagattgacaccaagaaaagccctgtaa

SEQ ID NO: 127 - protein, 531 aa, Hesseltinella vesiculosa
MLTQYLQFAADRLGQKKTLDQLQAVVTSKQGVVGITTAVALIALANHFRSPKIDRGCPQVEGKGWFGYATEEFRENPGKFLSEWHEKLGPVYGVKIFGHYATVVSGPYVREVFLDDRFSFIAAITKLFDPNLMTDSGHSSEQTAKNAADSIKRFLSPNLKHYTPRVIEHLNLGIEDWCGEVPAEGIEIENAFPFLQHLVARASASVFVGIELAKNEELVDSFQNMVSNISSGLKPKPWLEYFPSLTKLGMYMIGKTNPAVKRHRTQMANALRPEVDRRLKAMASNDTNWDRPDDMLQHILESYPAPEGLDVITYLINWMTQLIFAALHTTSENGKVVLYRLLQHPEVMEELYAEQNEVLAAAGYDESAGPEVFDREMLNKFVKLDSAVREACRLKNEFVGLPHENTTDKTLTLSNGAVILPGEFVYINQFVNHRDPELQAAIDDVHQFKPFRFVGTDHNAAKVSEGFVFFGMGRHACPGRWFAIQEIKTIVSLLLRKYKVEPIDPIVFSNQERDAFPIGPCRIRLTPRKAL

SEQ ID NO: 128 - DNA, 1554 bp, Nicotiana sylvetris
  source 1..1554: mol_type = other DNA
atggtgtctccagttgaagctatcgttggtttggttactttggctttgttgttctacttcatcaggaccaagaaatcccaaaaaccatctaaaccattgccaccaaaaattccaggtggttggccagttattggtcacttgttttacttcgatgatgactccgatgatagaccattggctagaaaattgggtgatttggctgataagtacggtccagtttttactttcagattgggtttgccattggtcttggttgtttcatcttacgaagctatcaaggattgcttctctaccaacgatgctatcttttctaatagaccagctttcttgtacggtgagtatttgggttataacaacgccatgttgttcttgactaagtatggtccatattggaggaagaacagaaagttggttatccaagaggttttgtgcgcttctagattggaaaaattgaagcacgttaggttcggtgaaatccaaacctctattaagaacttgtacaccagaatcgacggtaactcttctactattaacttgaccgattggctggaagagttgaattttggtttgatcgttaagatgatcgccggtaagaattacgaatctggtaaaggtgatgaacaggtcgaaagattcagaaaggctttcaaggatttcatcatcctgtccatggaattcgttttgtgggatgcttttccaattcctttgttcaagtgggttgatttccaaggtcatgttaaggctatgaagagaaccttcaaggatatcgactctgttttccaaaactggttggaagaacacgtcaaaaagaaagaaaagatggaagttaacgccgaaggtaacgaacaagatttcatcgatgttgtgctgtccaagatgtctaacgaatatttggatgaaggttactccagagataccgttattaaggctactgttttctccttggttttggatgctgctgatactgttgcattgcatatgaattggggtatggccctgttgattaacaatcaacatgctttgaagaaggcccaagaagaaatcgacaaaaaggttggtaaagacagatgggttgaagagtccgatattaaggatttggtttacttgcagaccatcgtcaaagaagttttgagattatatccaccaggtcctttgttggttccacacgaaaatgttgaagattgcgttgtttccggttaccatattccaaagggtactagattattcgccaacgtcatgaagttgcaaagggatccaaaattgtggtctaacccagataagttcgatccagaaagatttttcgctgccgatattgatttcagaggtcaacattacgaattcatcccatttggttctggtagaagatcttgtccaggtatgacttatgctatgcaagttgaacatttgactatcgcccatttgatccaaggtttcaattacaagactccaaacgatgaaccactggatatgaaggaaggtgctggtttgacaattagaaaggttaacccaatcgaagttgtcatcactccaagattgactccagagttgtactga

SEQ ID NO: 129 - protein, 517 aa, Nicotiana sylvestris
MVSPVEAIVGLVTLALLFYFIRTKKSQKPSKPLPPKIPGGWPVIGHLFYFDDDSDDRPLARKLGDLADKYGPVFTFRLGLPLVLVVSSYEAIKDCFSTNDAIFSNRPAFLYGEYLGYNNAMLFLTKYGPYWRKNRKLVIQEVLCASRLEKLKHVRFGEIQTSIKNLYTRIDGNSSTINLTDWLEELNFGLIVKMIAGKNYESGKGDEQVERFRKAFKDFIILSMEFVLWDAFPIPLFKWVDFQGHVKAMKRTFKDIDSVFQNWLEEHVKKKEKMEVNAEGNEQDFIDVVLSKMSNEYLDEGYSRDTVIKATVFSLVLDAADTVALHMNWGMALLINNQHALKKAQEEIDKKVGKDRWVEESDIKDLVYLQTIVKEVLRLYPPGPLLVPHENVEDCVVSGYHIPKGTRLFANVMKLQRDPKLWSNPDKFDPERFFAADIDFRGQHYEFIPFGSGRRSCPGMTYAMQVEHLTIAHLIQGFNYKTPNDEPLDMKEGAGLTIRKVNPIEVVITPRLTPELY

SEQ ID NO: 130 - DNA, 2121 bp, Lictheimia ramosa
  source 1..2121: mol_type = other DNA
atggctcaatctccaccagcattggatactttggatatcgttttcttgggtactatcggtttgggtacaattgcttggtttgctagaaggcaaattgctgaaagattattcggttccacctcttcctctgatgctaaatctaatggtcatgctactccagctccaccaaaaagagaaagaaacttcgttaagatcatgcaagagcaaggtagaaaggtcattttcttctacggttctcaaactggtactgctgaagattacgcttctagattggctaaagaatgctctcaaaagtacggtgttaactgtatgactgctgatttggagttgtacgacttgtcttacttggatactgttccagaagattgcctggtttttttcgttatggctacttatggtgaaggtgaacctactgataatgctgttgatttctgggatgtcttgtctgaagaagaaccacaattttctgaagccgaaggtgataagccattgcaaaatttgagatacttggttttcggcttgggtaacaagacttacgaacattataacgctgttgccagaaacgttgacaagagattggaagttttgggtgctcatagaattcatgaacgtggtgagggtgatgatgatggttctttggaagaagattttttggcctggcaagaaaacatgtggccagctttttgtgaagctttgggtgttgatgaatctaacgctcattctggtccaagacaagctacttattctgttgaagaattggtcgatgtcaacatggatgatgtttacttgggtgaattggccgaaaaacctaaagaaggtgctagagttatctacgatgctaagaggccttttaatgctccaattgccatttctcaagacttgttcactaacaccgatagacattgcttgcacatggaaatcgatatctccgattccaacttgtcataccaaaccggtgatcatattgctatttggccaactaactccgaaaacgaagttgctagactggcttctattttgggtttagctgataagttggataccgccatcaatgttaaggctattgatccagctgcttccaaaaagtacccatttccttgtccagctacttacagagctattttcagacattacttggacatttgcgctgccgtttctagacaatctttgatggcttttgttgaatacgctccaaccgaagaatccaaagaaagattgagacaattggccaaggataaggatgagtacagattgactgttggtgaagctgttagaaatttgggtgaagtattggaaatcgttgctggtaatgatgttaagccaggtttcttttcttccgttccattcgatttggttgtcgaatctatttccagattgcaacctaggtactactccatttcttcatctgcaaaagagtccccaaaaaagatcgctgttactgctgttacattgtcctatcaaccagatccaactccacaaagaactgtttatggtgtcaacactaattacttgtggcgtattcataccgcctctaagcaacaatctactgaagctgatttgccaacctatgatttgtcaggtccaagaaatgctttacatggtactaagttgccagttcacgttagaagatctcaattcaagttgccaagaaacccaaccgttccagttattatggttggtccaggtactggtgttgctccttttagaggttttgttagagaaagagccttgcagaaatctgaaggtaaaccagttggtccaactttgttgtttttcggttgtagaaactccgaacaggacttcttgtacaaagatgaatggcctgctttgtttgacaccttaggtgaatcctctagaattattaccgccttctcaagagaaaccgctcaaaaagtttacgtccagcatagattgcaagagaacggtcaagaagtttggggtttgttgcaaaggggtgcttatatctatgtttgtggtgatgcaaagaacatggccagagatgttcaacaaactttcgttaacttcggtatcgaattcggtggtttgtctgatgataaggctcatgattttgtcaagaacttgagaaacaccggtagataccaagaagatgtttggtcttga

SEQ ID NO: 131 - protein, 706 aa, Lichtheimia ramosa
MAQSPPALDTLDIVFLGTIGLGTIAWFARRQIAERLFGSTSSSDAKSNGHATPAPPKRERNFVKIMQEQGRKVIFFYGSQTGTAEDYASRLAKECSQKYGVNCMTADLELYDLSYLDTVPEDCLVFFVMATYGEGEPTDNAVDFWDVLSEEEPQFSEAEGDKPLQNLRYLVFGLGNKTYEHYNAVARNVDKRLEVLGAHRIHERGEGDDDGSLEEDFLAWQENMWPAFCEALGVDESNAHSGPRQATYSVEELVDVNMDDVYLGELAEKPKEGARVIYDAKRPFNAPIAISQDLFTNTDRHCLHMEIDISDSNLSYQTGDHIAIWPTNSENEVARLASILGLADKLDTAINVKAIDPAASKKYPFPCPATYRAIFRHYLDICAAVSRQSLMAFVEYAPTEESKERLRQLAKDKDEYRLTVGEAVRNLGEVLEIVAGNDVKPGFFSSVPFDLVVESISRLQPRYYSISSSAKESPKKIAVTAVTLSYQPDPTPQRTVYGVNTNYLWRIHTASKQQSTEADLPTYDLSGPRNALHGTKLPVHVRRSQFKLPRNPTVPVIMVGPGTGVAPFRGFVRERALQKSEGKPVGPTLLFFGCRNSEQDFLYKDEWPALFDTLGESSRIITAFSRETAQKVYVQHRLQENGQEVWGLLQRGAYIYVCGDAKNMARDVQQTFVNFGIEFGGLSDDKAHDFVKNLRNTGRYQEDVWS

SEQ ID NO: 132 - DNA, 1083 bp, Papaver somniferum
  source 1..1083: mol_type = other DNA
atggaaactcctatactaattaagttgggaaacggtctaagtattccttcagtgcaggagttagcaaaattaacattagcggagatcccaagccgatacacgtgcaccggcgaatccccattgaataacatcggtgcttcagtcaccgatgacgagaccgttcccgtcatcgacctacagaacttgctttctccagagccggtagtaggaaagttggagttagataagttacacagcgcatgcaaggagtggggtttctttcagctagtaaatcacggagtagatgctttactaatggataatattaagtccgagataaagggcttctttaatctgccaatgaatgagaagaccaaatacggacagcaagacggggatttcgagggcttcggccaaccttacatcgagagcgaggaccagcgtcttgactggacagaggtattttccatgctctcactgccacttcacctgaggaagccgcatttattccctgaattgccgctgccttttagagaaactctagaatcttatctatcgaaaatgaagaagttatcgacggtggttttcgaaatgttagagaagagcctacagctagtcgagattaaaggaatgacagacttattcgaggacggccttcagacaatgcgtatgaactactatccaccatgtcccagacctgagctagttttaggtttgacgtctcactcagacttttcaggtctgaccatcctgttacaactgaatgaggtcgaaggtctacagatacgcaaagaggagagatggatctctataaagcccctacccgacgctttcattgtgaatgtgggagatatattggagataatgacgaacgggatatacagatccgttgagcaccgcgctgtcgtaaactcaaccaaggaaaggttgagtatagctacgtttcacgattcgaaattagagagcgagataggccctattagttccttagtcactcccgagaccccggccttattcaaaagagggcgttacgaggatatacttaaagaaaacttaagtcgaaagctggatgggaaatcatttcttgattacatgcgaatgtag

SEQ ID NO: 133 - protein, 360 aa, Papaver somniferum
METPILIKLGNGLSIPSVQELAKLTLAEIPSRYTCTGESPLNNIGASVTDDETVPVIDLQNLLSPEPVVGKLELDKLHSACKEWGFFQLVNHGVDALLMDNIKSEIKGFFNLPMNEKTKYGQQDGDFEGFGQPYIESEDQRLDWTEVFSMLSLPLHLRKPHLFPELPLPFRETLESYLSKMKKLSTVVFEMLEKSLQLVEIKGMTDLFEDGLQTMRMNYYPPCPRPELVLGLTSHSDFSGLTILLQLNEVEGLQIRKEERWISIKPLPDAFIVNVGDILEIMTNGIYRSVEHRAVVNSTKERLSIATFHDSKLESEIGPISSLVTPETPALFKRGRYEDILKENLSRKLDGKSFLDYMRM

SEQ ID NO: 134 - DNA, 62 bp, synthetic construct
  source 1..62: mol_type = other DNA
aagcagtggtatcaacgcagagtggccagaatggcctttgttttttctttttttttttttvn

SEQ ID NO: 135 - DNA, 21 bp, synthetic construct
  source 1..21: mol_type = other DNA
agtggcctgcagggccggggg

SEQ ID NO: 136 - DNA, 23 bp, synthetic construct
  source 1..23: mol_type = other DNA
aagcagtggtatcaacgcagagt

(19) United States
(12) Patent Application Publication
(10) Pub. No.: US 2024/0150804 A1
(43) Pub. Date: May 9, 2024

(54) Demethylation of Reticuline and Derivatives Thereof with Fungal Cytochrome P450

(71) Applicant: RIVER STONE BIOTECH, LLC, Cambridge, MA (US)

(72) Inventors: Markus Schwab, Reinach (CH); Sumire Honda Malca, Reinach (CH); Philipp Friedrich Berninger, Reinach (CH); Jon Richard Heal, Reinach (CH); Joseph Michael Sheridan, Reinach (CH); Laura Tatjer Recorda, Copenhagen (DK); Rubini Maya Kannangara, Copenhagen (DK); Esben Halkjaer Hansen, Copenhagen (DK); Angela Manuela Ribeiro de Carvalho, Copenhagen (DK)

(21) Appl. No.: 18/452,054

(22) Filed: Aug. 18, 2023

Related U.S. Application Data
(63) Continuation of application No. 16/623,052, filed on Dec. 16, 2019, now Pat. No. 11,781,164.

(30) Foreign Application Priority Data
Jun. 16, 2017 (DK) PA201770473

(51) Int. Cl. C12P 17/18

(52) U.S. Cl. CPC C12P 17/18

(57) ABSTRACT

The invention relates to recombinant host cells that expresses one or more genes encoding a cytochrome P450 enzyme capable of N-demethylating and/O-demethylating reticuline and/or derivatives thereof, and also methods of producing a N-demethylated and/or O-demethylated reticuline and/or derivatives thereof, comprising cultivating the recombinant host of the invention in a culture medium under conditions in which the one or more genes encoding the cytochrome P450 enzymes is/are expressed. The reticuline and derivatives thereof are useful for providing access to naturally unavailable and chemically difficult-to-produce starting materials for opioids.

This application is a continuation of U.S. application Ser. No. 16/623,052, filed Dec. 16, 2019, which is a U.S. national phase application of International Patent Application No. PCT/EP2018/066155, filed on Jun. 18, 2018, which claims the benefit of Danish Patent Application PA 2017 70473 filed on Jun. 16, 2017, each of which is incorporated by reference in their entirety.

### REFERENCE TO AN ELECTRONIC SEQUENCE LISTING

A computer readable form of the Sequence Listing is filed with this application by electronic submission and is hereby incorporated by reference in its entirety. The Sequence Listing is contained in the electronic file created on Jan. 9, 2024, having the name “19-2315-WO-US-CON_Sequence-Listing.xml” and is 262,433 bytes in size.

### FIELD OF THE INVENTION

The invention relates to recombinant host cells that express one or more genes encoding a cytochrome P450 enzyme capable of N-demethylating and/or O-demethylating reticuline and/or derivatives thereof, and also methods of producing N-demethylated and/or O-demethylated reticuline and/or derivatives thereof, comprising cultivating the recombinant host of the invention in a culture medium under conditions in which the one or more genes encoding the cytochrome P450 enzymes is/are expressed. The invention also relates to in vitro bioconversion processes that produce N-demethylated and/or O-demethylated reticuline and/or derivatives thereof. The reticuline and derivatives thereof are useful for providing access to naturally unavailable and chemically difficult-to-produce starting materials for opioids.

### BACKGROUND OF THE INVENTION

Thebaine and oripavine extracted from plant material are starting materials for chemical synthesis of semisynthetic marketed opioids including buprenorphine, naltrexone, naloxone and nalbuphine.

Chemical synthesis of buprenorphine, naltrexone, naloxone and nalbuphine involves N-alkylation which is preceeded by N-demethylation of thebaine or oripavine or a derivative thereof. This step is one of the most critical in the chemical synthesis of the above-mentioned compounds as it has low efficiency and produces highly toxic waste products.

N-demethylation of thebaine and oripavine or derivatives thereof by fungi belonging to the order Mucorales has been described previously by Madyastha et al. (J. Chem. Soc. Perkin. Trans., vol. 3, p. 911), by K. Madyastha, et al. (Indian J. Chem., vol. 39, pp. 377-381, 2000), and by Chaudhary et al. (Collect. Czechoslov. Chem. Commun., vol. 74, no. 7-8, pp. 1179-1193, 2009).

Furthermore, opiate demethylation including thebaine has previously been demonstrated with human CYP3A4 and CYP3A5 by Kramlinger et al. (J Biol Chem, vol. 290, no. 33, pp. 20200-20210, 2015), and by Lalovic et al. (Drug Metab. Dispos., vol. 32, no. 4, pp. 447-454, 2004). Also, variants of the cytochrome P450 BM3 from Bacillus megaterium have been reported to possess similar activities (Lewis et al. (Chembiochem, vol. 11, no. 8, pp 2502-2505, 2010))

However, the activity demonstrated so far for the human/bacterial P450 enzymes and naturally active fungi are not anywhere close to being efficient enough for a commercially relevant enzymatic/biological demethylation process and there is a concrete need for isolation of more active enzymes which are suitable for expression in heterologous hosts. Without knowing the gene sequence and thereby the amino acid sequence of the enzymes responsible for the demethylation reactions such expression in heterologous hosts is not possible. Before the present invention it was not known which type of enzyme was responsible for the N-demethylation reaction in fungi of the mucorales order and sequences of the responsible enzymes not isolated. Filamentous fungi can like plants typically have more than hundred Cytochrome P450 and dioxygenase enzymes, which could all be candiates for being N-demethylases. The first isolation of the specific enzyme responsible for this reaction from a specific fungus is therefore a very complex task, and in particular when the species is not genome sequenced. Finding more homologs of the first isolated gene in related sequenced species using BLAST search is on the other hand less difficult.

An efficient setup for the production of reticuline and/or derivatives thereof is needed in order to pursue chemical synthesis of the semisynthetic marketed opioids.

Such setup would provide access to naturally unavailable and chemically difficult to produce starting materials for the opioids, such as northebaine and nororipavine, in an economic and sustainable process.

### SUMMARY OF THE INVENTION

The invention relates to a recombinant host cell that expresses one or more genes encoding a cytochrome P450 enzyme capable of N-demethylating and/or O-demethylating reticuline and/or derivatives thereof, wherein at least one of the genes is a recombinant gene.

Certain embodiments of the invention relate to host cells of the invention wherein reticuline and derivatives thereof can be (S)-reticuline, 1,2 dehydroreticuline, (R)-reticuline, salutaridine, salutaridinol, thebaine, oripavine, neopinone, codeinone, codeine, morphinone, morphine, hydrocodone, 14-hydroxycodeinone, 7-O-acetyl-salutaridinol or oxycodone.

In certain embodiments of the host cells of the invention, the reticuline derivative is thebaine or oripavine.

In certain embodiments of the host cells of the invention, cytochrome P450 enzymes capable of N-demethylating and/or O-demethylating reticuline and/or derivatives thereof have at least 20% sequence identity with P450_DN15259_c0_g1_i7 (SEQ ID NO: 3), such as 30% sequence identity, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 95% sequence identity, such as 97% sequence identity, such as 98% sequence identity, such as 99% sequence identity.

In certain embodiments of the host cells of the invention, cytochrome P450 enzymes capable of N-demethylating and/or O-demethylating reticuline and/or derivatives thereof have at least 20% sequence identity with CYPDN8 (SEQ ID NO: 72), such as 30% sequence identity, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 95% sequence identity, such as 97% sequence identity, such as 98% sequence identity, such as 99% sequence identity.

In additional embodiments of the invention, host cells further express one or more cytochrome P450 reductase(s) (CPR(s)). This one or more reductase can be endogenous or heterologous, or there can be one or more of both.

The invention provides host cells, wherein the cell is a yeast cell including but not limited to Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

The invention also relates to methods for producing reticuline or derivatives thereof, comprising cultivating the recombinant host cell of the invention in a culture medium under conditions in which the one or more genes encoding the cytochrome P450 enzymes is/are expressed and wherein at least one of the genes is recombinant.

In certain embodiments of the host cell of the invention, reticuline or derivatives thereof include but are not limited to (S)-reticuline, 1,2 dehydroreticuline, (R)-reticuline, salutaridine, salutaridinol, thebaine, oripavine, neopinone, codeinone, codeine, 7-O-acetyl-salutaridinol, morphinone, morphine, hydrocodone, 14-hydroxycodeinone or oxycodone.

In certain embodiments of the methods of the invention, the recombinant host of the invention are cultivated in a culture medium under conditions in which the one or more genes encoding the cytochrome P450 reductase is/are expressed and wherein at least one of the genes is recombinant.

The invention further relates to compositions comprising compounds that are reticuline or derivatives thereof that can be obtained from the methods according to the invention, wherein said methods further comprise elements from a fungal fermentation broth and/or at least one fungal specific metabolite Another aspect of the disclosure relates to a method of preparing buprenorphine, or a salt thereof, from Compound HO—I—H, or a salt thereof:

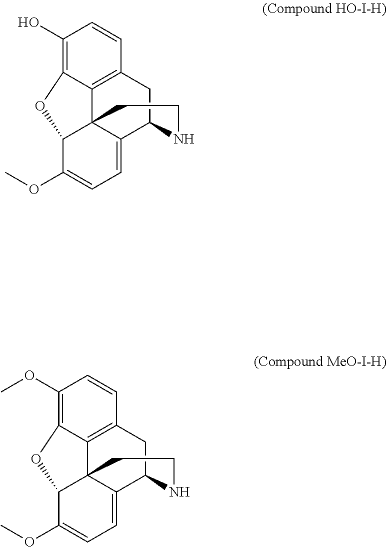 [SMILES: COC1=CC=C2[C@H]3Cc4ccc(O)c5c4[C@@]2(CCN3)[C@H]1O5 ; COC1=CC=C2[C@H]3Cc4ccc(OC)c5c4[C@@]2(CCN3)[C@H]1O5]

comprising reacting compound HO—I—H (Nororipavine) or compound MeO—I—H (Northebaine) through a series of steps to provide buprenorphine.

Aspects and embodiments of the disclosure related to methods of preparing buprenorphine from Compound MeO—I—H, or HO—I—H provide improved routes to buprenorphine that can be shorter, more efficient, and/or produce less toxic waste than, e.g., current commercial routes to buprenorphine. As a result, these aspects and embodiments can be well-suited for commercial (e.g., kg-scale) production of buprenorphine. Further, in certain aspects and embodiments, the synthetic routes disclosed herein advantageously avoid the harsh conditions and/or toxic byproducts of an N-demethylation step and can accordingly be particularly well-suited for producing buprenorphine on a commercial, e.g., kg, scale.

### BRIEF DESCRIPTION OF THE FIGURES

FIG. 1: Bioconversion of thebaine to northebaine by Cunninghamella echinulata and Thamnostylum piriforme. Thebaine in a final concentration of 0.5 mg/mL was added after 48 h of growth. Samples were taken 11 days after thebaine addition.

FIG. 2: Bioconversion of thebaine to northebaine by Thamnostylum piriforme. Thebaine in a final concentration of 0.5 mg/mL was added after 48 h of growth. Total RNA was extracted from biomass sampled on day 0, 6 and 9 after thebaine addition.

FIG. 3: Screening of thebaine N-demethylation by S. cerevisiae strains expressing cytochrome P450 candidates from Thamnostylum piriforme in combination with CPRs from Cunninghamella elegans, Gibberella fujikuroi and S. cerevisiae. Cells were fed with 0.1 mM (31 mg/L) thebaine in non-buffered selective medium and grown at 30° C. with shaking at 300 rpm for 72 h.

FIG. 4: N-demethylation of thebaine by S. cerevisiae strains expressing Thamnostylum piriforme P450_DN15259_c0_g1_i7_A in combination with codon-optimized CPRs from Cunninghamella elegans and Gibberella fujikuroi or native CPRs from S. cerevisiae and T. piriforme (CPR_DN2505_c0_g1_i1, CPR_DN5866_c0_g1_i1 and CPR_DN10898_c0_g1_i1). NC: Negative control strain. Cells were fed with 0.1 mM (31 mg/L) thebaine in selective medium containing 0.1 M potassium phosphate buffer pH 7 and grown at 30° C. with shaking at 300 rpm for 72 h.

FIG. 5A-5B: N-demethylation of thebaine and oripavine by S. cerevisiae strains expressing Thamnostylum piriforme P450_DN15259_c0_g1_i7_A in combination with the codon-optimized CPR from Cunninghamella elegans or native CPRs from T. piriforme. NC: Negative control strain. Cells were fed with 0.1 mM or 0.5 mM thebaine (A) or oripavine (B) in selective medium containing 0.1 M potassium phosphate buffer pH 7 and grown at 30° C. with shaking at 300 rpm for 72 h.

FIG. 6: Screening of oripavine N-demethylation by S. cerevisiae strains expressing cytochrome P450 candidates from Thamnostylum piriforme in combination with CPRs from Cunninghamella elegans, Gibberella fujikuroi and S. cerevisiae. Cells were fed with 0.5 mM oripavine in selective medium containing 0.1 M potassium phosphate pH 7 and grown at 30° C. with shaking at 300 rpm for 72 h.

FIG. 7: N-demethylation of thebaine by S. cerevisiae strains expressing either native Thamnostylum piriforme P450_DN15259_c0_g1_i7_A, P450_DN12791_c0_g1_i1_C or codon-optimized P450_DN15259_co, P450_DN12791_co, Lr_P450_co (from Lichtheimia ramosa) in combination with the CPR from Cunninghamella elegans. Cells were fed with 0.5 mM thebaine in selective medium containing 0.1 M potassium phosphate buffer pH 7 and grown at 30° C. with shaking at 300 rpm for 72 h.

FIG. 8: N-demethylation of oripavine by S. cerevisiae strains expressing either native Thamnostylum piriforme P450_DN15259_c0_g1_i7_A, P450_DN12791_c0_g1_i1_C or codon-optimized P450_DN15259_co, P450_DN12791_co, Lr_P450_co (from Lichtheimia ramosa) in combination with the CPR from Cunninghamella elegans. NC: Negative control strain. Cells were fed with 0.5 mM oripavine in selective medium containing 0.1 M potassium phosphate buffer pH 7 and grown at 30° C. with shaking at 300 rpm for 72 h.

FIG. 9: In vitro N-demethylation of salutaridine by mammalian cytochrome P450s of families 3A4 and 2C8. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM salutaridine, for 48 h at 30° C. The control strain lacked cytochrome P450.

FIG. 10: In vitro N-demethylation of salutaridinol by mammalian cytochrome P450s of families 3A4 and 2C8. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM salutaridinol, for 48 h at 30° C. The control strain lacked cytochrome P450.

FIG. 11: In vitro N-demethylation of thebaine by mammalian cytochrome P450s of families 3A4 and 2C8. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM thebaine, for 48 h at 30° C. The control strain lacked cytochrome P450.

FIG. 12: In vitro N-demethylation of oripavine by mammalian cytochrome P450s of families 3A4 and 2C8. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM oripavine, for 48 h at 30° C. The control strain lacked cytochrome P450.

FIG. 13: In vitro N-demethylation of codeine by mammalian cytochrome P450s of families 3A4 and 2C8. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM codeine, for 48 h at 30° C. The control strain lacked cytochrome P450.

FIG. 14: In vitro N-demethylation of salutaridine by mammalian cytochrome P450s of family 3A5. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM salutaridine, for 48 h at 30° C. The control strain lacked cytochrome P450.

FIG. 15: In vitro N-demethylation of salutaridinol by mammalian cytochrome P450s of family 3A5. Crude cell lysates were prepared from yeast strains overexpressing a mammalian cytochrome P450 together with a human NADPH cytochrome P450 reductase, and a human cytochrome b5. The reactions occurred at pH7 in the presence of 200 μM salutaridinol, for 48 h at 30° C. The control strain lacked cytochrome P450.

FIG. 16: In vitro N-demethylation of thebaine by mammalian cytochrome P450s of family 3A5. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM thebaine, for 48 h at 30° C. The control strain lacked cytochrome P450.

FIG. 17: In vitro N-demethylation of oripavine by mammalian cytochrome P450s of family 3A5. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM oripavine, for 48 h at 30° C. The control strain lacked cytochrome P450.

FIG. 18: In vitro N-demethylation of codeine by mammalian cytochrome P450s of family 3A5. Crude cell lysates were prepared from yeast strains overexpressing a mammalian cytochrome P450 together with a human NADPH cytochrome P450 reductase, and a human cytochrome b5. The reactions occurred at pH 7 in the presence of 200 μM codeine, for 48 h at 30° C. The control strain lacked cytochrome P450.

FIG. 19: Thebaine N-demethylation by new fungal candidates. Four new fungal cythochrome P450 candidates (P450_DN16393_co, LCOR_01865_co, LCOR_09548_co and Ar_ORZ22410_co) were co-expressed with three different CPRs in S. cerevisiae. Cells were fed with 0.5 mM thebaine in selective medium containing 0.1 M potassium phosphate buffer pH 7 and grown at 30° C. with shaking at 300 rpm for 72 h. The error bar represents the standard deviation of 4 different biological replicates.

FIG. 20: Oripavine N-demethylation by new fungal candidates. Four new fungal cythochrome P450 candidates (P450_DN16393_co, LCOR_01865_co, LCOR_09548_co and Ar_ORZ22410_co) were co-expressed with three different CPRs in S. cerevisiae.

Cells were fed with 0.5 mM oripavine in selective medium containing 0.1 M potassium phosphate buffer pH 7 and grown at 30° C. with shaking at 300 rpm for 72 h. The error bar represents the standard deviation of 4 different biological replicates.

FIG. 21: Thebaine N-demethylation by codon-optimized Lr_P450 and Mc_S2JT25. The cytochrome P450 candidate Mc_S2JT25_co from Mucor circinelloides and the cytochrome P450 Lr_P450_co from L. ramosa (used as positive control) were co-expressed with different CPRs. Cells were fed with 0.5 mM thebaine in selective medium containing 0.1 M potassium phosphate buffer pH 7 and grown at 30° C., 300 rpm for 72 h. The error bar represents the standard deviation of 4 different biological replicates.

FIG. 22: Oripavine N-demethylation by codon-optimized Lr_P450 and Mc_S2JT25. The cytochrome P450 candidate Mc_S2JT25_co from Mucor circinelloides and the cytochrome P450 Lr_P450_co from L. ramosa (used as positive control) were co-expressed with different CPRs. Cells were fed with 0.5 mM oripavine in selective medium containing 0.1 M potassium phosphate buffer pH 7 and grown at 30° C., 300 rpm for 72 h. The error bar represents the standard deviation of 4 different biological replicates.

FIG. 23: Thebaine N-demethylation by codon-optimized C. echinulata cytochrome P450s along with Cel_CPR_co. Seven yeast codon-optimized cytochrome P450 candidates from Cunninghamella echinulata were co-expressed with Cel_CPR, the CPR from C. elegans. Cells were fed with 0.5 mM thebaine in selective medium containing 0.1 M potassium phosphate buffer pH 7 and grown at 30° C., 300 rpm for 72 h. The error bar represents the standard deviation of 4 different biological replicates. (PC, Positive control).

FIG. 24: Oripavine N-demethylation by codon-optimized C. echinulata P450 along with Cel_CPR_co. Seven yeast codon-optimized cytochrome P450 candidates from Cunninghamella echinulata were co-expressed with Cel_CPR, the CPR from C. elegans. Cells were fed with 0.5 mM oripavine in selective medium containing 0.1 M potassium phosphate buffer pH 7 and grown at 30° C., 300 rpm for 72 h. The error bar represents the standard deviation of 4 different biological replicates. (PC, Positive control).

FIG. 25: Northebaine content in leaves of N. benthamiana. N. benthamiana leaves were infiltrated with a demethylase gene of Lichtheimia ramosa (Lr_P450_co), Mucor circinelloides (Mc_S2JT25_co) or Papaver somniferum (Ps_CODM_co) and the cytochrome P450 reductase gene of Thamnostylum piriforme (CPR_DN10898_c0_g1_i1_co) or Cunninghamella elegans (Cel_CPR_co). Leaves were then re-infiltrated with thebaine solution after 4 days and the northebaine content was measured 1 day after with LC-MS. Data shown as the mean of two individual plants, each sampled with 2 different leaf discs. Error bars represent the standard error of the mean.

FIG. 26: Thebaine content in leaves of N. benthamiana.

N. benthamiana leaves were co-infiltrated with either a demethylase gene of Lichtheimia ramosa (Lr_P450_co), Mucor circinelloides (Mc_S23T25_co) or Papaver somniferum (Ps_CODM_co) and the cytochrome P450 reductase gene of Thamnostylum piriforme (CPR_DN10898_c0_g1_i1_co) or Cunninghamella elegans (Cel_CPR_co). Leaves were re-infiltrated with thebaine solution after 4 days and the content was assessed 1 day after with LC-MS. Data shown as the mean of two individual plants, each sampled with 2 different leaf discs. Error bars represent the standard error of the mean.

FIG. 27: Oripavine content in leaves of N. benthamiana.

N. benthamiana leaves were co-infiltrated with either a demethylase gene of Lichtheimia ramosa (Lr_P450_co), Mucor circinelloides (Mc_S23T25_co) or Papaver somniferum (Ps_CODM_co) and the cytochrome P450 reductase gene of Thamnostylum piriforme (CPR_DN10898_c0_g1_i1_co) or Cunninghamella elegans (Cel_CPR_co). Leaves were re-infiltrated with thebaine after 4 days and the oripavine content was assessed 1 day after with LC-MS. Data shown as the mean of two individual plants, each sampled with 2 different leaf discs. Error bars represent the standard error of the mean.

FIG. 28: Northebaine content in Aspergillus nidulans

Northebaine content was measured in the supernatant by LC-MS after 84 h of incubation at 30° C. Data for samples Lr_P450_co, Mc_S2JT25_co, Tp_P450_DN12791_c0_g1_i1_co and Ps_CODM_coare shown as the mean of 5 biological replicates. For the vector control, the data is shown as the mean of 3 biological replicates. A single measurement was performed for the media control. The error bars represent the standard error of the mean.

FIG. 29A-29H: Growth media effect on northebaine production by yeast strains expressing new fungal candidates. Twenty-four different cytochrome P450s from fungi belonging to the Mucorales order were co-expressed with four different CPR genes from either T. piriforme (Tp_CPR_10898_co and Tp_CPR_5866_co), L. ramosa (POR1) or C. elegans (Cel_CPR_co). Cells were fed with 0.5 mM thebaine in selective (Sc) or DELFT medium containing 0.1 M potassium phosphate buffer pH 7 and grown at 30° C., 300 rpm for 72 h. The error bar represents the standard deviation of 2 different biological replicates. NC: Negative control strain.

FIG. 30A-30D: Oripavine production by yeast strains expressing new fungal candidates. Ten different cytochrome P450s from fungi belonging to the Mucorales order and one cytochrome P450 from plant (Ps_CODM_co—used as a positive control) were co-expressed with four different CPR genes from either T. piriforme (Tp_CPR_10898_co and Tp_CPR_5866_co), L. ramosa (POR1) or C. elegans (Cel_CPR_co). Cells were fed with 0.5 mM thebaine in selective (Sc) or DELFT medium containing 0.1 M potassium phosphate buffer pH 7 and grown at 30° C., 300 rpm for 72 h. The error bar represents the standard deviation of 2 different biological replicates. NC: Negative control strain.

FIG. 31A-31D: Nororipavine production by yeast strains expressing new fungal candidates. Ten different cytochrome P450s from fungi belonging to the Mucorales order were co-expressed with four different CPR genes from either T. piriforme (Tp_CPR_10898_co and Tp_CPR_5866_co), L. ramosa (POR1) or C. elegans (Cel_CPR_co). Cells were fed with 0.5 mM thebaine in selective medium (Sc) or DELFT medium containing 0.1 M potassium phosphate buffer pH 7 and grown at 30° C., 300 rpm for 72 h. The error bar represents the standard deviation of 2 different biological replicates. NC: Negative control strain.

FIG. 32: Conversion of thebaine to northebaine in non-conventional yeasts.

Northebaine content was measured in the supernatant by LC-MS after 4 days of incubation at 30° C. The N-demethylation of thebaine to northebaine was tested in K. marxianus (IBT 42, IBT82, IBT86), O. thermomethanolica (CBS 8099) and S. paradoxus (CBS 2908). The data shown was obtained from a single measurement.

### DETAILED DESCRIPTION OF THE INVENTION

The invention describes a new way for N-demethylation, and optionally additionally 0-demethylation, of reticuline and/or derivatives thereof, for example thebaine and oripavine, by a bioconversion process using a microbial host. This provides access to the naturally unavailable and chemically difficult-to-produce starting materials, such as northebaine and nororipavine, in an economic and sustainable process.

The invention is exemplified by identification and functional analyses of several fungal cytochrome P450 enzymes from the Mucorales order for example Thamnostylum piriforme, and used when produced recombinantly in S. cerevisiae, and plants like tobacco, for a thebaine and oripavine bioconversion process to northebaine or nororipavine, respectively. Along the same lines cytochrome P450 enzymes from human or ape have been evaluated for N-demethylation activity towards thebaine, oripavine, salutaridine, salutaridinol and codeine.

Thus, the invention relates to a recombinant host cell that expresses one or more genes encoding a cytochrome P450 enzyme capable of N-demethylating and/or O-demethylating reticuline and/or derivatives thereof, wherein at least one of the genes is a recombinant gene.

It is a unique feature of the present invention that the inventors have identified that the cytochrome P450 enzymes from the fungal Mucorales order are capable of N-demethylating recituline and derivatives hereof.

The host cell, accordingly can further express one or more cytochrome P450 reductase(s) (CPR(s)). These one or more reductases can be endogenous or heterologous, or there can be one or more of both.

General

All publications, patents and patent applications cited herein are hereby expressly incorporated by reference for all purposes.

Methods well known to those skilled in the art can be used to construct genetic expression constructs and recombinant cells according to this invention. These methods include in vitro recombinant DNA techniques, synthetic techniques, in vivo recombination techniques, and PCR techniques. See, for example, techniques as described in Maniatis et al., 1989, MOLECULAR CLONING: A LABORATORY MANUAL, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

Before describing the invention in detail, a number of terms will be defined. As used herein, the singular forms “a”, “an”, and “the” include plural referents unless the context clearly dictates otherwise. For example, reference to a “nucleic acid” means one or more nucleic acids.

It is noted that terms like “preferably”, “commonly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the invention.

For the purposes of describing and defining the invention it is noted that the terms “substantial” or “substantially” are utilized herein to represent the inherent degree of uncertainty that can be attributed to any quantitative comparison, value, measurement, or other representation. The terms “substantial” or “substantially” are also utilized herein to represent the degree by which a quantitative representation can vary from a stated reference without resulting in a change in the basic function of the subject matter at issue.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.”

As used herein, the terms “polynucleotide”, “nucleotide”, “oligonucleotide”, and “nucleic acid” can be used interchangeably to refer to nucleic acid comprising DNA, RNA, derivatives thereof, or combinations thereof.

As used herein, the terms “microorganism,” “microorganism host,” “microorganism host cell,” “host cell,” “recombinant host,” “recombinant microorganism host,” and “recombinant host cell” can be used interchangeably. As used herein, the term “recombinant host” is intended to refer to a host, the genome of which has been augmented by at least one DNA sequence. Such DNA sequences include but are not limited to genes that are not naturally present, DNA sequences that are not normally transcribed into RNA or translated into a protein (“expressed”), and other genes or DNA sequences which one desires to introduce into the non-recombinant host. It will be appreciated that typically the genome of a recombinant host described herein is augmented through stable introduction of one or more recombinant genes.

Generally, introduced DNA is not originally resident in the host that is the recipient of the DNA, but it is within the scope of this disclosure to isolate a DNA segment from a given host, and to subsequently introduce one or more additional copies of that DNA into the same host, e.g., to enhance production of the product of a gene or alter the expression pattern of a gene. In some instances, the introduced DNA will modify or even replace an endogenous gene or DNA sequence by, e.g., homologous recombination or site-directed mutagenesis. Suitable recombinant hosts include microorganisms.

As used herein, the term “recombinant gene” refers to a gene or DNA sequence that is introduced into a recipient host, regardless of whether the same or a similar gene or DNA sequence can already be present in such a host. “Introduced,” or “augmented” in this context, is known in the art to mean introduced or augmented by the hand of man. Thus, a recombinant gene can be a DNA sequence from another species or can be a DNA sequence that originated from or is present in the same species but has been incorporated into a host by recombinant methods to form a recombinant host. It will be appreciated that a recombinant gene that is introduced into a host can be identical to a DNA sequence that is normally present in the host being transformed, and is introduced to provide one or more additional copies of the DNA to thereby permit overexpression or modified expression of the gene product of that DNA. Said recombinant genes are particularly encoded by cDNA.

As used herein, the term “engineered biosynthetic pathway” refers to a biosynthetic pathway that occurs in a recombinant host, as described herein. In some aspects, one or more steps of the biosynthetic pathway do not naturally occur in an unmodified host. In some embodiments, a heterologous version of a gene is introduced into a host that comprises an endogenous version of the gene.

As used herein, the term “endogenous” gene refers to a gene that originates from and is produced or synthesized within a particular organism, tissue, or cell. In some embodiments, the endogenous gene is a yeast transporter. In some embodiments, the transporter is endogenous to S. cerevisiae, including, but not limited to S. cerevisiae strain S288C. In some embodiments, an endogenous yeast transporter gene is overexpressed. As used herein, the term “overexpress” is used to refer to the expression of a gene in an organism at levels higher than the level of gene expression in a wild type organism. See, e.g., Prelich, 2012, Genetics 190:841-54. In some embodiments, an endogenous yeast transporter gene is deleted. See, e.g., Giaever & Nislow, 2014, Genetics 197(2):451-65. As used herein, the terms “deletion,” “deleted,” “knockout,” and “knocked out” can be used interchangeably to refer to an endogenous gene that has been manipulated to no longer be expressed in an organism, including, but not limited to, S. cerevisiae. In some embodiments, a deleted/knocked out gene is a transporter gene or a transcription factor gene that regulates expression of a transporter gene.

As used herein, the terms “heterologous sequence” and “heterologous coding sequence” are used to describe a sequence derived from a species other than the recombinant host or a sequence from the host that has been inserted into the host recombinantly. In some embodiments one or more wild type sequence is inserted to generate an overexpression of the specific gene. The overexpression can come from manipulation of for example the promoter. In some embodiments, the recombinant host is an S. cerevisiae cell, and a heterologous sequence is derived from an organism other than S. cerevisiae. A heterologous coding sequence, for example, can be from a prokaryotic microorganism, a eukaryotic microorganism, a plant, an animal, an insect, or a fungus different than the recombinant host expressing the heterologous sequence. In some embodiments, a coding sequence is a sequence that is native to the host.

A “selectable marker” can be one of any number of genes that complement host cell auxotrophy, provide antibiotic resistance, or result in a color change. Linearized DNA fragments of the gene replacement vector then are introduced into the cells using methods well known in the art (see below). Integration of the linear fragments into the genome and the disruption of the gene can be determined based on the selection marker and can be verified by, for example, PCR or Southern blot analysis.

Subsequent to its use in selection, a selectable marker can be removed from the genome of the host cell by, e.g., Cre-LoxP systems (see, e.g., Gossen et al., 2002, Ann. Rev. Genetics 36:153-173 and U.S. 2006/0014264). Alternatively, a gene replacement vector can be constructed in such a way as to include a portion of the gene to be disrupted, where the portion is devoid of any endogenous gene promoter sequence and encodes none, or an inactive fragment of, the coding sequence of the gene.

As used herein, the terms “variant” and “mutant” are used to describe a protein sequence that has been modified at one or more amino acids, compared to the wild type sequence of a particular protein.

Chemical terms used herein can be preceded and/or followed by a single dash, “-”, or a double dash, “=”, to indicate the bond order of the bond between the named substituent and its parent moiety; a single dash indicates a single bond and a double dash indicates a double bond or a pair of single bonds in the case of a spiro-substituent. In the absence of a single or double dash it is understood that a single bond is formed between the substituent and its parent moiety; further, substituents are intended to be read “left to right” with reference to the chemical structure referred to unless a dash indicates otherwise. For example, arylalkyl, arylalkyl-, and -alkylaryl indicate the same functionality.

For simplicity, chemical moieties are defined and referred to throughout primarily as univalent chemical moieties (e.g., alkyl, aryl, etc.). Nevertheless, such terms are also used to convey corresponding multivalent moieties under the appropriate structural circumstances clear to those skilled in the art. For example, while an “alkyl” moiety can refer to a monovalent radical (e.g. CH₃—CH₂—), in some circumstances a bivalent linking moiety can be “alkyl,” in which case those skilled in the art will understand the alkyl to be a divalent radical (e.g., —CH₂—CH₂—), which is equivalent to the term “alkylene.” (Similarly, in circumstances in which a divalent moiety is required and is stated as being “aryl,” those skilled in the art will understand that the term “aryl” refers to the corresponding divalent moiety, arylene). All atoms are understood to have their normal number of valences for bond formation (i.e., 4 for carbon, 3 for N, 2 for O, and 2, 4, or 6 for S, depending on the oxidation state of the S). Nitrogens in the presently disclosed compounds can be hypervalent, e.g., an N-oxide or tetrasubstituted ammonium salt. On occasion a moiety can be defined, for example, as —B-(A)ₐ, wherein a is 0 or 1. In such instances, when a is 0 the moiety is —B and when a is 1 the moiety is —B-A.

As used herein, the term “alkyl” includes a saturated hydrocarbon having a designed number of carbon atoms, such as 1 to 40 carbons (i.e., inclusive of 1 and 40), 1 to 35 carbons, 1 to 25 carbons, 1 to 20 carbons, or 1, 2, 3, 4, 5, 6, 7, 8, 9, 10, 11, 12, 13, 14, 15, 16, 17, or 18. Alkyl group can be straight or branched and depending on context, can be a monovalent radical or a divalent radical (i.e., an alkylene group).

For example, the moiety “—(C₁-C₆alkyl)-O—” signifies connection of an oxygen through an alkylene bridge having from 1 to 6 carbons and C₁-C₃alkyl represents methyl, ethyl, and propyl moieties. Examples of “alkyl” include, for example, methyl, ethyl, propyl, isopropyl, butyl, iso-, sec- and tert-butyl, pentyl, and hexyl.

The term “alkoxy” represents an alkyl group of indicated number of carbon atoms attached to the parent molecular moiety through an oxygen bridge. Examples of “alkoxy” include, for example, methoxy, ethoxy, propoxy, and isopropoxy.

The term “alkenyl” as used herein, unsaturated hydrocarbon containing from 2 to 10 carbons (i.e., inclusive of 2 and 10), 2 to 8 carbons, 2 to 6 carbons, or 2, 3, 4, 5 or 6, unless otherwise specified, and containing at least one carbon-carbon double bond.

Alkenyl group can be straight or branched and depending on context, can be a monovalent radical or a divalent radical (i.e., an alkenylene group). For example, the moiety “—(C₂-C₆ alkenyl)-O—” signifies connection of an oxygen through an alkenylene bridge having from 2 to 6 carbons. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkynyl” as used herein, unsaturated hydrocarbon containing from 2 to 10 carbons (i.e., inclusive of 2 and 10), 2 to 8 carbons, 2 to 6 carbons, or 2, 3, 4, 5 or 6 unless otherwise specified, and containing at least one carbon-carbon triple bond.

Alkynyl group can be straight or branched and depending on context, can be a monovalent radical or a divalent radical (i.e., an alkynylene group). For example, the moiety “—(C₂-C₆ alkynyl)-O—” signifies connection of an oxygen through an alkynylene bridge having from 2 to 6 carbons. Representative examples of alkynyl include, but are not limited to, acetylenyl, 1-propynyl, 2-propynyl, 3-butynyl, 2-pentynyl, and 1-butynyl.

The term “aryl” represents an aromatic ring system having a single ring (e.g., phenyl) which is optionally fused to other aromatic hydrocarbon rings or non-aromatic hydrocarbon or heterocyclic rings. “Aryl” includes ring systems having multiple condensed rings and in which at least one is carbocyclic and aromatic, (e.g., 1,2,3,4-tetrahydronaphthyl, naphthyl). Examples of aryl groups include phenyl, 1-naphthyl, 2-naphthyl, indanyl, indenyl, dihydronaphthyl, fluorenyl, tetralinyl, and 6,7,8,9-tetrahydro-5H-benzo[a]cycloheptenyl. “Aryl” also includes ring systems having a first carbocyclic, aromatic ring fused to a nonaromatic heterocycle, for example, 1H-2,3-dihydrobenzofuranyl and tetrahydroisoquinolinyl. The aryl groups herein are unsubstituted or, when specified as “optionally substituted”, can unless stated otherwise be substituted in one or more substitutable positions with various groups as indicated.

The term “heteroaryl” refers to an aromatic ring system containing at least one aromatic heteroatom selected from nitrogen, oxygen and sulfur in an aromatic ring. Most commonly, the heteroaryl groups will have 1, 2, 3, or 4 heteroatoms. The heteroaryl can be fused to one or more non-aromatic rings, for example, cycloalkyl or heterocycloalkyl rings, wherein the cycloalkyl and heterocycloalkyl rings are described herein. In one embodiment of the present compounds the heteroaryl group is bonded to the remainder of the structure through an atom in a heteroaryl group aromatic ring. In another embodiment, the heteroaryl group is bonded to the remainder of the structure through a non-aromatic ring atom. Examples of heteroaryl groups include, for example, pyridyl, pyrimidinyl, quinolinyl, benzothienyl, indolyl, indolinyl, pyridazinyl, pyrazinyl, isoindolyl, isoquinolyl, quinazolinyl, quinoxalinyl, phthalazinyl, imidazolyl, isoxazolyl, pyrazolyl, oxazolyl, thiazolyl, indolizinyl, indazolyl, benzothiazolyl, benzimidazolyl, benzofuranyl, furanyl, thienyl, pyrrolyl, oxadiazolyl, thiadiazolyl, benzo[1,4]oxazinyl, triazolyl, tetrazolyl, isothiazolyl, naphthyridinyl, isochromanyl, chromanyl, isoindolinyl, isobenzothienyl, benzoxazolyl, pyridopyridinyl, purinyl, benzodioxolyl, triazinyl, pteridinyl, benzothiazolyl, imidazopyridinyl, imidazothiazolyl, benzisoxazinyl, benzoxazinyl, benzopyranyl, benzothiopyranyl, chromonyl, chromanonyl, pyridinyl-N-oxide, isoindolinonyl, benzodioxanyl, benzoxazolinonyl, pyrrolyl N-oxide, pyrimidinyl N-oxide, pyridazinyl N-oxide, pyrazinyl N-oxide, quinolinyl N-oxide, indolyl N-oxide, indolinyl N-oxide, isoquinolyl N-oxide, quinazolinyl N-oxide, quinoxalinyl N-oxide, phthalazinyl N-oxide, imidazolyl N-oxide, isoxazolyl N-oxide, oxazolyl N-oxide, thiazolyl N-oxide, indolizinyl N-oxide, indazolyl N-oxide, benzothiazolyl N-oxide, benzimidazolyl N-oxide, pyrrolyl N-oxide, oxadiazolyl N-oxide, thiadiazolyl N-oxide, triazolyl N-oxide, tetrazolyl N-oxide, benzothiopyranyl S-oxide, benzothiopyranyl S,S-dioxide. Preferred heteroaryl groups include pyridyl, pyrimidyl, quinolinyl, indolyl, pyrrolyl, furanyl, thienyl and imidazolyl, pyrazolyl, indazolyl, thiazolyl and benzothiazolyl. In certain embodiments, each heteroaryl is selected from pyridyl, pyrimidinyl, pyridazinyl, pyrazinyl, imidazolyl, isoxazolyl, pyrazolyl, oxazolyl, thiazolyl, furanyl, thienyl, pyrrolyl, oxadiazolyl, thiadiazolyl, triazolyl, tetrazolyl, isothiazolyl, pyridinyl-N-oxide, pyrrolyl N-oxide, pyrimidinyl N-oxide, pyridazinyl N-oxide, pyrazinyl N-oxide, imidazolyl N-oxide, isoxazolyl N-oxide, oxazolyl N-oxide, thiazolyl N-oxide, pyrrolyl N-oxide, oxadiazolyl N-oxide, thiadiazolyl N-oxide, triazolyl N-oxide, and tetrazolyl N-oxide. Preferred heteroaryl groups include pyridyl, pyrimidyl, quinolinyl, indolyl, pyrrolyl, furanyl, thienyl, imidazolyl, pyrazolyl, indazolyl, thiazolyl and benzothiazolyl. The heteroaryl groups herein are unsubstituted or, when specified as “optionally substituted”, can unless stated otherwise be substituted in one or more substitutable positions with various groups, as indicated.

The term “heterocycloalkyl” refers to a non-aromatic ring or ring system containing at least one heteroatom that is preferably selected from nitrogen, oxygen and sulfur, wherein said heteroatom is in a non-aromatic ring. The heterocycloalkyl can have 1, 2, 3 or 4 heteroatoms. The heterocycloalkyl can be saturated (i.e., a heterocycloalkyl) or partially unsaturated (i.e., a heterocycloalkenyl). Heterocycloalkyl includes monocyclic groups of three to eight annular atoms as well as bicyclic and polycyclic ring systems, including bridged and fused systems, wherein each ring includes three to eight annular atoms. The heterocycloalkyl ring is optionally fused to other heterocycloalkyl rings and/or non-aromatic hydrocarbon rings. In certain embodiments, the heterocycloalkyl groups have from 3 to 7 members in a single ring. In other embodiments, heterocycloalkyl groups have 5 or 6 members in a single ring. In some embodiments, the heterocycloalkyl groups have 3, 4, 5, 6 or 7 members in a single ring. Examples of heterocycloalkyl groups include, for example, azabicyclo[2.2.2]octyl (in each case also “quinuclidinyl” or a quinuclidine derivative), azabicyclo[3.2.1]octyl, 2,5-diazabicyclo[2.2.1]heptyl, morpholinyl, thiomorpholinyl, thiomorpholinyl S-oxide, thiomorpholinyl S,S-dioxide, 2-oxazolidonyl, piperazinyl, homopiperazinyl, piperazinonyl, pyrrolidinyl, azepanyl, azetidinyl, pyrrolinyl, tetrahydropyranyl, piperidinyl, tetrahydrofuranyl, tetrahydrothienyl, 3,4-dihydroisoquinolin-2(1H)-yl, isoindolindionyl, homopiperidinyl, homomorpholinyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, imidazolidonyl, tetrahydrothienyl S-oxide, tetrahydrothienyl S,S-dioxide and homothiomorpholinyl S-oxide. Especially desirable heterocycloalkyl groups include morpholinyl, 3,4-dihydroisoquinolin-2(1H)-yl, tetrahydropyranyl, piperidinyl, aza-bicyclo[2.2.2]octyl, γ-butyrolactonyl (i.e., an oxo-substituted tetrahydrofuranyl), γ-butryolactamyl (i.e., an oxo-substituted pyrrolidine), pyrrolidinyl, piperazinyl, azepanyl, azetidinyl, thiomorpholinyl, thiomorpholinyl S,S-dioxide, 2-oxazolidonyl, imidazolidonyl, isoindolindionyl, piperazinonyl. The heterocycloalkyl groups herein are unsubstituted or, when specified as “optionally substituted”, can unless stated otherwise be substituted in one or more substitutable positions with various groups, as indicated.

The term “cycloalkyl” refers to a non-aromatic carbocyclic ring or ring system, which can be saturated (i.e., a cycloalkyl) or partially unsaturated (i.e., a cycloalkenyl). The cycloalkyl ring optionally fused to or otherwise attached (e.g., bridged systems) to other cycloalkyl rings. Certain examples of cycloalkyl groups present in the disclosed compounds have from 3 to 7 members in a single ring, such as having 5 or 6 members in a single ring. In some embodiments, the cycloalkyl groups have 3, 4, 5, 6 or 7 members in a single ring. Examples of cycloalkyl groups include, for example, cyclohexyl, cyclopentyl, cyclobutyl, cyclopropyl, tetrahydronaphthyl and bicyclo[2.2.1]heptane. The cycloalkyl groups herein are unsubstituted or, when specified as “optionally substituted”, can be substituted in one or more substitutable positions with various groups, as indicated.

The term “ring system” encompasses monocycles, as well as fused and/or bridged polycycles.

The terms “halogen” or “halo” indicate fluorine, chlorine, bromine, and iodine. In certain embodiments of each and every embodiment described herein, the term “halogen” or “halo” refers to fluorine or chlorine. In certain embodiments of each and every embodiment described herein, the term “halogen” or “halo” refers to fluorine.

The term “halide” indicates fluoride, chloride, bromide, and iodide. In certain embodiments of each and every embodiment described herein, the term “halide” refers to bromide or chloride.

The term “substituted,” when used to modify a specified group or radical, means that one or more hydrogen atoms of the specified group or radical are each, independently of one another, replaced with the same or different substituent groups as defined below, unless specified otherwise.

Specific protecting groups can be used to protect reactive functionalities of a starting material or intermediate to prepare a desired product. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/I, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups can be removed at a convenient subsequent stage using methods known from the art.

Reticuline and Derivatives Thereof

Reticuline is a chemical compound found in a variety of plants including Lindera aggregata, Annona squamosa, and Ocotea fasciculate. It is based on the benzylisoquinoline structure:

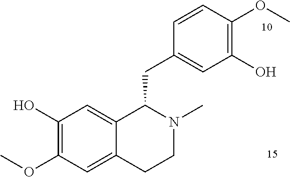

Reticuline is one of the alkaloids found in opium, and it is the precursor of morphine and many other alkaloids and opioids.

In an embodiment of the invention reticuline and/or derivatives thereof comprises (S)-reticuline, 1,2 dehydroreticuline, (R)-reticuline, salutaridine, salutaridinol, thebaine, oripavine, 7-O-acetyl-salutaridinol, neopinone, codeinone, codeine, morphinone, morphine, hydrocodone, 14-hydroxycodeinone or oxycodone.

In a further embodiment of the invention the reticuline derivative is thebaine.

In another embodiment of the invention the reticuline derivative is oripavine.

Cytochrome P450 Enzymes

Cytochromes P450 enzymes (CYPs) are proteins of the superfamily containing heme as a cofactor and, therefore, are hemoproteins. CYPs use a variety of small and large molecules as substrates in enzymatic reactions. They are, in general, the terminal oxidase enzymes in electron transfer chains, broadly categorized as P450-containing systems. The term P450 is derived from the spectrophotometric peak at the wavelength of the absorption maximum of the enzyme (450 nm) when it is in the reduced state and complexed with carbon monoxide.

Most CYPs require a protein partner to deliver one or more electrons to reduce the iron (and eventually molecular oxygen). Based on the nature of the electron transfer proteins, CYPs can be classified into several groups: Microsomal P450 systems, in which electrons are transferred from NADPH via cytochrome P450 reductase (variously CPR, POR, or CYPOR). Cytochrome b5 (cyb5) can also contribute reducing power to this system after being reduced by cytochrome b5 reductase (CYB5R). Mitochondrial P450 systems, which employ adrenodoxin reductase and adrenodoxin to transfer electrons from NADPH to P450. Bacterial P450 systems, which employ a ferredoxin reductase and a ferredoxin to transfer electrons to P450. CYB5R/cyb5/P450 systems, in which both electrons required by the CYP come from cytochrome b5. FMN/Fd/P450 systems, originally found in Rhodococcus species, in which a FMN-domain-containing reductase is fused to the CYP.

P450-only systems do not require external reducing power. Notable ones include thromboxane synthase (CYP5), prostacyclin synthase (CYP8), and CYP74A (allene oxide synthase).

In an embodiment of the invention the cytochrome P450 enzyme capable of N-demethylating reticuline and derivatives thereof comprises a mammalian P450 3A4 enzymes, mammalian P450 3A5 enzymes, and mammalian P450 2C8 enzymes.

In an embodiment of the invention the cytochrome P450 enzyme capable of N-demethylating reticuline and derivatives thereof comprises a fungal cytochrome P450 enzymes.

In an embodiment of the invention the cytochrome P450 enzyme capable of N-demethylating and/or O-demethylating reticuline and/or derivatives thereof comprises a fungal cytochrome P450 enzymes, mammalian P450 3A4 enzymes, mammalian P450 3A5 enzymes, and mammalian P450 2C8 enzymes.

The cytochrome P450 enzyme capable of N-demethylating and/or O-demethylating reticuline and/or derivatives thereof can originate from a fungal organism. The organism can be Thamnostylum piriforme, Lichtheimia ramosa, Cunninghamella echinulata, Cunninghamella dalmatica, Cunninghamella polymorpha or Rhizopus nigricans.

The cytochrome P450 enzyme capable of N-demethylating reticuline and derivatives thereof can originate from an organism including Cunninghamella echinulata, Rhizopus nigricans, and Mucor piriformis.

The cytochrome P450 enzyme capable of N-demethylating and/or O-demethylating reticuline and/or derivatives thereof can originate from a mammalian organism, including but not limited to Homo sapiens, Pongo abelii, Papio anubis, Gorilla gorilla gorilla, Canis lupus familiaris, Pan troglodytes, Callithrix jacchus, Macaca fascicularis and Chlorocebus aethiops.

Fungal Cytochrome P450 Enzymes

In a further embodiment of the invention the cytochrome P450 enzyme capable of N-demethylating and/or O-demethylating reticuline and/or derivatives thereof is a fungal cytochrome P450 enzyme or functional homologs or variants hereof.

In some embodiments of the invention is the cytochrome P450 enzyme capable of N-demethylating and/or O-demethylating reticuline and/or derivatives thereof from the Mucorales order. The Mucorales is the largest and best studied order of zygomycete fungi. Members of this order are sometimes called pin molds.

Being able to express the demethylases with strong promoters in a heterologous host is key for using these enzymes in a commercial process where turnover has to be fast and efficient. Without knowing the gene sequence and thereby the amino acid sequence of the enzymes responsible for the demethylation reactions such expression in heterologous hosts is not possible. Before the present invention it was not known which type of enzyme was responsible for the N-demethylation reaction in fungi of the mucorales order and sequences of the responsible enzymes not isolated. Filamentous fungi have like plants can typically have several hundred Cytochrome P450 and dioxygenase enzymes, which could all be candidates for being N-demethylases. The first isolation of the specific enzyme responsible for this reaction from a specific fungus is therefore a very complex task, and in particular when the species is not genome sequenced. Finding more homologs of the first isolated gene in related sequenced species using BLAST search is on the other hand less difficult.

It is therefore a unique feature of the present invention that the inventors have identified that the cytochrome P450 enzymes from the fungal Mucorales order are capable of N-demethylating recituline and derivatives hereof.

The fungal cytochrome P450 enzyme can be P450_DN15259_c0_g1_i7 (SEQ ID NO: 1), P450_DN12791_c0_g1_i1 (SEQ ID NO: 4) and/or A0A077WEM0 (SEQ ID NO: 7), or functional homologs or variants hereof.

The fungal cytochrome P450 enzyme can also be P450_DN15259_c0_g1_i7 (SEQ ID NO: 1) or functional homologs or variants hereof. P450_DN15259_c0_g1_i7 is encoded by SEQ ID NO: 2 and the sequence optimized for S. cerevisiae is SEQ ID NO: 3.

The fungal cytochrome P450 enzyme can also be P450_DN12791_c0_g1_i1 (SEQ ID NO: 4) or functional homologs or variants hereof. P450_DN12791_c0_g1_i1 is encoded by SEQ ID NO: 5 and the sequence optimized for S. cerevisiae is SEQ ID NO: 6.

The fungal cytochrome P450 enzyme can also be A0A077WEM0 (SEQ ID NO: 7) or functional homologs or variants hereof. A0A077WEM0 is encoded by SEQ ID NO: 8.

The fungal Mucorales cytochrome P450 enzyme can also be selected from the group consisting of:
i) CYPDN8 (SEQ ID NO: 72), ii) Mc_S2JT25 (SEQ ID NO: 52), iii) CYPDN17 (SEQ ID NO: 90), iv) CYPDN12 (SEQ ID NO: 80), v) Lr_P450 (SEQ ID NO: 8), vi) CYPDN29 (SEQ ID NO: 114), vii) CYPDN14 (SEQ ID NO: 84), vii) P450_DN15259_c0_g1_i7 (SEQ ID NO: 3), ix) LCOR_01865 (SEQ ID NO: 54), x) P450_DN5615_c2_g1_i9 (SEQ ID NO: 62), xi) P450_DN12791_c0_g1_i1 (SEQ ID NO: 5), xii) CYPDN16 (SEQ ID NO: 88), xiii) CYPDN18 (SEQ ID NO: 92), xiv) CYPDN27 (SEQ ID NO: 110), xv) CYPDN35 (SEQ ID NO: 126), xvi) CYPDN5 (SEQ ID NO: 66), xvii) CYPDN6 (SEQ ID NO: 68), xviii) CYPDN7 (SEQ ID NO: 70), xix) CYPDN10 (SEQ ID NO: 76), xx) CYPDN11 (SEQ ID NO: 78), xxi) CYPDN24 (SEQ ID NO: 104), xxii) CYPDN28 (SEQ ID NO: 112), xxiii) CYPDN13 (SEQ ID NO: 82), xxiv) CYPDN31 (SEQ ID NO: 118), xxv) CYPDN34 (SEQ ID NO: 124), xxvi) CYPDN22 (SEQ ID NO: 100), xxvii) CYPDN21 (SEQ ID NO: 98), xxviii) CYPDN30 (SEQ ID NO: 116), xxix) Ar_ORZ22410 (SEQ ID NO: 58), xxx) CYPDN20 (SEQ ID NO: 96), xxxi) CYPDN17 (SEQ ID NO: 90), and xxxii) CYPDN8 (SEQ ID NO: 72).

Thus, the P450 enzyme can be YPDN8 (SEQ ID NO: 72). The P450 enzyme can also be Mc_S2JT25 (SEQ ID NO: 52). The P450 enzyme can also be CYPDN17 (SEQ ID NO: 90). The P450 enzyme can also beCYPDN12 (SEQ ID NO: 80). The P450 enzyme can also be Lr_P450 (SEQ ID NO: 8). The P450 enzyme can also be CYPDN29 (SEQ ID NO: 114). The P450 enzyme can also be CYPDN14 (SEQ ID NO: 84). The P450 enzyme can also be P450_DN15259_c0_g1_i7 (SEQ ID NO: 3). The P450 enzyme can also beCOR_01865 (SEQ ID NO: 54). The P450 enzyme can also beP450_DN5615_c2_g1_i9 (SEQ ID NO: 62). The P450 enzyme can also beP450_DN12791_c0_g1_i1 (SEQ ID NO: 5). The P450 enzyme can also be CYPDN16 (SEQ ID NO: 88). The P450 enzyme can also beCYPDN18 (SEQ ID NO: 92). The P450 enzyme can also be CYPDN27 (SEQ ID NO: 110). The P450 enzyme can also be CYPDN35 (SEQ ID NO: 126). The P450 enzyme can also be CYPDN5 (SEQ ID NO: 66). The P450 enzyme can also be CYPDN6 (SEQ ID NO: 68). The P450 enzyme can also be CYPDN7 (SEQ ID NO: 70). The P450 enzyme can also be CYPDN10 (SEQ ID NO: 76). The P450 enzyme can also be CYPDN11 (SEQ ID NO: 78). The P450 enzyme can also be CYPDN24 (SEQ ID NO: 104). The P450 enzyme can also be CYPDN28 (SEQ ID NO: 112). The P450 enzyme can also beCYPDN13 (SEQ ID NO: 82). The P450 enzyme can also be CYPDN31 (SEQ ID NO: 118). The P450 enzyme can also be CYPDN34 (SEQ ID NO: 124). The P450 enzyme can also be CYPDN22 (SEQ ID NO: 100). The P450 enzyme can also be CYPDN21 (SEQ ID NO: 98). The P450 enzyme can also be CYPDN30 (SEQ ID NO: 116). The P450 enzyme can also be Ar_ORZ22410 (SEQ ID NO: 58). The P450 enzyme can also be YPDN20 (SEQ ID NO: 96), The P450 enzyme can also be CYPDN17 (SEQ ID NO: 90). The P450 enzyme can also be (SEQ ID NO: 72).

The fungal Mucorales cytochrome P450 enzyme selected from the group consisting of: i) CYPDN8 (SEQ ID NO: 72), ii) Mc_S2JT25 (SEQ ID NO: 52), iii) CYPDN17 (SEQ ID NO: 90), iv) CYPDN12 (SEQ ID NO: 80), v) Lr_P450 (SEQ ID NO: 8), vi) CYPDN29 (SEQ ID NO: 114), vii) CYPDN14 (SEQ ID NO: 84), vii) P450_DN15259_c0_g1_i7 (SEQ ID NO: 3), ix) LCOR_01865 (SEQ ID NO: 54), x) P450_DN5615_c2_g1_i9 (SEQ ID NO: 62), xi) P450_DN12791_c0_g1_i1 (SEQ ID NO: 5), xii) CYPDN16 (SEQ ID NO: 88), xiii) CYPDN18 (SEQ ID NO: 92), xiv) CYPDN27 (SEQ ID NO: 110), xv) CYPDN35 (SEQ ID NO: 126), xvi) CYPDN5 (SEQ ID NO: 66), xvii) CYPDN6 (SEQ ID NO: 68), xviii) CYPDN7 (SEQ ID NO: 70), xix) CYPDN10 (SEQ ID NO: 76), xx) CYPDN11 (SEQ ID NO: 78), xxi) CYPDN24 (SEQ ID NO: 104), xxii) CYPDN28 (SEQ ID NO: 112), xxiii) CYPDN13 (SEQ ID NO: 82), xxiv) CYPDN31 (SEQ ID NO: 118), xxv) CYPDN34 (SEQ ID NO: 124), xxvi) CYPDN22 (SEQ ID NO: 100), xxvii) CYPDN21 (SEQ ID NO: 98), xxviii) CYPDN30 (SEQ ID NO: 116), xxix) Ar_ORZ22410 (SEQ ID NO: 58), and xxx) CYPDN20 (SEQ ID NO: 96)
have all been tested in the examples of the present invention, and they share the N-demethylating activity on reticuline and/or derivatives hereof.

The fungal Mcorales cytochrome P450 enzyme selected from the group consisting of: xxxi) CYPDN17 (SEQ ID NO: 90), and xxxii) CYPDN8 (SEQ ID NO: 72) share the N-demethylating and O-demethylating activity on reticuline and/or derivatives hereof.

Mammalian P450 3A4 Enzymes

In another embodiment of the invention the cytochrome P450 enzyme capable of N-demethylating reticuline and derivatives thereof is a mammalian P450 3A4 enzyme or functional homologs or variants hereof.

In certain embodiments of the invention, the mammalian P450 3A4 enzyme can be one or more of P08684 (SEQ ID NO: 22), H2PLK4 (SEQ ID NO: 24), A0A096NZ89 (SEQ ID NO: 26), G3SB46 (SEQ ID NO: 28), F1PDL2 (SEQ ID NO: 30) and functional homologs or variants hereof.

The mammalian P450 3A4 enzyme can also be P08684 (SEQ ID NO: 22) or functional homologs or variants hereof. P08684 (SEQ ID NO: 22) is encoded by SEQ ID NO: 23.

The mammalian P450 3A4 enzyme can also be H2PLK4 (SEQ ID NO: 24) or functional homologs or variants hereof. H2PLK4 (SEQ ID NO: 24) is encoded by SEQ ID NO: 25.

The mammalian P450 3A4 enzyme can also be A0A096NZ89 (SEQ ID NO: 26) or functional homologs or variants hereof. A0A096NZ89 (SEQ ID NO: 26) is encoded by SEQ ID NO: 27.

The mammalian P450 3A4 enzyme can also be G3SB46 (SEQ ID NO: 28) or functional homologs or variants hereof. G3SB46 (SEQ ID NO: 28) is encoded by SEQ ID NO: 29.

The mammalian P450 3A4 enzyme can also be F1PDL2 (SEQ ID NO: 30) or functional homologs or variants hereof. F1PDL2 (SEQ ID NO: 30) is encoded by SEQ ID NO: 31.

Mammalian P450 3A5 Enzymes

In yet another embodiment of the invention the cytochrome P450 enzyme capable of N-demethylating reticuline and derivatives thereof is a mammalian P450 3A5 enzyme or functional homologs or variants hereof.

The mammalian P450 3A5 enzyme can be one or more of Cytochrome P450 3A5 (P20815) (SEQ ID NO: 40), Cytochrome P450 3A5 (A4ZZ70) (SEQ ID NO: 42), Cytochrome P450 3A5 (A8CBR0) (SEQ ID NO: 44), and Cytochrome P450 3A5 (U3ECK3) (SEQ ID NO: 46) and functional homologs or variants hereof.

The mammalian P450 3A5 enzyme can also be Cytochrome P450 3A5 (P20815) (SEQ ID NO: 40) or functional homologs or variants hereof. Cytochrome P450 3A5 (P20815) (SEQ ID NO: 40) is encoded by SEQ ID NO: 41.

The mammalian P450 3A5 enzyme can also be Cytochrome P450 3A5 (A4ZZ70) (SEQ ID NO: 42) or functional homologs or variants hereof. Cytochrome P450 3A5 (A4ZZ70) (SEQ ID NO: 42) is encoded by SEQ ID NO: 43.

The mammalian P450 3A5 enzyme can also be Cytochrome P450 3A5 (A8CBR0) (SEQ ID NO: 44) or functional homologs or variants hereof Cytochrome P450 3A5 (A8CBR0) (SEQ ID NO: 44) is encoded by SEQ ID NO: 45.

The mammalian P450 3A5 enzyme can also be Cytochrome P450 3A5 (U3ECK3) (SEQ ID NO: 46) or functional homologs or variants hereof. Cytochrome P450 3A5 (U3ECK3) (SEQ ID NO: 46) is encoded by SEQ ID NO: 47.

Mammalian P450 2C8 Enzymes.

In another embodiment of the invention the cytochrome P450 enzyme capable of N-demethylating reticuline and derivatives thereof is a mammalian P450 2C8 enzyme.

The mammalian P450 2C8 enzyme can be one or more of P10632 (SEQ ID NO: 32), H2Q2B (SEQ ID NO: 34), H2NB34 (SEQ ID NO: 36), and Q4U0S8 (SEQ ID NO: 38) and functional homologs or variants hereof.

The mammalian P450 2C8 enzyme can also be P10632 (SEQ ID NO: 32) or functional homologs or variants hereof. P10632 (SEQ ID NO: 32) is encoded by SEQ ID NO: 33.

The mammalian P450 2C8 enzyme can also be H2Q2B (SEQ ID NO: 34) or functional homologs or variants hereof. H2Q2B (SEQ ID NO: 34) is encoded by SEQ ID NO: 35.

The mammalian P450 2C8 enzyme can also be H2NB34 (SEQ ID NO: 36) or functional homologs or variants hereof. H2NB34 (SEQ ID NO: 36) is encoded by SEQ ID NO: 37.

The mammalian P450 2C8 enzyme can also be Q4U0S8 (SEQ ID NO: 38) or functional homologs or variants hereof. Q4U0S8 (SEQ ID NO: 38) is encoded by SEQ ID NO: 39.

Cytochrome P450 Reductases

The recombinant host cell can further express one or more cytochrome P450 reductase(s) (CPR(s)).

The cytochrome P450 reductase(s) can originate from a fungal or mammalian organism. The organism can be one or more of Mucor piriformis, Thamnostylum piriforme, Cunninghamella elegans, Gibberella fujikuroi, Saccharomyces cerevisiae, Homo sapiens, Pongo abelii, Papio anubis, Gorilla gorilla gorilla, Canis lupus familiaris, Pan troglodytes, Callithrix jacchus, Macaca fasciculans and Chlorocebus aethiops The cytochrome P450 enzyme capable of N-demethylating reticuline and derivatives thereof can also originate from an organism that is Thamnostylum piriforme, Lichtheimia ramosa, Cunninghamella echinulata, Cunninghamella dalmatica, Cunninghamella polymorpha and Rhizopus nigricans.

The cytochrome P450 reductase can originate from a fungal organism. The organism can be one or more of Thamnostylum piriforme, Lichtheimia ramosa, Cunninghamella echinulata, Cunninghamella dalmatica, Cunninghamella polymorpha, Rhizopus nigricans, Gibberella fujikuroi, and Saccharomyces cerevisiae.

The cytochrome P450 reductase can originate from a mammalian organism. The organism can be Homo sapiens.

The cytochrome P450 reductase can be one or more of CPR_DN2505_c0_g1_i1 (SEQ ID NO: 15), CPR_DN5866_c0_g1_i1 (SEQ ID NO: 9), CPR_DN10898_c0_g1_i1 (SEQ ID NO: 12), NADPH-dependent cytochrome P450 oxidoreductase (AAF89958) (SEQ ID NO: 16), Cytochrome P450 oxidoreductase (Q7Z8R1) (SEQ ID NO: 18), NADPH-cytochrome P450 reductase (P16603) (SEQ ID NO: 20), NADPH-cytochrome P450 reductase (BAB18572.1) (SEQ ID NO: 50), and Cytochrome b5 isoform 1 (NP_683725) (SEQ ID NO: 48) or functional homologs or variants hereof.

The cytochrome P450 reductase can also be CPR_DN2505_c0_g1_i1 (SEQ ID NO: 15) or functional homologs or variants hereof.

The cytochrome P450 reductase can also be CPR_DN5866_c0_g1_i1 (SEQ ID NO: 9) or functional homologs or variants hereof. CPR_DN5866_c0_g1_i1 is encoded by SEQ ID NO: 10 and the sequence optimized for S. cerevisiae is SEQ ID NO: 11.

The cytochrome P450 reductase can also be CPR_DN10898_c0_g1_i1 (SEQ ID NO: 12) or functional homologs or variants hereof. CPR_DN10898_c0_g1_i1 (SEQ ID NO: 12) is encoded by SEQ ID NO: 13 and the sequence optimized for S. cerevisiae is SEQ ID NO: 14.

The cytochrome P450 reductase can also be NADPH-dependent cytochrome P450 oxidoreductase (AAF89958) (SEQ ID NO: 16) or functional homologs or variants hereof. The NADPH-dependent cytochrome P450 oxidoreductase (AAF89958) (SEQ ID NO: 16) sequence optimized for S. cerevisiae is encoded by SEQ ID NO: 17.

The cytochrome P450 reductase can also be NADPH-cytochrome P450 reductase (P16603) (SEQ ID NO: 20) or functional homologs or variants hereof. NADPH-cytochrome P450 reductase (P16603) (SEQ ID NO: 20) is encoded by SEQ ID NO: 21.

The cytochrome P450 reductase can also be NADPH-cytochrome P450 reductase (BAB18572.1) (SEQ ID NO: 50) or functional homologs or variants hereof. NADPH-cytochrome P450 reductase (BAB18572.1) (SEQ ID NO: 50) is encoded by SEQ ID NO: 51.

The cytochrome P450 reductase can also be Cytochrome b5 isoform 1 (NP_683725) (SEQ ID NO: 48) or functional homologs or variants hereof. NADPH-cytochrome P450 reductase (BAB18572.1) (SEQ ID NO: 48) is encoded by SEQ ID NO: 49.

Combinations of Cytochrome P450 Enzymes and CPRs

Specific combinations of P450 enzyme capable of N-demethylating reticuline and derivatives thereof and cytochrome P450 reductase(s) (CPR(s)) have been experimentally shown to have advantageous effects in the examples of the present disclosure.

In an embodiment of the invention is the cytochrome P450 enzyme capable of N-demethylating reticuline and derivatives thereof P450_DN15259_c0_g1_i7 (SEQ ID NO: 3) and the cytochrome P450 reductase is one or more of AAF89958 (SEQ ID NO: 17), Q7Z8R1 (SEQ ID NO: 19) and P16603 (SEQ ID NO: 21).

In an embodiment of the invention is the cytochrome P450 enzyme capable of N-demethylating reticuline and derivatives thereof P450_DN15259_c0_g1_i7 (SEQ ID NO: 3) and the cytochrome P450 reductase is AAF89958 (SEQ ID NO: 17).

In an embodiment of the invention is the cytochrome P450 reductase CPR_DN5866_c0_g1_i1 (SEQ ID NO: 9) and/or CPR_DN10898_c0_g1_i1 (SEQ ID NO: 12).

In one embodiment of the invention is the cytochrome P450 reductase POR1 (SEQ ID NO: 131 and 132).

The reductases that are added to the P450 enzyme can be one or more of the reductases discloses herein.

In an embodiment of the invention is the P450 enzyme capable of N-demethylating reticuline and derivatives thereof P450_DN15259_c0_g1_i7 (SEQ ID NO: 3) and the cytochrome P450 reductase is CPR_DN10898_c0_g1_i1 (SEQ ID NO: 14) and/or Q7Z8R1 (SEQ ID NO: 19).

In an embodiment of the invention is the cytochrome P450 enzyme capable of N-demethylating reticuline and derivatives thereof P450_DN15259_c0_g1_i7 (SEQ ID NO: 3) and the cytochrome P450 reductase is Q7Z8R1 (SEQ ID NO: 19).

In an embodiment of the invention is the cytochrome P450 enzyme capable of N-demethylating reticuline and derivatives thereof is P450_DN15259_c0_g1_i7 (SEQ ID NO: 3) and the cytochrome P450 reductase CPR_DN5866_c0_g1_i1 (SEQ ID NO: 9) and/or CPR_DN10898_c0_g1_i1 (SEQ ID NO: 12).

In an embodiment of the invention is the derivative salutaridine and the cytochrome P450 enzyme is a P450 2C8 enzyme.

In an embodiment of the invention is the derivative salutaridine and the cytochrome P450 enzyme is one or more of P10632 (SEQ ID NO: 23), H2Q2B (SEQ ID NO: 25), H2NB34 (SEQ ID NO: 27), Q4U0S8 (SEQ ID NO: 39).

In an embodiment of the invention is the derivative salutaridinol and the cytochrome P450 enzyme is a P450 2C8 enzyme.

In an embodiment of the invention is the derivative salutaridinol and the cytochrome P450 enzyme is one or more of P10632 (SEQ ID NO: 33), H2Q2B (SEQ ID NO: 35), H2NB34 (SEQ ID NO: 37), and Q4U0S8 (SEQ ID NO: 39).

In an embodiment of the invention is the compound thebaine and the cytochrome P450 enzyme is a P450 34A enzyme.

In an embodiment of the invention is the compound thebaine and the cytochrome P450 enzyme is A4ZZ70 (SEQ ID NO: 43).

In an embodiment of the invention is the derivative oripavine and the cytochrome P450 enzyme is a P450 2C8 enzyme.

In an embodiment of the invention is the derivative oripavine and the cytochrome P450 enzyme is Q4U0S8 (SEQ ID NO: 39).

In an embodiment of the invention is the derivative morphine and the cytochrome P450 enzyme is a P450 2C8 enzyme or a P450 3A4 enzyme.

In an embodiment of the invention is the derivative morphine and the cytochrome P450 enzyme is one or more of P20815 (SEQ ID NO: 41), A4ZZ70 (SEQ ID NO: 43), P10632 (SEQ ID NO: 33), H2Q2B (SEQ ID NO: 35), H2NB34 (SEQ ID NO: 37), and Q4U0S8 (SEQ ID NO: 39).

In an embodiment of the invention is the compound thebaine and the cytochrome P450 enzyme is a P450 3A4 enzyme.

In an embodiment of the invention is the compound thebaine and the cytochrome P450 enzyme is A0A096NZ89 (SEQ ID NO: 27).

Functional Homologs and Genetic Variation

Functional homologs of the polypeptides described above are also suitable for use in producing the compounds mentioned herein in a recombinant host. A functional homolog is a polypeptide that has sequence similarity to a reference polypeptide, and that carries out one or more of the biochemical or physiological function(s) of the reference polypeptide.

A functional homolog and the reference polypeptide can be natural occurring polypeptides, and the sequence similarity can be due to convergent or divergent evolutionary events. As such, functional homologs are sometimes designated in the literature as homologs, or orthologs, or paralogs. Variants of a naturally occurring functional homolog, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”).

Techniques for modifying genes encoding functional the polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide: polypeptide interactions in a desired manner.

Such modified polypeptides are considered functional homologs. The term “functional homolog” is sometimes applied to the nucleic acid that encodes a functionally homologous polypeptide.

Functional homologs can be identified by analysis of nucleotide and polypeptide sequence alignments. For example, performing a query on a database of nucleotide or polypeptide sequences can identify homologs of polypeptides described herein.

Sequence analysis can involve BLAST, Reciprocal BLAST, or PSI-BLAST analysis of nonredundant databases using the amino acid sequence of interest as the reference sequence. Amino acid sequence is, in some instances, deduced from the nucleotide sequence. Those polypeptides in the database that have greater than 40% sequence identity are candidates for further evaluation for suitability as polypeptide useful in the synthesis of compounds described herein. Amino acid sequence similarity allows for conservative amino acid substitutions, such as substitution of one hydrophobic residue for another or substitution of one polar residue for another. When desired, manual inspection of such candidates can be carried out in order to narrow the number of candidates to be further evaluated. Manual inspection can be performed by selecting those candidates that appear to have conserved functional domains.

Conserved regions can be identified by locating a region within the primary amino acid sequence of a polypeptide described herein that is a repeated sequence, forms some secondary structure (e.g., helices and beta sheets), establishes positively or negatively charged domains, or represents a protein motif or domain. See, e.g., the Pfam web site describing consensus sequences for a variety of protein motifs and domains on the World Wide Web at sanger.ac.uk/Software/Pfam/ and pfam.janelia.org/. The information included at the Pfam database is described in Sonnhammer et al., Nucl. Acids Res., 26:320-322 (1998); Sonnhammer et al., Proteins, 28:405-420 (1997); and Bateman et al., Nucl. Acids Res., 27:260-262 (1999). Conserved regions also can be determined by aligning sequences of the same or related polypeptides from closely related species. Closely related species preferably are from the same family. In some aspects, alignment of sequences from two different species can be adequate.

Typically, polypeptides that exhibit at least about 20% amino acid sequence identity are useful to identify conserved regions. Conserved regions of related polypeptides exhibit at least 25% amino acid sequence identity e.g., at least 30%, at least 40%, at least 55%, at least 50%, at least 60%, at least 70%, at least 80%, or at least 90% amino acid sequence identity. In some aspects, a conserved region exhibits at least 92%, 94%, 96%, 98%, or 99% amino acid sequence identity. The conserved region can be considered to be the entire protein or nucleic acid sequence.

An aspect of the invention relates to a functional homologue that has at least 20% sequence identity with an amino acid or nucleic acid sequence mentioned herein, such as 30% sequence identity, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 75% sequence identity, such as 97% sequence identity, such as 98% sequence identity, such as 99% sequence identity.

In an embodiment relates to a functional homolog that has at least 30% sequence identity with an amino acid or nucleic acid sequence mentioned herein. In an embodiment relates to a functional homolog that has at least 40% sequence identity with an amino acid or nucleic acid sequence mentioned herein. In an embodiment relates to a functional homolog that has at least 50% sequence identity with an amino acid or nucleic acid sequence mentioned herein. In an embodiment relates to a functional homolog that has at least 60% sequence identity with an amino acid or nucleic acid sequence mentioned herein. In an embodiment relates to a functional homolog that has at least 70% sequence identity with an amino acid or nucleic acid sequence mentioned herein. In an embodiment relates to a functional homolog that has at least 80% sequence identity with an amino acid or nucleic acid sequence mentioned herein. In an embodiment relates to a functional homolog that has at least 90% sequence identity with an amino acid or nucleic acid sequence mentioned herein. In an embodiment relates to a functional homolog that has at least 95% sequence identity with an amino acid or nucleic acid sequence mentioned herein.

The functional homolog can have an equal or better function compared to the patent enzyme. Thus, functional variants of the cytochrome P450 enzyme capable of N-demethylating reticuline and derivatives thereof can be measured for effect by any of the methods mentioned herein. The effect can therefore be the ability to N-demethylate.

The cytochrome P450 enzyme capable of N-demethylating reticuline and derivatives thereof can be one or more of P450_DN15259_c0_g1_i7 (SEQ ID NO: 3), P450_DN12791_c0_g1_i1 (SEQ ID NO: 6), A0A077WEM0 (SEQ ID NO: 8), P08684 (SEQ ID NO: 23), H2PLK4 (SEQ ID NO: 25), A0A096NZ89 (SEQ ID NO: 27), G3SB46 (SEQ ID NO: 29), F1PDL2 (SEQ ID NO: 31), P20815 (SEQ ID NO: 41), A4ZZ70 (SEQ ID NO: 43), A8CBR0 (SEQ ID NO: 45), U3ECK3 (SEQ ID NO: 47), P10632 (SEQ ID NO: 33), H2Q2B (SEQ ID NO: 35), H2NB34 (SEQ ID NO: 37), Q4U0S8 (SEQ ID NO: 39).

The cytochrome P450 enzyme capable of N-demethylating reticuline and derivatives thereof can have at least 20% sequence identity with P450_DN15259_c0_g1_i7 (SEQ ID NO: 3), such as 30% sequence identity, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 95% sequence identity, such as 97% sequence identity, such as 98% sequence identity, such as 99% sequence identity.

The cytochrome P450 enzyme capable of N-demethylating reticuline and derivatives thereof can have at least 40% sequence identity with P450_DN15259_c0_g1_i7 (SEQ ID NO: 3). The 40% sequence identity can also be with SEQ ID NO: 1 or SEQ ID NO: 2.

The cytochrome P450 enzyme capable of N-demethylating reticuline and derivatives thereof can have at least 50% sequence identity with P450_DN15259_c0_g1_i7 (SEQ ID NO: 3). The 50% sequence identity can also be with SEQ ID NO: 1 or SEQ ID NO: 2.

The cytochrome P450 enzyme capable of N-demethylating reticuline and derivatives thereof can have at least 60% sequence identity with P450_DN15259_c0_g1_i7 (SEQ ID NO: 3). The 60% sequence identity can also be with SEQ ID NO: 1 or SEQ ID NO: 2.

The cytochrome P450 enzyme capable of N-demethylating reticuline and derivatives thereof can have at least 70% sequence identity with P450_DN15259_c0_g1_i7 (SEQ ID NO: 3). The 70% sequence identity can also be with SEQ ID NO: 1 or SEQ ID NO: 2.

The cytochrome P450 enzyme capable of N-demethylating reticuline and derivatives thereof can have at least 80% sequence identity with P450_DN15259_c0_g1_i7 (SEQ ID NO: 3). The 80% sequence identity can also be with SEQ ID NO: 1 or SEQ ID NO: 2.

The cytochrome P450 enzyme capable of N-demethylating reticuline and derivatives thereof can have at least 90% sequence identity with P450_DN15259_c0_g1_i7 (SEQ ID NO: 3). The 90% sequence identity can also be with SEQ ID NO: 1 or SEQ ID NO: 2.

The cytochrome P450 enzyme capable of N-demethylating reticuline and derivatives thereof can have at least 95% sequence identity with P450_DN15259_c0_g1_i7 (SEQ ID NO: 3). The 95% sequence identity can also be with SEQ ID NO: 1 or SEQ ID NO: 2.

The cytochrome P450 enzyme capable of N-demethylating reticuline and derivatives thereof can have at least 98% sequence identity with P450_DN15259_c0_g1_i7 (SEQ ID NO: 3). The 98% sequence identity can also be with SEQ ID NO: 1 or SEQ ID NO: 2.

The cytochrome P450 enzyme capable of N-demethylating reticuline and derivatives thereof can have at least 99% sequence identity with P450_DN15259_c0_g1_i7 (SEQ ID NO: 3). The 99% sequence identity can also be with SEQ ID NO: 1 or SEQ ID NO: 2.

The Mucorales cytochrome P450 enzyme capable of N-demethylating reticuline and derivatives thereof can have at least 99% sequence identity with CYPDN8 (SEQ ID NO: 72. The 99% sequence identity can also be with SEQ ID NO: 72 or SEQ ID NO: 73. The sequence identity can also be at least 95%. The sequence identity can also be at least 90%. The sequence identity can also be at least 80%. The sequence identity can also be at least 70%. The sequence identity can also be at least 60%.

The fungal Mucorales cytochrome P450 enzyme capable of N-demethylating and/or O-demethylating reticuline and/or derivatives thereof can have at least 50% sequence identity with any of the sequences selected from
i) CYPDN8 (SEQ ID NO: 72), ii) Mc_S2JT25 (SEQ ID NO: 52), iii) CYPDN17 (SEQ ID NO: 90), iv) CYPDN12 (SEQ ID NO: 80), v) Lr_P450 (SEQ ID NO: 8), vi) CYPDN29 (SEQ ID NO: 114), vii) CYPDN14 (SEQ ID NO: 84), vii) P450_DN15259_c0_g1_i7 (SEQ ID NO: 3), ix) LCOR_01865 (SEQ ID NO: 54), x) P450_DN5615_c2_g1_i9 (SEQ ID NO: 62), xi) P450_DN12791_c0_g1_i1 (SEQ ID NO: 5), xii) CYPDN16 (SEQ ID NO: 88), xiii) CYPDN18 (SEQ ID NO: 92), xiv) CYPDN27 (SEQ ID NO: 110), xv) CYPDN35 (SEQ ID NO: 126), xvi) CYPDN5 (SEQ ID NO: 66), xvii) CYPDN6 (SEQ ID NO: 68), xviii) CYPDN7 (SEQ ID NO: 70), xix) CYPDN10 (SEQ ID NO: 76), xx) CYPDN11 (SEQ ID NO: 78), xxi) CYPDN24 (SEQ ID NO: 104), xxii) CYPDN28 (SEQ ID NO: 112), xxiii) CYPDN13 (SEQ ID NO: 82), xxiv) CYPDN31 (SEQ ID NO: 118), xxv) CYPDN34 (SEQ ID NO: 124), xxvi) CYPDN22 (SEQ ID NO: 100), xxvii) CYPDN21 (SEQ ID NO: 98), xxviii) CYPDN30 (SEQ ID NO: 116), xxix) Ar_ORZ22410 (SEQ ID NO: 58), xxx) CYPDN20 (SEQ ID NO: 96), xxxi) CYPDN17 (SEQ ID NO: 90), and xxxii) CYPDN8 (SEQ ID NO: 72).
such as 30% sequence identity, such as 40% sequence identity, such as 50% sequence identity, such as 60% sequence identity, such as 70% sequence identity, such as 75% sequence identity, such as 80% sequence identity, such as 85% sequence identity, such as 90% sequence identity, such as 95% sequence identity, such as 97% sequence identity, such as 98% sequence identity, such as 99% sequence identity.

The sequence identify of any one of the sequences of the present invention can be at least 50% to any one of the sequences disclosed herein. The sequence identity can also be at least 95%. The sequence identity can also be at least 90%. The sequence identity can also be at least 80%. The sequence identity can also be at least 70%. The sequence identity can also be at least 60%.

A percent identity for any candidate nucleic acid or polypeptide relative to a reference nucleic acid or polypeptide can be determined as follows. A reference sequence (e.g., a nucleic acid sequence or an amino acid sequence) is aligned to one or more candidate sequences using the computer program ClustalW (version 1.83, default parameters), which allows alignments of nucleic acid or polypeptide sequences to be carried out across their entire length (global alignment). See Chenna et al., Nucleic Acids Res., 31 (13):3497-500 (2003).

ClustalW calculates the best match between a reference and one or more candidate sequences, and aligns them so that identities, similarities, and differences can be determined. Gaps of one or more residues can be inserted into a reference sequence, a candidate sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw).

To determine percent identity of a candidate nucleic acid or amino acid sequence to a reference sequence, the sequences are aligned using ClustalW, the number of identical matches in the alignment is divided by the length of the reference sequence, and the result is multiplied by 100. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

It will be appreciated that polypeptides described herein can include additional amino acids that are not involved in other enzymatic activities carried out by the enzyme, and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some aspects, a polypeptide includes an amino acid sequence that functions as a reporter, e.g., a green fluorescent protein or yellow fluorescent protein.

A recombinant gene encoding a polypeptide described herein comprises the coding sequence for that polypeptide, operably linked in sense orientation to one or more regulatory regions suitable for expressing the polypeptide. Because many microorganisms are capable of expressing multiple gene products from a polycistronic mRNA, multiple polypeptides can be expressed under the control of a single regulatory region for those microorganisms, if desired. A coding sequence and a regulatory region are considered to be operably linked when the regulatory region and coding sequence are positioned so that the regulatory region is effective for regulating transcription or translation of the sequence. Typically, the translation initiation site of the translational reading frame of the coding sequence is positioned between one and about fifty nucleotides downstream of the regulatory region for a monocistronic gene.

In some aspects, the coding sequence for a polypeptide described herein is identified in a species other than the recombinant host, i.e., is a heterologous gene.

Thus, if the recombinant host is a microorganism, the coding sequence can be from other prokaryotic or eukaryotic microorganisms, from plants or from animals. In some cases, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant gene construct. In addition, stably transformed exogenous genes typically are integrated at positions other than the position where the native sequence is found.

As disclosed herein, a “regulatory region” (prokaryotic and eukaryotic) refers to a nucleic acid having nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof.

A regulatory region typically comprises at least a core (basal) promoter. A regulatory region also can include at least one control element, such as an enhancer sequence, an upstream element, or an upstream activation region (UAR). A regulatory region is operably linked to a coding sequence by positioning the regulatory region and the coding sequence so that the regulatory region is effective for regulating transcription or translation of the sequence. For example, to operably link a coding sequence and a promoter sequence, the translation initiation site of the translational reading frame of the coding sequence is typically positioned between one and about fifty nucleotides downstream of the promoter. A regulatory region can, however, be positioned as much as about 5,000 nucleotides upstream of the translation initiation site or about 2,000 nucleotides upstream of the transcription start site.

The choice of regulatory regions to be included depends upon several factors, including, but not limited to, efficiency, selectability, inducibility, desired expression level, and preferential expression during certain culture stages. It is a routine matter for one skilled in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements.

One or more genes can be combined in a recombinant nucleic acid construct in “modules” useful for a discrete aspect of production of a compound described herein. Combining a plurality of genes in a module, particularly a polycistronic module, facilitates the use of the module in a variety of species.

It will be appreciated that because of the degeneracy of the genetic code, a number of nucleic acids can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated nucleic acids, these modified sequences can exist as purified molecules and can be incorporated into a vector or a virus for use in constructing modules for recombinant nucleic acid constructs.

Host Cells and Cultivation

At least one of the genes mentioned herein can be a recombinant gene, the particular recombinant gene(s) depending on the species or strain selected for use. Additional genes or biosynthetic modules can be included in order to increase compound yield, improve efficiency with which energy and carbon sources are used to produce the target compounds mentioned herein, and/or to enhance productivity from the cell culture or plant.

The cytochrome P450 reductase can originate from an organism that is Thamnostylum piriforme, Cunninghamella elegans, Lichtheimia ramosa, Gibberella fujikuroi, Saccharomyces cerevisiae, Mucor piriformis, Aspergillus sp., Homo sapiens, Pongo abelii, Papio anubis, Gorilla gorilla gorilla, Canis lupus familiaris, Pan troglodytes, Callithrix jacchus, Macaca fascicularis or Chlorocebus aethiops.

The cytochrome P450 enzyme capable of N-demethylating reticuline and derivatives thereof can also originate from an organism that is Thamnostylum piriforme, Lichtheimia ramosa, Cunninghamella echinulata, Cunninghamella dalmatica, Cunninghamella polymorpha, Rhizopus nigricans, Gibberella fujikuroi, or Saccharomyces cerevisiae.

In certain aspects of this invention, the recombinant host comprises a yeast cell, a plant cell, a mammalian cell, an insect cell, a fungal cell, an algal cell, a cyanobacteria or a bacterial cell.

In some aspects, the yeast cell is a cell from Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.

In some aspects, the yeast cell is a Saccharomycete.

In some aspects, the yeast cell is a Saccharomyces cerevisiae cell.

In some aspects, the algal cell is a cell from Blakeslea trispora, Dunaliella salina, Haematococcus pluvialis, Chlorella sp., Undaria pinnatifida, Sargassum, Laminaria japonica, Scenedesmus almeriensis species.

In some aspects, the cyanobacerial cell is a cell from Phormidium laminosum, Microcystis sp., Synechococcus sp., Pantoea sp., Flavobacterium sp.

In certain aspects of this invention, the recombinant host cell is a plant cell, a filamentous fungus, or a yeast cell. The host cell can also be a fungus cell.

In some aspects, the cell is a plant cell. The plant cell can be a Papaver sp. (e.g. Papaver somniferum or Papaver bracteatum cells), Nicotiana sp. (e.g. Nicotiana benthamiana cells), Arabidopsis sp., Physcomitrella sp., Thalictrum sp. (e.g. Thalictrum flavum), Coptis sp. (e.g Coptis japonica), Lindera sp. (Lindera aggregate), Annona sp. (e.g. Annona squamosa or Annona muricata), Ocotea sp. (e.g. Ocotea fasciculate), Duguetia sp., Sinomenium sp., Berberis sp., Corydalis sp., Ceratocapnos palaestinus, Anomianthus dulcis, Dicentra spectabilis, Glaucium flavum, Eschscholzia californica, Caulophyllum thalicroides, Chelidonium majus, Cocculus laurifolius, Delphinium pentagynum, Cinnamomum camphora, Clematis parviloba, Phylica rogersii, Phellodendron chinensis, Hypecoum lactiflorum, Fumaria officinalis, Croton celtidifolius, Mahonia aquifolium, Illigera parviflora, Aniba canelilla, Cryptocarya odorata, Litsea sp., Machilus thunbergii, Nectandra salicifolia, Neolitsea sp., Phoebe minutiflora, Strychnos holstii, Tinospora cordifolia, or Siparuna tonduziana cell.

The recombinant host cell can be a Nicotiana sp. cell. In certain aspects of this invention, the Nicotiana sp. cell is a Nicotiana benthamiana cell.

In some aspects, the recombinant host cell is a Physcomitrella sp. cell.

In certain aspects of this invention, the recombinant host cell is a filamentous fungus cell. The filamentous fungus cell can be Aspergillus nidulans, Aspergillus sydowii, Aspergillus terreus, Aspergillus oryzae, Aspergillus caelatus, Aspergillus chevalieri, Aspergillus longivesica, Aspergillus parvulus, Aspergillus amylovorus, Aspergillus niger, Aspergillus niger, Aspergillus aculeatus, Aspergillus ellipticus, Aspergillus violaceofuscus, Aspergillus brunneoviolaceus, Aspergillus japonicus, Aspergillus brasiliensis, Aspergillus brasiliensis, Aspergillus aculeatinus, Aspergillus thermomutatus, Aspergillus implicatus, Aspergillus acristatus, Penicillium bilaiae, Penicillium rubens, Penicillium chrysogenum, Penicillium expansum, Penicillium antarcticum, Trichoderma reesei, Talaromyces atroroseus, Asteromyces cruciatus, or Neurospora crassa.

In certain aspects of this invention, the recombinant host cell is a yeast cell comprising Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., Schwanniomyces occidentalis, Sporidiobolus salmonicolor, Starmerella bacillaris, Sugiyamaella americana, Talaromyces atroroseus, Torulaspora delbrueckii, Trichoderma reesei, Wickerhamia fluorescens, Wickerhamiella sorbophila, Wickerhamiella versatilis, Zygosaccharomyces rouxii, Zygotorulaspora Florentina, Saccharomyces cerevisiae var. ellipsoideus, Saccharomyces paradoxus, Saccharomyces pastorianus, Saccharomyces uvarum, Saccharomycodes ludwigii var. ludwigii, Saitoella complicate, Schizosaccharomyces japonicus, Schizosaccharomyces octosporus, Asteromyces cruciatus, Aureobasidium pullulans, Candida cylindracea, Candida albicans, Cutaneotrichosporon curvatus, Cyberlindnera jadinii, Debaromyces hansenii, Dekkera bruxellensis, Diutina rugosa, Eremothecium gossypii, Galactomyces candidus, Geotrichum candidum, Geotrichum fermentans, Hanseniaspora uvarum, Hanseniaspora vineae, Issatchenkia orientalis, Kazachstania exigua, Kazachstania servazzii, Kluyveromyces lactis, Kluyveromyces marxianus, Komagataella phaffli, Lachancea thermotolerans, Lipomyces starkeyi, Moesziomyces antarcticus, Naumovozyma castellii, Naumovozyma dairenensis, Ogataea polymorpha, Ogataea thermomethanolica, Pachysolen tannophilus, Papiliotrema laurentii, Penicillium arizonense, Pichia fermentans, Rhodotorula mucilaginosa, Saccharomyces bayanus or Rhodospiridium sp.

In certain aspects of this invention, microorganisms can include, but are not limited to, S. cerevisiae and E. coli. The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

The constructed and genetically engineered microorganisms provided by the invention can be cultivated using conventional fermentation processes, including, inter alia, chemostat, batch, fed-batch cultivations, continuous perfusion fermentation, and continuous perfusion cell culture.

A number of prokaryotes and eukaryotes are suitable for use in constructing the recombinant microorganisms described herein, e.g., gram-negative bacteria, yeast and fungi. A species and strain selected for use as a strain for production of the compounds described herein is first analyzed to determine which production genes are endogenous to the strain and which genes are not present. Genes for which an endogenous counterpart is not present in the strain are assembled in one or more recombinant constructs, which are then transformed into the strain in order to supply the missing function(s).

Exemplary prokaryotic and eukaryotic species are described in more detail below. However, it will be appreciated that other species can be suitable. For example, suitable species can be Agaricus, Aspergillus, Bacillus, Candida, Corynebacterium, Escherichia, Fusarium/Gibberella, Kluyveromyces, Laetiporus, Lentinus, Phaffia, Phanerochaete, Pichia, Physcomitrella, Rhodoturula, Saccharomyces, Schizosaccharomyces, Sphaceloma, Xanthophyllomyces and Yarrowia. Exemplary species from such genera include Lentinus tigrinus, Laetiporus sulphureus, Phanerochaete chrysosporium, Pichia pastoris, Physcomitrella patens, Rhodoturula glutinis 32, Rhodoturula mucilaginosa, Phaffia rhodozyma UBV-AX, Xanthophyllomyces dendrorhous, Fusarium fujikuroi, Gibberella fujikuroi, Candida utilis and Yarrowia lipolytics. In some aspects, a microorganism can be an Ascomycete such as Gibberella fujikuroi, Kluyveromyces lactis, Schizosaccharomyces pombe, Aspergillus niger, or Saccharomyces cerevisiae.

In some aspects, a microorganism can be a prokaryote such as Escherichia coli, Rhodobacter sphaeroides, or Rhodobacter capsulatus. It will be appreciated that certain microorganisms can be used to screen and test genes of interest in a high throughput manner, while other microorganisms with desired productivity or growth characteristics can be used for large-scale production of the compounds described herein.

In other aspects, the recombinant host cell can be a plant cell, a filamentous fungus, or a yeast cell.

The recombinant host cell of the present invention can be a plant cell. For example, suitable plant species can be Papaver sp. (e.g. Papaver somniferum or Papaver bracteatum cells), Nicotiana sp. (e.g. Nicotiana benthamiana cells), Arabidopsis sp., Physcomitrella sp., Thalictrum sp. (e.g. Thalictrum flavum), Coptis sp. (e.g Coptis japonica), Lindera sp. (Lindera aggregate), Annona sp. (e.g. Annona squamosa or Annona muricata), Ocotea sp. (e.g. Ocotea fasciculate), Duguetia sp., Sinomenium sp., Berberis sp., Corydalis sp., Ceratocapnos palaestinus, Anomianthus dulcis, Dicentra spectabilis, Glaucium flavum, Eschscholzia californica, Caulophyllum thalicroides, Chelidonium majus, Cocculus laurifolius, Delphinium pentagynum, Cinnamomum camphora, Clematis parviloba, Phylica rogersii, Phellodendron chinensis, Hypecoum lactiflorum, Fumaria officinalis, Croton celtidifolius, Mahonia aquifolium, Illigera parviflora, Aniba canelilla, Cryptocarya odorata, Litsea sp., Machilus thunbergii, Nectandra salicifolia, Neolitsea sp., Phoebe minutiflora, Strychnos holstii, Tinospora cordifolia, or Siparuna tonduziana, In some aspects, the cell can be a Papaver sp. cell. The Papaver sp. cell can be a Papaver somniferum or a Papaver bracteatum cell.

In other aspects, the recombinant host cell can be a Nicotiana sp. cell. The Nicotiana sp. cell can be a Nicotiana benthamiana cell.

The recombinant host cell can also be an Arabidopsis sp. cell.

In some aspects, the cell can be a Physcomitrella sp. cell.

Physcomitrella spp.

Physcomitrella mosses, when grown in suspension culture, have characteristics similar to yeast or other fungal cultures. This genera can be used for producing plant secondary metabolites, which can be difficult to produce in other types of cells.

The recombinant host cell of the present invention can be a filamentous fungus cell.

For example, suitable filamentous fungus cell species can be Aspergillus nidulans, Aspergillus sydowii, Aspergillus terreus, Aspergillus oryzae, Aspergillus caelatus, Aspergillus chevalieri, Aspergillus longivesica, Aspergillus parvulus, Aspergillus amylovorus, Aspergillus niger, Aspergillus niger, Aspergillus aculeatus, Aspergillus ellipticus, Aspergillus violaceofuscus, Aspergillus brunneoviolaceus, Aspergillus japonicus, Aspergillus brasiliensis, Aspergillus brasiliensis, Aspergillus aculeatinus, Aspergillus thermomutatus, Aspergillus implicatus, Aspergillus acristatus, Penicillium bilaiae, Penicillium rubens, Penicillium chrysogenum, Penicillium expansum, Penicillium antarcticum, Trichoderma reesei, Talaromyces atroroseus, Asteromyces cruciatus, or Neurospora crassa.

The recombinant host cell of the present invention can be a yeast cell. For example, suitable yeast cell species can be Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., Schwanniomyces occidentalis, Sporidiobolus salmonicolor, Starmerella bacillaris, Sugiyamaella americana, Talaromyces atroroseus, Torulaspora delbrueckii, Trichoderma reesei, Wickerhamia fluorescens, Wickerhamiella sorbophila, Wickerhamiella versatilis, Zygosaccharomyces rouxii, Zygotorulaspora Florentina, Saccharomyces cerevisiae var. ellipsoideus, Saccharomyces paradoxus, Saccharomyces pastorianus, Saccharomyces uvarum, Saccharomycodes ludwigii var. ludwigii, Saitoella complicate, Schizosaccharomyces japonicus, Schizosaccharomyces octosporus, Asteromyces cruciatus, Aureobasidium pullulans, Candida cylindracea, Candida albicans, Cutaneotrichosporon curvatus, Cyberlindnera jadinii, Debaromyces hansenii, Dekkera bruxellensis, Diutina rugosa, Eremothecium gossypii, Galactomyces candidus, Geotrichum candidum, Geotrichum fermentans, Hanseniaspora uvarum, Hanseniaspora vineae, Issatchenkia orientalis, Kazachstania exigua, Kazachstania servazzii, Kluyveromyces lactis, Kluyveromyces marxianus, Komagataella phaffli, Lachancea thermotolerans, Lipomyces starkeyi, Moesziomyces antarcticus, Naumovozyma castellii, Naumovozyma dairenensis, Ogataea polymorpha, Ogataea thermomethanolica, Pachysolen tannophilus, Papiliotrema laurentii, Penicillium arizonense, Pichia fermentans, Rhodotorula mucilaginosa, Saccharomyces bayanus or Rhodospiridium sp.

Saccharomyces cerevisiae

Saccharomyces cerevisiae is a widely used organism in synthetic biology, and can be used as the recombinant microorganism platform. There are libraries of mutants, plasmids, detailed computer models of metabolism and other information available for S. cerevisiae, allowing for rational design of various modules to enhance product yield. Methods are known for making recombinant microorganisms.

The genes described herein can be expressed in yeast using any of a number of known promoters. Strains that overproduce terpenes are known and can be used to increase the amount of geranylgeranyl diphosphate available for production of the compounds described herein.

In some aspects, auxotrophic markers for cloning include, but are not limited to, HIS3, URA3, TRP1, LEU2, LYS2, ADE2, and GAL, which allow for selection of recombinant strains with an inserted gene of interest. For example, one or more of the auxotrophic markers of strains EYS583-7a (MAT alpha lys2 ADE8 his3 ura3 leu2 trpl), EFSC1772 (MAT alpha ura3 (×2) his3 leu2), EYS4853 (MATalpha his3Δ0 leu2Δ0 ura3Δ0 ho GAL2 CAT5(J91M) MIP1(A661T) SAL1(G403L) YORWA22::npBIOlnt-npBIO6nt) or EVST25898 (MATalpha his3Δ0 leu2Δ0 ura3Δ0 aro3A::pTEF1-ARO4(K229L)-tCYC1::pPGK1-ARO7(T266L)-tADH1::KI CAT5-91Met GAL2 ho MIP1-661Thr SAL1-1 YORWA22::npBIOlnt-npBIO6nt) can be used during cloning.

Auxotrophic markers can be optionally removed from the yeast genome using methods not limited to Cre-Lox recombination or negative selection with 5-fluoroorotic acid (5-FOA). In other aspects, antibiotic resistance, such as kanamycin, can be used as selection marker for construction of recombinant strains.

E. coli

E. coli, another widely used platform organism in synthetic biology, can also be used as the recombinant microorganism platform. Similar to Saccharomyces, there are libraries of mutants, plasmids, detailed computer models of metabolism and other information available for E. coli, allowing for rational design of various modules to enhance product yield. Methods similar to those described above for Saccharomyces can be used to make recombinant E. coli microorganisms.

Arxula adeninivorans (Blastobotrys adeninivorans)

Arxula adeninivorans is dimorphic yeast (it grows as budding yeast like the baker's yeast up to a temperature of 42° C., above this threshold it grows in a filamentous form) with unusual biochemical characteristics. It can grow on a wide range of substrates and can assimilate nitrate. It has successfully been applied to the generation of strains that can produce natural plastics or the development of a biosensor for estrogens in environmental samples.

Yarrowia lipolytica

Yarrowia lipolytica is dimorphic yeast (see Arxula adeninivorans) and belongs to the family Hemiascomycetes. The entire genome of Yarrowia lipolytica is known. Yarrowia species is aerobic and considered to be non-pathogenic. Yarrowia is efficient in using hydrophobic substrates (e.g. alkanes, fatty acids, oils) and can grow on sugars. It has a high potential for industrial applications and is an oleaginous microorgamism.

Yarrowia lipolyptica can accumulate lipid content to approximately 40% of its dry cell weight and is a model organism for lipid accumulation and remobilization. See e.g., Nicaud, 2012, Yeast 29(10):409-18; Beopoulos et al., 2009, Biochimie 91(6):692-6; Bankar et al., 2009, Appl Microbiol Biotechnol. 84(5):847-65.

Rhodotorula sp.

Rhodotorula is unicellular, pigmented yeast. The oleaginous red yeast, Rhodotorula glutinis, has been shown to produce lipids and carotenoids from crude glycerol (Saenge et al., 2011, Process Biochemistry 46(1):210-8). Rhodotorula toruloides strains have been shown to be an efficient fed-batch fermentation system for improved biomass and lipid productivity (Li et al., 2007, Enzyme and Microbial Technology 41:312-7).

Rhodosporidium toruloides

Rhodosporidium toruloides is oleaginous yeast and useful for engineering lipid-production pathways (See e.g. Zhu et al., 2013, Nature Commun. 3:1112; Ageitos et al., 2011, Applied Microbiology and Biotechnology 90(4): 1219-27).

Candida boidinii

Candida boidinii is methylotrophic yeast (it can grow on methanol). Like other methylotrophic species such as Hansenula polymorpha and Pichia pastoris, it provides an excellent platform for producing heterologous proteins. Yields in a multigram range of a secreted foreign protein have been reported. A computational method, IPRO, recently predicted mutations that experimentally switched the cofactor specificity of Candida boidinii xylose reductase from NADPH to NADH. See, e.g., Mattanovich et al., 2012, Methods Mol Biol. 824:329-58; Khoury et al., 2009, Protein Sci. 18(10):2125-38.

Hansenula polymorpha (Pichia angusta)

Hansenula polymorpha is methylotrophic yeast (see Candida boidinii). It can furthermore grow on a wide range of other substrates; it is thermo-tolerant and can assimilate nitrate (see also Kluyveromyces lactis). It has been applied to producing hepatitis B vaccines, insulin and interferon alpha-2a for the treatment of hepatitis C, furthermore to a range of technical enzymes. See, e.g., Xu et al., 2014, Virol Sin. 29(6):403-9.

Kluyveromyces lactis

Kluyveromyces lactis is yeast regularly applied to the production of kefir. It can grow on several sugars, most importantly on lactose which is present in milk and whey. It has successfully been applied among others for producing chymosin (an enzyme that is usually present in the stomach of calves) for producing cheese. Production takes place in fermenters on a 40,000 L scale. See, e.g., van Ooyen et al., 2006, FEMS Yeast Res. 6(3):381-92.

Pichia pastoris

Pichia pastoris is methylotrophic yeast (see Candida boidinii and Hansenula polymorpha). It provides an efficient platform for producing foreign proteins. Platform elements are available as a kit and it is worldwide used in academia for producing proteins. Strains have been engineered that can produce complex human N-glycan (yeast glycans are similar but not identical to those found in humans). See, e.g., Piirainen et ai, 2014, N Biotechnol. 31 (6):532-7.

Methods for Production

The N-demethylated reticuline and derivatives thereof can be produced though methods comprising cultivation of the host cells of the invention in presence of a reticuline and derivative substrate. Thus, an aspect of the invention relates to a method of producing a N-demethylated and/or O-demethylated reticuline and/or derivatives thereof, comprising cultivating the recombinant host of the invention in a culture medium under conditions in which the one or more genes encoding the cytochrome P450 enzymes of the invention is/are expressed.

The N-demethylated compounds mentioned herein are also known as nor-compounds. N-demethylated thebaine is therefore also known as northebaine.

The reticuline and/or derivatives thereof can be one or more of (S)-reticuline, 1,2 dehydroreticuline, (R)-reticuline, salutaridine, salutaridinol, thebaine, 7-O-acetyl-salutaridinol, oripavine, neopinone, codeinone, codeine, morphinone, morphine, hydrocodone, 14-hydroxycodeinone and oxycodone.

The reticuline and/or derivatives thereof can be (S)-reticuline. The reticuline and derivatives thereof can be 1,2 dehydroreticuline. The reticuline and derivatives thereof can be (R)-reticuline. The reticuline and derivatives thereof can be salutaridine. The reticuline and derivatives thereof can be salutaridinol or 7-O-acetyl-salutaridinol. The reticuline and derivatives thereof can be thebaine. The reticuline and derivatives thereof can be oripavine. The reticuline and derivatives thereof can be neopinone. The reticuline and derivatives thereof can be codeinone. The reticuline and derivatives thereof can be codeine. The reticuline and derivatives thereof can be morphinone. The reticuline and derivatives thereof can be morphine. The reticuline and derivatives thereof can be hydrocodone. The reticuline and derivatives thereof can be 14-hydroxycodeinone. The reticuline and derivatives thereof can be oxycodone.

The method can further comprise cultivating the recombinant host of the invention in presence of reticuline or derivatives thereof in a culture medium under conditions in which the one or more genes encoding the cytochrome P450 reductase of the invention is/are expressed.

The reticuline or derivatives thereof can be added to the culture.

Another embodiment of the invention relates to an in vitro method for converting reticuline or derivatives thereof into its nor-version comprising contacting a crude cell extract, microsomal fraction or lysate of one or more host cells of the invention with reticuline or derivatives thereof to produce an N-demethylated nor-version of reticuline or derivatives thereof.

A further embodiment of the invention relates to an in vitro method for converting reticuline or derivatives thereof into its nor-version comprising purifying the one or more enzymes of the invention from a naturally producing or recombinant host and adding reticuline or derivatives thereof to a suitable reaction mixture containing NADPH or an NADPH regenerating system for N-demethylating and/or O-demethylated reticuline or derivatives thereof.

Another embodiment of the invention relates to purification of one or more of the enzymes of the current invention from a natural or a recombinant host, coupling them to a solid support and using them for N-demethylation of reticuline or derivatives thereof in presence of a suitable buffer system, an NADPH regenerating system.

An aspect of the invention relates to an in vitro method for N-demethylating and/or O-demethylated a reticuline or a derivative thereof, comprising contacting reticuline or a derivative thereof with a recombinant P450 enzyme capable of N-demethylating reticuline or a derivative thereof.

The method can further comprise cultivating a recombinant host cell of the invention in a culture medium in presence of reticuline or a derivative thereof, under conditions in which the one or more genes encoding the cytochrome P450 enzymes is/are expressed.

The method can further comprise cultivating the recombinant host cell of the invention in a culture medium under conditions in which the one or more genes encoding the cytochrome P450 reductase is/are expressed.

An embodiment of the invention relates to a composition comprising a compound selected from the group consisting of reticuline and derivatives thereof obtainable from the methods according to the invention, and further comprising elements from a fungal fermentation broth and/or at least one fungal specific metabolite.

A further embodiment of the invention relates to a composition comprising a N-demethylated reticuline or a derivative thereof, and a recombinant P450 enzyme capable of N-demethylating thebaine or oripavine.

DNA Molecules

The enzymes mentioned herein can be encoded by a DNA molecule. Thus, an aspect of the invention relates to a DNA molecule comprising a nucleic acid encoding one or more of the recombinant genes of the invention.

The DNA molecule can be an expression vector comprising the DNA molecule according to the invention, and a promoter suitable for expression of the DNA molecule in a cell.

The DNA molecule can be introduced into a host cell using techniques that are well-known to the person skilled in the art. Thus, an embodiment of the invention relates to a host cell comprising the DNA molecule of the invention.

Chemical Synthesis of buprenorphine

Aspects and embodiments of the disclosure related to methods of preparing buprenorphine from Compound MeO—I—H, or HO—I—H (as defined below) provide improved routes to buprenorphine that can be shorter, more efficient, and/or produce less toxic waste than, e.g., current commercial routes to buprenorphine. As a result, these aspects and embodiments can be well-suited for commercial (e.g., kg-scale) production of buprenorphine. Further, in certain aspects and embodiments, the synthetic routes disclosed herein advantageously avoid the harsh conditions and/or toxic byproducts of an N-demethylation step and can accordingly be particularly well-suited for producing buprenorphine on a commercial, e.g., kg, scale.

An aspect of the present invention relates to a method of preparing buprenorphine, or a salt thereof, from Compound HO—I—H, or a salt thereof, comprising contacting thebaine and/or oripavine with the recombinant host of the present invention to produce the Compound MeO—I—H or the Compound HO—I—H, and prepare buprenorphine by any one or more of the below chemical synthesis pathways.

The disclosure relates to methods for preparing buprenorphine:

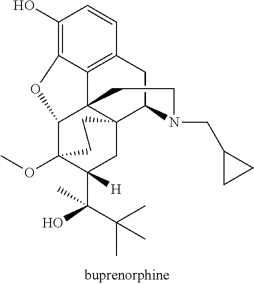 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

In various aspects and embodiments, the methods comprise a series of reaction steps to prepare buprenorphine from a compound of Formula I—H:

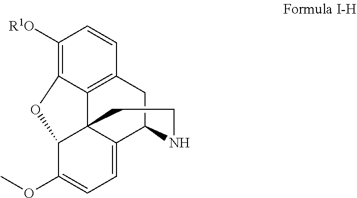

wherein R¹ is H (Compound HO—I—H; nororipavine), methyl (Compound MeO—I—H; northebaine). In certain embodiments, the compound of Formula I—H (e.g., nororipavine or northebaine) is produced by a method as otherwise described herein (e.g., a method comprising cultivating a recombinant host in a culture medium under conditions in which one or more genes encoding cytochrome P450 enzymes is/are expressed). Such methods provide an improved route to buprenorphine that can be shorter, more efficient, and/or produce less toxic waste than, e.g., current commercial routes to buprenorphine. As a result, these aspects and embodiments can be well-suited for commercial (e.g., kg-scale) production of buprenorphine. Further, such methods advantageously avoid the harsh conditions and/or toxic byproducts of an N-demethylation step and can accordingly be particularly well-suited for producing buprenorphine on a commercial, e.g., kg, scale.

As used herein, the term “benzyl” (“Bn”) includes unsubstituted (i.e., (C₆H₅)—CH₂—) and substituted benzyl (i.e., benzyl substituted at the 2-, 3-, and/or 4-position with C₁-C₈ alkyl or halide). The person of ordinary skill in the art will appreciate that oxygen protecting groups include alkoxycarbonyl, acyl, acetal, ether, ester, silyl ether, alkylsulfonyl, and arylsulfonyl. Exemplary oxygen protecting groups include allyl, triphenylmethyl (trityl or Tr), benzyl, methanesulfonyl, p-toluenesulfonyl, p-methoxybenzyl (PMB), p-methoxyphenyl (PMP), methoxymethyl (MOM), p-methoxyethoxymethyl (MEM), tetrahydropyranyl (THP), ethoxyethyl (EE), methylthiomethyl (MTM), 2-methoxy-2-propyl (MOP), 2-trimethylsilylethoxymethyl (SEM), benzoate (BZ), allyl carbonate, 2.2.2-trichloroethyl carbonate (Troc), 2-trimethylsilylethyl carbonate, trimethylsilyl (TMS), triethylsilyl (TES), triisopropylsilyl (TIPS), triphenylsilyl (TPS), t-butyldimethylsilyl (TBDMS), and t-butyldiphenylsilyl (TBDPS). A variety of protecting groups for the oxygen and the synthesis thereof can be found in “Protective Groups in Organic Synthesis” by T. W.

Greene and P. G. M. Wuts, John Wiley & Sons, 1999. In certain embodiments, an appropriate oxygen protecting group can be used in place of benzyl.

In some embodiments, the methods comprise reacting a compound of Formula I—H with cyclopropane carboxaldehyde followed by a hydride source; or reacting a compound of Formula I—H with cyclopropanecarboxylic acid halide followed by a reducing agent; or reacting a compound of Formula I—H with cyclopropylmethyl halide or activated cyclopropane methanol; to provide a compound of Formula I-MCP:

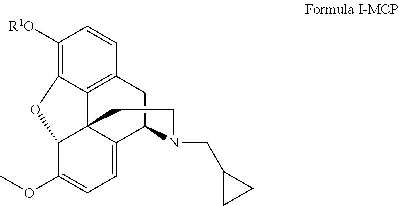

wherein R¹ is H (Compound HO—I-MCP), methyl (Compound MeO—I-MCP),

In some embodiments, the methods comprise reacting a compound of Formula I—H with benzyl halide, benzyl sulfonate, or activated benzyl alcohol to provide a compound of Formula I-Bn:

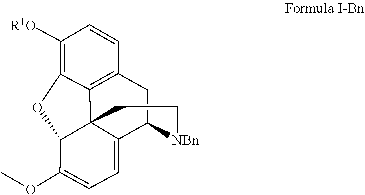

wherein R¹ is benzyl (Compound BnO—I—Bn). A preparation of Compound BnO—I—Bn, as an intermediate towards noroxymorphone and ultimately towards naltrexone and naloxone, was described in Helv. Chim. Acta 92:1359-65 (2009).

In some embodiments, the methods comprise reacting a compound of Formula I—H with acyl halide to provide a compound of Formula I—Ac:

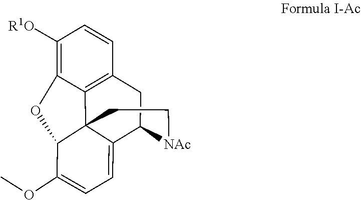

wherein R¹ is H (Compound HO—I—Ac), benzyl (Compound BnO—I—Ac), or acyl (Compound AcO—I—Ac).

As used herein, the term “acyl” includes C₁-C₈ aliphatic acyl groups (e.g., acetyl, ethanoyl, cyclopropanecarbonyl, etc.) and optionally substituted C₆-C₁₃ aromatic acyl groups (e.g., optionally substiututed benzoyl (“Bz”), e.g., benzoyl, 4-methylbenzoyl, 4-fluorobenzoyl, etc.). For example, in certain embodiments, the methods comprise reacting a compound of Formula I—H with benzoyl chloride to provide a compound of Formula I—Ac.

In some embodiments, the methods comprise reacting a compound of Formula I—Ac (e.g., Compound HO—I—Ac) with benzyl halide, benzyl sulfonate, or activated benzyl alcohol to provide another compound of Formula I—Ac (e.g., Compound BnO—I—Ac). In some embodiments, the methods comprise reacting a compound of Formula I—Ac (e.g., Compound AcO—I—Ac) with lithium aluminum hydride (LAH) to provide a compound of Formula I-Bn (e.g., Compound BnO—I—Bn).

In some embodiments, the methods comprise reacting a compound of Formula I-MCP (e.g., Compound HO—I-MCP) with benzyl halide, benzyl sulfonate, or activated benzyl alcohol to provide another compound of Formula I-MCP (e.g., Compound BnO-I-MCP).

In some embodiments, the methods comprise reacting a compound of Formula I-MCP with methyl vinyl ketone to provide a compound of Formula II-MCP:

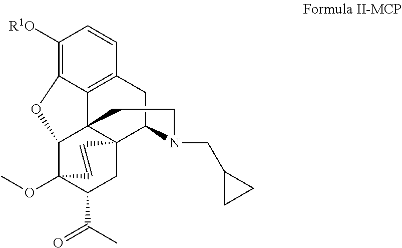

wherein R¹ is H (Compound HO—II-MCP), methyl (Compound MeO—II-MCP), or benzyl (Compound BnO—II-MCP).

In some embodiments, the methods comprise reacting a compound of Formula I-Bn with methyl vinyl ketone to provide a compound of Formula II-Bn:

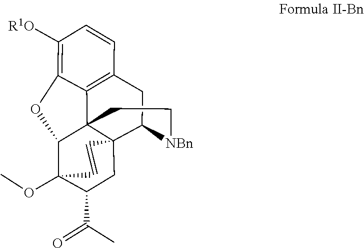

wherein R¹ is benzyl (Compound BnO—II-Bn).

In some embodiments, the methods comprise reacting a compound of Formula II-MCP (e.g., Compound HO—II-MCP) with benzyl halide, benzyl sulfonate, or activated benzyl alcohol to provide another compound of Formula II-MCP (e.g., Compound BnO—II-MCP).

In some embodiments, the methods comprise reacting a compound of Formula I—Ac with methyl vinyl ketone to provide a compound of Formula II—Ac:

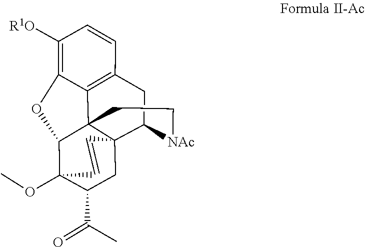

wherein R¹ is acyl (Compound AcO—II—Ac).

In some embodiments, the methods comprise reacting a compound of Formula II-MCP with H₂ in the presence of a hydrogenation catalyst to provide a compound of Formula IIIB-MCP:

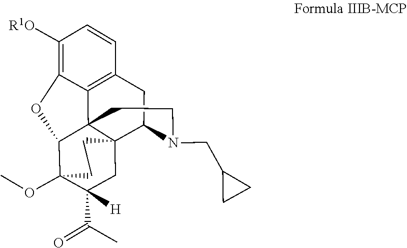

wherein R¹ is H (Compound HO—IIIB-MCP) or methyl (Compound MeO—IIIB-MCP).

In some embodiments, the methods comprise reacting a compound of Formula II—Ac with H₂ in the presence of a hydrogenation catalyst to provide a compound of Formula IIIB—Ac:

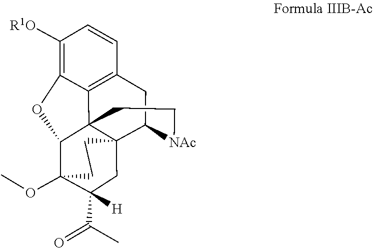

wherein R¹ is Ac (Compound AcO—IIIB—Ac).

In some embodiments, the methods comprise reacting a compound of Formula II-MCP with tert-butylmagnesium halide to provide a compound of Formula IIIA-MCP:

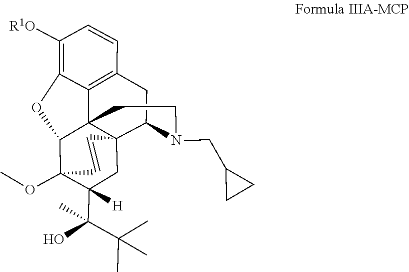

wherein R¹ is H (Compound HO—IIIA-MCP), methyl (Compound MeO—IIIA-MCP), or benzyl (Compound BnO—IIIA-MCP).

In some embodiments, the methods comprise reacting a compound of formula IIIA-MCP (e.g., Compound Me-IIIA-MCP) with a demethylating agent to provide another compound of IIIA-MCP (e.g., Compound HO—IIIA-MCP).

In some embodiments, the methods comprise reacting a compound of Formula II-Bn with tert-butylmagnesium halide to provide a compound of Formula IIIA-Bn:

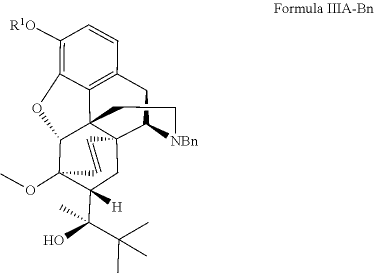

wherein R¹ is benzyl (Compound BnO—IIIA-Bn).

In some embodiments, the methods comprise reacting a compound of Formula II—Ac with tert-butylmagnesium halide to provide a compound of Formula IIIA-Ac:

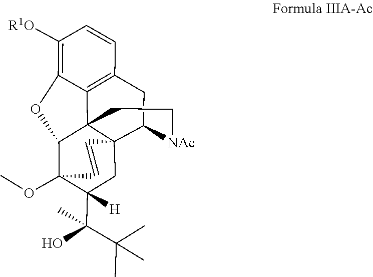

wherein R¹ is H (Compound HO—IIIA-Ac).

In some embodiments, the methods comprise reacting a compound of Formula IIIA-Ac (e.g., Compound HO—IIIA-Ac), wherein Ac is optionally substituted benzoyl, with lithium aluminum hydride (LAH) to provide a compound of Formula IIIA-Bn (e.g., Compound HO—IIIA-Bn).

In some embodiments, the methods comprise reacting a compound of Formula IIIB-MCP with tert-butylmagnesium halide to provide a compound of Formula IV-MCP:

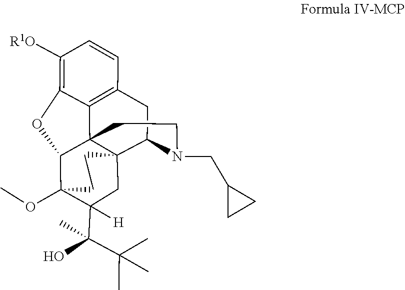

wherein R¹ is H (Compound HO—IV-MCP; buprenorphine) or methyl (Compound MeO—IV-MCP).

In some embodiments, the methods comprise reacting a compound of Formula IIIB—Ac with tert-butylmagnesium halide to provide a compound of Formula IV—Ac:

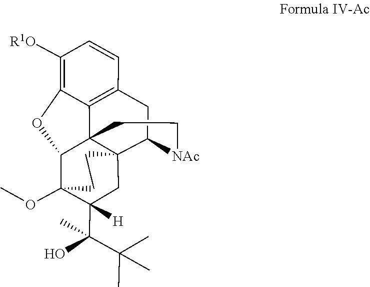

wherein R¹ is H (Compound HO—IV—Ac).

In some embodiments, the methods comprise reacting a compound of Formula IIIA-MCP with H₂ in the presence of a hydrogenation catalyst to provide a compound of Formula IV-MCP (see above), wherein R₁ is H (Compound HO—IV-MCP; buprenorphine) or methyl (Compound MeO—IV-MCP).

In some embodiments, the methods comprise reacting a compound of Formula IIIA-Ac with H₂ in the presence of a hydrogenation catalyst to provide a compound of Formula IV—Ac (see above), wherein R₁ is H (Compound HO—IV—Ac).

In some embodiments, the methods comprise reacting a compound of Formula IIIA-Bn with H₂ in the presence of a hydrogenation catalyst to provide a compound of Formula IV—H:

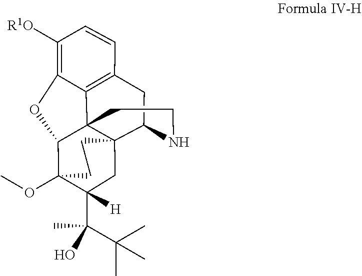

wherein R¹ is H (Compound HO—IV—H; norbuprenorphine).

In some embodiments, the methods comprise reacting a compound of Formula IV—Ac (e.g., compound HO—IV—Ac) with Schwartz's reagent (zirconocene hydrochloride) or base to provide a compound of Formula IV—H (e.g., compound HO—IV—H).

In some embodiments, the methods comprise reacting a compound of Formula IV-MCP (e.g., Compound Me-IV-MCP) with a demethylating agent to provide buprenorphine.

In some embodiments, the methods comprise reacting a compound of Formula IV—H (e.g., Compound HO—IV—H) with cyclopropane carboxaldehyde followed by a hydride source; or reacting a compound of Formula IV—H (e.g., Compound HO—IV—H) with cyclopropanecarboxylic acid halide followed by a reducing agent; or reacting a compound of Formula IV—H (e.g., Compound HO—IV—H) with cyclopropylmethyl halide or activated cyclopropane methanol; to provide buprenorphine.

Formula I—H→Formula I-MCP

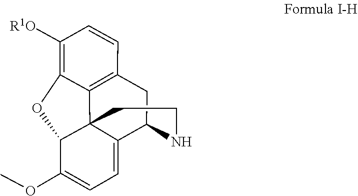

| R¹ of Formula I-H | Compound |
|---|---|
| H | Compound HO-I-H |
| Me | Compound MeO-I-H |

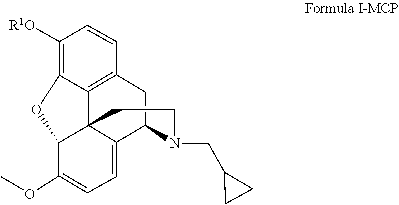

| R¹ of Formula I-MCP | Compound |
|---|---|
| H | Compound HO-I-MCP |
| Me | Compound MeO-I-MCP |
| Bn | Compound BnO-I-MCP |

Step (i)(A1)

In some embodiments, reacting a compound of Formula I—H with cyclopropane carboxaldehyde followed by a hydride source provides a compound of Formula I-MCP. In certain embodiments, reacting Compound HO—I—H with cyclopropane carboxaldehyde followed by a hydride source provides Compound HO—I-MCP. In certain embodiments, reacting Compound MeO—I—H with cyclopropane carboxaldehyde followed by a hydride source provides Compound MeO—I-MCP. In certain embodiments, reacting See Examples 12 and 23.

In some embodiments, the hydride source is formic acid, hydrogen, sodium cyanoborohydride, sodium borohydride, or sodium triacetoxy borohydride. In some embodiments, the hydride source is formic acid. In some embodiments, the reaction is catalyzed by a ruthenium(I) complex or a ruthenium(II) complex, e.g., a dichloro(p-cymene)ruthenium(II) dimer. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof. In some embodiments, the reaction is performed in the presence of a trialkylamine, e.g., triethylamine, diisopropylethylamine, 4-methyl-morpholine, or N-methyl-piperidine.

In some embodiments, the cyclopropane carboxaldehyde is reacted at a temperature within the range of about 30° C. to about 90° C., e.g., about 35° C. to about 90° C., or about 40° C. to about 90° C., or about 45° C. to about 90° C., or about 50° C. to about 90° C., or about 55° C. to about 90° C., or about 60° C. to about 90° C., or about 65° C. to about 90° C., or about 70° C. to about 90° C., or about 30° C. to about 85° C., or about 30° C. to about 80° C., or about 30° C. to about 75° C., or about 30° C. to about 70° C., or about 30° C. to about 65° C., or about 30° C. to about 60° C., or about 30° C. to about 55° C., or about 30° C. to about 50° C., or about 35° C. to about 85° C., or about 40° C. to about 80° C., or about 45° C. to about 75° C., or about 50° C. to about 70° C., or about 55° C. to about 65° C. In some embodiments, the cyclopropane carboxaldehyde is reacted for a period of time within the range of about 30 minutes to about 5 hours, e.g., about 1 hour to about 5 hours, or about 1.5 hours to about 5 hours, or about 2 hours to about 5 hours, or about 2.5 hours to about 5 hours, or about 3 hours to about 5 hours, or about 3.5 hours to about 5 hours, or about 4 hours to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 30 minutes to about 2 hours, or about minutes to about 1.5 hours.

Step (i)(A2)

In some embodiments, reacting a compound of Formula I—H with cyclopropanecarboxylic acid halide followed by a reducing agent provides a compound of Formula I-MCP. In certain embodiments, reacting Compound HO—I—H with cyclopropanecarboxylic acid halide followed by a reducing agent provides Compound HO—I-MCP. In certain embodiments, reacting Compound MeO—I—H with cyclopropanecarboxylic acid halide followed by a reducing agent provides Compound MeO—I-MCP. In certain embodiments, reacting Compound BnO—I—H with cyclopropanecarboxylic acid halide followed by a reducing agent provides Compound BnO-I-MCP. See Examples 13 and 24.

In some embodiments, the cyclopropanecarboxylic acid halide is cyclopropanecarboxylic acid chloride, cyclopropanecarboxylic acid anhydride, cyclopropanecarboxylic acid bromide, or an activated cyclopropanecarboxylic acid (e.g., an activated cyclopropanecarboxylic acid formed by reaction with an alcohol such as pentafluorophenol, 4-nitrophenol, N-hydroxysuccinimide, N-hydroxymaleimide, 1-Hydroxybenzotriazole, or 1-hydroxy-7-azabenzotriazole). In some embodiments, the reducing agent is LiAlH₄ or NaBH₄. In some embodiments, the reaction with cyclopropanecarboxylic acid halide is performed in a solvent comprising a nonpolar solvent, e.g., dichloromethane, chloroform, toluene, 1,4-dioxane, diethyl ether, benzene, or a mixture thereof. In some embodiments, the reaction with a reducing agent is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the cyclopropanecarboxylic acid halide is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the cyclopropanecarboxylic acid halide is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days. In some embodiments, the reducing agent is reacted at a temperature within the range of about 35° C. to about 85° C., e.g., about 40° C. to about 85° C., or about 45° C. to about 85° C., or about 50° C. to about 85° C., or about 55° C. to about 85° C., or about 60° C. to about 85° C., or about 65° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 40° C. to about 80° C., or about 45° C. to about 75° C., or about 50° C. to about 70° C., or about 55° C. to about 65° C. In some embodiments, the reducing agent is reacted for a period of time within the range of about 5 minutes to about 3 hours, e.g., or about 10 minutes to about 3 hours, or about 15 minutes to about 3 hours, or about 30 minutes to about 3 hours, or about 45 minutes to about 3 hours, or about 1 hour to about 3 hours, or about 1.25 hours to about 3 hours, or about 1.5 hours to about 3 hours, or about 1.75 hours to about 3 hours, or about 2 hours to about 3 hours, or about 5 minutes to about 2.75 hours, or about 5 minutes to about 2.5 hours, or about 5 minutes to about 2.25 hours, or about 5 minutes to about 2 hours, or about 5 minutes to about 1.75 hours, or about 5 minutes to about 1.5 hours, or about 5 minutes to about 1.25 hours, or about 5 minutes to about 1 hour, or about 10 minutes to about 2.75 hours, or about 15 minutes to about 2.5 hours, or about 30 minutes to about 2.25 hours, or about 45 minutes to about 2 hours, or about 1 hour to about 1.75 hours.

Step (i)(A3)

In some embodiments, reacting a compound of Formula I—H with cyclopropylmethyl halide or activated cyclopropane methanol (e.g., activated with a sulfonate group such as a p-toluene sulfonyl group or a methyl sulfonyl group, or with triphenylphosphine) provides a compound of Formula I-MCP. In certain embodiments, reacting Compound HO—I—H with cyclopropylmethyl halide or activated cyclopropane methanol provides Compound HO—I-MCP. In certain embodiments, reacting Compound MeO—I—H with cyclopropylmethyl halide or activated cyclopropane methanol provides Compound MeO—I-MCP. In certain embodiments, reacting Compound BnO—I—H with cyclopropylmethyl halide or activated cyclopropane methanol provides Compound BnO-I-MCP. See Examples 14, 25, and 34.

In some embodiments, the cyclopropylmethyl halide is cyclopropylmethyl chloride or cyclopropylmethyl bromide. In some embodiments, the reaction is performed in the presence of a trialkylamine, e.g., triethylamine, diisopropylethylamine, 4-methyl-morpholine, or N-methyl-piperidine. In some embodiments, the reaction is performed in a solvent comprising a polar protic solvent, e.g., n-butanol, isopropanol, ethanol, methanol, water, or a mixture thereof.

In some embodiments, the cyclopropylmethyl halide or activated cyclopropane methanol is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the cyclopropylmethyl halide or activated cyclopropane methanol is reacted for a period of time within the range of about 30 minutes to about 6 hours, e.g., about 1 hours to about 6 hours, or about 1.5 hours to about 6 hours, or about 2 hours to about 6 hours, or about 2.5 hours to about 6 hours, or about 3 hours to about 6 hours, or about 3.5 hours to about 6 hours, or about 4 hours to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 1 hours to about 5.5 hours, or about 1.5 hours to about 5 hours, or about 2 hours to about 4.5 hours, or about 2.5 hours to about 4 hours.

Formula I—H→Formula I-Bn

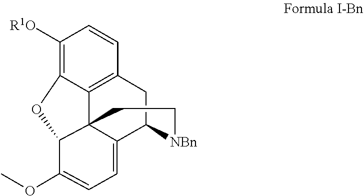

| R¹ of Formula I-Bn | Compound |
|---|---|
| Bn | Compound BnO-I-Bn |

Step (i)(F)

In some embodiments, reacting a compound of Formula I—H with benzyl halide, benzyl sulfonate, or activated benzyl alcohol (e.g., activated with a sulfonate group such as a p-toluene sulfonyl group or a methyl sulfonyl group, or with triphenylphosphine) provides a compound of Formula I-Bn. In certain embodiments, reacting Compound HO—I—H with benzyl halide, benzyl sulfonate, or activated benzyl alcohol provides Compound BnO—I—Bn. See Example 40.

In some embodiments, the benzyl halide is benzyl chloride or benzyl bromide. In some embodiments, the reaction is performed in the presence of a strong base, e.g., an alkali metal hydride. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days.

Formula I—H→Formula I—Ac

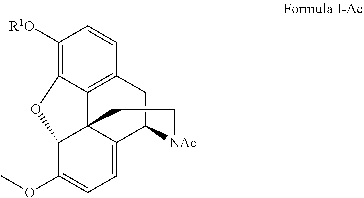

| R¹ of Formula I-Ac | Compound |
|---|---|
| H | Compound HO-I-Ac |
| Ac | Compound AcO-I-Ac |
| Bn | Compound BnO-I-Ac |

Step (i)(G)

In some embodiments, reacting a compound of Formula I—H with acyl halide provides a compound of Formula I—Ac. In certain embodiments, reacting Compound HO—I—H with acyl halide provides Compound HO—I—Ac. See Example 45. In certain embodiments, reacting Compound HO—I—H with acyl halide provides Compound AcO—I—Ac. See Example 48.

In some embodiments, the acyl halide is optionally substituted C₆-C₁₃ aromatic acyl halide, e.g, optionally substituted benzoyl halide. In some embodiments, the acyl halide is aliphatic acylc halide, e.g., acetyl chloride. In some embodiments, the reaction is performed in the presence of a trialkylamine, e.g., triethylamine, diisopropylethylamine, 4-methyl-morpholine, or N-methyl-piperidine. In some embodiments, the reaction is performed in a solvent comprising a nonpolar solvent, e.g., dichloromethane, chloroform, toluene, 1,4-dioxane, diethyl ether, benzene, or a mixture thereof.

In some embodiments, the acyl halide is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the acyl halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 1.5 hours to about 6.5 hours, or about 1.5 hours to about 6 hours, or about 1.5 hours to about 5.5 hours.

Formula I—Ac→Formula I—Ac

Step (ii)(F)

In some embodiments, reacting a compound of Formula I—Ac with benzyl halide, benzyl sulfonate, or activated benzyl alcohol (e.g., activated with a sulfonate group such as a p-toluene sulfonyl group or a methyl sulfonyl group, or with triphenylphosphine) provides another compound of Formula I—Ac. In certain embodiments, reacting Compound HO—I—Ac with benzyl halide, benzyl sulfonate, or activated benzyl alcohol provides Compound BnO—I—Ac. See Example 46.

In some embodiments, the benzyl halide is benzyl chloride or benzyl bromide. In some embodiments, the reaction is performed in the presence of a strong base, e.g., an alkali metal hydride. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days.

Formula I—Ac→Formula I-Bn

Step (iii)(H)

In some embodiments, reacting a compound of Formula I—Ac with lithium aluminum hydride provides a compound of Formula I-Bn. In certain embodiments, reacting Compound BnO—I—Ac with lithium aluminum hydride provides Compound BnO—I—Bn. See Example 47.

In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the lithium aluminum hydride is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the lithium aluminum hydride is reacted for a period of time within the range of about 10 minutes to about 8 hours, e.g., about 20 minutes to about 8 hours, about 30 minutes to about 8 hours, about 1 hour to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours.

Formula I-MCP→Formula I-MCP

Step (ii)(F)

In some embodiments, reacting a compound of Formula I-MCP with benzyl halide, benzyl sulfonate, or activated benzyl alcohol (e.g., activated with a sulfonate group such as a p-toluene sulfonyl group or a methyl sulfonyl group, or with triphenylphosphine) provides another compound of Formula I-MCP. In certain embodiments, reacting Compound HO—I-MCP with benzyl halide, benzyl sulfonate, or activated benzyl alcohol provides Compound BnO-I-MCP. See Example 33.

In some embodiments, the benzyl halide is benzyl chloride or benzyl bromide. In some embodiments, the reaction is performed in the presence of a strong base, e.g., an alkali metal hydride. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days.

Formula I-MCP→Formula II-MCP

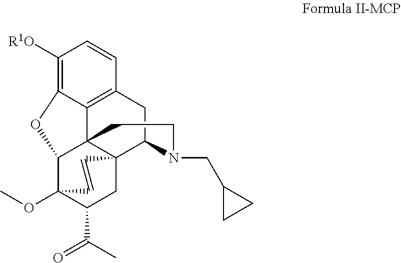

| R¹ of Formula II-MCP | Compound |
|---|---|
| H | Compound HO-II-MCP |
| Me | Compound MeO-II-MCP |
| Bn | Compound BnO-II-MCP |

Step (ii)(B)

In some embodiments, reacting a compound of Formula I-MCP with methyl vinyl ketone provides a compound of Formula II-MCP. In certain embodiments, reacting Compound HO—I-MCP with methyl vinyl ketone provides Compound HO—II-MCP. In certain embodiments, reacting Compound MeO—I-MCP with methyl vinyl ketone provides Compound MeO—II-MCP. See Examples 15 and 26.

In some embodiments, the reaction is performed in a solvent comprising a nonpolar solvent, e.g., dichloromethane, chloroform, toluene, 1,4-dioxane, diethyl ether, benzene, or a mixture thereof.

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

Step (iii)(B)

In some embodiments, reacting a compound of Formula I-MCP with methyl vinyl ketone provides a compound of Formula II-MCP. In certain embodiments, reacting Compound BnO-I-MCP with methyl vinyl ketone provides Compound BnO—II-MCP. See Example 36.

In some embodiments, the reaction is performed in a solvent comprising a nonpolar solvent, e.g., dichloromethane, chloroform, toluene, 1,4-dioxane, diethyl ether, benzene, or a mixture thereof.

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

Formula I-Bn→Formula II-Bn

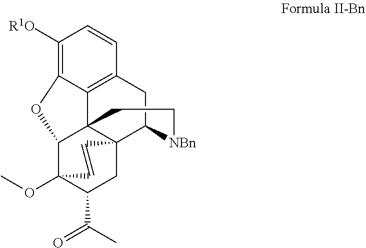

| R¹ of Formula II-Bn | Compound |
|---|---|
| Bn | Compound BnO-II-Bn |

Step (ii)(B), Step (iv)(B)

In some embodiments, reacting a compound of Formula I-Bn with methyl vinyl ketone provides a compound of Formula II-Bn. In certain embodiments, reacting Compound BnO—I—Bn with methyl vinyl ketone provides Compound BnO—II-Bn. See Example 41.

In some embodiments, the reaction is performed in a solvent comprising a nonpolar solvent, e.g., dichloromethane, chloroform, toluene, 1,4-dioxane, diethyl ether, benzene, or a mixture thereof.

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

Formula I—Ac→Formula II—Ac

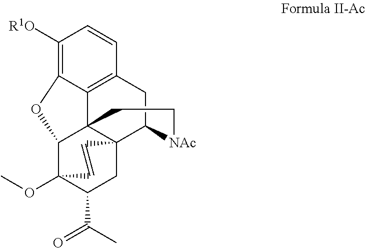

| R¹ of Formula II-Ac | Compound |
|---|---|
| Ac | Compound AcO-II-Ac |

In some embodiments, reacting a compound of Formula I—Ac with methyl vinyl ketone provides a compound of Formula II—Ac. In certain embodiments, reacting Compound AcO—I—Ac with methyl vinyl ketone provides Compound AcO—II—Ac. See Example 49.

In some embodiments, the reaction is performed in a solvent comprising a nonpolar solvent, e.g., dichloromethane, chloroform, toluene, 1,4-dioxane, diethyl ether, benzene, or a mixture thereof.

In some embodiments, the methyl vinyl ketone is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the methyl vinyl ketone is reacted for a period of time within the range of about 2 hours to about 2 days, e.g., about 4 hours to about 2 days, or about 6 hours to about 2 days, or about 12 hours to about 2 days, or about 18 hours to about 2 days, or about 1 days to about 2 days, or about 1.25 days to about 2 days, or about 1.5 days to about 2 days, or about 2 hours to about 1.75 days, or about 2 hours to about 1.5 days, or about 2 hours to about 1.25 days, or about 2 hours to about 1 day, or about 2 hours to about 18 hours, or about 2 hours to about 12 hours, or about 4 hours to about 1.75 days, or about 6 hours to about 1.5 days, or about 12 hours to about 1.25 days, or about 18 hours to about 1 day.

Formula II-MCP→Formula II-MCP

Step (iii)(F)

In some embodiments, reacting a compound of Formula II-MCP with benzyl halide, benzyl sulfonate, or activated benzyl alcohol provides another compound of Formula II-MCP. In certain embodiments, reacting Compound HO—II-MCP with benzyl halide, benzyl sulfonate, or activated benzyl alcohol provides Compound BnO—II-MCP. See Example 35.

In some embodiments, the benzyl halide is benzyl chloride or benzyl bromide. In some embodiments, the reaction is performed in the presence of a strong base, e.g., an alkali metal hydride. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the benzyl halide, benzyl sulfonate, or activated benzyl alcohol is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the benzyl halide, benzyl sulfonate, or activated benzyl alcohol is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days.

Formula II-MCP→Formula IIIB-MCP

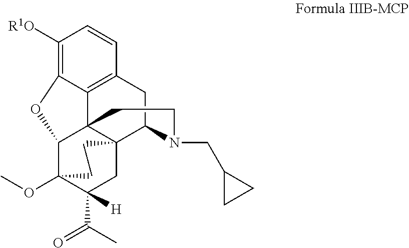

| R¹ of Formula IIIB-MCP | Compound |
|---|---|
| H | Compound HO-IIIB-MCP |
| Me | Compound MeO-IIIB-MCP |

Step (iii)(C)

In some embodiments, reacting a compound of Formula II-MCP with H₂ in the presence of a hydrogenation catalyst provides a compound of Formula IIIB-MCP. In certain embodiments, reacting Compound HO—II-MCP with H2 in the presence of a hydrogenation catalyst provides Compound HO—IIIB-MCP. In certain embodiments, reacting Compound MeO—II-MCP with H2 in the presence of a hydrogenation catalyst provides Compound MeO—IIIB-MCP. See Examples 16, 27, and 28.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Formula II—Ac→Formula IIIB—Ac

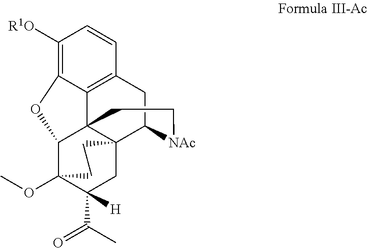

| R¹ of Formula IIIB-Ac | Compound |
|---|---|
| Ac | Compound AcO-IIIB-Ac |

Step (iii)(C)

In some embodiments, reacting a compound of Formula II—Ac with H₂ in the presence of a hydrogenation catalyst provides a compound of Formula IIIB—Ac. In certain embodiments, reacting Compound AcO—II—Ac with H₂ in the presence of a hydrogenation catalyst provides Compound AcO—IIIB—Ac. See, Example 53.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Formula II-MCP→Formula IIIA-MCP

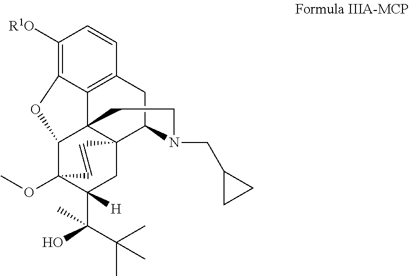

| R¹ of Formula IIIA-MCP | Compound |
|---|---|
| H | Compound HO-IIIA-MCP |
| Me | Compound MeO-IIIA-MCP |
| Bn | Compound BnO-IIIA-MCP |

Step (iii)(D)

In some embodiments, reacting a compound of Formula II-MCP with tert-butylmagnesium halide provides a compound of Formula IIIA-MCP. In certain embodiments, reacting Compound HO—II-MCP with tert-butylmagnesium halide provides Compound HO—IIIA-MCP. In certain embodiments, reacting Compound MeO—II-MCP with tert-butylmagnesium halide provides Compound MeO—IIIA-MCP. In certain embodiments, reacting Compound BnO—II-MCP with tert-butylmagnesium halide provides Compound BnO—IIIA-MCP. See Examples 17 and 29.

In some embodiments, the tert-butylmagnesium halide is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium halide is reacted at a temperature within the range of about 15° C. to about 40° C., e.g., about 20° C. to about 40° C., or about 25° C. to about 40° C., or about 30° C. to about 40° C., or about 15° C. to about 35° C., or about 15° C. to about 30° C., or about 15° C. to about 25° C., or about 20° C. to about 35° C., or about 25° C. to about 30° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

Formula II-Bn→Formula IIIA-Bn

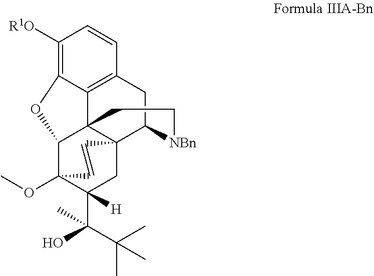

| R¹ of Formula IIIA-Bn | Compound |
|---|---|
| Bn | Compound BnO-IIIA-Bn |
| H | Compound HO-IIIA-Bn |

Step (iii)(D), Step (v)(D)

In some embodiments, reacting a compound of Formula II-Bn with tert-butylmagnesium halide provides a compound of Formula IIIA-Bn. In certain embodiments, reacting Compound BnO—II-Bn with tert-butylmagnesium halide provides Compound BnO—IIIA-Bn. See Example 42.

In some embodiments, the tert-butylmagnesium halide is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium halide is reacted at a temperature within the range of about 15° C. to about 100° C., e.g., about 20° C. to about 100° C., or about 25° C. to about 100° C., or about 30° C. to about 100° C., or about 15° C. to about 95° C., or about 15° C. to about 90° C., or about 15° C. to about 85° C., or about 20° C. to about 95° C., or about 25° C. to about 90° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

Formula II—Ac→Formula IIIA-Ac

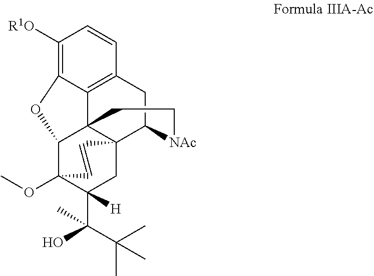

| R¹ of Formula IIIA-Bn | Compound |
|---|---|
| H | Compound HO-IIIA-Ac |

Step (iii)(D)

In some embodiments, reacting a compound of Formula II—Ac with tert-butylmagnesium halide provides a compound of Formula IIIA-Ac. In certain embodiments, reacting Compound AcO—II—Ac with tert-butylmagnesium halide provides Compound HO—IIIA-Ac. See Example 50.

In some embodiments, the tert-butylmagnesium halide is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium halide is reacted at a temperature within the range of about 15° C. to about 100° C., e.g., about 20° C. to about 100° C., or about 25° C. to about 100° C., or about 30° C. to about 100° C., or about 15° C. to about 95° C., or about 15° C. to about 90° C., or about 15° C. to about 85° C., or about 20° C. to about 95° C., or about 25° C. to about 90° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

Formula IIIA-Ac→Formula IIIA-Bn

Step (iv)(H)

In some embodiments, reacting a compound of Formula IIIA-Ac with lithium aluminum hydride provides a compound of Formula IIIA-Bn. In certain embodiments, reacting Compound HO—IIIA-Ac with lithium aluminum hydride provides Compound HO—IIIA-Bn. See Example 51.

In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the lithium aluminum hydride is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the lithium aluminum hydride is reacted for a period of time within the range of about 10 minutes to about 8 hours, e.g., about 20 minutes to about 8 hours, about 30 minutes to about 8 hours, about 1 hour to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours.

Formula IIIA-MCP→Formula IIIA-MCP

Step (iv)(E)

In some embodiments, reacting a compound of Formula IIIA-MCP with a demethylating agent provides another compound of Formula IIIA-MCP. In certain embodiments, reacting Compound MeO—IIIA-MCP with a demethylating agent provides Compound HO—IIIA-MCP. See Example 20.

In some embodiments, the demethylating agent is a thiolate, e.g., a dodecane thiolate. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the demethylating agent is reacted at a temperature within the range of about 50° C. to about 190° C., e.g., about 60° C. to about 190° C., or about 70° C. to about 190° C., or about 80° C. to about 190° C., or about 90° C. to about 190° C., or about 100° C. to about 190° C., or about 110° C. to about 190° C., or about 120° C. to about 190° C., or about 130° C. to about 190° C., or about 140° C. to about 190° C., or about 150° C. to about 190° C., or about 50° C. to about 180° C., or about 50° C. to about 170° C., or about 50° C. to about 160° C., or about 50° C. to about 150° C., or about 50° C. to about 140° C., or about 50° C. to about 130° C., or about 50° C. to about 120° C., or about 50° C. to about 110° C., or about 50° C. to about 100° C., or about 50° C. to about 90° C., or about 60° C. to about 180° C., or about 70° C. to about 170° C., or about 80° C. to about 160° C., or about 90° C. to about 150° C., or about 100° C. to about 140° C. In some embodiments, the demethylating agent is reacted for a period of time within the range of about 4 hours to about 2 days, e.g., about 8 hours to about 2 days, or about 12 hours to about 2 days, or about 16 hours to about 2 days, or about 20 hours to about 2 days, or about 1 day to about 2 days, or about 1.25 days to about 2 days, or about 1.5 days to about 2 days, or about 4 hours to about 1.75 days, or about 4 hours to about 1.5 days, or about 4 hours to about 1.25 days, or about 4 hours to about 1 day, or about 4 hours to about 20 hours, or about 4 hours to about 16 hours, or about 4 hours to about 12 hours, or about 8 hours to about 1.75 days, or about 12 hours to about 1.5 days, or about 16 hours to about 1.25 days.

Formula IIIB-MCP→Formula IV-MCP

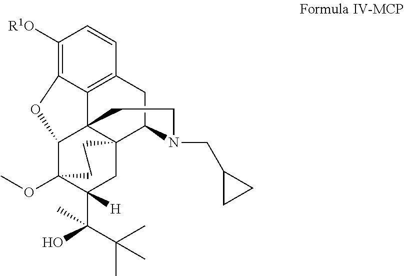

| R¹ of Formula IV-MCP | Compound |
|---|---|
| H | buprenorphine |
| Me | Compound MeO-IV-MCP |

Step (iv)(D)

In some embodiments, reacting a compound of Formula IIIB-MCP with tert-butylmagnesium halide provides a compound of Formula IV-MCP. In certain embodiments, reacting Compound HO—IIIB-MCP with tert-butylmagnesium halide provides buprenorphine. In certain embodiments, reacting Compound MeO—IIIB-MCP with tert-butylmagnesium halide provides Compound MeO—IV-MCP. See Examples 18, 30, 31, and 37.

In some embodiments, the tert-butylmagnesium halide is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium halide is reacted at a temperature within the range of about 15° C. to about 40° C., e.g., about 20° C. to about 40° C., or about 25° C. to about 40° C., or about 30° C. to about 40° C., or about 15° C. to about 35° C., or about 15° C. to about 30° C., or about 15° C. to about 25° C., or about 20° C. to about 35° C., or about 25° C. to about 30° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

Formula IIIA-MCP→Formula IV-MCP

Step (iv)(C), Step (v)(C)

In some embodiments, reacting a compound of Formula IIIA-MCP with H₂ in the presence of a hydrogenation catalyst provides a compound of Formula IV-MCP. In certain embodiments, reacting Compound HO—IIIA-MCP with H₂ in the presence of a hydrogenation catalyst provides buprenorphine. In certain embodiments, reacting Compound MeO—IIIA-MCP with H2 in the presence of a hydrogenation catalyst provides Compound MeO—IV-MCP. In certain embodiments, reacting Compound BnO—IIIA-MCP with H₂ in the presence of a hydrogenation catalyst provides buprenorphine. See Examples 19, 22, 32, and 38.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Formula IIIB—Ac→Formula IV—Ac

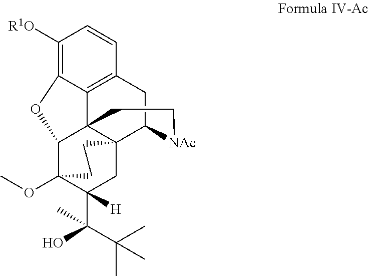

| R¹ of Formula IV-Ac | Compound |
|---|---|
| H | Compound HO-IV-Ac |

Step (iv)(D)

In some embodiments, reacting a compound of Formula IIIB—Ac with tert-butylmagnesium halide provides a compound of Formula IV—Ac. In certain embodiments, reacting Compound AcO—IIIB—Ac with tert-butylmagnesium halide provides Compound HO—IV—Ac. See, Example 54.

In some embodiments, the tert-butylmagnesium halide is tert-butylmagnesium chloride or tert-butylmagnesium bromide. In some embodiments, the reaction is performed in a solvent comprising a nonpolar solvent, e.g., tert-butylmethyl ether, 2-methyl-tetrahydrofuran, diethyl ether, dimethoxymethane, benzene, toluene, or a mixture of thereof.

In some embodiments, the tert-butylmagnesium halide is reacted at a temperature within the range of about 15° C. to about 40° C., e.g., about 20° C. to about 40° C., or about 25° C. to about 40° C., or about 30° C. to about 40° C., or about 15° C. to about 35° C., or about 15° C. to about 30° C., or about 15° C. to about 25° C., or about 20° C. to about 35° C., or about 25° C. to about 30° C. In some embodiments, the tert-butylmagnesium halide is reacted for a period of time within the range of about 30 minutes to about 8 hours, e.g., about 1 hours to about 8 hours, or about 1.5 hours to about 8 hours, or about 2 hours to about 8 hours, or about 2.5 hours to about 8 hours, or about 3 hours to about 8 hours, or about 3.5 hours to about 8 hours, or about 4 hours to about 8 hours, or about 4.5 hours to about 8 hours, or about 5 hours to about 8 hours, or about 30 minutes to about 7.5 hours, or about 30 minutes to about 7 hours, or about 30 minutes to about 6.5 hours, or about 30 minutes to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 1 hour to about 7.5 hours, or about 1.5 hours to about 7 hours, or about 2 hours to about 6.5 hours, or about 2.5 hours to about 6 hours, or about 3 hours to about 5.5 hours.

Formula IIIA-MCP→Formula IV—Ac

Step (iv)(C)

In some embodiments, reacting a compound of Formula IIIA-Ac with H₂ in the presence of a hydrogenation catalyst provides a compound of Formula IV—Ac. In certain embodiments, reacting Compound HO—IIIA-Ac with H₂ in the presence of a hydrogenation catalyst provides Compound HO—IV—Ac. See, Example 55.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Formula IIIA-Bn→Formula IV—H

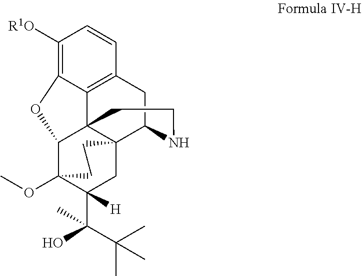

| R¹ of Formula IV-H | Compound |
|---|---|
| H | HO-IV-H |

Step (iv)(C), Step (v)(C), Step (vi)(C)

In some embodiments, reacting a compound of Formula IIIA-Bn with H₂ in the presence of a hydrogenation catalyst provides a compound of Formula IV-Bn. In certain embodiments, reacting Compound BnO—IIIA-Bn with H₂ in the presence of a hydrogenation catalyst provides Compound HO—IV—H. See Example 43. In certain embodiments, reacting Compound HO—IIIA-Bn with H₂ in the presence of a hydrogenation catalyst provides Compound HO—IV—H. See Examples 43 and 52.

In some embodiments, the hydrogenation catalyst comprises nickel, palladium, platinum, rhodium, or ruthenium. In some embodiments, the hydrogenation catalyst comprises platinum or palladium, supported on carbon. In some embodiments, the reaction is performed in a solvent comprising a polar protic or aprotic solvent, e.g., n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.

In some embodiments, the hydrogen is reacted at a temperature within the range of about 15° C. to about 120° C., e.g., about 20° C. to about 120° C., or about 30° C. to about 120° C., or about 40° C. to about 120° C., or about 15° C. to about 115° C., or about 20° C. to about 110° C., or about 30° C. to about 105° C., or about 40° C. to about 115° C., or about 50° C. to about 110° C. In some embodiments, the hydrogen is reacted for a period of time within the range of about 6 hours to about 3 days, e.g., about 12 hours to about 3 days, or about 18 hours to about 3 days, or about 1 day to about 3 days, or about 1.25 days to about 3 days, or about 1.5 days to about 3 days, or about 6 hours to about 2.75 days, or about 6 hours to about 2.5 days, or about 6 hours to about 2.25 days, or about 6 hours to about 2 day, or about 6 hours to about 36 hours, or about 12 hours to about 2.5 days, or about 24 hours to about 2 days. In some embodiments, the hydrogen is reacted at a pressure within the range of about 1 atm to about 3 atm, e.g., about 1.25 atm to about 3 atm, or about 1.5 atm to about 3 atm, or about 1.75 atm to about 3 atm, or about 2 atm to about 3 atm, or about 1 atm to about 2.75 atm, or about 1 atm to about 2.5 atm, or about 1 atm to about 2.25 atm, or about 1 atm to about 2 atm, or about 1.25 atm to about 2.75 atm, or about 1.5 atm to about 2.5 atm, or about 1.75 atm to about 2.25 atm.

Formula IV—Ac→Formula IV—H

Step (v)(I)

In some embodiments, reacting a compound of Formula IV—Ac with Schwartz's reagent (zirconocene hydrochloride) or base provides a compound of Formula IV—H. In certain embodiments, reacting Compound HO—IV—Ac with Schwartz's reagent or base provides Compound HO—IV—H. See Examples 56 and 57.

In some embodiments, the reaction with Schwartz's reagent is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the Schwartz's reagent is reacted at a temperature within the range of about 15° C. to about 40° C., e.g., about 20° C. to about 40° C., or about 25° C. to about 40° C., or about 30° C. to about 40° C., or about 15° C. to about 35° C., or about 15° C. to about 30° C., or about 15° C. to about 25° C., or about 20° C. to about 35° C., or about 25° C. to about 30° C. In some embodiments, the Schwartz's reagent is reacted for a period of time within the range of about 5 minutes to about 3 hours, e.g., or about 10 minutes to about 3 hours, or about 15 minutes to about 3 hours, or about 30 minutes to about 3 hours, or about 45 minutes to about 3 hours, or about 1 hour to about 3 hours, or about 1.25 hours to about 3 hours, or about 1.5 hours to about 3 hours, or about 1.75 hours to about 3 hours, or about 2 hours to about 3 hours, or about 5 minutes to about 2.75 hours, or about 5 minutes to about 2.5 hours, or about 5 minutes to about 2.25 hours, or about 5 minutes to about 2 hours, or about 5 minutes to about 1.75 hours, or about 5 minutes to about 1.5 hours, or about 5 minutes to about 1.25 hours, or about 5 minutes to about 1 hour, or about 10 minutes to about 2.75 hours, or about 15 minutes to about 2.5 hours, or about 30 minutes to about 2.25 hours, or about 45 minutes to about 2 hours, or about 1 hour to about 1.75 hours.

In some embodiments, the base is an inorganic base, e.g., potassium hydroxide or sodium hydroxide. In some embodiments, the reaction with base is performed in a solvent comprising a high-boiling-point polar protic or aprotic solvent, e.g., ethylene glycol, diethylene glycol, N-methylpyrrolidone, dimethylformamide, or dimethylsulfoxide.

In some embodiments, the base is reacted at a temperature within the range of about 50° C. to about 240° C., e.g., about 60° C. to about 240° C., or about 70° C. to about 240° C., or about 80° C. to about 240° C., or about 90° C. to about 240° C., or about 100° C. to about 240° C., or about 110° C. to about 240° C., or about 120° C. to about 240° C., or about 130° C. to about 240° C., or about 140° C. to about 240° C., or about 150° C. to about 240° C., or about 50° C. to about 230° C., or about 50° C. to about 220° C., or about 50° C. to about 2100° C., or about 50° C. to about 2000° C., or about 50° C. to about 190° C., or about 50° C. to about 180° C., or about 90° C. to about 210° C., or about 100° C. to about 200° C. In some embodiments, the base is reacted for a period of time within the range of about 4 hours to about 2 days, e.g., about 8 hours to about 2 days, or about 12 hours to about 2 days, or about 16 hours to about 2 days, or about 20 hours to about 2 days, or about 1 day to about 2 days, or about 1.25 days to about 2 days, or about 1.5 days to about 2 days, or about 4 hours to about 1.75 days, or about 4 hours to about 1.5 days, or about 4 hours to about 1.25 days, or about 4 hours to about 1 day, or about 4 hours to about 20 hours, or about 4 hours to about 16 hours, or about 4 hours to about 12 hours, or about 8 hours to about 1.75 days, or about 12 hours to about 1.5 days, or about 16 hours to about 1.25 days.

Formula IV—H→Formula IV-MCP

Step (v)(A1), Step (vi)(A1)

In some embodiments, reacting a compound of Formula IV—H with cyclopropane carboxaldehyde followed by a hydride source provides a compound of Formula IV-MCP. In certain embodiments, reacting Compound HO—IV—H with cyclopropane carboxaldehyde followed by a hydride source provides buprenoprhine. See Example 44.

In some embodiments, the hydride source is formic acid, hydrogen, sodium cyanoborohydride, sodium borohydride, or sodium triacetoxy borohydride. In some embodiments, the hydride source is formic acid. In some embodiments, the reaction is catalyzed by a ruthenium(I) complex or a ruthenium(II) complex, e.g., a dichloro(p-cymene)ruthenium(II) dimer. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof. In some embodiments, the reaction is performed in the presence of a trialkylamine, e.g., triethylamine, diisopropylethylamine, 4-methyl-morpholine, or N-methyl-piperidine.

In some embodiments, the cyclopropane carboxaldehyde is reacted at a temperature within the range of about 30° C. to about 90° C., e.g., about 35° C. to about 90° C., or about 40° C. to about 90° C., or about 45° C. to about 90° C., or about 50° C. to about 90° C., or about 55° C. to about 90° C., or about 60° C. to about 90° C., or about 65° C. to about 90° C., or about 70° C. to about 90° C., or about 30° C. to about 85° C., or about 30° C. to about 80° C., or about 30° C. to about 75° C., or about 30° C. to about 70° C., or about 30° C. to about 65° C., or about 30° C. to about 60° C., or about 30° C. to about 55° C., or about 30° C. to about 50° C., or about 35° C. to about 85° C., or about 40° C. to about 80° C., or about 45° C. to about 75° C., or about 50° C. to about 70° C., or about 55° C. to about 65° C. In some embodiments, the cyclopropane carboxaldehyde is reacted for a period of time within the range of about 30 minutes to about 5 hours, e.g., about 1 hour to about 5 hours, or about 1.5 hours to about 5 hours, or about 2 hours to about 5 hours, or about 2.5 hours to about 5 hours, or about 3 hours to about 5 hours, or about 3.5 hours to about 5 hours, or about 4 hours to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 30 minutes to about 2 hours, or about 30 minutes to about 1.5 hours.

Step (v)(A2), Step (vi)(A2)

In some embodiments, reacting a compound of Formula IV—H with cyclopropanecarboxylic acid halide followed by a reducing agent provides a compound of Formula IV-MCP. In certain embodiments, reacting Compound HO—IV—H with cyclopropanecarboxylic acid halide followed by a reducing agent provides buprenorphine.

In some embodiments, the cyclopropanecarboxylic acid halide is cyclopropanecarboxylic acid chloride, cyclopropanecarboxylic acid anhydride, cyclopropanecarboxylic acid bromide, or an activated cyclopropanecarboxylic acid (e.g., an activated cyclopropanecarboxylic acid formed by reaction with an alcohol such as pentafluorophenol, 4-nitrophenol, N-hydroxysuccinimide, N-hydroxymaleimide, 1-Hydroxybenzotriazole, or 1-hydroxy-7-azabenzotriazole). In some embodiments, the reducing agent is LiAlH₄ or NaBH₄. In some embodiments, the reaction with cyclopropanecarboxylic acid halide is performed in a solvent comprising a nonpolar solvent, e.g., dichloromethane, chloroform, toluene, 1,4-dioxane, diethyl ether, benzene, or a mixture thereof. In some embodiments, the reaction with a reducing agent is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the cyclopropanecarboxylic acid halide is reacted at a temperature within the range of about −20° C. to about 40° C., e.g., about −20° C. to about 35° C., or about −20° C. to about 30° C., or about −20° C. to about 25° C., or about −20° C. to about 20° C., or about −20° C. to about 15° C., or about −20° C. to about 10° C., or about −20° C. to about 5° C., or about −20° C. to about 0° C., or about −15° C. to about 40° C., or about −10° C. to about 40° C., or about −5° C. to about 40° C., or about 0° C. to about 40° C., or about 5° C. to about 20° C., or about 10° C. to about 40° C., or about 15° C. to about 40° C., or about 20° C. to about 40° C., or about −15° C. to about 35° C., or about −10° C. to about 30° C., or about −5° C. to about 25° C., or about 0° C. to about 20° C., or about 5° C. to about 15° C. In some embodiments, the cyclopropanecarboxylic acid halide is reacted for a period of time within the range of about 6 hours to about 2 days, e.g., about 12 hours to about 2 days, or about 18 hours to about 2 days, or about 1 day to about 2 days, or about 1.25 days to about 2 days, or about 1.5 days to about 2 days, or about 6 hours to about 1.75 days, or about 6 hours to about 1.5 days, or about 6 hours to about 1.25 days, or about 6 hours to about 1 day, or about 6 hours to about 18 hours, or about 12 hours to about 1.75 days, or about 18 hours to about 1.5 days. In some embodiments, the reducing agent is reacted at a temperature within the range of about 35° C. to about 85° C., e.g., about 40° C. to about 85° C., or about 45° C. to about 85° C., or about 50° C. to about 85° C., or about 55° C. to about 85° C., or about 60° C. to about 85° C., or about 65° C. to about 85° C., or about 35° C. to about 80° C., or about 35° C. to about 75° C., or about 35° C. to about 70° C., or about 35° C. to about 65° C., or about 35° C. to about 60° C., or about 35° C. to about 55° C., or about 40° C. to about 80° C., or about 45° C. to about 75° C., or about 50° C. to about 70° C., or about 55° C. to about 65° C. In some embodiments, the reducing agent is reacted for a period of time within the range of about 5 minutes to about 3 hours, e.g., or about 10 minutes to about 3 hours, or about 15 minutes to about 3 hours, or about 30 minutes to about 3 hours, or about 45 minutes to about 3 hours, or about 1 hour to about 3 hours, or about 1.25 hours to about 3 hours, or about 1.5 hours to about 3 hours, or about 1.75 hours to about 3 hours, or about 2 hours to about 3 hours, or about 5 minutes to about 2.75 hours, or about 5 minutes to about 2.5 hours, or about 5 minutes to about 2.25 hours, or about 5 minutes to about 2 hours, or about 5 minutes to about 1.75 hours, or about 5 minutes to about 1.5 hours, or about 5 minutes to about 1.25 hours, or about 5 minutes to about 1 hour, or about 10 minutes to about 2.75 hours, or about 15 minutes to about 2.5 hours, or about 30 minutes to about 2.25 hours, or about 45 minutes to about 2 hours, or about 1 hour to about 1.75 hours.

Step (v)(A3), Step (vi)(A3)

In some embodiments, reacting a compound of Formula IV—H with cyclopropylmethyl halide or activated cyclopropane methanol (e.g., activated with a sulfonate group such as a p-toluene sulfonyl group or a methyl sulfonyl group, or with triphenylphosphine) provides a compound of Formula IV-MCP. In certain embodiments, reacting Compound HO—IV—H with cyclopropylmethyl halide or activated cyclopropane methanol provides buprenorphine.

In some embodiments, the cyclopropylmethyl halide is cyclopropylmethyl chloride or cyclopropylmethyl bromide. In some embodiments, the reaction is performed in the presence of a trialkylamine, e.g., triethylamine, diisopropylethylamine, 4-methyl-morpholine, or N-methyl-piperidine. In some embodiments, the reaction is performed in a solvent comprising a polar protic solvent, e.g., n-butanol, isopropanol, ethanol, methanol, water, or a mixture thereof.

In some embodiments, the cyclopropylmethyl halide or activated cyclopropane methanol is reacted at a temperature within the range of about 40° C. to about 120° C., e.g., about 45° C. to about 120° C., or about 50° C. to about 120° C., or about 55° C. to about 120° C., or about 60° C. to about 120° C., or about 65° C. to about 120° C., or about 70° C. to about 120° C., or about 75° C. to about 120° C., or about 80° C. to about 120° C., or about 85° C. to 120° C., or about 90° C. to about 120° C., or about 40° C. to about 115° C., or about 40° C. to about 110° C., or about 40° C. to about 105° C., or about 40° C. to about 100° C., or about 40° C. to about 95° C., or about 40° C. to about 90° C., or about 40° C. to about 85° C., or about 40° C. to about 80° C., or about 40° C. to about 75° C., or about 40° C. to about 70° C., or about 45° C. to about 115° C., or about 50° C. to about 110° C., or about 55° C. to about 105° C., or about 60° C. to about 100° C., or about 65° C. to about 95° C., or about 70° C. to about 90° C. In some embodiments, the cyclopropylmethyl halide or activated cyclopropane methanol is reacted for a period of time within the range of about 30 minutes to about 6 hours, e.g., about 1 hours to about 6 hours, or about 1.5 hours to about 6 hours, or about 2 hours to about 6 hours, or about 2.5 hours to about 6 hours, or about 3 hours to about 6 hours, or about 3.5 hours to about 6 hours, or about 4 hours to about 6 hours, or about 30 minutes to about 5.5 hours, or about 30 minutes to about 5 hours, or about 30 minutes to about 4.5 hours, or about 30 minutes to about 4 hours, or about 30 minutes to about 3.5 hours, or about 30 minutes to about 3 hours, or about 30 minutes to about 2.5 hours, or about 1 hours to about 5.5 hours, or about 1.5 hours to about 5 hours, or about 2 hours to about 4.5 hours, or about 2.5 hours to about 4 hours.

Formula IV-MCP→Formula IV-MCP

Step (v)(E)

In some embodiments, reacting a compound of Formula IV-MCP with a demethylating agent provides another compound of Formula IV-MCP. In certain embodiments, reacting Compound MeO—IV-MCP with a demethylating agent provides buprenorphine. See Example 21.

In some embodiments, the demethylating agent is a thiolate, e.g., a dodecane thiolate. In some embodiments, the reaction is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.

In some embodiments, the demethylating agent is reacted at a temperature within the range of about 50° C. to about 190° C., e.g., about 60° C. to about 190° C., or about 70° C. to about 190° C., or about 80° C. to about 190° C., or about 90° C. to about 190° C., or about 100° C. to about 190° C., or about 110° C. to about 190° C., or about 120° C. to about 190° C., or about 130° C. to about 190° C., or about 140° C. to about 190° C., or about 150° C. to about 190° C., or about 50° C. to about 180° C., or about 50° C. to about 170° C., or about 50° C. to about 160° C., or about 50° C. to about 150° C., or about 50° C. to about 140° C., or about 50° C. to about 130° C., or about 50° C. to about 120° C., or about 50° C. to about 110° C., or about 50° C. to about 100° C., or about 50° C. to about 90° C., or about 60° C. to about 180° C., or about 70° C. to about 170° C., or about 80° C. to about 160° C., or about 90° C. to about 150° C., or about 100° C. to about 140° C. In some embodiments, the demethylating agent is reacted for a period of time within the range of about 4 hours to about 2 days, e.g., about 8 hours to about 2 days, or about 12 hours to about 2 days, or about 16 hours to about 2 days, or about 20 hours to about 2 days, or about 1 day to about 2 days, or about 1.25 days to about 2 days, or about 1.5 days to about 2 days, or about 4 hours to about 1.75 days, or about 4 hours to about 1.5 days, or about 4 hours to about 1.25 days, or about 4 hours to about 1 day, or about 4 hours to about 20 hours, or about 4 hours to about 16 hours, or about 4 hours to about 12 hours, or about 8 hours to about 1.75 days, or about 12 hours to about 1.5 days, or about 16 hours to about 1.25 days.

Formula I—H→buprenorphine

In one aspect, the method of preparing buprenorphine comprises the series of steps provided in Table 17:

TABLE 17
4-step buprenorphine route

| No. | Substrate | Step | Product |
|---|---|---|---|
| i | Compound HO-I-H | (A1), (A2), | Compound HO-I-MCP |
| | | or (A3) | |
| ii | Compound HO-I-MCP | (B) | Compound HO-II-MCP |
| iii | Compound HO-II-MCP | (C) | Compound HO-IIIB-MCP |
| iv | Compound HO-IIIB- | (D) | buprenorphine |
| | MCP | | |

In another aspect, the method of preparing buprenorphine comprises the series of steps provided in Table 18:

TABLE 18
4-step buprenorphine route

| No. | Substrate | Step | Product |
|---|---|---|---|
| i | Compound HO-I-H | (A1), (A2), | Compound HO-I-MCP |
| | | or (A3) | |
| ii | Compound HO-I-MCP | (B) | Compound HO-II-MCP |
| iii | Compound HO-II-MCP | (D) | Compound HO-IIIA-MCP |
| iv | Compound HO-IIIA- | (C) | buprenorphine |
| | MCP | | |

In another aspect, the method of preparing buprenorphine comprises the series of steps provided in Table 20:

TABLE 20
5-step buprenorphine route

| No. | Substrate | Step | Product |
|---|---|---|---|
| i | Compound MeO-I-H | (A1), (A2), | Compound MeO-I-MCP |
| | | or (A3) | |
| ii | Compound MeO-I-MCP | (B) | Compound MeO-II-MCP |
| iii | Compound MeO-II-MCP | (C) | Compound MeO-IIIB- |
| | | | MCP |
| iv | Compound MeO-IIIB- | (D) | Compound MeO-IV- |
| | MCP | | MCP |
| v | Compound MeO-IV-MCP | (E) | buprenorphine |

In another aspect, the method of preparing buprenorphine comprises the series of steps provided in Table 21:

TABLE 21
5-step buprenorphine route

| No. | Substrate | Step | Product |
|---|---|---|---|
| i | Compound MeO-I-H | (A1), (A2), | Compound MeO-I-MCP |
| | | or (A3) | |
| ii | Compound MeO-I-MCP | (B) | Compound MeO-II-MCP |
| iii | Compound MeO-II-MCP | (D) | Compound MeO-IIIA- |
| | | | MCP |
| iv | Compound MeO-IIIA- | (C) | Compound MeO-IV- |
| | MCP | | MCP |
| v | Compound MeO-IV-MCP | (E) | buprenorphine |

In another aspect, the method of preparing buprenorphine comprises the series of steps provided in Table 22:

TABLE 22
5-step buprenorphine route

| No. | Substrate | Step | Product |
|---|---|---|---|
| i | Compound MeO-I-H | (A1), (A2), | Compound MeO-I- |
| | | or (A3) | MCP |
| ii | Compound MeO-I-MCP | (B) | Compound MeO-II- |
| | | | MCP |
| iii | Compound MeO-II-MCP | (D) | Compound MeO-IIIA- |
| | | | MCP |
| iv | Compound MeO-IIIA- | (E) | Compound HO-IIIA- |
| | MCP | | MCP |
| v | Compound HO-IIIA-MCP | (C) | buprenorphine |

In another aspect, the method of preparing buprenorphine comprises the series of steps provided in Table 23:

TABLE 23
5-step buprenorphine route

| No. | Substrate | Step | Product |
|---|---|---|---|
| i | Compound HO-I-H | (A1), (A2), | Compound HO-I-MCP |
| | | or (A3) | |
| ii | Compound HO-I-MCP | (B) | Compound HO-II-MCP |
| iii | Compound HO-II-MCP | (F) | Compound BnO-II-MCP |
| iv | Compound BnO-II-MCP | (D) | Compound BnO-IIIA- |
| | | | MCP |
| v | Compound BnO-IIIA- | (C) | buprenorphine |
| | MCP | | |

In another aspect, the method of preparing buprenorphine comprises the series of steps provided in Table 24:

TABLE 24
5-step buprenorphine route

| No. | Substrate | Step | Product |
|---|---|---|---|
| i | Compound HO-I-H | (A1), (A2), | Compound HO-I-MCP |
| | | or (A3) | |
| ii | Compound HO-I-MCP | (F) | Compound BnO-I-MCP |
| iii | Compound BnO-I-MCP | (B) | Compound BnO-II-MCP |
| iv | Compound BnO-II-MCP | (D) | Compound BnO-IIIA-MCP |
| v | Compound BnO-IIIA- | (C) | buprenorphine |
| | MCP | | |

In another aspect, the method of preparing buprenorphine comprises the series of steps provided in Table 25:

TABLE 25
5-step buprenorphine route

| No. | Substrate | Step | Product |
|---|---|---|---|
| i | Compound HO-I-H | (F) | Compound BnO-I-Bn |
| ii | Compound BnO-I-Bn | (B) | Compound BnO-II-Bn |
| iii | Compound BnO-II-Bn | (D) | Compound BnO-IIIA-Bn |
| iv | Compound BnO-IIIA-Bn | (C) | Compound HO-IV-H |
| v | Compound HO-IV-H | (A1), (A2), | buprenorphine |
| | | or (A3) | |

In another aspect, the method of preparing buprenorphine comprises the series of steps provided in Table 26:

TABLE 26
7-step buprenorphine route

| No. | Substrate | Step | Product |
|---|---|---|---|
| i | Compound HO-I-H | (G) | Compound HO-I-Ac |
| ii | Compound HO-I-Ac | (F) | Compound BnO-I-Ac |
| iii | Compound BnO-I-Ac | (H) | Compound BnO-I-Bn |
| iv | Compound BnO-I-Bn | (B) | Compound BnO-II-Bn |
| v | Compound BnO-II-Bn | (D) | Compound BnO-IIIA-Bn |
| vi | Compound BnO-IIIA-Bn | (C) | Compound HO-IV-H |
| vii | Compound HO-IV-H | (A1), (A2), | buprenorphine |
| | | or (A3) | |

In another aspect, the method of preparing buprenorphine comprises the series of steps provided in Table 27:

TABLE 27
6-step buprenorphine route

| No. | Substrate | Step | Product |
|---|---|---|---|
| i | Compound HO-I-H | (G) | Compound AcO-I-Ac |
| ii | Compound AcO-I-Ac | (B) | Compound AcO-II-Ac |
| iii | Compound AcO-II-Ac | (D) | Compound HO-IIIA-Ac |
| iv | Compound HO-IIIA-Ac | (H) | Compound HO-IIIA-Bn |
| v | Compound HO-IIIA-Bn | (C) | Compound HO-IV-H |
| vi | Compound HO-IV-H | (A1), (A2), | Buprenorphine |
| | | or (A3) | |

In another aspect, the method of preparing buprenorphine comprises the series of steps provided in Table 28:

TABLE 28
6-step buprenorphine route

| No. | Substrate | Step | Product |
|---|---|---|---|
| i | Compound HO-I-H | (G) | Compound AcO-I-Ac |
| ii | Compound AcO-I-Ac | (B) | Compound AcO-II-Ac |
| iii | Compound AcO-II-Ac | (D) | Compound HO-IIIA-Ac |
| iv | Compound HO-IIIA-Ac | (C) | Compound HO-IV-Ac |
| v | Compound HO-IV-Ac | (I) | Compound HO-IV-H |
| vi | Compound HO-IV-H | (A1), (A2), | Buprenorphine |
| | | or (A3) | |

In another aspect, the method of preparing buprenorphine comprises the series of steps provided in Table 29:

TABLE 29
6-step buprenorphine route

| No. | Substrate | Step | Product |
|---|---|---|---|
| i | Compound HO-I-H | (G) | Compound AcO-I-Ac |
| ii | Compound AcO-I-Ac | (B) | Compound AcO-II-Ac |
| iii | Compound AcO-II-Ac | (C) | Compound AcO-IIIB-Ac |
| iv | Compound AcO-IIIB-Ac | (D) | Compound HO-IV-Ac |
| v | Compound HO-IV-Ac | (I) | Compound HO-IV-H |
| vi | Compound HO-IV-H | (A1), (A2), | Buprenorphine |
| | | or (A3) | |

Product-by-Process

The methods described herein all have favourable characteristics. The same goes for the products produced by the methods.

This, one aspect of the present invention relates to a product obtainable from a method of the present invention.

One embodiment of the present invention relates to to an N-demethylated compound obtainable from a method of the present invention. These compounds can be any N-demethylated retiduline derivative described herein.

Another embodiment of the present invention relates to an N- and O-demethylated compounds obtainable from a method of the present invention.

A further embodiment of the present invention relates to buprenorphine obtainable from a method disclosed herein.

Additional Steps

The methods of the present invention can optionally comprise one or more additional steps. These steps can for example be directed toward isolation and/or purification of the compounds from the host cells before the chemical synthesis.

A number of different methods can be used to isolate and purify Northebaine and Nororipavine produced by the methods disclosed herein. For example, the isolating steps may comprise: (a) contacting the cell culture comprising the Nor-compounds (Oripavine/Thebaine) with: (i) one or more adsorbent resins in a packed column in order to bind at least a portion of the Nor-alkaloid compounds to the resin, thereby isolating the nor compound; or (ii) one or more ion exchange or reversed-phase chromatography columns in order to bind at least a portion of the nor compound in the column, thereby isolating the nor-alkaloid compound; or (b) crystallizing and/or organic solvent extracting the Nor-alkaloid compounds from the cell culture, thereby isolating the northebaine and nororipavine compound; (i) contacting the cell culture with an organic solvent immiscible with water and separating the organic phase enriched in nor-alkaloids (c) separating the cell culture into a solid phase and a liquid phase, wherein the liquid phase comprises of the Nor-alkaloids; and (i) contacting the liquid phase with one or more adsorbent resins in order to bind at least a portion of the nor compound to the resin, thereby isolating the products; (ii) contacting the liquid phase with one or more ion exchange or reversed-phase chromatography columns in order to bind at least a portion of the Nor-alkaloid compound in the column, thereby isolating the pure products; or (iii) crystallizing and/or extracting the nor-alkaloids from the liquid phase, thereby isolating the Nororipavine and Northebaine in pure form.

The isolating step can comprise, separating the solid phase from the liquid phase using a process comprising tangential flow filtration with diafiltration membranes to generate a permeate stream comprising the nor-alkaloid compounds (Northebaine/Nororipavine), wherein the membranes used in the tangential flow filtration are ultrafiltration or nanofiltration membranes. In an embodiment, the permeate stream is extracted by an organic solvent which phase-separates from the aqueous phase to generate an extracted nor-alkaloid product (Northebaine or Nororipavine) in the organic solvent.

Optionally the permeate stream containing the nor-alkaloids product could be concentrated by some evaporation to produce a crystallized Nor-alkaloid compound. The aqueous permeate or the concentrate can be extracted by an organic solvent which phase-separates from the aqueous phase. The solvent extraction could be performed in a counter-current extraction centrifuge such as a Podbelniak extractor, or in a counter-current extraction column such as a Karr or Scheibel column. This yields the Northebaine or Nororipavine products in an organic solvent suitable for subsequent purification processing.

It will be understood that organic solvent extraction can be replaced with a series of process operations which yield a similar organic solution of nor-alkaloid. The series of process operations would include (a) precipitation of nor-alkaloid from the aqueous concentrate produced by addition of acid until acidic pH; (b) filtration and optionally water-washing of the resulting solids; and (c) dissolution of the filtered nor-alkaloid containing solids into an organic solvent suitable for further purification.

Optionally the organic extract can be contacted with carbon to adsorb impurities and color bodies. Optionally the carbon contacting can be done by mixing carbon in the organic extract and filtering the carbon out of the resulting suspension, or by feeding the organic extract to a column or filter containing a fixed bed of carbon and collecting a purified effluent stream. The organic extract can be crystallized by concentrating the solution evaporatively. The resulting nor-alkaloid products crystals can be filtered, washed, and dried to yield a high-purity Northebaine or Nororipavine product.

The reaction mixture can be filtered in order to remove the solid in the media (cell debris etc.). The resulting aqueous solution can be extracted repeatedly with an organic solvent not miscible with water (This can be Chloroform, Toluene, Dichloromethane, Ethyl acetate, etc.). The resulting organic phase can be concentrated into small quantity (resulting into a syrup;). The aqueous phase can be discarded. The resulting residue (Nor crude material) can be then crystallized from any short chain alcohol, such as methanol or it can be purified with other suitable purification technique such as Chromatography or other standard techniques. Another possible procedure to extract the alkaloids from the poppy straw, can be a caustic wash of the poppy straw followed by a filtration in order to remove the plant material. The alkaloids can then be precipitated from the basic solution as salt after adjusting the pH to acidic with addition of acid (f. ex. Sulphuric acid or hydrochloric acid, etc.). The nor-alkaloids can be extracted from the poppy straw trough percolation via an organic solvent. The resulting organics can be concentrated into small quantity. The resulting residue can be purified with other suitable purification technique such as crystallization and/or chromatography or other standard techniques.

### EXAMPLES

### Example 1. Fungal Bioconversion of Thebaine

Cunninghamella echinulata ATCC 9244 was propagated on potato dextrose agar at room temperature. Mature cultures on plates (7-10 days) were used for the preparation of concentrated spore suspensions. Spores were harvested by flooding the lawn culture with a sterile solution containing 0.9% sodium chloride and 0.01% Tween 80 (10 ml/plate), scratching them from the hyphae with the aid of a sterile loop and filtering the suspension through sterile cotton to remove hyphal fragments. Thamnostylum piriforme ATCC 8992 was propagated on malt extract agar (CM0059) at room temperature and spore suspensions were prepared as indicated above. C. echinulata and T. piriforme were cultivated in 0.25 L baffled flasks containing 25 ml of modified media based on Chaudhary et al., 2009 (For C. echinulata: 20 g/l glucose, 8 g/l Difco nutrient broth, 2 g/L yeast extract, pH 6.0 and for T. piriforme: 30 g/L glucose, 8 g/L Difco nutrient broth, 2 g/L yeast extract, 2 g/L potassium phosphate dibasic, 1 g/L potassium phosphate monobasic, 2 g/L sodium nitrate, 0.5 g/L potassium chloride, 0.5 g/L magnesium sulfate heptahydrate 0.02 g/L iron (II) sulfate heptahydrate, pH 6.0). Cultures were incubated at 28° C. with vigorous aeration (200 rpm on an orbital shaker).

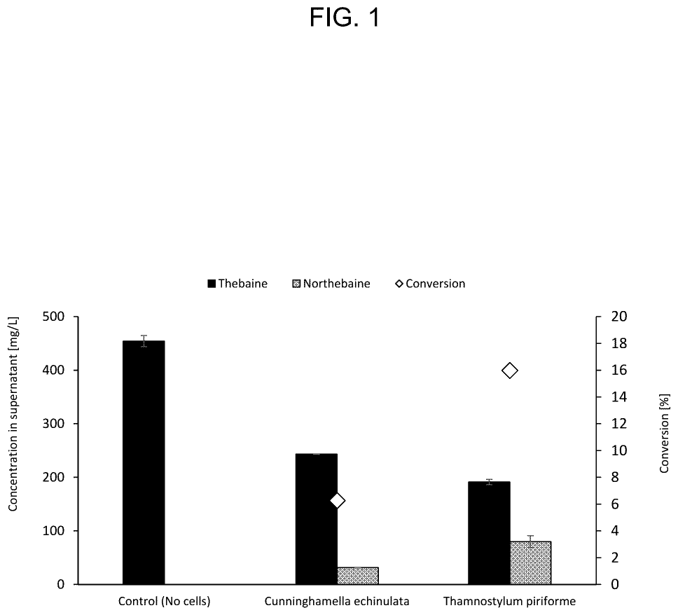

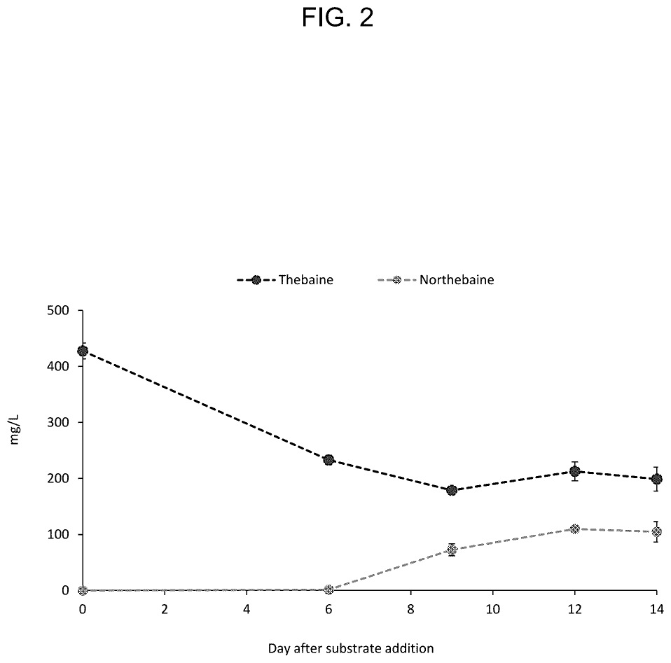

Media was inoculated with 0.25 ml of freshly-prepared spore suspensions. After 48 h of growth, thebaine was added to the cultures in a final concentration of 0.5 mg/ml from a 50 mg/ml stock solution prepared in methanol-1 M HCl (1:1). Eleven days after thebaine addition, supernatants were collected and analyzed by LC-MS. Thebaine was N-demethylated to northebaine by both C. echinulata and T. piriforme, reaching 6% and 16% conversion, respectively (FIG. 1). New T. piriforme cultures were set up in duplicates including negative controls for the purpose of inducing thebaine conversion activity and harvesting the biomass for RNA extraction and differential transcriptome analysis. Supernatant and biomass samples of induced and non-induced cultures were withdrawn from the flasks at time point 0 and on days 6 and 9 after thebaine addition. Thebaine and northebaine were monitored in the supernatants, reaching a conversion level of 25% on day 12 after substrate addition (FIG. 2).

### Example 2. mRNA Preparation from Fungi and Sequencing

Total RNA was isolated from T. piriforme biomass samples according to a standard protocol using the RNeasy Plant Mini Kit (Qiagen), taking care that all materials, buffers and solutions were RNAse-free. Around 100 mg of biomass were harvested in screw-cap microcentrifuge tubes containing Zirconia beads with the aid of a sterile spatula, flash-frozen with liquid nitrogen and lysed using a Precellys homogenizer (3×15 s) keeping the temperature at 4° C. Total RNA was eluted in 50 μl RNAse-free water. RNA purity and integrity were verified by using the Qubit® BR RNA kit, NanoDrop spectroscopy and 1% agarose gel electrophoresis. Five RNA-seq samples, corresponding to day 0, day 6 (non-induced), day 6 (induced), day 9 (non-induced) and day 9 (induced), were sent to GATC Biotech AG (Konstanz, Germany) for sequencing.

### Example 3. Bioinformatic Transcriptome Analysis

After Illumina sequencing, fastq files from all 5 RNA-Seq libraries were clipped and quality filtered with Trimmomatic (version 0.33, paramteres: SLIDINGWINDOW:4:28, MINLEN:75 LEADING:28 TRAILING:28), Bolger et al, 2014, Bioinformatics 30(15):2114-20).

Transcriptome was afterwards assembled with Trinity (version 2.2.0 parameters: —KMER_SIZE 31, —normalize_reads, —SS_lib_type, —min_kmer_cov 2, Grabherr et al, 2011, Nat Biotechnol., 29(7):644-52). Obtained transcripts were annotated via Trinotate (version 3, https://trinotate.github.io/) and transcripts containing either PFAM domain code PF00067 or COG2124 were considered as putative p450 enzymes. Similarly, CPRs were identified from the Trinotate annotation in case the closest hit was a fungal P450 reductase.

Expression levels (transcripts per million, TPM) of all transcripts were estimated with RSEM Li and Dewey, 2011, BMC Bioinformatics 4; 12:323) with default parameters, and afterwards a shortlist of P450 candidates was created by selecting those P450 candidates which had a minimal expression of 10 transcripts per million and showed at least 20% upregulation in the thebaine samples at day points 6 and 9 when compared to the non-induced samples, resulting in a list of 17 P450 candidates. Other 6 candidates were handpicked and checked afterwards for completeness by blasting the sequences against NCBI non-redundant database.

### Example 4. Cloning of Fungal CYP450 Enzymes and CPRs

cDNA Synthesis

cDNA was obtained according to a standard protocol using the Mint-2 cDNA synthesis kit (Evrogen). For T. piriforme, RNA samples from days 6 and 9 after induction were heated at 65° C. for 1-2 min and utilized for cDNA synthesis. One microgram total RNA was combined with 10 μM primer EV2424 (CDS-4M Adapter: 5′-AAGCAGTGGTATCAACGCAGAGTGGCCAGAATGGCCTTTGTTTTTTCTTTTTTTTTTTTVN-3′; SEQ ID NO:134) and 10 μM primer EV2425 (Short Oligo-Adapter: 5′-AGTGGCCTGCAGGGCCGGGGG-3′; SEQ ID NO:135), incubated in a thermocycler at 70° C. for 2 min and then at 42° C. for 3 min. Five microliter of reverse transcriptase mix made of 2 μl 5× First-strand buffer, 1 μl DTT (20 mM), 1 μl dNTP (10 mM each), 0.5 μl RNase inhibitor and 1 μl Mint reverse transcriptase, was added to each RNA sample at 42° C. without removing them from the thermocycler. After incubation at 42° C. for 30 min, 5 μl of IP-solution was added and incubated at the same temperature for 1.5 h.

First-strand cDNA samples were amplified using 10 μM PCR primer-M1 (5′-AAGCAGTGGTATCAACGCAGAGT-3′; SEQ ID NO:136) and adequate volumes of dNTPs, 10× Encyclo buffer and Encyclo polymerase mix, following the cycling program and temperatures described in the protocol. Optimal cycling conditions were determined by 1% agarose gel electrophoresis. Full-scale preparation of ds cDNA was performed using 18 and 21 cycles for RNA samples from days 6 and 9 after induction, respectively. Ds cDNA was column-purified and stored at −20° C.

Cloning of Target Genes into S. cerevisiae Expression Vectors

Twenty-six transcript sequences putatively encoding 20 cytochrome P450s (23 individual sequences, 3 isoforms with different N-termini) and 3 CPRs (Table 1) were amplified from T. piriforme ds cDNA and cloned into vectors for episomal yeast expression. The cytochrome P450 genes were inserted by In-Fusion Cloning (Takara Bio, USA) into pEVE2120 (URA3) and all CPRs were cloned similarly into pEVE3307 (HIS3) or pEVE3308 (LEU2). Forward and reverse primers for amplification of cytochrome P450 genes contained SfiI and SacII sites, with the exception of the reverse primer for amplification of transcript Tp_P450_6 (P450_DN9560_c0_g1_i1), which contained a BamHI site instead. Forward and reverse primers for amplification of CPR genes contained PmeI and PacI sites. To ensure cytochrome P450 oxidation activity, CPR from S. cerevisiae (Sc_CPR) and other yeast codon-optimized CPR genes from Cunninghamella elegans (Cel_CPR_co) and Gibberella fujikuroi (Gf_CPR_co) (Table 1) were synthesized (GeneArt, Thermo Fisher Scientific) to be cloned alone or in combination with the CPRs from T. piriforme. Constructs containing Sc_CPR, Cel_CPR and Gf_CPR were obtained by standard restriction site digestion and ligation (Table 2). Oligonucleotide sequences used for the amplification of all target genes are shown in Table 3.

Amplification of gene candidates was performed using Q5 High-Fidelity DNA polymerase and the primers from table B. Thermocycler programs were set according to the user manual (annealing temperatures ranging from 54 to 61° C., extension: 60 s for cytochrome P450s and 75 s for CPRs). Following agarose gel and column purification, 1 μl (50-200 ng) of each PCR-amplified fragment was mixed with 1 μl linearized vector (50 ng), 1 μl In-Fusion Enzyme and 1 μl millipore water. Samples were incubated at 50° C. for 15 min. Competent E. coli cells (50 μl) were transformed with 2.5 μl of the reaction mix and plated on LB-Amp. Finally, 3-4 clones were picked from each plate, assigned letters A to D, grown in LB-Amp for plasmid isolation and sent out for DNA sequencing (Microsynth). The resulting plasmids are indicated in Table 4.

Selection of Cytochrome P450 Homolog and Cloning of codon-Optimized Cytochrome P450 Genes

A putative cytochrome P450 gene from Lichtheimia ramosa (SEQ ID NO: 7) was identified by protein similarity to P450_DN15259_c0_g1_i7 (60%). Two cytochrome P450 sequences from T. piriforme, P450_DN15259_c0_g1_i7 and P450_DN12791_c0_g1_i1, as well as the cytochrome P450 from L. ramosa (Table 5) were codon-optimized for S. cerevisiae, synthesized (GeneArt, Thermo Fisher Scientific) and cloned by restriction site digestion with HindIII/SacII into pEVE3306 (URA3) for expression under the control of the PGK1 promoter (Table 6).

### Example 5. Expression of Fungal CYP450 Enzymes and CPRs in Yeast

Expression of T. piriforme Cytochrome P450 Genes Along with CPRs from C. elegans, G. Fujikuroi and S. cerevisiae.

Yeast strain EVST25898 was transformed with plasmids containing each cytochrome P450 gene (pEV31493-31564) along with two other plasmids containing three CPRs: pEV31215 (Cel_CPR_co) and pEV31308 (Sc_CPR/Gf_CPR_co). A negative control strain containing pEV31215, pEV31308 and pEVE2120 (empty URA) was also created. Cells were grown in SC-His-Leu-Ura medium at 30° C. with shaking at 300 rpm for 20-24 h and utilized as pre-cultures for in vivo bioconversion assays.

Expression of P450_DN15259_c0_g1_i7 in Combination with CPRs from T. piriforme

Yeast strain EVST25898 was transformed with pEV31541 (P450_DN15259_c0_g1_i7_A) along with plasmids containing a CPR alone or in combination (Table 7). Eight constructs based on the three CPRs from T. piriforme were selected due to their match with the corresponding sequences obtained from the transcriptome analysis (low number or complete absence of SNPs). Plasmids pEVE3307 (empty HIS) or pEVE3308 (empty LEU) were used to replace the absence of one CPR gene. Cells were grown in SC-His-Leu-Ura medium at 30° C. with shaking at 300 rpm for 20-24 h and utilized as pre-cultures for in vivo bioconversion assays.

Expression of P450_DN12791_c0_g1_i1 in Combination with CPR from C. elegans

Yeast strain EVST25898 was transformed with pEV31522 (P450_DN12791_c0_g1_i1_C) together with pEV31215 (Cel_CPR_co). Cells were grown in SC-His-Leu-Ura medium at 30° C., 300 rpm for 20-24 h and utilized as pre-cultures for in vivo bioconversion assays.

Expression of codon Optimized P450s in Combination with CPR from C. elegans

Yeast strain EVST25898 was transformed with either pEV32226 (P450_DN15259_co), pEV32227 (P450_DN12791_co) or pEV32228 (Lr_P450_co) together with pEV31215 (Cel_CPR_co) and pEVE3308 (empty LEU) to yield strains EVST29159, EVST29160 and EVST29161, respectively. A negative control strain containing pEV3306 (empty URA), pEVE3308 (empty LEU) and pEV31215 was also created (EVST29162). Cells were grown in SC-His-Leu-Ura medium at 30° C., 300 rpm for 20-24 h and utilized as pre-cultures for in vivo bioconversion assays.

### Example 6. Bioconversion Assay with thebaine or oripavine

Bioconversion of Thebaine by S. cerevisiae Harboring T. piriforme Cytochrome P450 Candidates and CPRs from C. elegans, G. Fujikuroi and S. cerevisiae.

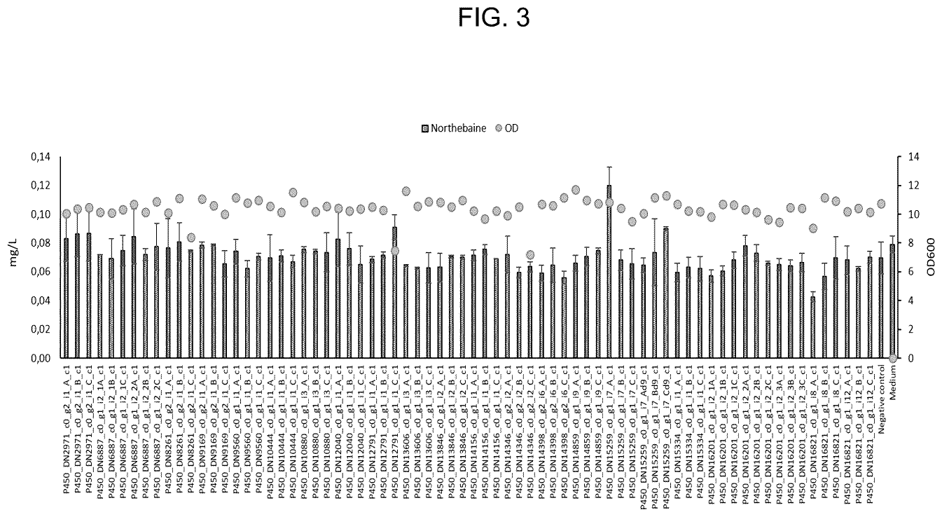

Yeast strain EVST25898 expressing T. piriforme cytochrome P450 gene candidates together with the CPRs from C. elegans, G. fujikuroi and S. cerevisiae as well as a negative control strain lacking P450 were assayed in a 96-deep-well-plate (DWP) format. Cells were grown in 0.5 ml SC-His-Leu-Ura medium containing 0.1 mM thebaine added from a 25 mM stock solution in DMSO. After 72 h of growth at 30° C. with shaking at 300 rpm, 100 μl-supernatants were harvested and spiked with 1 mg/I caffeine as internal standard. Northebaine was analyzed by LC-MS. Cytochrome P450 DN_15259_c0_g1_i7_A was identified as a thebaine N-demethylase (FIG. 3) and thus further investigated under optimized culture conditions.

Bioconversion of thebaine and oripavine by S. cerevisiae Harboring P450_DN15259_c0_g1_i7A in Combination with CPRs from T. piriforme

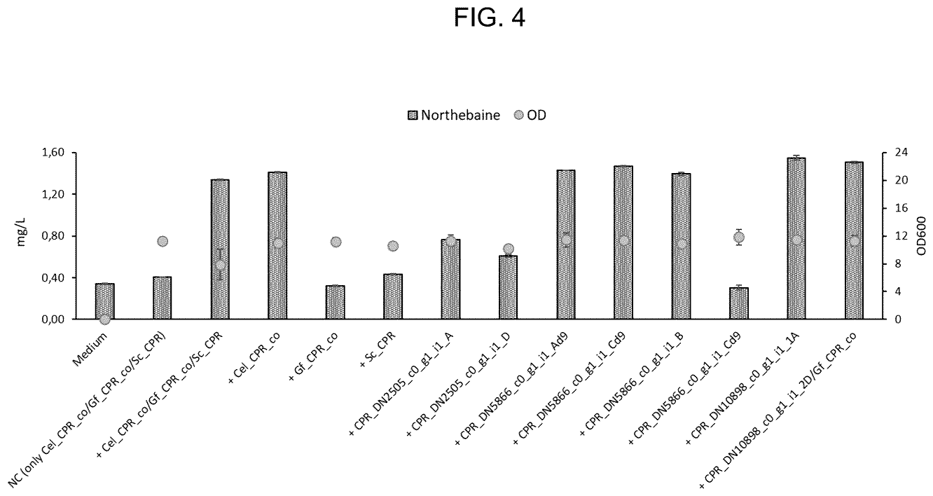

Yeast strain EVST25898 expressing P450_DN15259_c0_g1_i7_A along with the CPRs evaluated in the previous section or the native CPR candidates from T. piriforme were assayed in a 96-DWP format. Cells were grown in 0.5 ml SC-His-Leu-Ura medium containing 0.1 M potassium phosphate buffer pH 7 and 0.1 mM thebaine. After 72 h of growth at 30° C. with shaking at 300 rpm, 100 μl-supernatants were harvested, spiked with 1 mg/I caffeine as internal standard and analyzed by LC-MS. Cunninghamella elegans CPR (Cel_CPR_co) was found to support N-demethylase activity of P450_DN15259_c0_g1_i7_A. In addition, strains harboring CPR_DN5866_c0_g1_i1_Cd9 and CPR_DN10898_c0_g1_i1_1A yielded more northebaine compared to those containing CPR_DN2505_c0_g1_i1_A (FIG. 4).

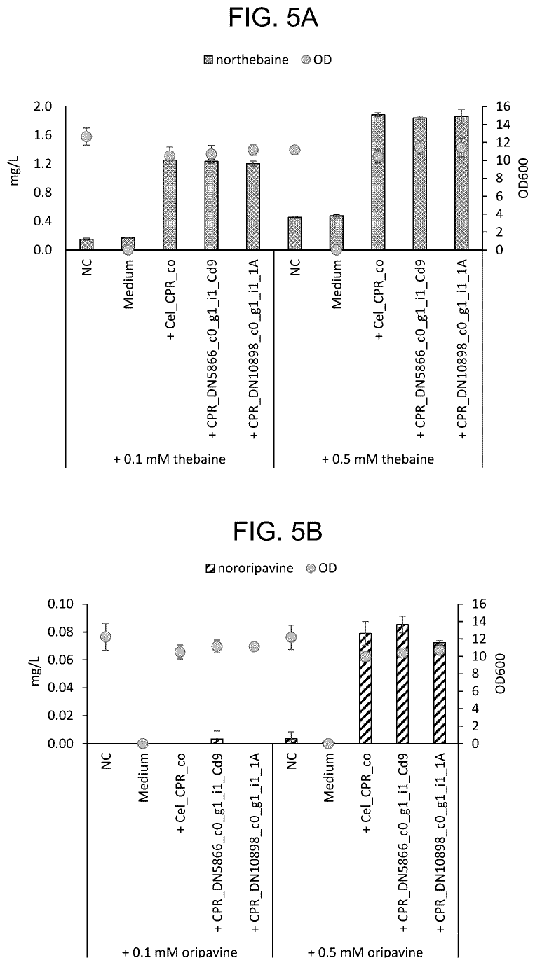

Yeast strain EVST25898 containing P450_DN15259_c0_g1_i7_A and separately co-expressing Cel_CPR_co, CPR_DN5866_c0_g1_i1_Cd9 and CPR_DN10898_c0_g1_i1_1A were grown in 0.5 ml SC-His-Leu-Ura medium containing 0.1 M potassium phosphate buffer pH 7 and either 0.1 or 0.5 mM thebaine or oripavine. After 72 h of growth at 30° C. with shaking at 300 rpm, 100 μl-supernatants were treated as described above and subjected to LC-MS analysis. Northebaine and nororipavine titers are shown in FIGS. 5A and 5B, respectively. The three CPRs performed similarly and increasing thebaine concentration to 0.5 mM caused a 50% increase in northebaine titers (FIG. 5A). P450_DN15259_c0_g1_i7_A was able to N-demethylate oripavine, though this activity was detected only when feeding cells with 0.5 mM oripavine (FIG. 5B). Nororipavine titers were lower than those of northebaine by 20-fold.

Bioconversion of oripavine by S. cerevisiae Harboring T. piriforme cytochrome P450 Candidates in Combination with the CPR from C. elegans

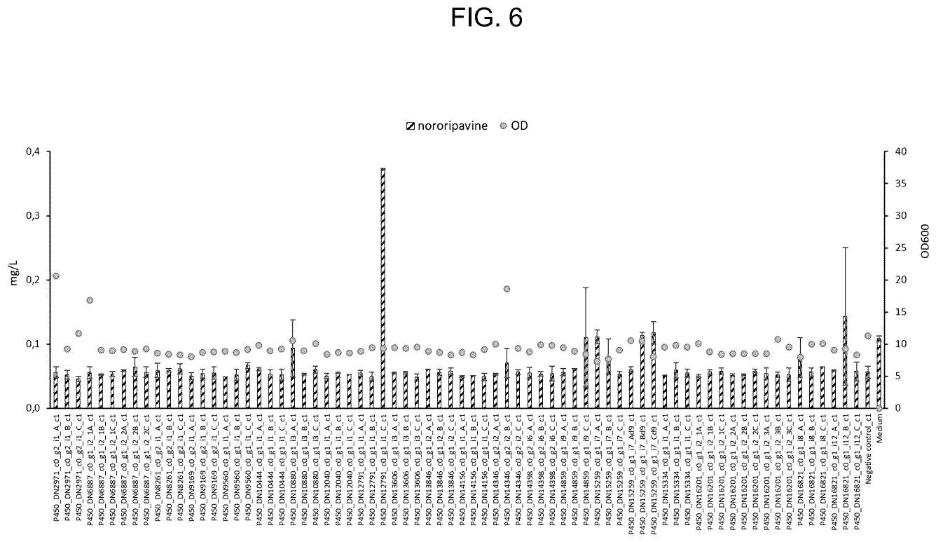

Yeast strain EVST25898 expressing T. piriforme cytochrome P450 gene candidates together with the CPRs from C. elegans, G. fujikuroi and S. cerevisiae were grown in 0.5 ml SC-His-Leu-Ura medium containing 0.1 M potassium phosphate buffer pH 7 and 0.5 mM oripavine. After 72 h of growth at 30° C. with shaking at 300 rpm, 100 μl-supernatants were harvested, treated as described above and subjected to LC-MS analysis of nororipavine. P450_DN12791_c0_g1_i1_C was identified as an oripavine N-demethylase (FIG. 6). Other potential candidates such as P450_DN10880_c0_g1_i3_A, P450_DN14346_c0_g2_i2_B, P450_DN14859_c0_g1_i9_C and P450_DN16821_c0_g1_i12_B were re-tested for oripavine N-demethylation and found not to perform better than the negative control strain under the conditions used (data not shown).

Bioconversion of thebaine and oripavine by S. cerevisiae Harboring Native and codon Optimized Fungal N-demethylase Candidates in Combination with the CPR from C. elegans

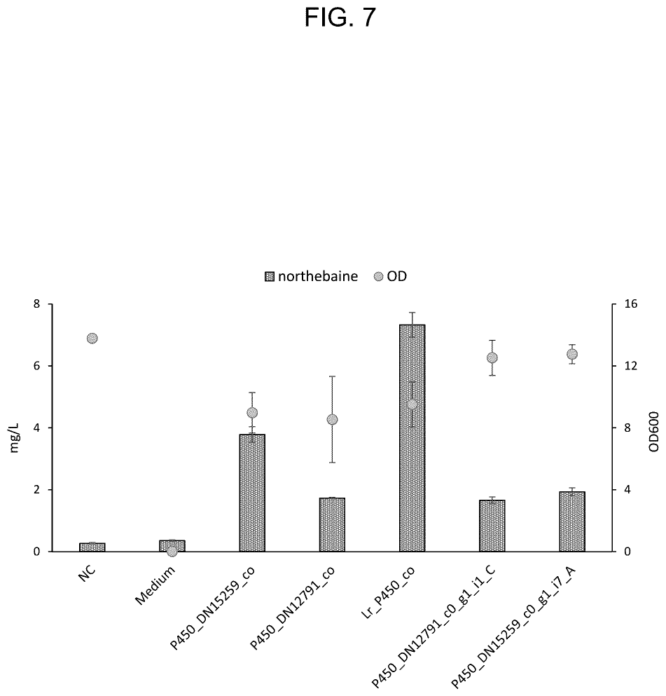

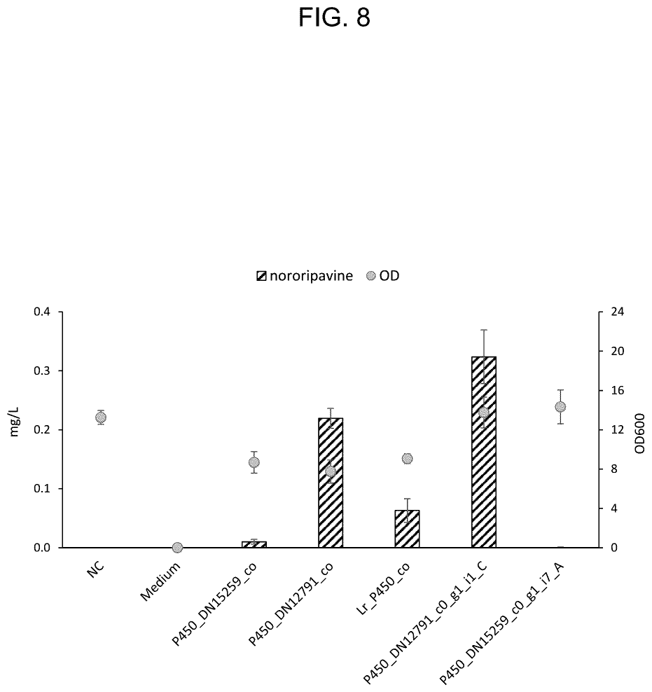

Yeast strain EVST25898 co-expressing Cel_CPR_co and five cytochrome P450 genes separately, namely P450_DN15259_c0_g1_i7_A, P450_DN12791_c0_g1_i1_C, P450_DN15259_co, P450_DN12791_co or P450 from Lichtheimia ramosa (Lr_P450_co), were grown in SC-His-Leu-Ura medium containing 0.1 M potassium phosphate buffer pH 7 and either 0.5 mM thebaine or oripavine. After 72 h of growth at 30° C. with shaking at 300 rpm, 100 μl-supernatants were harvested, treated as described above and subjected to LC-MS analysis. Northebaine and nororipavine titers are shown in FIGS. 7 and 8, respectively. Yeast codon-optimized P450_DN15259_co yielded 2-fold higher northebaine titers than the native P450_DN15259_c0_g1_i7_A. Lr_P450_co yielded more than 7 mg/L northebaine, equivalent to 2-fold higher northebaine titers compared to P450_DN15259_co. Thebaine N-demethylation activity of P450_DN12791_co was lower, with no significant difference between the codon optimized and non-codon optimized versions (FIG. 7). N-demethylase activity of the five tested P450 candidates towards oripavine was about 20-fold lower compared to that towards thebaine, with the highest nororipavine titers being obtained from yeast strains harboring P450_DN12791_c0_g1_i1_C and P450_DN12791_co (FIG. 8).

### Example 7. Bioinformatic Identification of Mammalian CYP450 Enzymes

Human CYP3A4, CYP3A5 and CYP2C8 have been reported as having opioid N-demethylation activity in studies with liver microsomes. Initially a set of immediate homologs of the human sequences were explored for sequence activity relationships.

BLAST searches were conducted with human CYP3A4, CYP3A5 and CYP2C8. The top 250 sequences in each case were aligned with Clustal Omega and a phylogenetic tree generated. Genes were then selected based on phylogeny positions.

To this end, 5 CYP3A4, 4 CYP3A5 and 4 CYP2C8 gene sequences have been selected for evaluation as a first part in this project.

### Example 8. Sourcing of Mammalian CYP450 Enzymes, Cloning and Expression in Yeast

The mammalian cytochrome P450s belonging to families 3A4 and 2C8 identified above (Tables 8, 10) were codon optimized for Saccharomyces cerevisiae and synthesized by TWIST Bioscience. The mammalian cytochrome P450s belonging to family 3A5 (Table 9) were codon optimized for Saccharomyces cerevisiae and synthesized by GeneArt® Gene Synthesis (ThermoFischer Scientific). Each cytochrome P450 enzyme was co-expressed with a human NADPH cytochrome P450 reductase (Table 1) and with the cytochrome b5 isoform 1 from Homo sapiens (Table 11), both codon optimized for Saccharomyces cerevisiae and synthesized by GeneArt® Gene Synthesis (ThermoFischer Scientific).

Each of the cytochrome P450 genes and the human NADPH cytochrome P450 reductase (CPR) were cloned into the replicative plasmid pEVE3307 containing a chromosomal replication origin for yeast (ARS), a centromere (CEN) of a yeast chromosome, a HIS3 yeast selection marker, an expression cassette consisting of the constitutive promoter PGK1 and CYC1 terminator, and an expression cassette consisting of the TEF1 promoter and ADH1 terminator. More specifically, the human CPR was PCR amplified with primers EVPR18206 and EVPR18207 (Table 12) containing the restriction sites HindIII and SacII, respectively. The PCR fragment was subsequently digested with SacII, followed by treatment with DNA Polymerase I, Large (Klenow) Fragment to generate blunt ends, followed by digestion with HindIII.

The obtained fragment was then cloned into pEVE3307 previously digested with AarI and PmeI, placing the human CPR between TEF1 promoter and CYC1 terminator, and generating plasmid pEV30967.

The cytochrome P450 genes were provided flanked by HindIII and SacII restriction sites by the synthesis companies. The fragments were digested with HindIII/SacII and cloned into pEV30967 previously digested with the same restriction enzymes, placing the human CPR between PGK1 promoter and ADH1 terminator, and originating plasmids pEV31021, pEV31022, pEV31023, pEV31024, pEV31025, pEV31026, pEV31027, pEV31028, pEV31029, pEV32390, pEV32391, pEV32392, pEV32393.

The human cytochrome b5 was cloned into the replicative plasmid pEVE2120 containing a chromosomal replication origin for yeast (ARS), a centromere (CEN) of a yeast chromosome, a URA3 yeast selection marker and an expression cassette consisting of the constitutive promoter PGK1 and ADH2 terminator. More specifically, a HindIII/SacII digested cytochrome b5 was cloned into pEVE2120 previously digested with the same restriction enzymes, generating plasmid pEV31030.

Plasmids were transformed into strain background EVST25898 (genotype MATalpha his3Δ0 leu2Δ0 ura3Δ0 aro3Δ::pTEF1-ARO4(K229L)-tCYC1::pPGK1-ARO7(T266L)-tADH1::KI CAT5-91Met GAL2 ho MIP1-661Thr SAL1-1 YORWΔ22::npBIO1nt-npBIO6nt) using the lithium acetate method (Gietz et al. 2002. Methods Enzymol. Vol 350, p 87-96). Transformants were selected in synthetic complete (SC) medium lacking histidine and uracil.

### Example 9. Cultivation of Yeast Strains

Yeast cells expressing the mammalian cytochrome P450s (Tables 8, 9, 10), the human CPR (Table 1) and the human cytochrome b5 (Table 11) were grown in Erlenmeyer shake flasks containing 20 mL SC medium lacking histidine and uracil. After approximately 20 hours at 30° C. with shaking at 160 rpm, an OD600 of 15-17 was reached. A number of cells equivalent to 220-240 OD units was harvested by centrifugation at 3000 g for 5 minutes, washed and resuspended in 3 mL water. The cell suspension was divided in three screw-cap-tubes (each containing approximately 75-80 OD units), centrifuged at 8000 g for 3 minutes and the supernatant removed. Cells were kept on ice to continue with in vitro assays, or frozen at −80° C. for further experiments.

### Example 10. In Vitro Assays for Bioconversion of Several Substrates

Approximately 75-80 OD units of cell pellets kept on ice were disrupted in 0.8 mL lysis buffer (100 mM potassium phosphate buffer pH 7.5, 1.2 M sorbitol, 100 mM NaCl, 0.5 mM fresh PMSF, 1 mM DTT, 1 tablet protease cocktail inhibitor and water to final 50 mL) and 0.5 mL glass beads. Cells were disrupted in a Precellys homogenizer for 3×25s keeping the temperature at 5-10° C.

The in vitro reactions were done in a final volume of 0.2 mL with 0.096 mL reaction buffer (100 mM potassium phosphate buffer pH 7, 5 mM NADP+, 25 mM glucose-6-phosphate, 5 μg/mL G6P dehydrogenase, 1 mM MgCl₂) and 0.1 mL crude cell extract. The reactions were initiated by addition of 0.2 mM substrate in DMSO, and incubated for approximately 20 hours at 30° C. The substrates tested for N-demethylation include: salutaridine, salutaridinol, thebaine, oripavine, morphine and codeine. The assays were terminated by addition of 0.2 mL methanol with 1% acetic acid, followed by centrifugation at 20000 g for 2 minutes. A volume of 0.1 mL of supernatant was analysed by LC-MS for the respective N-demethylated compounds.

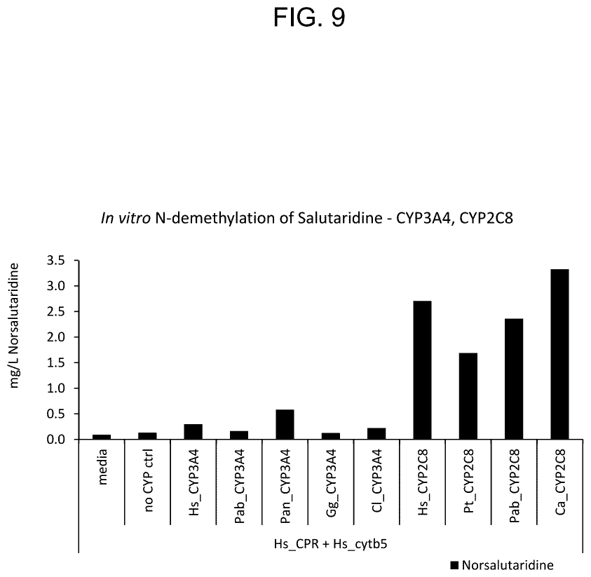

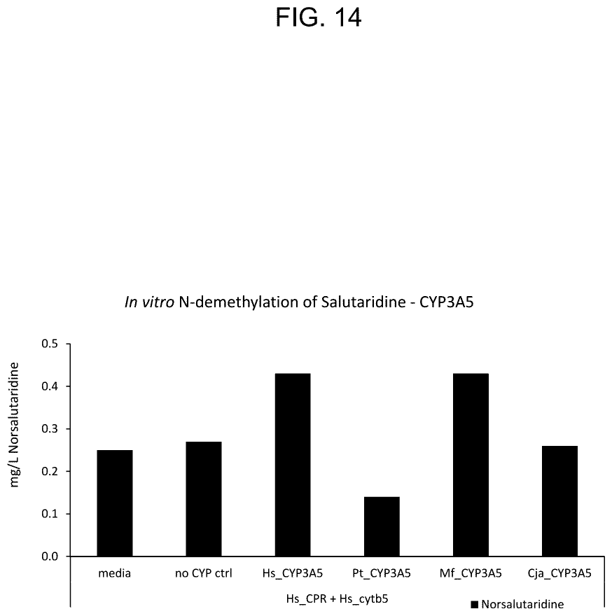

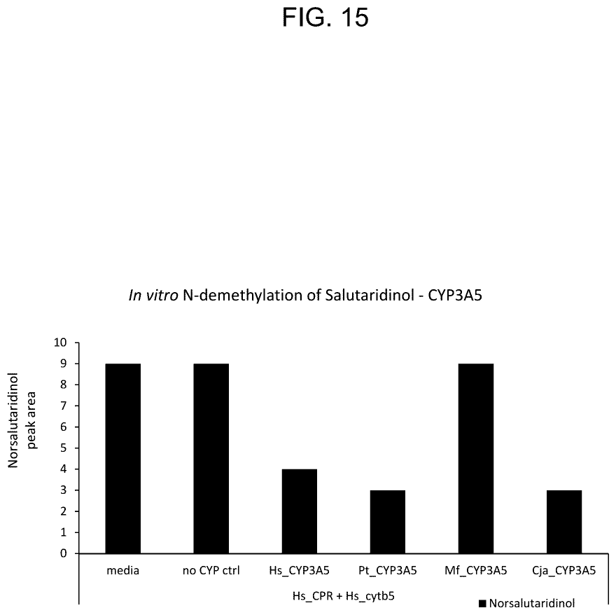

In vitro N-demethylation activity of the tested mammalian cytochrome P450s on salutaridine was detected for Hs_CYP3A4, Pan_CYP3A4, Hs_CYP2C8, Pt_CYP2C8, Pab_CYP2C8, Ca_CYP2C8 (FIG. 9), Hs_CYP3A5, Mf_CYP3A5 (FIG. 14) compared to the negative control strain without cytochrome P450s.

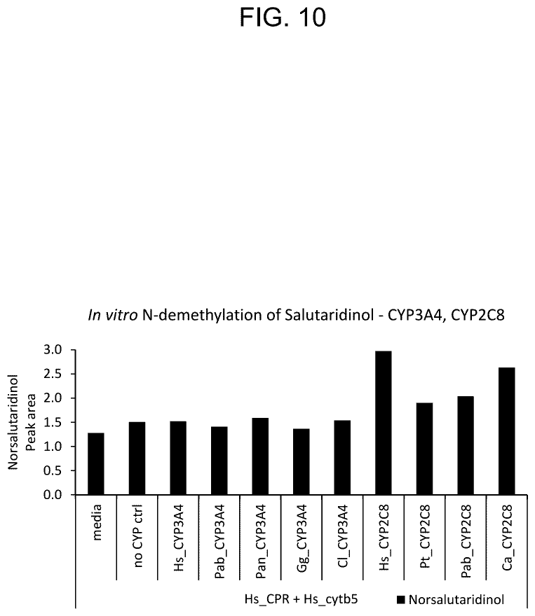

In vitro N-demethylation activity of the tested mammalian cytochrome P450s on salutaridinol was detected for Hs_CYP2C8, Ca_CYP2C8 (FIG. 10) compared to the negative control strain without cytochrome P450s.

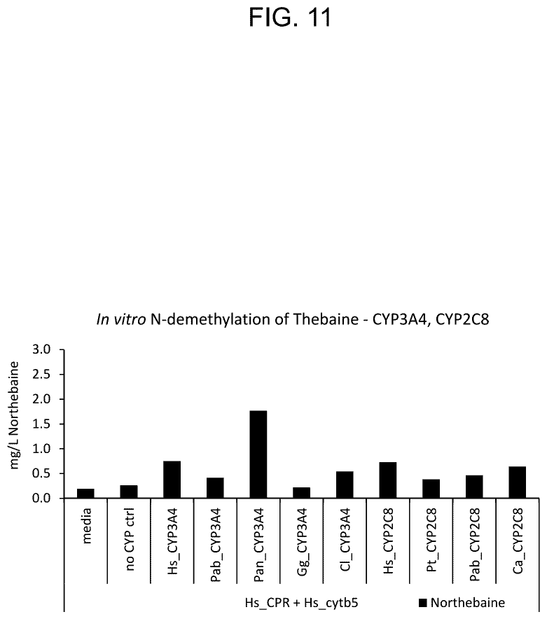

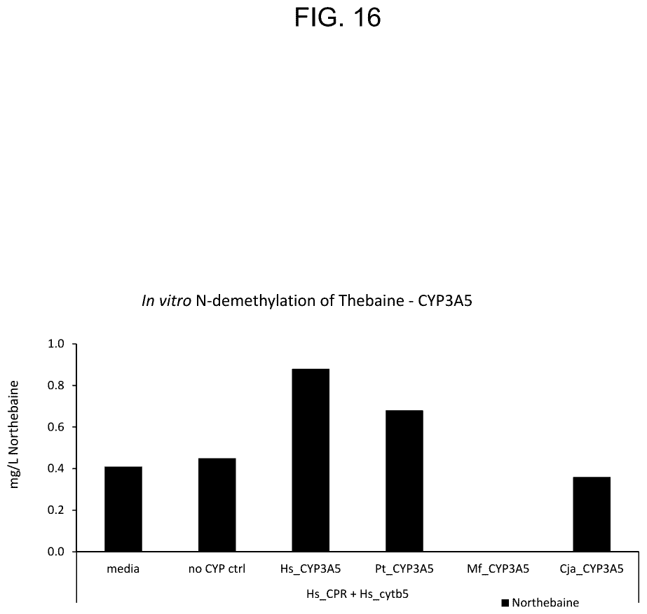

In vitro N-demethylation activity of the tested mammalian P450s on Thebaine was detected for Hs_CYP3A4, Pab_CYP3A4, Pan_CYP3A4, Cl_CYP3A4, Hs_CYP2C8, Pab_CYP2C8, Ca_CYP2C8 (FIG. 11), Hs_CYP3A5, Pt_CYP3A5 (FIG. 16) compared to the negative control strain without P450s.

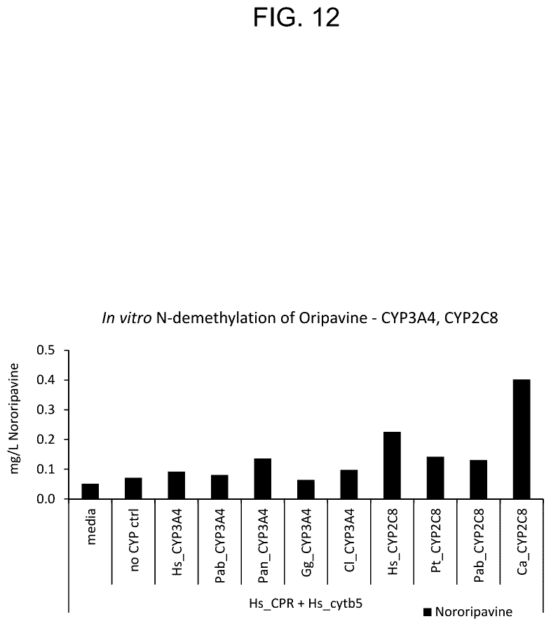

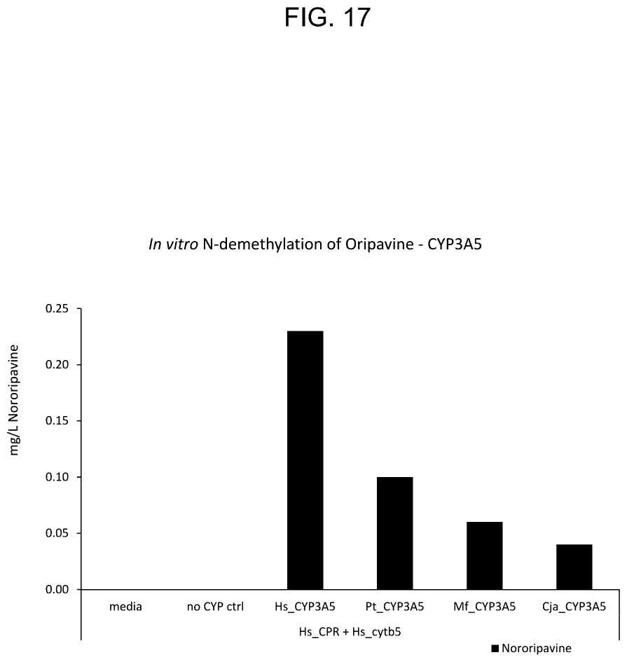

In vitro N-demethylation activity of the tested mammalian cytochrome P450s on oripavine was detected for Hs_CYP3A4, Pan_CYP3A4, Cl_CYP3A4, Hs_CYP2C8, Pt_CYP2C8, Pab_CYP2C8, Ca_CYP2C8 (FIG. 12), Hs_CYP3A5, Pt_CYP3A5, Mf_CYP3A5, Cja_CYP3A5 (FIG. 17) compared to the negative control strain without cytochrome P450s.

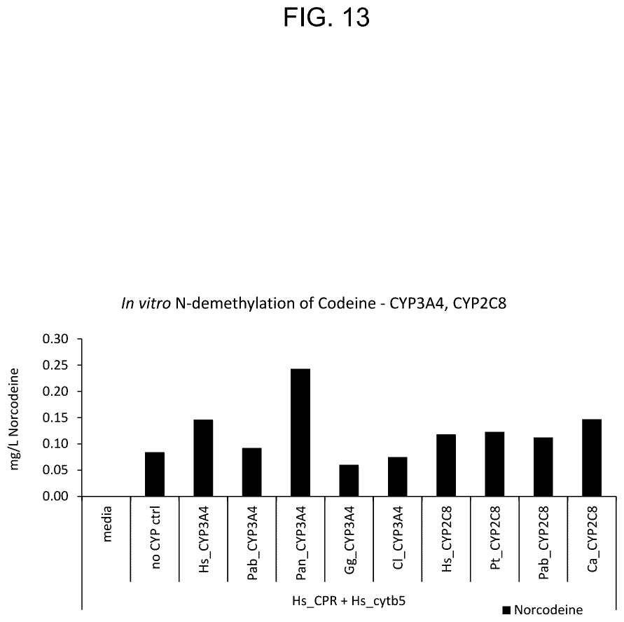

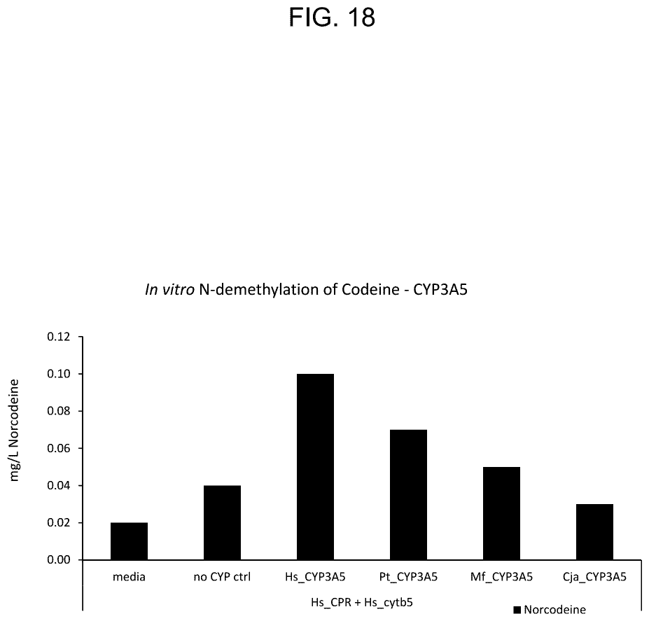

In vitro N-demethylation activity of the tested mammalian cytochrome P450s on codeine was detected for Hs_CYP3A4, Pan_CYP3A4, Hs_CYP2C8, Pt_CYP2C8, Pab_CYP2C8, Ca_CYP2C8 (FIG. 13), Hs_CYP3A5, Pt_CYP3A5, Mf_CYP3A5 (FIG. 18) compared to the negative control strain without cytochrome P450s.

### Example 11. Measurements of Bioconversion Substrates and Products by LC/MS LC-MS/MS Analysis of Nor-Compounds

Stock solutions for all compounds were prepared in DMSO at 1 g/L. A series of calibration solutions at 4 mg/L, 2 mg/L, 1 mg/L, 0.5 mg/L, 0.25 mg/L, 0.125 mg/L, 62.5 μg/L and 31.25 μg/L in the culture medium was prepared from this stock solution. Caffeine (Sigma) was added as internal standard to a concentration of 1 mg/L and samples were injected into the UPLC-TQD (Waters).

The LC-MS method was as follows: Mobile Phase A: water+0.1% formic acid; Mobile Phase B: acetonitrile+0.1% formic acid; Column: Aquity BEH C18100×2.1 mm (Waters).

The elution gradient is shown in Table 3 and the LC-MS conditions are given in Table 14.

Table 15 shows the mass spectrometer source and detector parameters.

Table 16 shows the target compounds, their parent ion, daughter ion (MRM) as well as dwell times, cone voltages and collision energies used.

### Example 12. Preparation of Compound MeO—I-MCP (Step A1)

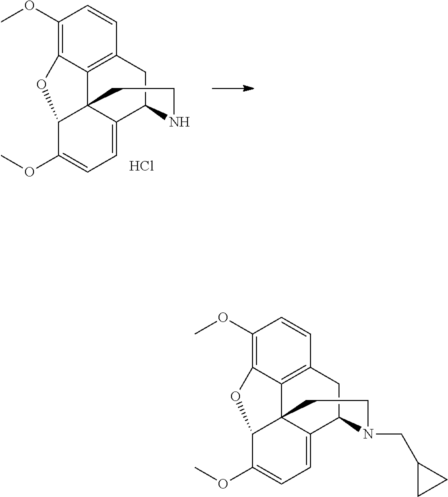 [SMILES: COC1=CC=C2[C@H]3Cc4ccc(OC)c5c4[C@@]2(CCN3)[C@H]1O5 ; Cl ; COC1=CC=C2[C@H]3Cc4ccc(OC)c5c4[C@@]2(CCN3CC2CC2)[C@H]1O5]

A 100 mL 3-necked flask was charged with Compound MeO—I—H (5.5 g, 16.5 mmol), cyclopropane carboxaldehyde (2.5 mL, 33 mmol), dichloro(p-cymene)ruthenium(II) dimer (100 mg, 0.165 mmol), triethylamine (13.75 mL, 99 mmol), and acetonitrile (50 mL) under a nitrogen atmosphere. The suspension was stirred at room temperature. Formic acid (7.78 mL, 206 mmol) was added slowly. The resulting mixture was heated at 60° C. for 2.5 h. The mixture was cooled to room temperature and concentrated under vacuum. The residue was partitioned between toluene and a 1 N NaOH aqueous solution. The aqueous layer was extracted twice with toluene. The combined organic layers were washed twice with water and then concentrated under vacuum to afford quantitatively Compound MeO—I-MCP (6.2 g).

### N-cyclopropylmethyl-northebaine

HPLC 92.5% at 215 nm.

MS (ES-API pos) m/z 352.2 (M+H).

¹H NMR (300 MHz, CDCl₃) δ [ppm] 6.64 (d, J=8.2 Hz, 1H), 6.57 (d, J=8.2 Hz, 1H), 5.54 (d, J=6.5 Hz, 1H), 5.27 (s, 1H), 5.02 (d, J=6.5 Hz, 1H), 3.91 (d, J=6.4 Hz, 1H), 3.83 (s, 3H), 3.58 (s, 3H), 3.24 (d, J=18H, 1H), 2.65-2.87 (m, 3H), 2.47 (d, J=6.0 Hz, 2H), 2.19 (dt, J=5.8 and 12.3 Hz, 1H), 1.70 (d, J=12 Hz, 1H), 0.90 (m, 1H), 0.54 (m, 2H), 0.15 (m, 2H).

¹³C NMR (75 MHz, CDCl₃) δ [ppm] 152.5, 142.8, 133.6, 132.6, 127.8, 119.2, 112.8, 111.7, 96.0, 89.2, 59.1, 58.6, 56.4, 54.9, 46.6, 44.3, 36.8, 30.6, 9.5, 3.9, 3.7.

### Example 13. Preparation of Compound MeO—I-MCP (Step A2)

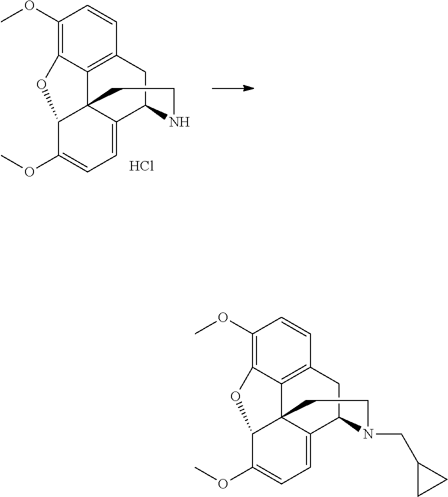 [SMILES: COC1=CC=C2[C@H]3Cc4ccc(OC)c5c4[C@@]2(CCN3)[C@H]1O5 ; Cl ; COC1=CC=C2[C@H]3Cc4ccc(OC)c5c4[C@@]2(CCN3CC2CC2)[C@H]1O5]

Triethylamine (1.6 mL, 12 mmol) was added to a suspension of Compound MeO—I—H (1.0 g, 3 mmol) in dichloromethane (25 mL). The mixture was cooled in an ice-water bath and cyclopropanecarboxylic acid chloride (0.35 mL, 3.6 mmol) was added dropwise. The cooling bath was removed and the mixture was stirred at room temperature overnight. The mixture was washed with a 1 N HCl aqueous solution, then with brine, dried with sodium sulfate and concentrated to a brown solid. The residue was dissolved in dry THF (10 mL) and slowly added to a stirred slurry of LiAlH₄ (0.20 g, 5.4 mmol) in anhydrous THF. The reaction mixture was heated at 60° C. for 1 h and then cooled in an ice-water bath. Wet diethyl ether was added to the mixture until there was no more bubbling. The mixture was filtered and the precipitate was washed several times with THF. The filtrate was concentrated under vacuum to give Compound MeO—I-MCP (0.80 g, 76%).

### N-cyclopropylmethyl-northebaine

HPLC 89.8% at 215 nm.

NMR and MS data were in agreement with those obtained from Example 12.

### Example 14. Preparation of Compound MeO—I-MCP (Step A3)

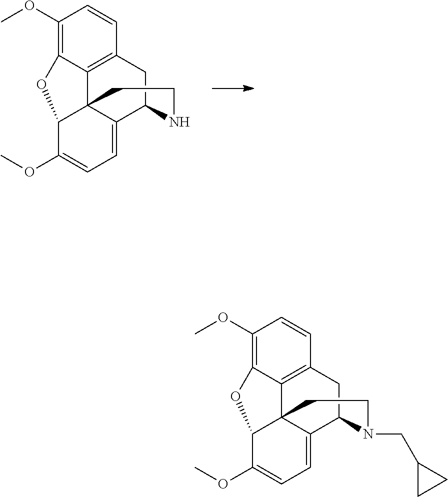 [SMILES: COC1=CC=C2[C@H]3Cc4ccc(OC)c5c4[C@@]2(CCN3)[C@H]1O5 ; COC1=CC=C2[C@H]3Cc4ccc(OC)c5c4[C@@]2(CCN3CC2CC2)[C@H]1O5]

A 50 mL 3-necked flask was charged with Compound MeO—I—H (0.59 g, 2 mmol), cyclopropylmethylbromide (0.54 g, 4 mmol), triethylamine (0.5 g, 5 mmol) and ethanol (15 mL). The mixture was heated to reflux for 3 h. The ethanol was removed under vacuum and the residue was partitioned between dichloromethane and water. The organic layer was dried with sodium sulfate and concentrated under vacuum to obtain Compound MeO—I-MCP as light brown solid (0.60 g, 85% yield).

### N-cyclopropylmethyl-northebaine

HPLC purity 97% at 215 nm.

MS (ES-API pos) m/z 352.3 (M+H).

NMR data was in agreement with those obtained from Example 12.

### Example 15. Preparation of Compound MeO—II-MCP (Step B)

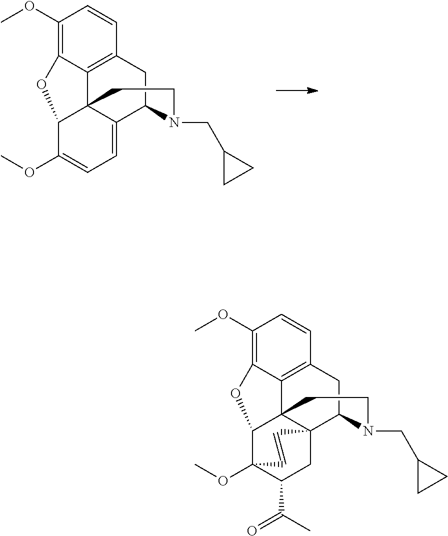 [SMILES: COC1=CC=C2[C@H]3Cc4ccc(OC)c5c4[C@@]2(CCN3CC2CC2)[C@H]1O5 ; COc1ccc2c3c1O[C@H]1[C@@]4(OC)C=C[C@@]5(C[C@@H]4C(C)=O)[C@@H](C2)N(CC2CC2)CC[C@]315]

A solution of Compound MeO—I-MCP (5.8 g, 16.5 mmol) and methyl vinyl ketone (12 mL, 144 mmol) in toluene (100 mL) was heated at 80° C. for 16 h. After cooling to room temperature the mixture was concentrated under vacuum to give a brown oily residue (6.5 g), which was purified by column chromatography (120 g SiO₂, elution with 0-20% EtOAc in heptane, R_{f} 0.3) to afford Compound MeO—II-MCP as a colorless solid (6.2 g, 89% yield).

### 7α-Acetyl-17-cyclopropylmethyl-6,14-endo(etheno)tetrahydro-northebaine

HPLC-purity 92.3% at 215 nm.

MS (ES-API pos) m/z 422.2 (M+H).

¹H NMR (300 MHz, CDCl₃) δ [ppm] 6.61 (d, J=8.2 Hz, 1H), 6.50 (d, J=8.2 Hz, 1H), 5.89 (d, J=8.8 Hz, 1H), 5.58 (d, J=8.8 Hz, 1H), 4.57 (s, 1H), 3.80 (s, 3H), 3.59 (s, 3H), 3.54 (d, J=6.4 Hz, 1H), 3.10 (d, J=18H, 1H), 2.89-3.03 (m, 2H), 2.66-2.72 (dd, J=4.7 and 11.8 Hz, 1H), 2.29-2.46 (m, 4H), 2.13 (s, 3H), 1.95 (dt, J=5.0 and 12.0 Hz, 1H), 1.83 (dd, J=2.3 and 12.9 Hz, 1H), 1.35 (dd, J=5.9 and 12.3 Hz, 1H), 0.81 (m, 1H), 0.51 (m, 2H), 0.12 (m, 2H).

¹³C NMR (75 MHz, CDCl₃) δ [ppm] 209.2, 148.0, 141.7, 136.2 (−), 134.3, 128.3, 125.8 (−), 119.3 (−), 113.5 (−), 95.4 (−), 81.3, 59.8, 57.0 (−), 56.6 (−), 53.5 (−), 50.7 (−), 48.2, 44.0, 43.2, 33.6, 30.5 (−), 30.0, 23.2, 9.5 (−), 4.1, 3.4.

### Example 16. Preparation of Compound MeO—IIIB-MCP (Step C)

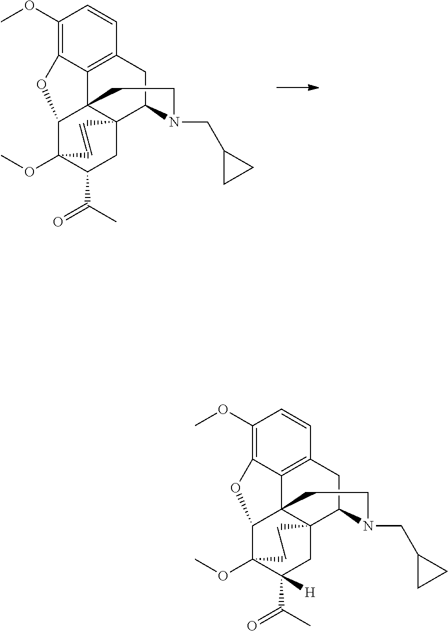 [SMILES: COc1ccc2c3c1O[C@H]1[C@@]4(OC)C=C[C@@]5(C[C@@H]4C(C)=O)[C@@H](C2)N(CC2CC2)CC[C@]315 ; COc1ccc2c3c1O[C@H]1[C@@]4(OC)CC[C@@]5(C[C@@H]4C(C)=O)[C@@H](C2)N(CC2CC2)CC[C@]315]

A vigorously stirred mixture of Compound MeO—II-MCP (1.1 g, 2.61 mmol) and Pd/C (10%, 50 mg) in iPrOH (20 mL) was hydrogenated at 80° C. for 16 h under 1 atm. H2 using a hydrogen-filled balloon. The mixture was filtered over Celite and the solid washed with iPrOH. The filtrate was concentrated to 1.1 g oil, which was purified by column chromatography (40 g SiO₂, elution 0-25% EtOAc in heptane) to yield Compound MeO—IIIB-MCP (1.0 g, 90% yield).

### 7α-Acetyl-17-cyclopropylmethyl-6,14-endo(ethano)tetrahydro-northebaine

HPLC-purity 89.3% at 215 nm.

MS (ES-API pos) m/z 424.2 (M+H).

¹H NMR (300 MHz, CDCl₃) δ [ppm] 6.70 (d, J=7.8 Hz, 1H), 6.56 (d, J=7.8 Hz, 1H), 4.48 (s, 1H), 3.87 (s, 3H), 3.43 (s, 3H), 2.95-3.07 (m, 3H), 2.59-2.78 (m, 2H), 2.21-2.36 (m, 3H), 2.26 (s, 3H), 2.19 (dt, J=5.8 and 12.3 Hz, 1H), 1.51-1.76 (m, 4H), 1.25-1.35 (m, 2H), 0.65-0.85 (m, 2H), 0.48 (m, 2H), 0.09 (m, 2H).

¹³C NMR (75 MHz, CDCl₃) δ [ppm] 210.9, 146.8, 141.7, 132.7, 128.8, 119.1, 114.0, 94.7, 77.5, 59.8, 58.4, 56.7, 52.2, 49.7, 46.4, 43.7, 35.4, 35.3, 33.8, 30.3, 28.7, 22.8, 17.4, 9.5, 4.0, 3.4.

### Example 17. Preparation of Compound MeO—IIIA-MCP (Step D)

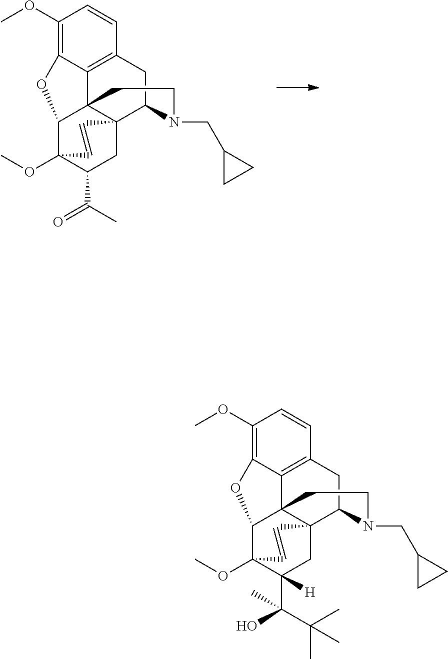 [SMILES: COc1ccc2c3c1O[C@H]1[C@@]4(OC)C=C[C@@]5(C[C@@H]4C(C)=O)[C@@H](C2)N(CC2CC2)CC[C@]315 ; COc1ccc2c3c1O[C@H]1[C@@]4(OC)C=C[C@@]5(C[C@@H]4[C@](C)(O)C(C)(C)C)[C@@H](C2)N(CC2CC2)CC[C@]315]

To a magnetically stirred solution of Compound MeO—II-MCP (2.1 g, 5 mmol) in toluene (50 mL) at room temperature was added a solution of tert-butylmagnesium chloride (1.7 M in THF, 20 mL, 34 mmol) over 5 min. The brown solution was stirred at room temperature for 4 h. The mixture was poured in a 10% ammonium chloride aqueous solution (100 mL) and the mixture was extracted with toluene. The extract was dried with sodium sulfate and concentrated to give a waxy solid. Purification by column chromatography (80 g SiO₂, 25% EtOAc in Heptane) gave Compound MeO—IIIA-MCP (1 g, 42% yield, R_{f} 0.6) as a solid. Some starting material (0.32 g, 15%, R_{f} 0.2) and reduced starting material (0.4 g, 18%, R_{f} 0.1) were also recovered.

### 7α-(2-(S)-hydroxy-3,3-dimethyl-2-butyl)-17-cyclopropylmethyl-6,14-endo(etheno)tetrahydro-northebaine

HPLC-purity 97.4% at 215 nm.

MS (ES-API pos) m/z 480.3 (M+H).

¹H NMR (300 MHz, CDCl₃) δ [ppm] 6.61 (d, J=8.2 Hz, 1H), 6.48 (d, J=8.2 Hz, 1H), 5.98 (d, J=8.8 Hz, 1H), 5.64 (s, 1H), 5.43 (d, J=8.8 Hz, 1H), 4.55 (s, 1H), 3.81 (s, 3H), 3.77 (s, 3H), 3.49 (d, J=6.4H, 1H), 3.09 (d, J=18 Hz, 1H), 2.97 (dd, J=12.3 and 8.8 Hz, 1H), 2.64 (m, 1H), 2.35-2.43 (m, 4H), 2.14 (t, J=8.8 Hz, 1H), 1.80-2.0 (m, 2H), 1.00 (s, 9H), 0.80-1.0 (m, 3H), 0.51 (m, 2H), 0.15 (m, 2H).

¹³C NMR (75 MHz, CDCl₃) δ [ppm] 148.1, 141.7, 135.5, 134.7, 128.5, 124.8, 119.2, 113.7, 99.0, 84.5, 78.4, 59.5, 56.7, 55.2, 47.1, 45.8, 44.1, 43.1, 39.7, 34.0, 32.2, 26.6, 23.1, 19.6, 9.5, 4.3, 3.2.

### Example 18. Preparation of Compound MeO—IV-MCP (Step D)

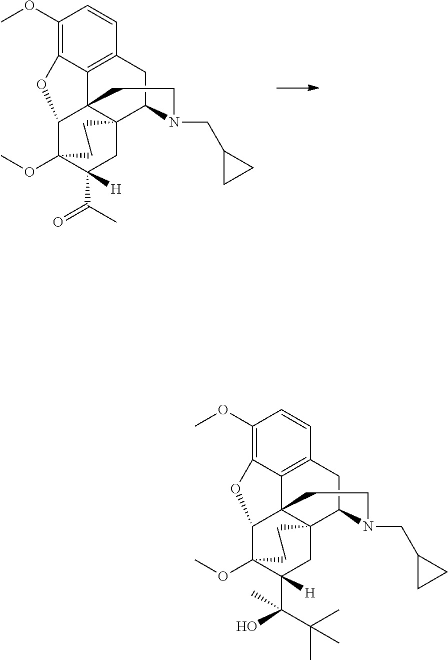 [SMILES: COc1ccc2c3c1O[C@H]1[C@@]4(OC)CC[C@@]5(C[C@@H]4C(C)=O)[C@@H](C2)N(CC2CC2)CC[C@]315 ; COc1ccc2c3c1O[C@H]1[C@@]4(OC)CC[C@@]5(C[C@@H]4[C@](C)(O)C(C)(C)C)[C@@H](C2)N(CC2CC2)CC[C@]315]

To a magnetically stirred solution of Compound MeO—IIIB-MCP (0.90 g, 2.1 mmol) in dry toluene (25 mL) at room temperature was added dropwise a solution of tert-butylmagnesium chloride (1.7 M solution in THF, 7.5 mL, 12.75 mmol). The reaction was quenched after 4 h by pouring the mixture into an aqueous solution made of 10% ammonium chloride (50 mL) and ice-water (50 mL). The layers were separated and the aqueous layer was extracted with toluene (3×25 mL). The combined organic layers were washed with brine, dried with sodium sulfate, and concentrated to an oil. Purification by column chromatography (80 g SiO₂, elution with 0-20% EtOAc in heptane, R_{f} 0.5) to yield Compound MeO—IV-MCP as a waxy solid (0.60 g, 60% yield).

### 7α-(2-(S)-hydroxy-3,3-dimethyl-2-butyl)-17-cyclopropylmethyl-6,14-endo(ethano)tetrahydro-northebaine

HPLC-purity 95.6% at 215 nm.

MS (ES-API pos) m/z 482.4 (M+H).

¹H NMR (300 MHz, CDCl₃) δ [ppm] 6.69 (d, J=8.2 Hz, 1H), 6.54 (d, J=8.2 Hz, 1H), 5.91 (s, 1H), 4.43 (s, 1H), 3.87 (s, 3H), 3.54 (s, 3H), 2.82-3.02 (m, 3H), 2.60 (dd, J=11.7 and 5.3H, 1H), 2.11-2.38 (m, 5H), 1.97 (dt, J=5.8 and 12.3 Hz, 1H), 1.60-1.85 (m, 3H), 1.36 (s, 3H), 1.25-1.30 (m, 1H), 1.00-1.12 (m, 1H), 1.03 (s, 9H), 0.70-0.83 (m, 2H), 0.48 (m, 2H), 0.10 (m, 2H).

¹³C NMR (75 MHz, CDCl₃) δ [ppm] 146.9, 141.6, 132.9, 128.9, 119.1, 114.0, 96.7, 80.7, 79.3, 59.5, 58.3, 56.9, 52.6, 46.2, 43.9, 43.7, 40.4, 35.9, 35.8, 33.4, 29.7, 26.4, 22.8, 20.0, 18.2, 9.5, 4.2, 3.2.

### Example 19. Preparation of Compound MeO—IV-MCP (Step C)

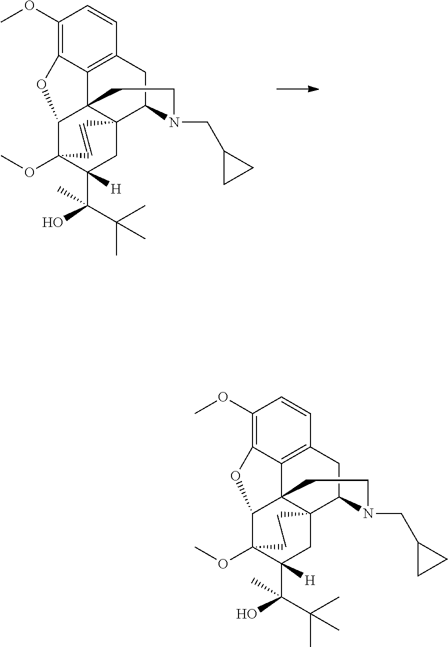 [SMILES: COc1ccc2c3c1O[C@H]1[C@@]4(OC)C=C[C@@]5(C[C@@H]4[C@](C)(O)C(C)(C)C)[C@@H](C2)N(CC2CC2)CC[C@]315 ; COc1ccc2c3c1O[C@H]1[C@@]4(OC)CC[C@@]5(C[C@@H]4[C@](C)(O)C(C)(C)C)[C@@H](C2)N(CC2CC2)CC[C@]315]

A vigorously stirred mixture of Compound MeO—IIIA-MCP (40 mg, 0.75 mmol), and Pd/C (10%, 10 mg) in iPrOH (10 mL) was hydrogenated at 80° C. for 16 h under 1 atmosphere of hydrogen. The mixture was filtered over Celite. The filtrate was concentrated to give Compound MeO—IV-MCP as a wax (40 mg, 100%).

### 7α-(2-(S)-hydroxy-3,3-dimethyl-2-butyl)-17-cyclopropylmethyl-6,14-endo(ethano)tetrahydro-northebaine

HPLC-purity 83% at 254 nm.

MS (ES-API pos) m/z 482.3 (M+H).

The NMR data were in agreement with those obtained for Example 18.

### Example 20. Preparation of Compound HO—IIIA-MCP (Step E)

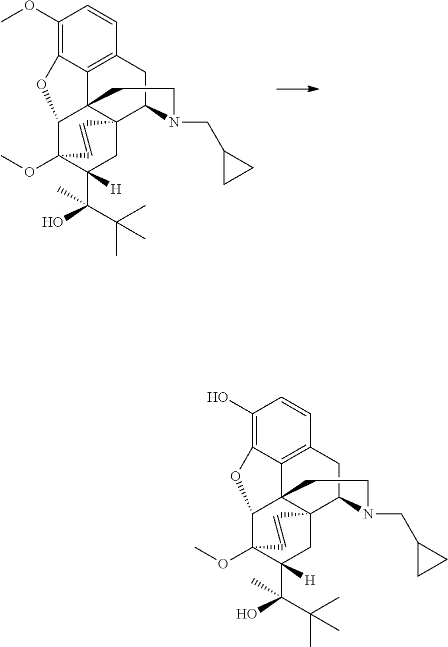 [SMILES: COc1ccc2c3c1O[C@H]1[C@@]4(OC)C=C[C@@]5(C[C@@H]4[C@](C)(O)C(C)(C)C)[C@@H](C2)N(CC2CC2)CC[C@]315 ; CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]

To a magnetically stirred solution of KOtBu (1.12 g, 10 mmol) and DMSO (10 mL) was added 1-dodecanethiol (2.03 g, 10 mmol). The resulting suspension was heated to 70° C. and a solution of Compound MeO—IIIA-MCP (0.90 g, 1.87 mmol) in DMSO (12 mL) was added. The resulting solution was heated at 110° C. for 16 h. The mixture was cooled to room temperature. Heptane (40 mL), EtOAc (10 mL) and a 1 N NH₄Cl aqueous solution (50 mL) were added. The layers were separated. The aqueous layer was washed twice with a heptane/EtOAc (4/1) mixture. The acidic aqueous layer was neutralized to pH 7-8 by careful addition of solid NaHCO₃ and extracted with EtOAc. The extract was washed with brine, dried with sodium sulfate and concentrated to an oil. Crystallization in MeOH and filtration afforded Compound HO—IIIA-MCP (240 mg, 28%) after drying. The mother liquor was concentrated and the residue purified by column chromatography to afford additional Compound HO—IIIA-MCP (270 mg, 31%), hence a total Compound HO—IIIA-MCP (510 mg, 59%) was obtained.

### 7α-(2-(S)-hydroxy-3,3-dimethyl-2-butyl)-17-cyclopropylmethyl-6,14-endo(etheno)tetrahydro-nororipavine

HPLC-purity 94.1% at 215 nm.

MS (ES-API pos) m/z 466.2 (M+1).

¹H NMR (300 MHz, CDCl₃) δ [ppm] 6.58 (d, J=8.2 Hz, 1H), 6.44 (d, J=8.2 Hz, 1H), 5.96 (d, J=8.8 Hz, 1H), 5.64 (s, 1H), 5.43 (d, J=8.8 Hz, 1H), 4.89 (br s, 1H), 4.58 (s, 1H), 3.75 (s, 3H), 3.49 (d, J=6.0H, 1H), 3.08 (d, J=18 Hz, 1H), 2.97 (dd, J=12.3 and 8.8 Hz, 1H), 2.65 (m, 1H), 2.31-2.43 (m, 4H), 2.15 (t, J=8.8 Hz, 1H), 1.80-2.0 (m, 2H), 1.00 (s, 9H), 0.80-1.0 (m, 3H), 0.51 (m, 2H), 0.15 (m, 2H).

¹³C NMR (75 MHz, CDCl₃) δ [ppm] 146.6, 137.2, 135.7, 134.5, 128.0, 124.4, 119.7, 116.0, 99.4, 84.5, 78.6, 59.5, 56.7, 55.2, 47.4, 45.8, 44.1, 43.1, 39.7, 33.9, 32.1, 26.6, 23.1, 19.6, 9.5, 4.3, 3.2

### Example 21. Preparation of buprenorphine (Step E)

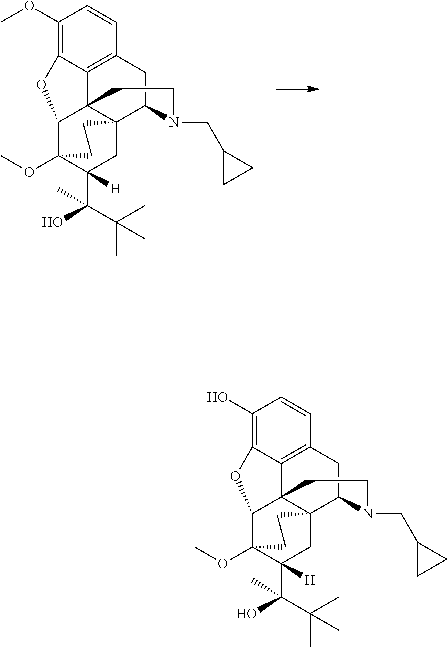 [SMILES: COc1ccc2c3c1O[C@H]1[C@@]4(OC)CC[C@@]5(C[C@@H]4[C@](C)(O)C(C)(C)C)[C@@H](C2)N(CC2CC2)CC[C@]315 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

A 100 mL 3-necked flask was charged with KOtBu (200 mg, 1.8 mmol) and DMF (10 mL) under a nitrogen atmosphere, and the mixture was heated to 50° C. After the addition of 1-dodecanethiol (0.43 mL, 0.364 mg, 1.8 mmol) a white suspension was formed. Then a solution of Compound MeO—IV-MCP (600 mg, 1.28 mmol) in DMF (10 mL) was added and the resulting solution was heated at 120° C. for 16 h. The mixture was quenched by addition of 50 mL of a 10% citric acid solution to reach pH 4. The mixture was poured in water (50 mL) and washed with toluene (3×25 mL). The aqueous layer was neutralized to pH 7 by the addition of NaOH and extracted with EtOAc (3×25 mL). The combined extracts were dried with sodium sulfate and concentrated to an oil (0.35 g, 59% yield, HPLC 79% purity). Crystallization from wet MeOH (10 mL) gave crystalline buprenorphine (50 mg). The mother liquor was purified by column chromatography (12 g SiO₂, elution with 0-25% EtOAc in heptane) and provided additional buprenorphine as white solid (190 mg). A total of 240 mg of buprenorphine (40% yield) was obtained. Analytical data were in agreement with the literature.

### buprenorphine

HPLC-purity 98.8% at 215 nm.

DSC-Melting point 216.7° C. (Lit. 216-218).

MS (ES-API pos) m/z 468.4 (M+H).

¹H NMR (300 MHz, CDCl₃) δ [ppm] 6.68 (d, J=8.2 Hz, 1H), 6.51 (d, J=8.2 Hz, 1H), 5.88 (s, 1H), 4.88 (br s, 1H), 4.45 (s, 1H), 3.53 (s, 3H), 2.82-3.02 (m, 3H), 2.60 (dd, J=11.8 and 4.7H, 1H), 2.12-2.36 (m, 5H), 1.97 (dt, J=5.3 and 12.3 Hz, 1H), 1.60-1.85 (m, 3H), 1.36 (s, 3H), 1.26-1.36 (m, 1H), 1.03-1.11 (m, 1H), 1.03 (s, 9H), 0.69-0.82 (m, 2H), 0.48 (m, 2H), 0.10 (m, 2H).

¹³C NMR (75 MHz, CDCl₃) δ [ppm] 145.4, 137.2, 132.6, 128.4, 119.6, 116.3, 97.1, 80.8, 79.5, 59.5, 58.3, 52.5, 46.5, 43.7, 43.7, 40.4, 36.0, 35.8, 33.4, 29.6, 26.4, 22.9, 20.1, 18.2, 9.5, 4.1, 3.2.

### Example 22. Preparation of buprenorphine (Step C)

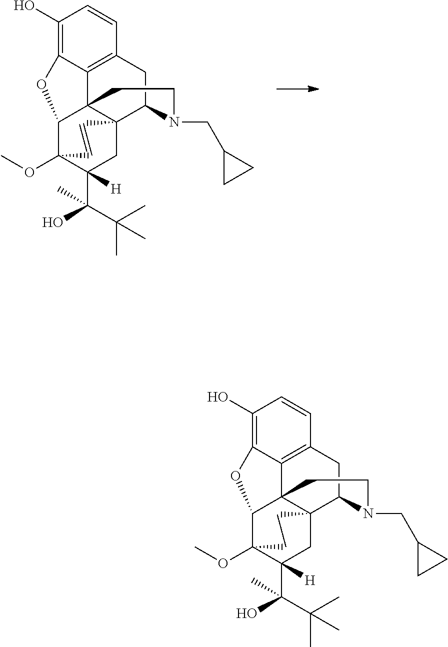 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

A vigorously stirred mixture of Compound HO—IIIA-MCP (350 mg, 0.75 mmol) and Pd/C (10%, 80 mg, 10 mol % Pd) in iPrOH (20 mL) and water (1 mL) was hydrogenated at 80° C. for 16 h under 1 atm. H2 using a hydrogen-filled balloon. The mixture was filtered over Celite. The filtrate was concentrated to a white foam, which was taken up in MeOH (5 mL) and stirred for 1 h. The solid was collected by filtration and dried under vacuum to give buprenorphine as solid (165 mg, 47%). The mother liquor was concentrated to give more buprenorphine as a solid (180 mg, 51%). A total of 345 mg of buprenorphine (98% yield) was obtained.

### buprenorphine

HPLC-purity 86%.

MS and NMR data were in agreement with those obtained for Example 21.

### Example 23. Preparation of Compound HO—I-MCP (Step A1)

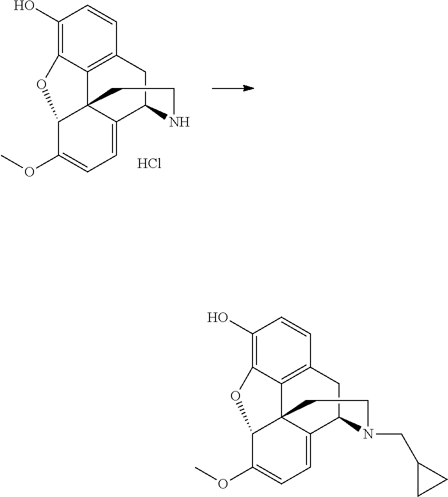 [SMILES: COC1=CC=C2[C@H]3Cc4ccc(O)c5c4[C@@]2(CCN3)[C@H]1O5 ; Cl ; COC1=CC=C2[C@H]3Cc4ccc(O)c5c4[C@@]2(CCN3CC2CC2)[C@H]1O5]

A 50 mL 3-neck round bottom flask was charged with Compound HO—I—H (910 mg, 3.21 mmol), cyclopropane carboxaldehyde (455 mg, 6.49 mmol), triethylamine (1.64 g, 16.22 mmol) and acetonitrile (9 mL), at room temperature and under a nitrogen atmosphere. To the stirred solution was added formic acid (2.4 mL) dropwise, over 10-15 min. After 10 min, di-p-chlorobis[(p-cymene)chlororuthenium(II)] (5 mg, 0.0082 mmol) was added and the mixture was stirred at 50° C. overnight. The volatiles were removed under vacuum and water (50 mL) was added to the resulting mixture.

A 25% NH40H aqueous solution (10 mL) was added and the aqueous mixture was extracted with CHCl₃ (3×50 mL). The combined organic layers were washed with brine (100 mL), dried over sodium sulfate, filtered off and the solvent was removed under vacuum. The crude product was purified by flash chromatography (0 to 10% MeOH in DCM) to afford Compound HO—I-MCP (1.07 g, 98%) was obtained as an off white solid.

### (4R,7aR,12bS)-3-(Cyclopropylmethyl)-7-methoxy-2,3,4,7a-tetrahydro-1H-4,12-methanobenzofuro[3,2-e]isoquinolin-9-ol

HPLC-purity 98.6% at 215 nm.

MS (ES-API pos) m/z 338.2 (M+H).

¹H NMR (300 MHz, CDCl₃) δ [ppm] 6.65 (d, J=8.4 Hz, 1H), 6.55 (d, J=8.4 Hz, 1H), 5.59 (d, J=6.6 Hz, 1H), 5.31 (s, 1H), 5.09 (d, J=6.6 Hz, 1H), 3.95 (d, J=6.6 Hz, 1H), 3.63 (s, 3H), 3.26 (d, J=18.0 Hz, 1H), 2.95 (dd, J=12.6, 4.2 Hz, 1H), 2.83 (m, 1H), 2.72 (dd, J=18.0, 7.2 Hz, 1H), 2.52 (m, 2H), 2.22 (dt, 1H), 1.75 (d, J=11.4 Hz, 1H), 0.93 (m, 1H), 0.56 (d, J=8.4 Hz, 2H), 0.18 (d, J=8.4 Hz, 2H).

¹³C NMR (75 MHz, CDCl₃) δ [ppm] 151.9, 142.9, 138.3, 133.2, 132.9, 127.3, 119.7, 116.0, 111.5, 96.5, 89.7, 59.0, 58.5, 55.0, 46.9, 44.2, 36.7, 30.6, 9.4, 3.9, 3.8.

### Example 24. Preparation of Compound HO—I-MCP (Step A2)

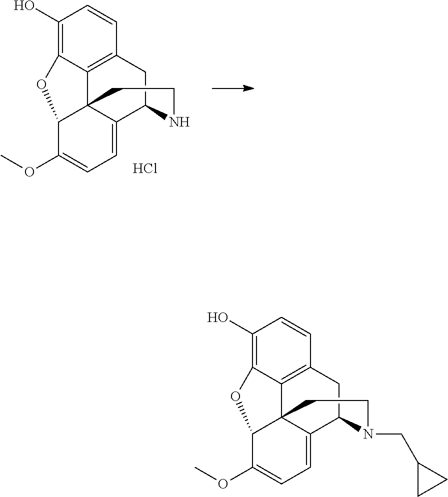 [SMILES: COC1=CC=C2[C@H]3Cc4ccc(O)c5c4[C@@]2(CCN3)[C@H]1O5 ; Cl ; COC1=CC=C2[C@H]3Cc4ccc(O)c5c4[C@@]2(CCN3CC2CC2)[C@H]1O5]

To a suspension of Compound HO—I—H (505 mg, 1.78 mmol) in CHCl₃ (14 mL) was added triethylamine (0.65 mL, 4.63 mmol) at room temperature and under a nitrogen atmosphere. The mixture was cooled to 0° C. with an ice/water bath and cyclopropane carboxylic acid chloride (440 mg, 4.12 mmol) dropwise. The mixture was stirred for 3 h at room temperature. The mixture was washed with a 1M HCl aqueous solution (30 mL), water (30 mL), dried over sodium sulfate and filtered off. The solvents were removed under vacuum. The brown residue was dissolved in THF (8 mL) then added dropwise to a slurry of LiAlH₄ (203 mg, 5.35 mmol) in THF (8 mL), at room temperature and under a nitrogen atmosphere. The mixture was then refluxed for 1.5 h. The mixture was cooled to 0° C. with an ice/water bath and carefully quenched with an ammonium chloride saturated aqueous solution. The mixture was diluted with THF (20 mL) and filtered off. The solid was washed with THF and the filtrate was concentrated under vacuum. The crude product Compound HO—I-MCP (500 mg, 83%) was obtained as an off white solid.

### (4R,7aR,12bS)-3-(Cyclopropylmethyl)-7-methoxy-2,3,4,7a-tetrahydro-1H-4,12-methanobenzofuro[3,2-e]isoquinolin-9-ol

HPLC-purity 94% at 215 nm.

NMR and MS data were in agreement with those obtained from Example 23.

### Example 25. Preparation of Compound HO—I-MCP (Step A3)

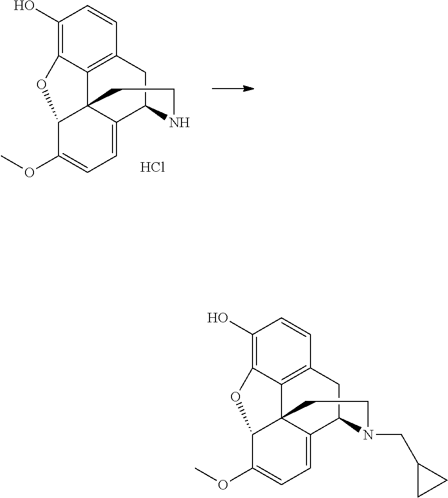 [SMILES: COC1=CC=C2[C@H]3Cc4ccc(O)c5c4[C@@]2(CCN3)[C@H]1O5 ; Cl ; COC1=CC=C2[C@H]3Cc4ccc(O)c5c4[C@@]2(CCN3CC2CC2)[C@H]1O5]

To a suspension of Compound HO—I—H (495 mg, 1.747 mmol) in EtOH (15 mL) were added triethylamine (0.61 mL, 4.37 mmol) and (bromomethyl)cyclopropane (0.35 mL, 3.494 mmol) at room temperature and under a nitrogen atmosphere. The mixture was refluxed overnight. The volatiles were removed under vacuum. Water (50 mL) and CHCl₃ (50 mL) were added. The aqueous phase was extracted with CHCl₃ (2×50 mL). The combined organic layers were dried over sodium sulfate, filtered off and the solvent was removed under vacuum. The crude product (510 mg) was purified by flash chromatography (0 to 10% MeOH in DCM) to afford Compound HO—I-MCP (370 mg, 63%) was obtained as an off white solid.

### (4R,7aR,12bS)-3-(Cyclopropylmethyl)-7-methoxy-2,3,4,7a-tetrahydro-1H-4,12-methanobenzofuro[3,2-e]isoquinolin-9-ol

HPLC-purity 94% at 215 nm.

NMR and MS data were in agreement with those obtained from Example 23.

### Example 26. Preparation of Compound HO—II-MCP (Step B)

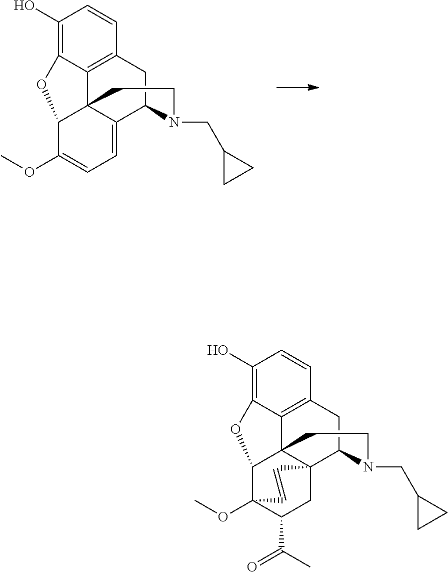 [SMILES: COC1=CC=C2[C@H]3Cc4ccc(O)c5c4[C@@]2(CCN3CC2CC2)[C@H]1O5 ; CO[C@]12C=C[C@@]3(C[C@@H]1C(C)=O)[C@H]1Cc4ccc(O)c5c4[C@@]3(CCN1CC1CC1)[C@H]2O5]

To a suspension of Compound HO—I-MCP (2.51 mg, 6.7 mmol) in toluene (50 mL) was added methyl vinyl ketone (12.2 mL, 139.1 mmol), at room temperature and under a nitrogen atmosphere. The reaction mixture was stirred at 80° C. overnight. The volatiles were removed under vacuum and the obtained crude material was triturated in hot EtOH, filtered off and washed with EtOH. Isolated Compound HO—II-MCP (1.88 g, 67%) was obtained as a beige solid. The mother liquor was concentrated under vacuum and the residue was purified by flash chromatography (0 to 5% MeOH in DCM). The obtained material was further triturated in hot EtOH and the solid was washed 3 times with EtOH prior to being isolated as additional Compound III-A (270 mg, 11%) as a beige solid (total amount: 2.15 g, 78%).

### 1-((4R,4aI,7I,7aI,12bI)-3-(Cyclopropylmethyl)-9-hydroxy-7-methoxy-1,2,3,4,7,7a-hexahydro-4a,7-ethano-4,12-methanobenzofuro[3,2-e]isoquinolin-14-yl)ethan-1-one

HPLC-purity at 215 nm: 95.9% (1.88 g batch); 97.1% (270 mg batch).

MS (ES-API pos) m/z 408.2 (M+H).

¹H NMR (300 MHz, CDCl₃) δ [ppm] 6.6 (d, J=7.8 Hz, 1H), 6.46 (d, J=7.8 Hz, 1H), 5.83 (d, J=9.0 Hz, 1H), 5.57 (d, J=9.0 Hz, 1H), 4.58 (s, 1H), 3.6-3.53 (m, 4H), 3.09 (d, J=18.6 Hz, 1H), 3.08-2.87 (m, 2H), 2.76-2.62 (dd, J=12.0, 4.8 Hz, 1H), 2.5-2.24 (m, 4H), 2.12 (s, 3H), 1.95 (dt, J=13.2, 5.4 Hz, 1H), 1.83 (dd, J=12.6, 2.4 Hz, 1H), 1.34 (dd, J=12.6, 6.6 Hz, 1H), 0.9-0.72 (m, 1H), 0.6-0.42 (m, 2H), 0.22-0.06 (m, 2H).

¹³C NMR (75 MHz, CDCl₃) δ [ppm] 209.3, 146.5, 137.6, 134.0, 127.5, 125.7, 119.9, 116.5, 94.8, 81.3, 59.7, 57.0, 52.9, 50.6, 48.4, 44.0, 43.2, 33.5, 30.1, 30.0, 23.2, 9.4, 4.1, 3.4.

### Example 27. Preparation of Compound HO—IIIB-MCP (Step C)

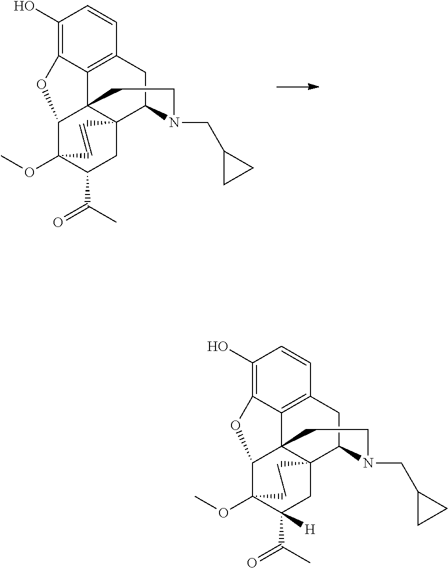 [SMILES: CO[C@]12C=C[C@@]3(C[C@@H]1C(C)=O)[C@H]1Cc4ccc(O)c5c4[C@@]3(CCN1CC1CC1)[C@H]2O5 ; CO[C@]12CC[C@@]3(C[C@@H]1C(C)=O)[C@H]1Cc4ccc(O)c5c4[C@@]3(CCN1CC1CC1)[C@H]2O5]

A 50 mL 3-neck round bottom flask was charged with Compound HO—II-MCP (800 mg, 1.963 mmol), tartaric acid (295 mg, 1.963 mmol), water (8 mL) and Pd/C (80 mg, 10% w/w). The mixture was then hydrogenated under 1 atmosphere of hydrogen at 80° C. for 12 h. The reaction mixture was filtered through Celite, while hot, and Celite was rinsed with some hot water. After cooling to room temperature, the pH of the aqueous solution was adjusted to 6.6-6.7 with 10% KOH. The aqueous solution was extracted with CHCl₃ (3×50 mL). The combined organic layers were dried over sodium sulfate, filtered off, and the solvent was removed under vacuum. Purification by flash chromatography (0 to 20% ethyl acetate in heptane) yielded Compound HO—IIIB-MCP (570 mg, 71%) as a white solid.

### 1-((4R,4aS,7R,7aR,12bS)-3-(Cyclopropylmethyl)-9-hydroxy-7-methoxy-1,2,3,4,5,6,7,7a-octahydro-4a,7-ethano-4,12-methanobenzofuro[3,2-e]isoquinolin-6-yl)ethan-1-one

HPLC-purity 92.5% at 215 nm.

MS (ES-API pos) m/z 410.2 (M+H).

¹H NMR (300 MHz, CDCl₃) δ [ppm] 6.7 (d, J=8.1 Hz, 1H), 6.52 (d, J=8.1 Hz, 1H), 4.49 (s, 1H), 3.41 (s, 3H), 3.11-3.01 (m, 2H), 2.96 (d, J=18.3 Hz, 1H), 2.74 (dt, J=13.5, 11.4, 3.9 Hz, 1H), 2.64 (dd, J=12.0, 5.1 Hz, 1H), 2.56-2.28 (m, 7H), 2.04 (dt, J=12.6, 5.7 Hz, 1H), 1.76-1.4 (m, 4H), 1.38-1.21 (m, 1H), 0.96-0.62 (m, 2H), 0.56-0.41 (m, 2H), 0.15-0.05 (m, 2H).

¹³C NMR (75 MHz, CDCl₃) δ [ppm] 210.9, 145.2, 137.4, 132.3, 128.1, 119.6, 116.6, 94.7, 77.8, 59.8, 58.3, 52.1, 49.5, 46.7, 43.7, 35.5, 35.1, 33.6, 30.4, 28.5, 22.8, 17.6, 9.4, 4.1, 3.3.

### Example 28. Preparation of Compound HO—IIIB-MCP (Step C)

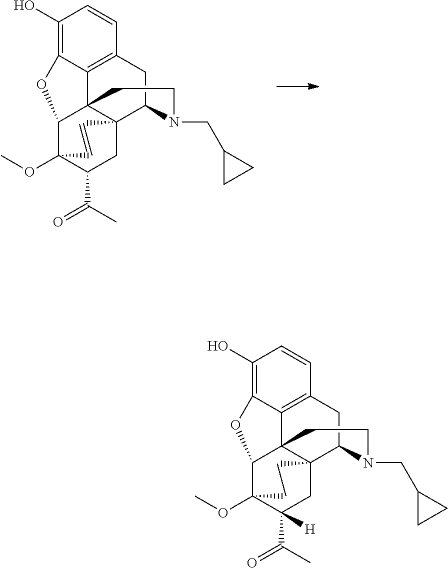 [SMILES: CO[C@]12C=C[C@@]3(C[C@@H]1C(C)=O)[C@H]1Cc4ccc(O)c5c4[C@@]3(CCN1CC1CC1)[C@H]2O5 ; CO[C@]12CC[C@@]3(C[C@@H]1C(C)=O)[C@H]1Cc4ccc(O)c5c4[C@@]3(CCN1CC1CC1)[C@H]2O5]

To a suspension of Compound HO—II-MCP (270 mg, 0.662 mmol) in a mixture of iPrOH (4.6 mL) and water (0.4 mL) was added Pd/C (30 mg, 10% w/w), at room temperature and under a nitrogen atmosphere. The mixture was then hydrogenated under 1 atmosphere of hydrogen at 80° C. overnight and was filtered off through Celite. Celite was rinsed with DCM. The filtrate was concentrated under vacuum and purification by flash chromatography (0 to 50% ethyl acetate in heptane) yielded Compound HO—IIIB-MCP (215 mg, 79%) as an off white solid.

### 1-((4R,4aS,7R,7aR,12bS)-3-(Cyclopropylmethyl)-9-hydroxy-7-methoxy-1,2,3,4,5,6,7,7a-octahydro-4a,7-ethano-4,12-methanobenzofuro[3,2-e]isoquinolin-6-yl)ethan-1-one

HPLC-purity 92.5% at 215 nm.

NMR and MS data were in agreement with those obtained with Example 27.

### Example 29. Preparation of Compound HO—IIIA-MCP (Step D)

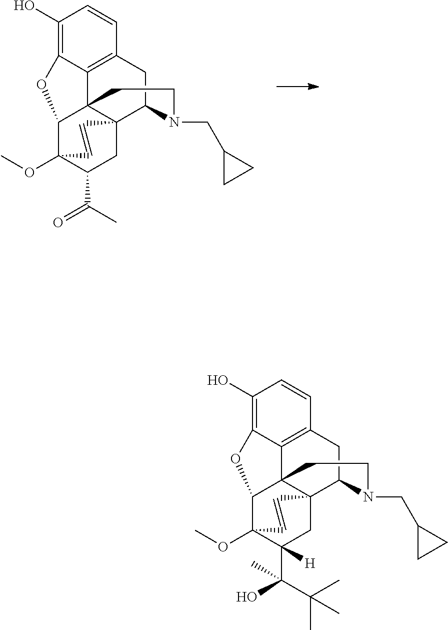 [SMILES: CO[C@]12C=C[C@@]3(C[C@@H]1C(C)=O)[C@H]1Cc4ccc(O)c5c4[C@@]3(CCN1CC1CC1)[C@H]2O5 ; CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]

Compound HO—II-MCP (750 mg, 1.84 mmol) dissolved in dioxane (8 mL) was added to a 2.0 M solution of tert-butylmagnesium chloride in ether (11 mL, 22 mmol) and TMEDA (3.31 mL, 22 mmol) dropwise, over 10 min, at room temperature and under a nitrogen atmosphere. Once the addition was complete the mixture was stirred at 60° C. for 4 h under a nitrogen atmosphere. The mixture was then cooled to 0° C. with an ice/water bath and carefully quenched with a saturated aqueous ammonium chloride solution over 15 min. Ethyl acetate (15 mL) was added. After separation the aqueous phase was extracted with ethyl acetate (2×15 mL). The combined organic layers were dried over sodium sulfate, filtered off and the solvents were removed under vacuum. Purification by flash chromatography (0 to 100% ethyl acetate in heptane) yielded Compound HO—IIIA-MCP (200 mg, 23%) as a white solid.

### (4R,4aR,7R,7aR,12bS)-3-(cyclopropylmethyl)-14-((S)-2-hydroxy-3,3-dimethylbutan-2-yl)-7-methoxy-1,2,3,4,7,7a-hexahydro-4a,7-ethano-4,12-methanobenzofuro[3,2-e]isoquinolin-9-ol

HPLC-purity 99.4% at 215 nm.

MS (ES-API pos) m/z 466.2 (M+1).

¹H NMR (300 MHz, CDCl₃) δ [ppm] 6.59 (d, 1H), 6.44 (d, 1H), 5.96 (d, 1H), 5.71 (s, 1H), 5.44 (d, 1H), 4.58 (s, 1H), 3.74 (s, 3H), 3.49 (d, 1H), 3.08 (d, 1H), 2.96 (dd, 1H), 2.66 (dd, 1H), 2.48-2.26 (m, 4H), 2.2-2.09 (t, 1H), 1.98-1.78 (m, 2H), 0.99 (s, 12H), 0.91-0.86 (m, 1H), 0.6-0.53 (m, 2H), 0.2-0.09 (m, 2H).

¹³C NMR (75 MHz, CDCl₃) δ [ppm] 146.6, 137.3, 135.6, 134.4, 127.8, 124.4, 119.7, 116.1, 99.3, 84.5, 78.7, 59.5, 56.7, 55.2, 47.4, 45.7, 44.1, 43.1, 39.6, 33.8, 32.1, 26.6, 23.1, 19.6, 9.4, 4.3, 3.1.

### Example 30. Preparation of buprenorphine (Step D)

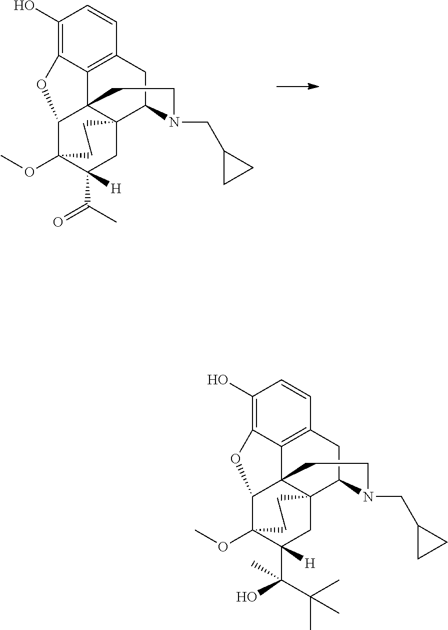 [SMILES: CO[C@]12CC[C@@]3(C[C@@H]1C(C)=O)[C@H]1Cc4ccc(O)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

To a stirred solution of Compound HO—IIIB-MCP (130 mg, 0.317 mmol) in a mixture of ether (11 mL) and toluene (5 mL), cooled to 0° C. with an ice/water bath and under a nitrogen atmosphere, was added a 2.0 M solution of tert-butylmagnesium chloride in ether (3.08 mL, 6.153 mmol) containing TMEDA (0.92 mL, 6.153 mmol) dropwise. After completion of the addition, the mixture was allowed to warm up to room temperature and was stirred for 1.5 h. The mixture was then poured into a mixture of ice/water (25 mL) and a saturated aqueous solution of ammonium chloride (25 mL). The aqueous phase was extracted with ethyl acetate (3×50 mL). The combined organic phases were dried over sodium sulfate, filtered off and the solvent was removed under vacuum. Purification by flash chromatography (0 to 100% ethyl acetate in heptane) yielded buprenorphine (99 mg, 41%) as a white solid.

### buprenorphine

HPLC-purity 98.9% at 215 nm.

MS (ES-API pos) m/z 468.3 (M+H).

¹H NMR (300 MHz, CDCl₃) δ [ppm] 6.67 (d, J=8.0 Hz, 1H), 6.49 (d, J=8.0 Hz, 1H), 6.02 (s, 1H), 5.78 (br, 1H), 4.43 (d, J=1.2 Hz, 1H), 3.51 (s, 3H), 3.01-2.82 (m, 3H), 2.6 (dd, J=11.9, 5.1 Hz, 1H), 2.38-2.21 (m, 3H), 2.20-2.10 (m, 2H), 1.97 (dt, J=12.6, 5.6 Hz, 1H), 1.9-1.7 (m, 2H), 1.65 (dd, J=12.8, 2.5 Hz, 1H), 1.36 (s, 3H), 1.29 (m, 1H), 1.12-0.96 (m, 10H), 0.9-0.63 (m, 2H), 0.56-0.4 (m, 2H), 0.2-0.07 (m, 2H).

¹³C NMR (75 MHz, CDCl₃) δ [ppm] 145.5, 137.4, 132.5, 128.1, 119.5, 116.5, 96.8, 80.8, 79.7, 59.5, 58.3, 52.5, 46.4, 43.7, 43.5, 40.3, 35.9, 35.6, 33.4, 29.6, 26.4, 22.8, 20.1, 18.2, 9.4, 4.1, 3.2.

### Example 31. Preparation of buprenorphine (Step D)

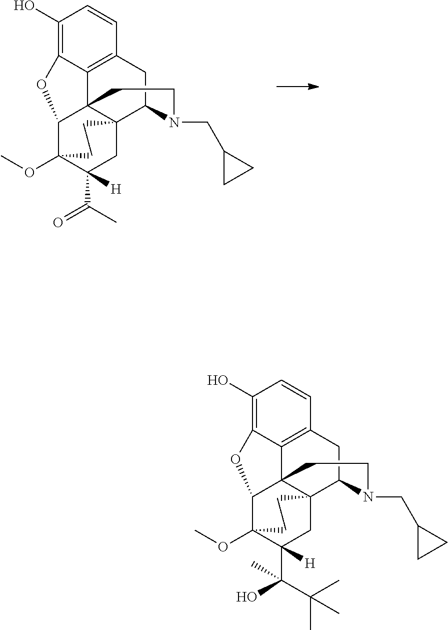 [SMILES: CO[C@]12CC[C@@]3(C[C@@H]1C(C)=O)[C@H]1Cc4ccc(O)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

To a stirred solution of Compound HO—IIIB-MCP (130 mg, 0.317 mmol) in a mixture of ether and toluene (3:2, 10 mL), cooled to 0° C. with an ice/water bath and under a nitrogen atmosphere, was added a 2.0 M solution of tert-butylmagnesium chloride in ether (2 mL, 4 mmol) dropwise. A white precipitate was obtained. The reaction mixture was allowed to warm to room temperature and the mixture was agitated for 15 h at room temperature. Water (10 mL) was carefully added to the reaction mixture, previously cooled to 0° C. with an ice/water bath, followed by the addition of a saturated aqueous solution of ammonium chloride (10 mL). The aqueous phase was extracted with ethyl acetate (3×50 mL). The combined organic phases were dried over sodium sulfate, filtered off and the solvent was removed under vacuum. Purification by flash chromatography (0 to 100% ethyl acetate in heptane) yielded buprenorphine (47 mg, 32%) as a white solid.

### buprenorphine

HPLC-purity 99.0% at 215 nm.

NMR and MS data were in agreement with those obtained from Example 30.

### Example 32. Preparation of buprenorphine (Step C)

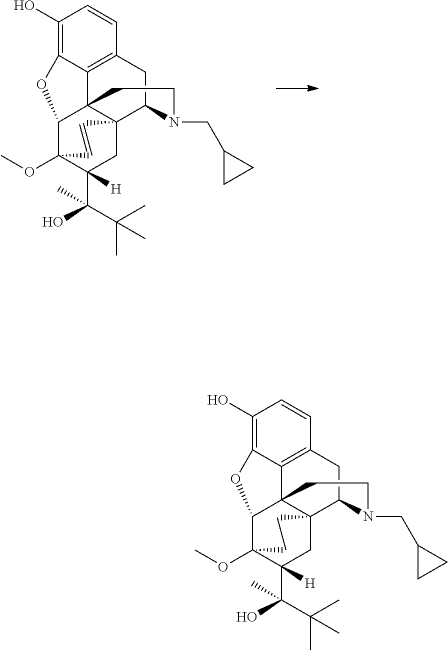 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

To a suspension of Compound HO—IIIA-MCP (250 mg, 0.537 mmol) in a mixture of isopropanol (4.6 mL) and water (0.4 mL) was added Pd/C (25 mg, 10% w/w) at room temperature. The mixture was then hydrogenated under 1 atmosphere of hydrogen at 80° C. overnight. The mixture was filtered through a plug of Celite and Celite was rinsed with CHCl₃. The mother liquor was concentrated under vacuum. Purification by flash chromatography (0 to 80% ethyl acetate in heptane) yielded intermediate buprenorphine (200 mg, 80%) was obtained as a white solid.

### buprenorphine

HPLC-purity 99.1% at 215 nm.

NMR and MS data were in agreement with those obtained for buprenorphine with method A and B previously reported

### Example 33. Preparation of Compound BnO-I-MCP (Step F)

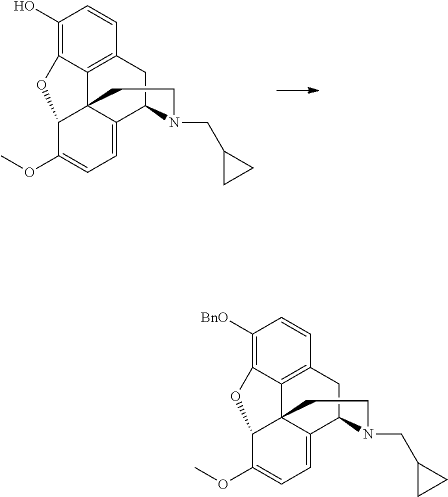 [SMILES: COC1=CC=C2[C@H]3Cc4ccc(O)c5c4[C@@]2(CCN3CC2CC2)[C@H]1O5 ; COC1=CC=C2[C@H]3Cc4ccc(OCc5ccccc5)c5c4[C@@]2(CCN3CC2CC2)[C@H]1O5]

To a solution of intermediate Compound HO—I-MCP (200 mg, 0.59 mmol) in DMF (5 mL) was added sodium hydride (36 mg, 0.89 mmol) at 0° C. and under a nitrogen atmosphere. The mixture was then stirred at 45° C. for 20 min and was cooled to 0° C. Benzyl bromide (130 mg, 0.741 mmol) was added and the mixture was stirred overnight at room temperature. The mixture was cooled to 0° C. with an ice/water bath and water (25 mL) was carefully added. The aqueous mixture was extracted with CHCl₃ (3×25 mL). The combined organic layers were washed with water (25 mL), brine (50 mL), dried over sodium sulfate, filtered off and the solvents were removed under vacuum. Purification by flash chromatography (0 to 5% MeOH in DCM) yielded Compound BnO-I-MCP (190 mg, 68%) as an orange/brownish oil.

### (4R,7aR,12bS)-9-(Benzyloxy)-3-(cyclopropylmethyl)-7-methoxy-2,3,4,7a-tetrahydro-1H-4,12-methanobenzofuro[3,2-e]isoquinoline

HPLC-purity 96.8% at 215 nm.

MS (ES-API pos) m/z 428.2 (M+H).

¹H NMR (300 MHz, CDCl₃) δ [ppm] 7.95 (br, 1H from DMF), 7.4 (d, 2H), 7.35-7.2 (m, 3H), 6.65 (d, 1H), 6.5 (d, 1H), 5.54 (d, 1H), 5.27 (s, 1H), 5.13 (dd, 2H), 5.02 (d, 1H), 3.94 (d, 1H), 3.55 (s, 3H), 3.26 (d, 1H), 2.95-2.77 (m, 2H+DMF), 2.7 (dd, 1H), 2.5 (d, 2H), 2.18 (dt, 1H), 1.7 (d, 1H), 1.00-0.8 (m, 1H), 0.6-0.47 (m, 2H), 0.2-0.1 (m, 2H).

¹³C NMR (75 MHz, CDCl₃) δ [ppm] 152.6, 145.1, 141.6, 137.5, 133.7, 131.7, 128.3, 128.0, 127.7, 127.6, 119.3, 115.8, 112.4, 95.9, 89.0, 71.6, 58.8, 58.6, 54.9, 46.3, 44.1, 36.4, 36.3, 30.8, 9.2, 4.0, 3.8.

### (4R,7aR,12bS)-9-(Benzyloxy)-3-(cyclopropylmethyl)-7-methoxy-2,3,4,7a-tetrahydro-1H-4,12-methanobenzofuro[3,2-e]isoquinoline

HPLC-purity 96.4% at 215 nm.

NMR and MS data were in agreement with those obtained for Example 33.

### Example 35. Preparation of Compound BnO—II-MCP (Step F)

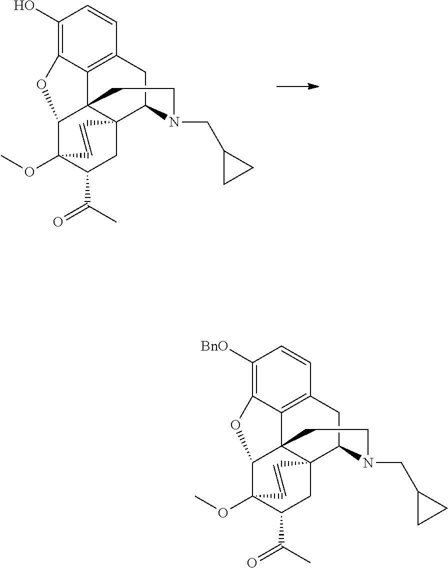 [SMILES: CO[C@]12C=C[C@@]3(C[C@@H]1C(C)=O)[C@H]1Cc4ccc(O)c5c4[C@@]3(CCN1CC1CC1)[C@H]2O5 ; CO[C@]12C=C[C@@]3(C[C@@H]1C(C)=O)[C@H]1Cc4ccc(OCc5ccccc5)c5c4[C@@]3(CCN1CC1CC1)[C@H]2O5]

To a suspension of Compound BnO-I-MCP (240 mg, 0.59 mmol) in CHCl₃ (3 mL) were added benzyl bromide (0.093 mL, 0.78 mmol) and potassium carbonate (450 mg, 3.26 mmol) at room temperature under a nitrogen atmosphere. The reaction mixture was then refluxed for 15 h. The mixture was cooled down to room temperature and filtered off. The solid was washed with DCM and the filtrate was concentrated under vacuum. Purification by flash chromatography (0 to 50% ethyl acetate in heptane) yielded Compound BnO—II-MCP (270 mg, 92%) as a colorless oil.

### 1-((4R,4aR,7R,7aR,12bS)-9-(Benzyloxy)-3-(cyclopropylmethyl)-7-methoxy-1,2,3,4,7,7a-hexahydro-4a,7-ethano-4,12-methanobenzofuro[3,2-e]isoquinolin-14-yl)ethan-1-one

HPLC-purity 96.4% at 215 nm.

MS (ES-API pos) m/z 498.4 (M+H).

¹H NMR (300 MHz, CDCl₃) δ [ppm] 7.45-7.23 (m, 5H), 6.65 (d, 1H), 6.47 (d, 1H), 5.91 (d, 1H), 5.59 (d, 1H), 5.19-5.05 (dd, 2H), 4.59 (s, 1H), 3.61 (s, 3H), 3.55 (d, 1H), 3.15-2.86 (m, 3H), 2.75-2.65 (dd, 1H), 2.47-2.28 (m, 4H), 2.15 (s, 3H), 2.05-1.91 (dt, 1H), 1.89-1.8 (dd, 1H), 1.71-1.58 (m, 1H), 1.4-1.31 (dd, 1H), 0.91-0.75 (m, 3H), 0.58-0.42 (m, 2H), 0.18-0.08 (m, 2H).

¹³C NMR (75 MHz, CDCl₃) δ [ppm] 209.3, 148.8, 140.6, 137.5, 136.5, 134.6, 129.0, 128.3, 127.7, 127.5, 125.5, 119.4, 116.7, 95.8, 81.4, 72.0, 59.8, 57.0, 53.7, 53.7, 50.8, 48.1, 43.9, 43.2, 33.6, 30.6, 29.9, 23.3, 9.4, 4.1, 3.4.

### Example 36. Preparation of Compound BnO—II-MCP (Step B)

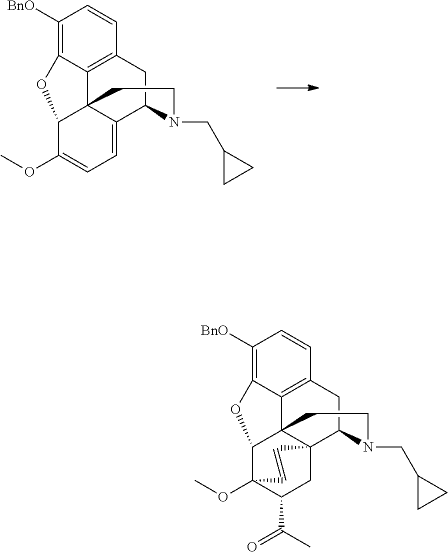 [SMILES: COC1=CC=C2[C@H]3Cc4ccc(OCc5ccccc5)c5c4[C@@]2(CCN3CC2CC2)[C@H]1O5 ; CO[C@]12C=C[C@@]3(C[C@@H]1C(C)=O)[C@H]1Cc4ccc(OCc5ccccc5)c5c4[C@@]3(CCN1CC1CC1)[C@H]2O5]

To a solution of Compound BnO-I-MCP (190 mg, 0.415 mmol) in toluene (3 mL) was added methyl vinyl ketone (0.73 mL, 8.35 mmol) at room temperature and under a nitrogen atmosphere. The mixture was stirred at 80° C. for 15 h and the volatiles were removed under vacuum. Purification by flash chromatography (0 to 60% ethyl acetate in heptane) yielded Compound BnO—II-MCP (170 mg, 82%) as a colorless oil.

### 1-((4R,4aR,7R,7aR,12bS)-9-(Benzyloxy)-3-(cyclopropylmethyl)-7-methoxy-1,2,3,4,7,7a-hexahydro-4a,7-ethano-4,12-methanobenzofuro[3,2-e]isoquinolin-14-yl)ethan-1-one

HPLC-purity 92.9% at 215 nm.

NMR and MS data were in agreement with those obtained for Example 35.

### Example 37. Preparation of Compound BnO—IIIA-MCP (Step D)

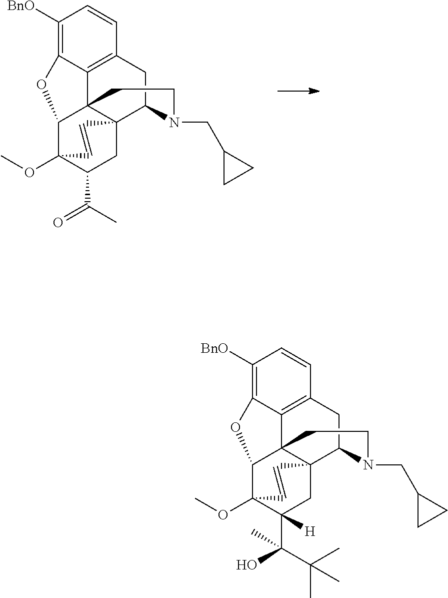 [SMILES: CO[C@]12C=C[C@@]3(C[C@@H]1C(C)=O)[C@H]1Cc4ccc(OCc5ccccc5)c5c4[C@@]3(CCN1CC1CC1)[C@H]2O5 ; CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5]

To a solution of Compound BnO—II-MCP (250 mg, 0.5 mmol) in dry toluene (6 mL) at room temperature and under a nitrogen atmosphere, was added a 1.7 M tert-butylmagnesium chloride solution in THF (1.77 mL, 3 mmol) dropwise. The mixture was stirred at room temperature for 18 h prior to further dropwise addition of a 1.7 M tert-butylmagnesium chloride solution in THF (1.77 mL, 3 mmol). The reaction mixture was stirred for 5 h and was poured into a mixture made of ice/water (50 mL) and of an ammonium chloride saturated aqueous solution (50 mL). The mixture was extracted with toluene (3×50 mL). The combined organic layers were washed with brine (50 mL), dried over sodium sulfate, filtered off and the solvents were removed under vacuum. Purification by flash chromatography (0 to 20% ethyl acetate in heptane) yielded Compound BnO—IIIA-MCP (107 mg, 38%) as a colorless oil.

### (2S)-2-((4R,4aR,7R,7aR,12bS)-9-(Benzyloxy)-3-(cyclopropylmethyl)-7-methoxy-1,2,3,4,7,7a-hexahydro-4a,7-ethano-4,12-methanobenzofuro[3,2-e]isoquinolin-14-yl)-3,3-dimethylbutan-2-ol

HPLC-purity 97.2% at 215 nm.

MS (ES-API pos) m/z 556.4 (M+H).

¹H NMR (300 MHz, CDCl₃) δ [ppm] 7.43-7.3 (m, 5H), 6.65 (d, 1H), 6.46 (d, 1H), 6.00 (d, 1H), 5.65 (s, 1H), 5.43 (d, 1H), 5.19-5.04 (dd, 2H), 4.58 (s, 1H), 3.79 (s, 3H), 3.5 (d, 1H), 3.1 (d, 1H), 2.9 (dd, 1H), 2.69 (dd, 1H), 2.47-2.3 (m, 4H), 2.21-2.12 (t, 1H), 2.01-1.82 (m, 2H), 1.55 (s, 3H), 1.01 (s, 9H), 0.99-0.8 (m, 2H), 0.62-0.43 (m, 2H), 0.22-0.12 (m, 2H).

¹³C NMR (75 MHz, CDCl₃) δ [ppm] 148.9, 140.6, 137.8, 137.6, 135.6, 135.1, 129.2, 129.0, 128.4, 128.2, 127.7, 127.5, 125.3, 124.7, 119.4, 116.7, 99.0, 84.5, 78.4, 72.1, 67.9, 59.5, 56.7, 55.2, 47.1, 45.9, 44.1, 43.1, 39.7, 34.0, 32.2, 26.7, 25.6, 23.2, 19.6, 9.5, 4.3, 3.2.

### Example 38. Preparation of buprenorphine (Step C)

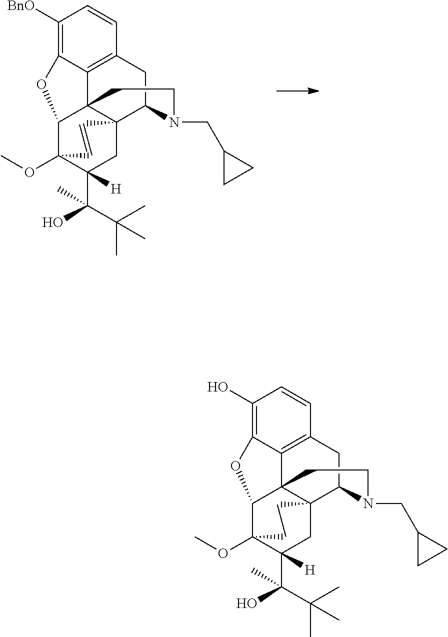 [SMILES: CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

To a solution of Compound BnO—IIIA-MCP (194 mg, 0.349 mmol) in a mixture of isopropanol (4.6 mL) and water (0.4 mL) was added Pd/C (20 mg, 10% w/w) at room temperature and under a nitrogen atmosphere. The mixture was then hydrogenated under 1 atmosphere of hydrogen at 80° C. for 15 min. The mixture was filtered through Celite with isopropanol and CHCl₃ used as eluents. The solvents were removed under vacuum. Purification by flash chromatography (0 to 60% ethyl acetate in heptane) yielded buprenorphine (115 mg, 70%) as a white solid.

### buprenorphine

HPLC-purity 96.3% at 215 nm.

NMR and MS data were in agreement with those obtained for Examples 21-22 and 30-32.

### Example 39. Preparation of buprenorphine-HCl from buprenorphine

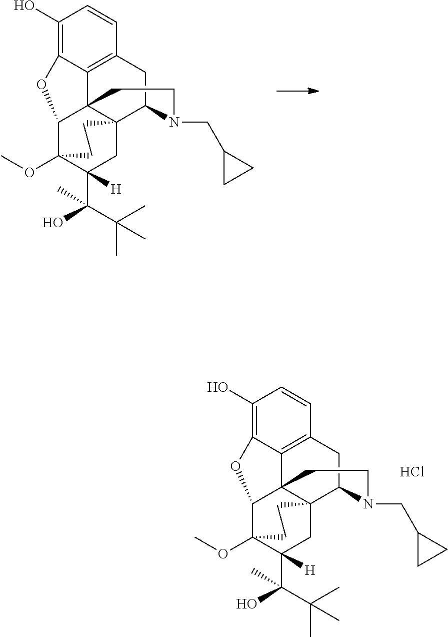 [SMILES: CO[C@]12CC[C@@]3(C[C@@H]1[C@](C)(O)C(C)(C)C)[C@H]1Cc4ccc(O)c5c4[C@@]3(CCN1CC1CC1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5 ; Cl]

Buprenorphine (100 mg, 0.21 mmol) was taken in EtOH (2 mL) and the mixture was heated until all solid had dissolved. To the warm solution was added 0.5 mL of a mixture of 95 mL EtOH and 5 mL 37% hydrochloric acid (approx. 0.3 mmol). The solution was cooled in the fridge overnight during which time crystals were formed.

The crystals were collected and dried under vacuum at 50° C. to yield buprenorphine hydrochloride (102 mg, 96%).

### buprenorphine-HCl

HPLC-purity 99.4% at 215 nm.

DSC-Melting point 267.84-275.26° C.

MS (ES-API pos) m/z 468.2 (M free base+H).

¹H NMR (300 MHz, CDCl₃/CD₃OD) δ [ppm] 6.68 (d, J=8.2 Hz, 1H), 6.50 (d, J=8.2 Hz, 1H), 4.44 (s, 1H), 3.82 (d, J=6.5 Hz, 1H), 3.47 (s, 3H), 3.18-3.35 (m, 4H), 3.0 (d, J=9.5 Hz, 1H), 2.70-2.88 (m, 3H), 2.40 (dt, J=5 and 14 Hz, 1H), 2.22 (t, J=8.8 Hz, 1H), 1.63-1.90 (m, 3H), 1.50 (dd, J=8 and 14 Hz, 1H), 1.29 (s, 3H), 1.20-1.25 (m, 1H), 1.03-1.18 (m, 1H), 1.00 (s, 9H), 0.60-0.85 (m, 4H), 0.38 (m, 1H).

### Example 40. Preparation of Compound BnO—I—Bn (Step F)

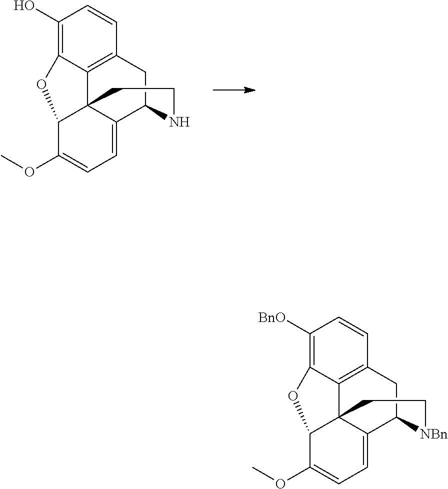 [SMILES: COC1=CC=C2[C@H]3Cc4ccc(O)c5c4[C@@]2(CCN3)[C@H]1O5 ; COC1=CC=C2[C@H]3Cc4ccc(OCc5ccccc5)c5c4[C@@]2(CCN3Cc2ccccc2)[C@H]1O5]

A 500 mL flask was charged with nororipavine (5.66 g, 20 mmol), MeOH (100 mL), and water (50 mL). The suspension was stirred at room temperature and NaOH-pellets (2.50 g, 60 mmol, 3 equiv) were added. After 10 min a light brown solution was obtained and benzyl bromide (8.50 g, 50 mmol, 2.5 equiv) was added over a period of 1 min. A slight exotherm was observed and after 10 min a precipitate was formed. After 2 h the mixture was rotary evaporated to remove most of the MeOH (65 mL). The residue (approximately 100 mL) was cooled in ice-water for 15 min and then filtered. The solid was washed with water (2×10 mL), then with MeOH (10 mL), and dried under vacuum to afford Compound BnO—I—Bn (8.6 g, 93%).

### N,O-Dibenzyl-nororipavine

HPLC-purity 95.7% at 254 nm.

MS (ES-API pos) m/z 464.4 (M+H).

¹H NMR (300 MHz, CDCl₃) δ [ppm] 7.49-7.24 (m, 10H), 6.68 (d, J=8.1 Hz, 1H), 6.54 (d, J=8.2 Hz, 1H), 5.49 (d, J=6.4 Hz, 1H), 5.32 (s, 1H), 5.22 (d, J=12.2 Hz, 1H), 5.15 (d, J=12.1 Hz, 1H), 5.06 (d, J=6.4 Hz, 1H), 3.77 (d, J=2.9 Hz, 2H), 3.63 (s, 4H), 3.33 (d, J=18.0 Hz, 1H), 2.96 (td, J=13.0, 3.5 Hz, 1H), 2.72 (m, 2H), 2.26 (td, J=12.6, 4.9 Hz, 1H), 1.70 (dd, J=12.6, 3.0 Hz, 1H).

¹³C NMR (75 MHz, CDCl₃) δ [ppm] 152.6, 145.7, 141.7, 138.7, 137.6, 132.9, 132.5, 129.0, 128.4, 127.7, 127.6, 127.1, 119.3, 115.9, 111.8, 96.0, 89.2, 71.7, 58.3, 58.2, 55.0, 46.6, 44.1, 36.5, 31.7.

### Example 41. Preparation of Compound BnO—II-Bn (Step B)

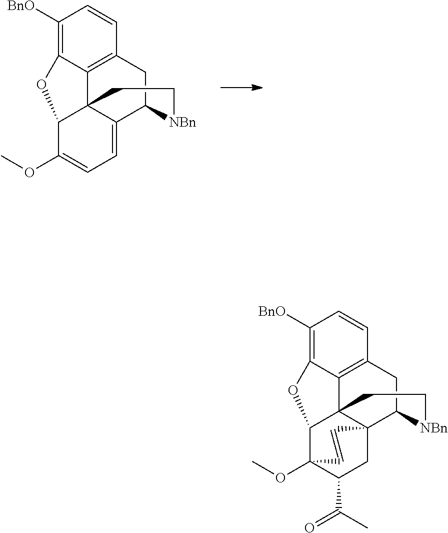 [SMILES: COC1=CC=C2[C@H]3Cc4ccc(OCc5ccccc5)c5c4[C@@]2(CCN3Cc2ccccc2)[C@H]1O5 ; CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]

A solution of Compound BnO—I—Bn (4.63 g, 10.0 mmol) and methyl vinyl ketone (8 mL, 100 mmol) in toluene (50 mL) was heated at 80° C. for 16 h. After cooling to room temperature the mixture was concentrated under vacuum to give a brown oily residue (5.5 g), which was purified by column chromatography (120 g SiO₂, elution with 0-20% EtOAc in heptane, R_{f} 0.3) to afford Compound BnO—II-Bn as a colorless solid (4.25 g, 77% yield).

### 7α-Acetyl-N,O-dibenzyl-6,14-endo(etheno)tetrahydro-nororipavine

HPLC-purity 97.3% at 215 nm.

MS (ES-API pos) m/z 534.4 (M+H).

¹H NMR (300 MHz, CDCl₃) δ [ppm] 7.45-7.20 (m, 10H), 6.68 (d, J=8.1 Hz, 1H), 6.51 (d, J=8.2 Hz, 1H), 5.89 (dt, J=8.9, 1.2 Hz, 1H), 5.53 (d, J=8.8 Hz, 1H), 5.13 (d, J=5.4 Hz, 2H), 4.60 (d, J=1.5 Hz, 1H), 3.66 (s, 2H), 3.62 (s, 3H), 3.27 (dd, J=12.5, 6.0 Hz, 2H), 3.09 (dd, J=12.6, 9.4 Hz, 1H), 2.95 (dd, J=9.4, 6.5 Hz, 1H), 2.67-2.38 (m, 3H), 2.16 (s, 3H), 2.00 (td, J=12.5, 5.9 Hz, 1H), 1.87 (ddd, J=13.1, 4.0, 1.8 Hz, 1H), 1.35 (dd, J=12.6, 6.5 Hz, 1H).

¹³C NMR (75 MHz, CDCl₃) δ [ppm] 209.35, 148.84, 140.76, 139.09, 137.57, 136.20, 134.56, 128.86, 128.65, 128.38, 127.77, 127.53, 127.10, 125.62, 119.54, 116.84, 95.69, 81.33, 72.08, 59.50, 57.04, 53.70, 50.98, 48.09, 43.81, 43.35, 33.60, 30.56, 29.89, 23.53.

### Example 42. Preparation of Compound BnO—IIIA-Bn (Step D)

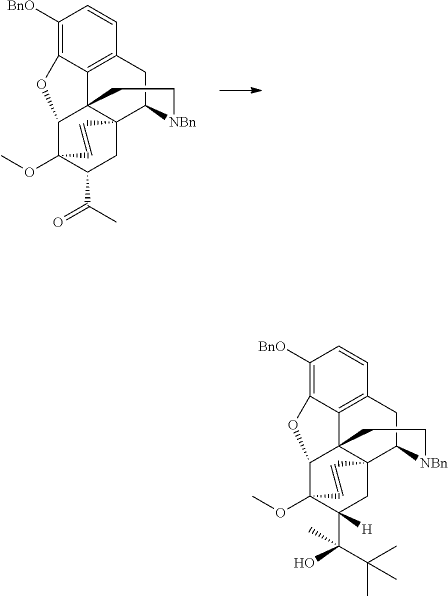 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5 ; CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]

A 50 mL flask was charged with a solution of tert-butylmagnesium chloride (1.7 M solution in THF, 5 mL, 8.5 mmol) and toluene (8 mL). The THF was evaporated in vacuo and to the residual Grignard solution in toluene (approximately 10 mL) was added a solution of Compound BnO—II-Bn (0.70 g, 1.3 mmol) in dry toluene (8 mL). The reaction mixture was heated to 60° C. for 2 h and then cooled in an ice-water bath and quenched by addition of 10% aqueous ammonium chloride (25 mL). The layers were separated and the aqueous layer was extracted with toluene (3×25 mL). The combined organic layers were washed with brine, dried with sodium sulfate, and concentrated to an oil. Purification by column chromatography (120 g SiO₂, elution with 0-20% EtOAc in heptane, R_{f} 0.6) afforded Compound BnO—III-Bn as white solid (0.38 g, 50%).

### N,O-Dibenzyl-7α-(2-(S)-hydroxy-3,3-dimethyl-2-butyl)-6,14-endo(etheno)tetrahydro-nororipavine (3)

HPLC-purity 95.6% at 215 nm.

MS (ES-API pos) m/z 492.4 (M+H).

¹H NMR (300 MHz, CDCl₃) δ [ppm] 7.43-7.30 (m, 10H), 6.66 (d, J=8.1 Hz, 1H), 6.48 (d, J=8.2 Hz, 1H), 5.95 (d, J=8.9 Hz, 1H), 5.60 (s, 1H), 5.34 (d, J=8.9 Hz, 1H), 5.14 (d, J=12.0 Hz, 1H), 5.07 (d, J=12.0 Hz, 1H), 4.58 (d, J=1.4 Hz, 1H), 3.76 (s, 3H), 3.68 (d, J=2.7 Hz, 2H), 3.22 (d, J=12 Hz, 1H), 3.17-3.01 (m, 2H), 2.70-2.52 (m, 2H), 2.39 (dd, J=18.5, 6.6 Hz, 1H), 2.17 (t, J=8.6 Hz, 1H), 1.99 (td, J=12.1, 11.3, 6.1 Hz, 1H), 1.89 (d, J=12.6 Hz, 1H), 1.04 (s, 9H), 0.98 (s, 3H), 1.01-0.82 (m, 1H).

¹³C NMR (75 MHz, CDCl₃) δ [ppm] 148.89, 140.64, 139.37, 137.56, 135.31, 135.00, 128.93, 128.61, 128.38, 128.32, 127.78, 127.46, 127.06, 124.71, 119.44, 116.71, 98.94, 84.46, 78.34, 72.11, 59.10, 56.04, 55.21, 47.00, 45.92, 44.28, 43.14, 39.70, 34.08, 32.22, 26.64, 23.39, 19.57

### Example 43. Preparation of Compound HO—IV—H (Step C)

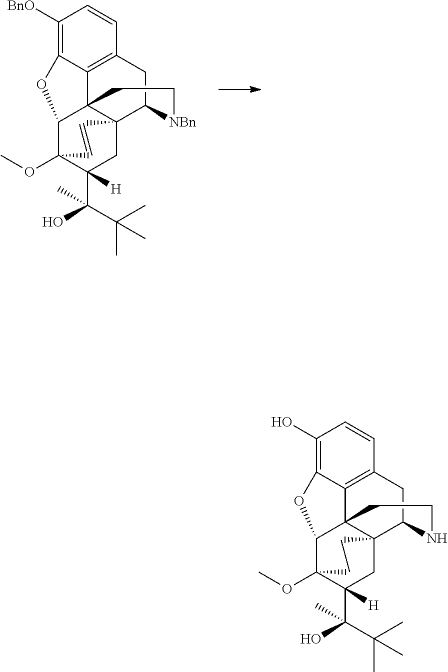 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5 ; CO[C@]12CC[C@@]3(C[C@@H]1[C@](C)(O)C(C)(C)C)[C@H]1Cc4ccc(O)c5c4[C@@]3(CCN1)[C@H]2O5]

A vigorously stirred mixture of Compound BnO—III-Bn (355 mg, 0.6 mmol), and Pd/C (10%, 30 mg) in iPrOH (10 mL), water (0.2 mL), and acetic acid (0.1 mL) was hydrogenated at 60° C. for 16 h under 1 atmosphere of hydrogen. IPC NMR showed that both benzyl groups were removed and the double bond was only partly reduced.

The catalyst was refreshed and hydrogenation was continued at 80° C. for 60 h. ICP NMR showed no more double bond signals. The mixture was filtered over Celite. The filter was flushed with iPrOH and DCM. The filtrate was concentrated to give Compound HO—IV—H as acetate salt (300 mg, 100%).

### norbuprenorphine

HPLC-purity 89% at 215 nm.

MS (ES-API pos) m/z 414.3 (M+H).

¹H NMR (300 MHz, CDCl₃) δ [ppm] 7.64 (br s, 2H), 6.76 (d, J=8.0 Hz, 1H), 6.49 (d, J=8.1 Hz, 1H), 5.80 (br s, 1H), 4.40 (s, 1H), 3.59 (d, J=6.4 Hz, 1H), 3.51 (s, 3H), 3.35-3.25 (m, 2H), 3.04 (t, J=13.5 Hz, 1H), 2.88 (dd, J=19.2, 6.4 Hz, 1H), 2.75 (t, J=13.5 Hz, 1H), 2.22-2.07 (m, 2H), 2.01 (s, 3H), 1.90-1.70 (m, 3H), 1.52 (dd, J=13.1, 9.0 Hz, 1H), 1.33 (s, 3H), 1.18 (m, 1H), 1.03 (s, 9H), 0.76 (t, J=12.3 Hz, 1H).

¹³C NMR (75 MHz, CDCl₃) δ [ppm] 145.91, 139.04, 129.99, 123.75, 120.29, 118.23, 95.53, 79.85, 79.62, 53.66, 52.69, 45.00, 42.97, 40.34, 34.40, 32.1, 31.8, 29.9, 29.1, 26.23, 22.9, 20.13, 17.8.

### Example 44. Preparation of buprenorphine (Step A1)

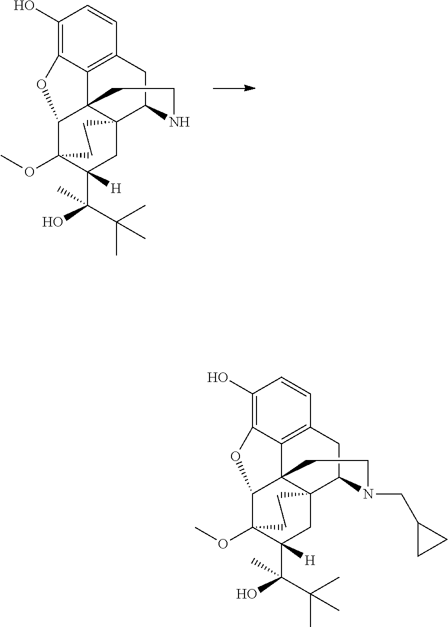 [SMILES: CO[C@]12CC[C@@]3(C[C@@H]1[C@](C)(O)C(C)(C)C)[C@H]1Cc4ccc(O)c5c4[C@@]3(CCN1)[C@H]2O5 ; CO[C@]12CC[C@@]3(C[C@@H]1[C@](C)(O)C(C)(C)C)[C@H]1Cc4ccc(O)c5c4[C@@]3(CCN1CC1CC1)[C@H]2O5]

A 50 mL flask was charged with Compound HO—I—H (210 mg, 0.44 mmol), cyclopropane carboxaldehyde (80 μL, 1 mmol), dichloro(p-cymene)ruthenium(II) dimer (10 mg, 0.016 mmol), triethylamine (0.42 mL, 3.1 mmol), and acetonitrile (5 mL). The mixture was stirred under nitrogen at room temperature and formic acid (0.24 mL, 6.2 mmol) was added dropwise. The resulting mixture was heated at 60° C. for 1 h. The mixture was cooled to room temperature and concentrated under vacuum. The residue was partitioned between toluene and 1 N aqueous NaOH. The aqueous layer was extracted twice with toluene. The combined organic layers were washed with brine, dried on sodium sulfate, and concentrated under vacuum to afford buprenorphine (160 mg, 78%).

### buprenorphine

HPLC-purity 85.6% at 215 nm.

MS and NMR data were in agreement with those obtained in previous examples

### Example 45. Preparation of Compound HO—I—Ac (Step G)

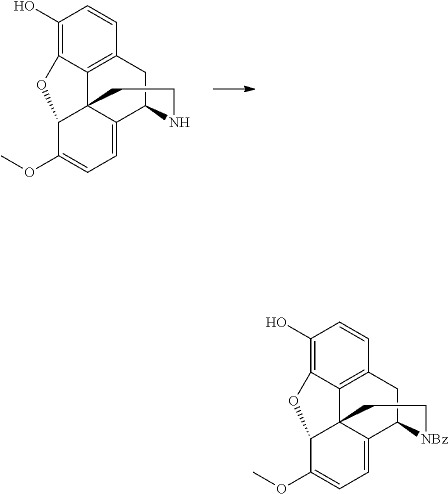

Under a nitrogen atmosphere benzoyl chloride (0.45 mL, 3.88 mmol) was added slowly to a stirred mixture of nororipavine (1.00 g, 3.53 mmol) and triethylamine (0.59 mL) in dichloromethane (10 mL). The resulting mixture was stirred for 50 minutes at room temperature. Dichloromethane (20 mL) was added. The mixture was extracted with water (2×10 mL). The organic layer was dried over Na₂SO₄, filtered and concentrated under reduced pressure. The residue was purified by column chromatography (4 g of silica, 0-60% EtOAc in heptanes) to afford Compound HO—I—Ac (0.82 g, 60%).

### ((12bS)-9-hydroxy-7-methoxy-1,2,4,7a-tetrahydro-3H-4,12-methanobenzofuro[3,2-e]isoquinolin-3-yl)(phenyl)methanone

MS (ES-API pos) m/z 388.3 (M+H).

¹H NMR (300 MHz, CDCl₃) δ [ppm] 7.44 and 7.40 (2×s, 5H), 6.69 (d, J=8.2 Hz, 1H), 6.59 and 6.54 (2×d, J=8.2 Hz, 1H), 5.76 (m, 1H), 5.54 (s, 1H), 5.33 (d, J=7.6 Hz, 1H), 5.11 (d, J=5.9 Hz, 0.5H), 5.02 (d, J=6.5 Hz, 0.5H), 4.69 (m, 1H), 3.70-3.51 (m, 1H), 3.62 (s, 3H), 3.28-2.95 (m, 3H), 2.25-1.60 (m, 2H).

### Example 46. Preparation of Compound BnO—I—Ac (Step F)

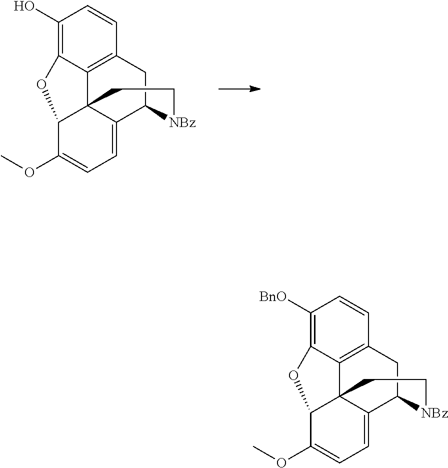

Under a nitrogen atmosphere a mixture of Compound BnO—I—Ac (826 mg, 2.13 mmol), benzyl bromide (0.38 mL, 3.20 mmol) and potassium carbonate (589 mg, 4.26 mmol) in acetone (6 mL) was heated to reflux for 18 h. The solvent was removed under reduced pressure. Water (20 mL) was added and the mixture was extracted with EtOAc (2×20 mL). The combined extracts were dried over Na₂SO₄, filtered and concentrated under reduced pressure. The residue was stirred with heptanes. The solvent was decanted and the residue was dried under reduced pressure at 50° C. to afford Compound BnO—I—Ac (1.13 g, quantitative yield).

### ((12bS)-9-(benzyloxy)-7-methoxy-1,2,4,7a-tetrahydro-3H-4,12-methanobenzofuro[3,2-e]isoquinolin-3-yl)(phenyl)methanone

MS (ES-API pos) m/z 478.3 (M+H).

¹H NMR (300 MHz, CDCl₃) δ [ppm] 8.22-7.28 (m, 10H), 6.71 (d, J=8.2 Hz, 1H), 6.58 and 6.52 (2×d, J=8.2 Hz, 1H), 5.77 (m, 1H), 5.34 (d, J=8.8 Hz, 1H), 5.19 (m, 2H), 5.10 (d, J=5.9 Hz, 0.5H), 5.01 (d, J=6.5 Hz, 0.5H), 4.69 (m, 1H), 3.70-3.46 (m, 1H), 3.64 (s, 3H), 3.31-2.95 (m, 3H), 2.21-1.65 (m, 2H).

### Example 47. Preparation of Compound BnO—I—Bn (Step H)

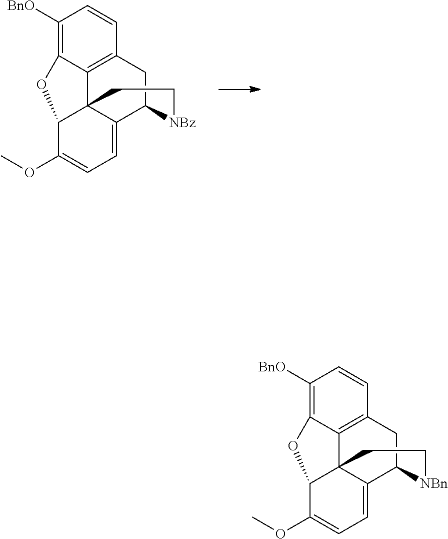

Under a nitrogen atmosphere lithium aluminium hydride (162 mg, 4.26 mmol) was added to a stirred solution of Compound BnO—I—Ac (1.02 g, 2.13 mmol) in THF (15 mL). The mixture was heated at 60° C. for 1.5 h. Water (0.16 mL), 15% aqueous NaOH (0.16 mL) and water (0.48 mL) were added. After stirring for 15 minutes EtOAc was added and the mixture was filtered over a pad of Celite. The filtrate was concentrated under reduced pressure and the residue was purified by column chromatography (25 g of silica, 0-90% EtOAc in heptanes to afford Compound BnO—I-Bn (694 mg, 70%) as an off-white solid.

### (12bS)-3-benzyl-9-(benzyloxy)-7-methoxy-2,3,4,7a-tetrahydro-1H-4,12-methanobenzofuro[3,2-e]isoquinoline

MS (ES-API pos) m/z 464.3 (M+H).

¹H NMR (300 MHz, CDCl₃) δ [ppm] 7.58-7.16 (m, 10H), 6.68 (d, J=8.1 Hz, 1H), 6.54 (d, J=8.2 Hz, 1H), 5.49 (d, J=6.4 Hz, 1H), 5.32 (s, 1H), 5.26-5.10 (m, 2H), 5.06 (d, J=6.4 Hz, 1H), 3.76 (m, 2H), 3.63 (s, 3H+m, 1H), 3.32 (d, J=17.9 Hz, 1H), 2.95 (td, J=13.0, 3.5 Hz, 1H), 2.76-2.66 (m, 2H), 2.26 (td, J=12.6, 5.0 Hz, 1H), 1.69 (d, J=12.3 Hz, 1H).

MS and NMR data were in agreement with those obtained in previous examples

### Example 48. Preparation of Compound AcO—I—Ac (Step G)

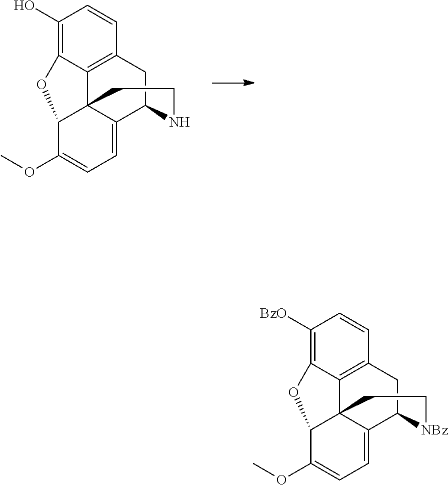

Under a nitrogen atmosphere benzoyl chloride (1.8 mL, 15.5 mmol) was added slowly to a stirred mixture of nor-oripavine (2.00 g, 7.06 mmol) and triethylamine (2.3 mL, 16.9 mmol) in dichloromethane (10 mL), while cooling in an ice-bath. The cooling bath was removed and the mixture was stirred at room temperature for 1.5 h. Dichloromethane (65 mL) was added and the mixture was extracted with water (2×30 mL). The organic layer was dried over Na₂SO₄, filtered and concentrated under reduced pressure. The residue was purified by column chromatography (40 g of silica, 0-85% EtOAc in heptanes) to afford Compound AcO—I—Ac (2.93 g, 84%).

### (12bS)-3-benzoyl-7-methoxy-2,3,4,7a-tetrahydro-1H-4,12-methanobenzofuro[3,2-e]isoquinolin-9-yl benzoate

MS (ES-API pos) m/z 492.2 (M+H).

¹H NMR (300 MHz, CDCl₃) δ [ppm] 8.20 (d, J=7.1 Hz, 2H), 7.62 (m, 1H), 7.51-7.42 (m, 7H), 6.94 (d, J=8.2 Hz, 1H), 6.73 and 6.68 (2×d, J=8.2 Hz, 1H), 5.80 (m, 1H), 5.36 (m, 1H), 5.11 (d, J=5.9 Hz, 0.5H), 5.02 (d, J=5.3 Hz, 0.5H), 4.73 (m, 1H), 3.73-3.49 (m, 1H), 3.61 (s, 3H), 3.37-3.03 (m, 3H), 2.26-1.82 (m, 2H).

### Example 49. Preparation of Compound AcO—II—Ac (Step B)

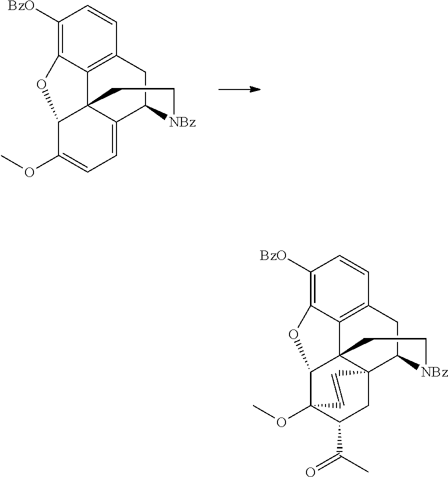

Under a nitrogen atmosphere a mixture of Compound AcO—I—Ac (2.93 g, 5.96 mmol) and methyl vinyl ketone (3.9 mL, 47.7 mmol) in toluene (25 mL) was heated at 80° C. for 16 h. After standing for 2 days at room temperature methyl vinyl ketone (3.9 mL, 47.7 mmol) was added. The mixture was heated at 80° C. for 16 h. The solvent was removed by evaporation under reduced pressure. The residue was purified by column chromatography (120 g of silica, 0-50% EtOAc in heptanes) to afford Compound AcO—II—Ac (2.89 g, 86%).

### (4R,4aR,7R,7aR,12bS,14S)-14-acetyl-3-benzoyl-7-methoxy-1,2,3,4,7,7a-hexahydro-7,4a-ethano-4,12-methanobenzofuro[3,2-e]isoquinolin-9-yl benzoate

MS (ES-API pos) m/z 562.2 (M+H).

¹H NMR (300 MHz, CDCl₃) δ [ppm] 8.15 (d, J=7.6 Hz, 2H), 7.62 (m, 1H), 7.52-7.39 (m, 7H), 6.91 (d, J=8.2 Hz, 1H), 6.70 and 6.66 (2×d, J=8.2 Hz, 1H), 6.10 (d, J=8.8 Hz, 0.5H), 5.97 (d, J=8.8 Hz, 0.5H), 5.73 (d, J=8.8 Hz, 0.5H), 5.52 (d, J=6.4 Hz, 0.5H), 5.43 (d, J=8.8 Hz, 0.5H), 4.75 (d, J=10.0 Hz, 0.5H), 4.60 (s, 1H), 4.40 (d, J=4.7 Hz, 0.5H), 3.71 (d, J=14.7 Hz, 0.5H), 3.55-3.26 (m, 1H), 3.50 (s, 3H), 3.20-3.03 (m, 2H), 2.93-2.84 (m, 1H), 2.38 (dd, J=12.9, 9.4 Hz, 1H), 2.18-2.02 (m, 4H), 1.91 (m, 1H), 1.71-1.56 (m, 1H).

### Example 50. Preparation of Compound HO—IIIA-Ac (Step D)

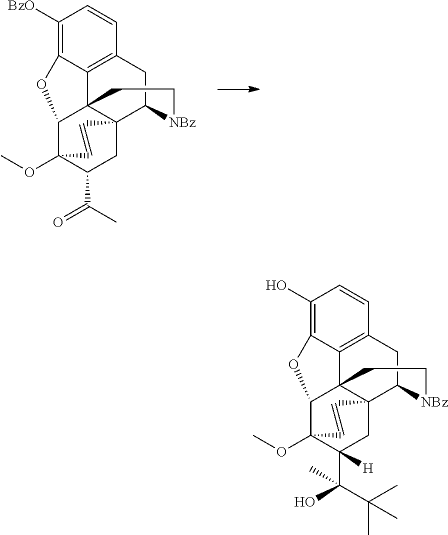

Dry toluene (120 mL) was added to a solution of tert-butylmagnesium chloride (1.7 M in THF, 27 mL). Part of the solvent was evaporated under reduced pressure at 50° C., leaving around 30 mL. Under a nitrogen atmosphere a solution of Compound AcO—II—Ac (1.69 g, 3.01 mmol) in dry toluene (12 mL) was added slowly by means of a syringe. The mixture was stirred at 60° C. for 3 h. After cooling to room temperature diethyl ether (50 mL) and water (75 mL) were added. The mixture was acidified with 1N aqueous HCl. Both layers were separated. The aqueous layer was extracted with EtOAc (2×50 mL). The combined organic layers were dried over Na₂SO₄, filtered and concentrated under reduced. The residue was purified by column chromatography (40 g of silica, 0-50% EtOAc in heptanes) to afford Compound HO—IIIA-Ac (1.10 g, 71%).

### ((4R,4aR,7R,7aR,12bS,14R)-9-hydroxy-14-(2-hydroxy-3,3-dimethylbutan-2-yl)-7-methoxy-1,2,7,7a-tetrahydro-7,4a-ethano-4,12-methanobenzofuro[3,2-e]isoquinolin-3(4H)-yl)(phenyl)methanone

MS (ES-API pos) m/z 516.3 (M+H).

¹H NMR (300 MHz, CDCl₃) δ [ppm] 7.44-7.40 (m, 5H), 6.65 (d, J=8.2 Hz, 1H), 6.54 and 6.49 (2×d, J=8.2 Hz, 1H), 6.07 and 5.99 (2×d, J=9.4 Hz, 1H), 5.54-5.42 (m, 2H), 5.23 (d, J=8.8 Hz, 0.5H), 4.90-4.71 (m, 1.5H), 4.60 (d, J=10.6 Hz, 1H), 4.28 (d, J=6.5 Hz, 0.5H), 3.76 and 3.74 (2×s, 3H), 3.70-3.65 (in, 0.5H), 3.44-3.33 (m, 0.5H), 3.26-2.94 (m, 2.5H), 2.39-2.27 (m, 1H), 2.20-2.11 (m, 1H), 2.08-1.88 (m, 1H), 1.88-1.78 (m, 1H), 1.38-1.20 (m, 1H), 1.01 (s, 9H), 0.92 (s, 3H).

### Example 51. Preparation of Compound HO—IIIA-Bn (Step H)

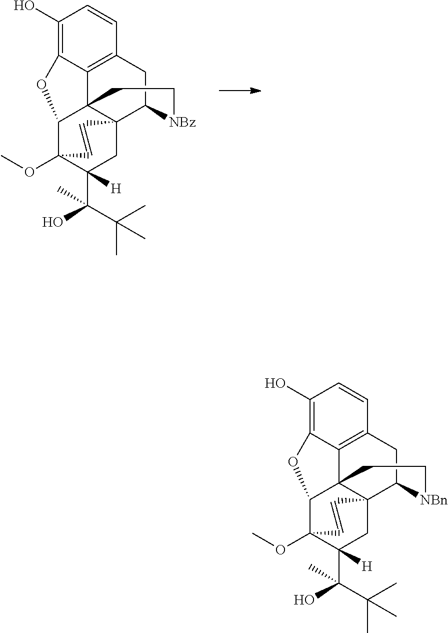

Under a nitrogen atmosphere Compound HO—II—Ac (1.01 g, 1.96 mmol) was dissolved in THF (25 mL). Lithium aluminum hydride (149 mg, 3.92 mmol) was added and the mixture was heated at 70° C. for 3 h. After standing for 18 h at room temperature water (70 mL) was added and the mixture was extracted with EtOAc (3×70 mL). The combined extracts were dried over Na₂SO₄, filtered and concentrated under reduced pressure. The residue was purified by column chromatography (24 g of silica, 0-30% EtOAc in heptanes) to afford Compound HO—IIIA-Bn (564 mg, 57%).

### (4R,4aR,7R,7aR,12bS,14R)-3-benzyl-14-(2-hydroxy-3,3-dimethylbutan-2-yl)-7-methoxy-1,2,3,4,7,7a-hexahydro-7,4a-ethano-4,12-methanobenzofuro[3,2-e]isoquinolin-9-ol

MS (ES-API pos) m/z 502.3 (M+H).

¹H NMR (300 MHz, CDCl₃) δ [ppm] 7.46-7.21 (m, 5H), 6.62 (d, J=8.0 Hz, 1H), 6.49 (d, J=8.1 Hz, 1H), 5.95 (d, J=8.9 Hz, 1H), 5.67 (s, 1H), 5.35 (d, J=8.9 Hz, 1H), 5.29 (s, 1H), 4.61 (s, 1H), 3.75 (s, 3H), 3.69 (s, 2H), 3.24 (d, J=18.4 Hz, 1H), 3.18-3.03 (m, 2H), 2.74-2.49 (m, 2H), 2.40 (dd, J=18.4, 6.7 Hz, 1H), 2.19 (t, J=8.6 Hz, 1H), 2.12-1.81 (m, 2H), 1.06 (s, 9H), 0.99 (s, 3H), 0.93 (dd, J=12.3, 8.8 Hz, 1H).

### Example 52. Preparation of Compound HO—IV—H (Step C)

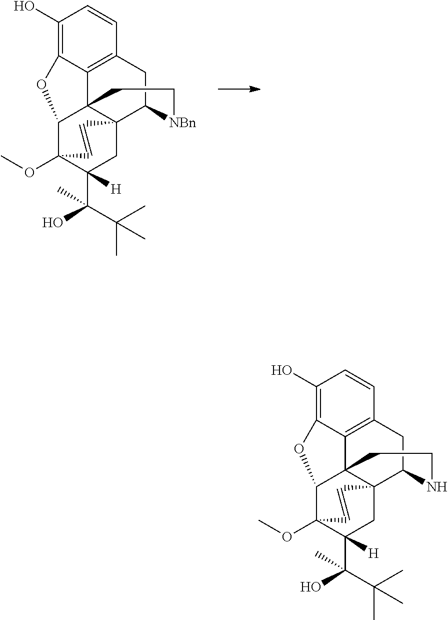 [SMILES: CO[C@]12C=C[C@@]3(C[C@@H]1[C@](C)(O)C(C)(C)C)[C@H]1Cc4ccc(O)c5c4[C@@]3(CCN1Cc1ccccc1)[C@H]2O5 ; CO[C@]12C=C[C@@]3(C[C@@H]1[C@](C)(O)C(C)(C)C)[C@H]1Cc4ccc(O)c5c4[C@@]3(CCN1)[C@H]2O5]

Compound HO—IIIA-Bn (560 mg, 1.12 mmol) was dissolved in 2-propanol (20 mL), followed by the addition of water (1 mL), 10% Pd/C (280 mg) and glacial acetic acid (0.2 mL). The mixture was reduced at 1 atmosphere of hydrogen pressure for 3 days. The reaction mixture was filtered over a pad of Celite and the filtrate was concentrated under reduced pressure. The residue was dissolved in a mixture of methanol (20 mL), water (1 mL) and glacial acetic acid (0.2 mL). After the addition of 10% Pd/C (280 mg) the mixture was reduced at 1 atmosphere of hydrogen pressure at 60° C. for 3 days. After cooling to room temperature the reaction mixture was filtered over a pad of Celite and the filtrate was concentrated under reduced pressure. The residue was purified by column chromatography (24 g of silica, 0-10% methanol in dichloromethane) to afford Compound HO—IV—H (228 mg, 49%).

### norbuprenorphine

MS and NMR data were in agreement with those obtained in previous examples.

### Example 53. Preparation of Compound AcO—IIIB—Ac (Step C)

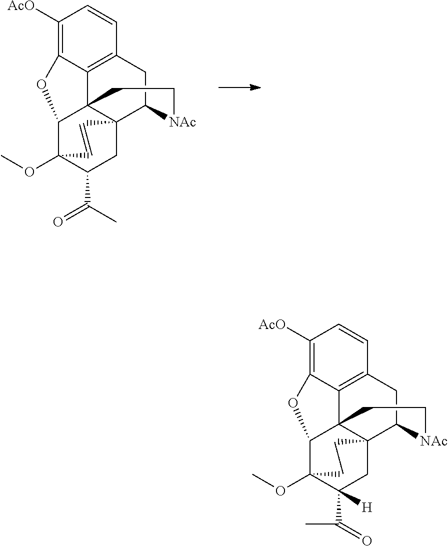

Compound AcO—II—Ac is dissolved in 2-propanol, followed by the addition of water, 10% Pd/C (10%) and glacial acetic acid. The mixture is reduced at 1 atmosphere of hydrogen pressure for 3 days at 80° C. After cooling to room temperature the reaction mixture is filtered over a pad of Celite and the filtrate is concentrated under reduced pressure. The residue is purified by column chromatography.

### Example 54. Preparation of Compound HO—IV—Ac (Step D)

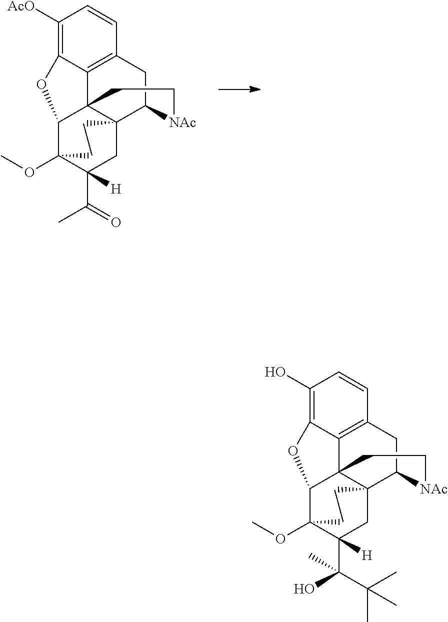

Dry toluene is added to a solution of tert-butylmagnesium chloride (1.7 M in THF). Under a nitrogen atmosphere a solution of Compound AcO—IIIB—Ac in dry toluene is added to the Grignard solution slowly by means of a syringe. The mixture is stirred at 60° C. for 3 h. After cooling to room temperature diethyl ether and water are added. The mixture is acidified with 1N aqueous HCl. Both layers are separated. The aqueous layer is extracted with EtOAc. The combined organic layers are dried over Na₂SO₄, filtered and concentrated under reduced. The residue is purified by column chromatography.

### Example 55. Preparation of Compound HO—IV—Ac (Step C)

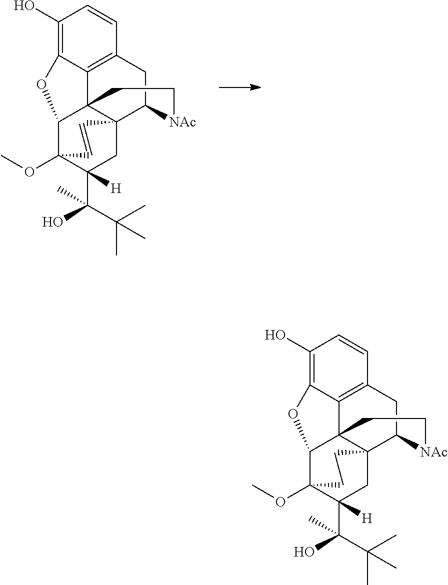

Compound HO—IIIA-Ac is dissolved in 2-propanol, followed by the addition of water, 10% Pd/C (10%) and glacial acetic acid). The mixture is reduced at 1 atmosphere of hydrogen pressure for 3 days at 80° C. After cooling to room temperature the reaction mixture is filtered over a pad of Celite and the filtrate is concentrated under reduced pressure. The residue is purified by column chromatography.

### Example 56. Preparation of Compound HO—IV—H (Step I)

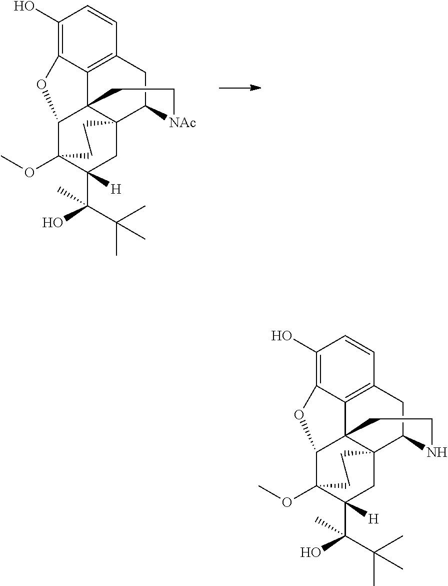

To a solution of HO—IV—Ac in THF at room temperature is added Schwartzs reagent in one portion. The resulting suspension is stirred under an argon atmosphere for 40 min, when the suspension turns pale red. The reaction mixture is evaporated to a thick oil, which is purified by column chromatography.

### Example 57. Preparation of Compound HO—IV—H (Step I)

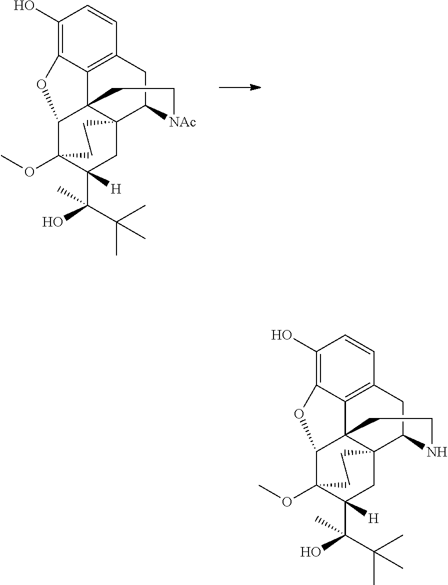

A mixture of HO—IV—Ac, KOH and diethylene glycol is stirred under an inert atmosphere at 170-180° C. for 7 h. The reaction mixture is then quenched with water (10 mL) and the products are extracted with dichloromethane. The combined organic layers are washed with water, brine, dried over Na₂SO₄ and concentrated. The product is isolated by column chromatography.

### Example 58. Thebaine and oripavine N-demethylation by new fungal candidates

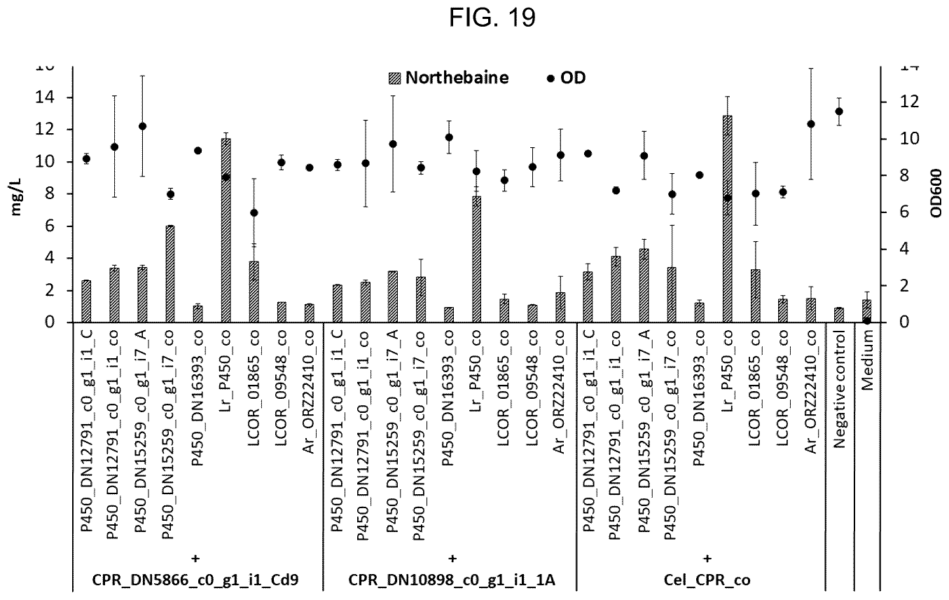

The yeast codon-optimized putative cytochrome P450 genes from Thamnostylum piriforme (P450_DN16393_co (SEQ ID NO: 60)), Lichtheimia corymbifera (LCOR_01865_co (SEQ ID NO: 54) and LCOR_09548_co (SEQ ID NO: 56)) and Absidia repens (Ar_ORZ22410_co (SEQ ID NO: 58)) (see Table 6 shown in Example 4) were expressed in S. cerevisiae strains in combination with the CPRs, CPR_DN5866_c0_g1_i1_Cd9 (SEQ ID NO: 10) and CPR_DN10898_c0_g1_i1_1A (SEQ ID NO: 13) from Thamnostylum piriforme (Table 4 shown in Example 4) or the yeast codon-optimized CPR from Cunninghamella elegans Cel_CPR_co (SEQ ID NO. 17). The Thamnostylum piriforme P450_DN15259_c0_g1_i7_A (SEQ ID NO: 3) and P450_DN12791_c0_g1_i1_C (SEQ ID NO: 5), together with their yeast codon-optimized versions (P450_DN15259_c0_g1_i7_co (SEQ ID NO: 3) and P450_DN12791_c0_g1_i1_co (SEQ ID NO: 6)) and the Lichtheimia ramosa cytochrome P450 (Lr_P450_co) were used as positive controls. Cells were fed with either 0.5 mM thebaine or oripavine which were both prepared from a 25 mM stock dissolved in DMSO. Yeast cell incubation was performed in selective medium containing 0.1 M potassium phosphate buffer pH 7. After 72 h of growth at 30° C. with shaking at 300 rpm, 100 μl-supernatants were harvested, spiked with 1 mg/I caffeine as internal standard and analyzed by LC-MS (as described in Example 11). As seen in FIG. 19, both LCOR_01865_co (SEQ ID NO: 54) and Ar_ORZ22410_co (SEQ ID NO: 58) displayed activity towards thebaine, converting it to northebaine when co-expressed with at least one of the tested CPRs. In this study it appeared that Lr_P450_co (SEQ ID NO: 8) was performing the best conversion of thebaine into northebaine. This was not the case for P450_DN16393_co (SEQ ID NO: 60) and LCOR_09548_co (SEQ ID NO: 56) when compared to the controls.

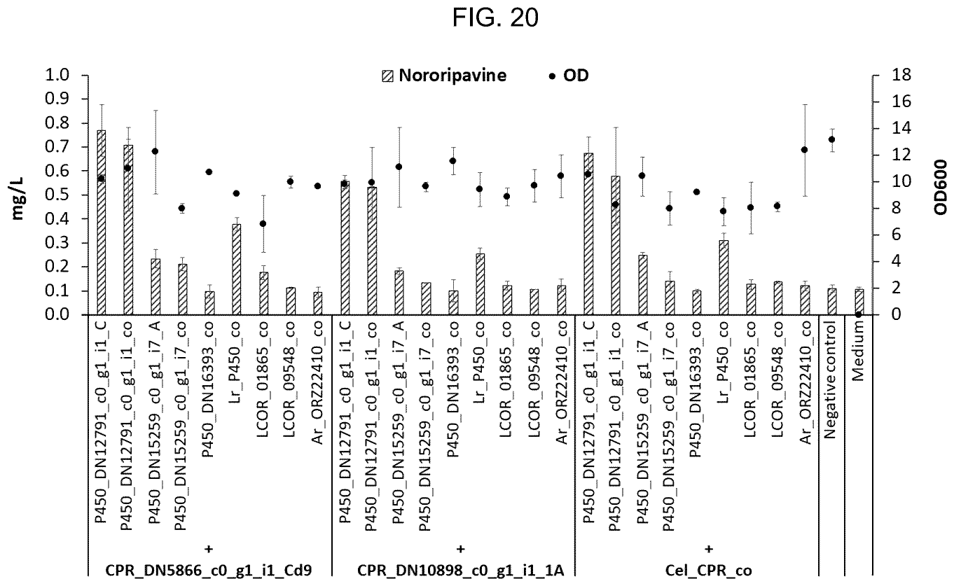

Furthermore, it was shown that both LCOR_01865_co (SEQ ID NO: 54), Ar_ORZ22410_co (SEQ ID NO: 58) and LCOR_09548_co (SEQ ID NO: 56) were capable of producing nororipavine when oripavine was administered (FIG. 20).

### Example 59. Thebaine or oripavine N-demethylation by codon-Optimized Lr_P450 and Mc_S2JT25 Co-Expressed with CPRs

Codon-optimized cytochrome P450 genes from Lichteimia ramosa (Lr_P450_co (SEQ ID NO: 8)) and the candidate homolog Mc_S2JT25_co (SEQ ID NO: 52) from Mucor circinelloides were co-expressed with two CPRs from Thamnostylum piriforme, that were either native or codon-optimized and one codon-optimized CPR, Cel_CPR from Cunninghamella elegans.

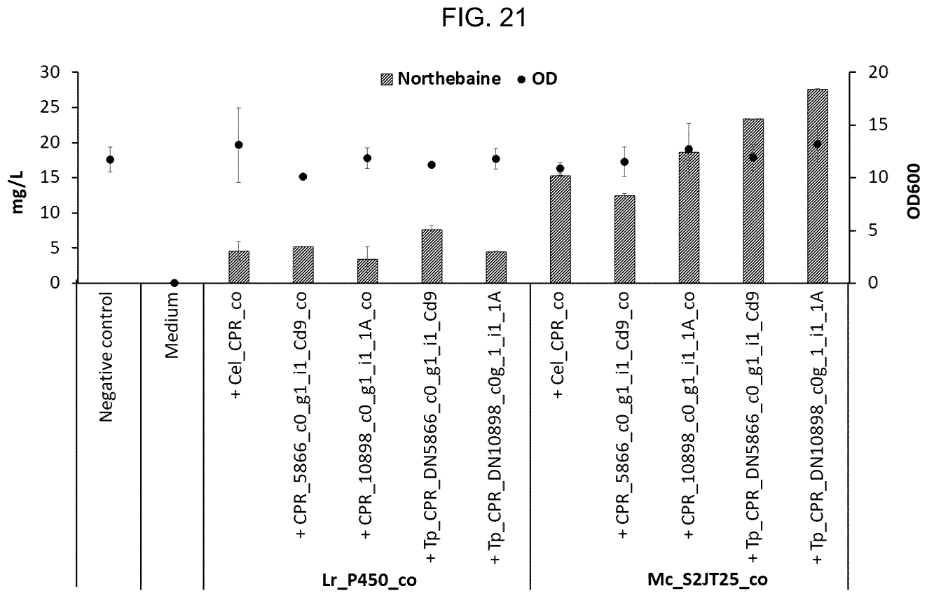

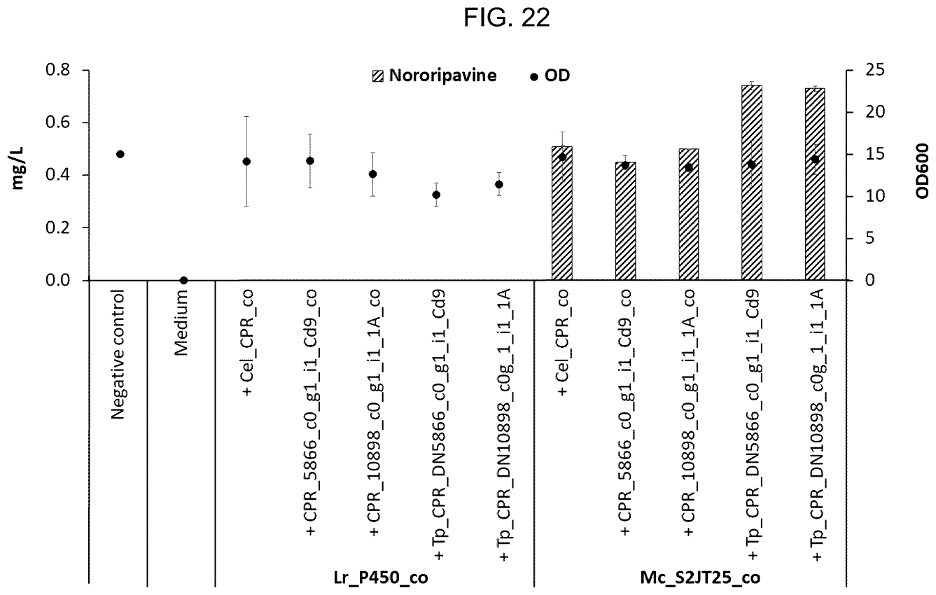

Cells were fed with either 0.5 mM thebaine or oripavine which were both prepared from a 25 mM stock dissolved in DMSO. Yeast cell incubation was performed in selective medium containing 0.1 M potassium phosphate buffer pH 7. After 72 h of growth at 30° C. with shaking at 300 rpm, 100 μl-supernatants were harvested, spiked with 1 mg/I caffeine as internal standard and analyzed by LC-MS (as described in Example 11). As seen in FIG. 21, the candidate homolog Mc_S2JT25_co (SEQ ID NO: 52) yielded the highest northebaine amount compared to Lr_P450_co (SEQ ID NO: 8), which previously was shown to have high thebaine conversion to northebaine. It was also shown that the codon usage of the CPR has an effect on northebaine production. Similarly, Mc_S2JT25_co (SEQ ID NO: 52) resulted in the highest amount of nororipavine compared to Lr_P450_co (SEQ ID NO: 8) when oripavine was administered (FIG. 22).

### Example 60. Thebaine or oripavine N-demethylation by codon-Optimized C. echinulata cytochrome P450 Co-Expressed with Cel_CPR_co

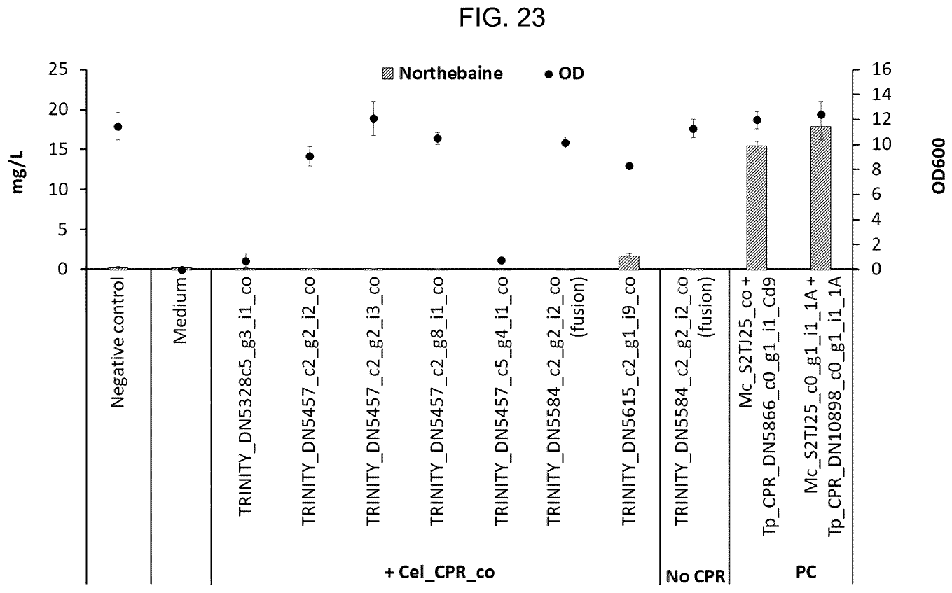

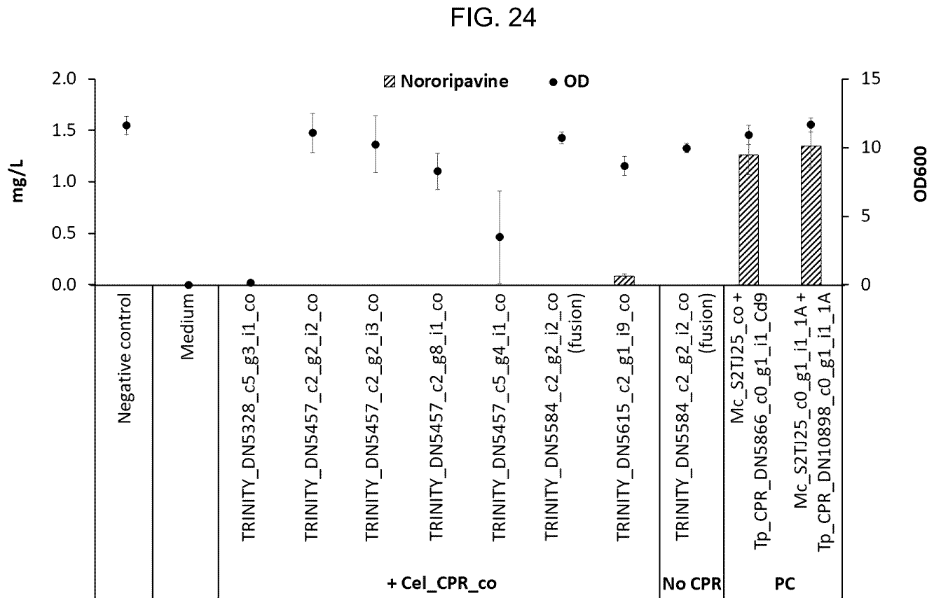

Seven yeast-codon optimized cytochrome P450 candidates from Cunninghamella echinulata (Table 6 shown in example 4) were co-expressed with the CPR, Cel_CPR (SEQ ID NO: 17) from Cunninghamella elegans in S. cerevisiae. Cells were fed with either 0.5 mM thebaine or oripavine which were both prepared from a 25 mM stock dissolved in DMSO. Yeast cell incubation was performed in selective medium containing 0.1 M potassium phosphate buffer pH 7. After 72 h of growth at 30° C. with shaking at 300 rpm, 100 μl-supernatants were harvested, spiked with 1 mg/I caffeine as internal standard and analyzed by LC-MS (as described in Example 11). As seen in FIGS. 23 and 24, only one of the tested candidates, P450_DN5615_c2_g1_i9_co (SEQ ID NO: 62), displayed demethylase activity towards thebaine and oripavine, producing northebaine and nororipavine, respectively. Although the activity was substantially below the in vivo demethylation activity observed for Mc_S2JT25_co (SEQ ID NO: 52).

### Example 61. In Planta Production of Either northebaine or oripavine by heterologous Expression of Genes Encoding N-demethylases and O-demethylases, Respectively

Transient Expression of Gene Constructs in Nicotiana benthamiana

Synthetic DNA fragments, codon optimized for Saccharomyces cerevisiae expression and encoding the demethylase enzymes Lr_P450_co (SEQ ID NO: 8), Mc_S2JT25_co (SEQ ID NO: 52) and Ps_CODM_co (SEQ ID NO: 132) and the cytochrome P450 reductase enzymes CPR_DN10898_c0_g1_i1_co (SEQ ID NO: 14) and Cel_CPR_co (SEQ ID NO: 17) were PCR amplified using standard deoxyuracil(dU)-containing primers. All amplified fragments were cloned into a modified version of the pCAMBIA130035Su plasmid under the control of the doubled enhancer element from CaMV 35S promoter, by using Uracil-Specific Excision Reagent (USER) cloning technology (Nour-Eldin et al., 2006). The modified pCAMBIA130035Su plasmid was generated by PCR amplifying the pCAMBIA130035Su plasmid using a standard deoxyuracil(dU)-containing primer pairand the amplified plasmid backbone was hereafter treated with DpnI (New England BioLabs). A synthetic DNA fragment encoding the OCS (Octapine Synthase) terminator from Agrobacterium tumefaciens (Genbank accession no. CP011249.1) was purchased from Integrated DNA Technologies and PCR amplified using a set of standard deoxyuracil(dU)-containing primers. The amplified OCS terminator was cloned in the DpnI-treated plasmid backbone with USER technology, yielding the modified pCAMBIA130035Su plasmid, pCAMBIA130035Su_MOD which was verified by DNA sequencing.

All plasmid-gene constructs along with a pCAMBIA130035Su_MOD plasmid containing the tomato p19 viral supressor gene (Baulcombe and Molnar, 2004) were transformed into the Agrobacterium tumefaciens strain, AGL-1 and infiltrated into leaves of Nicotiana benthamiana plants as described in (Bach et al., 2014). After 4 days, agroinfiltrated leaves were re-infiltrated with 0.5 mM thebaine which was prepared from a 110 mM thebaine stock dissolved in DMSO and diluted in water. Plants were hereafter left to grow for another 1 day in the green house.

Metabolite Extraction and LC-MS-MS Analysis

Metabolites were extracted from discs (0=3 cm) of agroinfiltrated N. benthamiana leaves. Leaf discs, excised with a cork borer, were flash frozen in liquid nitrogen. 0.5 ml of extraction buffer (60% (v/v) methanol, 0.1% (v/v) formic acid), equilibrated to 50° C., were added to each frozen leaf disc followed by incubation for 1 hour at 50° C., agitating at 600 rpm. The supernatant was isolated and passed through a Multiscreen_{HTS} HV 0.45 μm filter plate (Merck Milipore) before analysis by LC-MS-MS.

For all compounds (thebaine, northebaine and oripavine) stock solutions were prepared in DMSO at a concentration of 10 mM. Standard solutions were prepared at concentrations of 6 μM, 4 μM, 2 μM, 1 μM, 500 nM, 200 nM, 100 nM, 50 nM, 20 nM and 10 nM from the stock solutions. Samples were injected into the Agilent 1290 UPLC coupled to an Ultivo Triple Quadrupole. The LC-MS method was as follows: Mobile Phase A. H₂O+0.1% Formic acid; Mobile Phase B: Acetonitrile+0.1% Formic acid; Column: Phenomenex Kinetex 1.7 μm XB-C18 100 Å, 2.1×100 mm. The elution gradient is shown in Table 2 and the LC-MS conditions are given in Table 3. Table 4 shows the mass spectrometer source and detector parameters and Table 5 shows the target compounds, their retention times, their parent ion, transition ions (MRM) as well as dwell times, cone voltages and collision energies used.

TABLE 2
Gradient for LC-MS

| Time (min) | % B |
|---|---|
| 0 | 2 |
| 0.30 | 2 |
| 4.00 | 30 |
| 4.40 | 100 |
| 4.90 | 100 |
| 5 | 2 |
| 6 | 2 |

TABLE 3
LC-MS conditions

| Parameter | Value |
|---|---|
| Injection volume | 2 μl |
| Column Temperature | 30° C. ± 4° C. |
| Injection method | Flow through needle |
| Flow | 0.4 ml/min |
| Auto sampler temperature | 10° C. ± 2° C. |
| Reconditioning wash | 2% Acetonitrile (in H₂O), 5 sec |
| Weak wash | 20% Methanol (in H₂O), 5 sec |
| Strong wash | 30% Acetonitrile, 30% Methanol, |
| Seal wash | 30% 2-Propanol, 10% H₂O, 10 sec |
| | 20% 2-Propanol (in H₂O) |

TABLE 4
Mass spectrometer source and detector parameters
(Ultivo Triple Quadrupole)

| Source Parameter | Value |
|---|---|
| Ion Source | Electrospray Positive Mode (ESI+) |
| Capillary Voltage | 3.5 kV |
| Nozzle Voltage | 500 V |
| Source Gas Temperature | 290° C. |
| Source Gas Flow | 12 L/min |
| Source Sheath Gas Temperature | 380° C. |
| Source Sheath Gas Flow | 12 L/min |
| Nebulizer | 30 psi |
| Mode | MS/MS |
| Collision | See Table 4 |

TABLE 5
Multiple reaction monitoring targets and conditions (ESI+)

| | Retention | Parent | Daughter | Dwell | Fragment | Collision |
|---|---|---|---|---|---|---|
| Target | time | ion | ion | time | or voltage | energy |
| compound | (min) | (m/z) | (m/z) | (ms) | (V) | (V) |
| Oripavine | 2.59 | 298 | 237 | 64.05 | 110 | 5 |
| Northebaine | 3.53 | 298 | 249 | 55.03 | 100 | 20 |
| Thebaine | 3.6 | 312 | 58 | 61.53 | 110 | 10 |

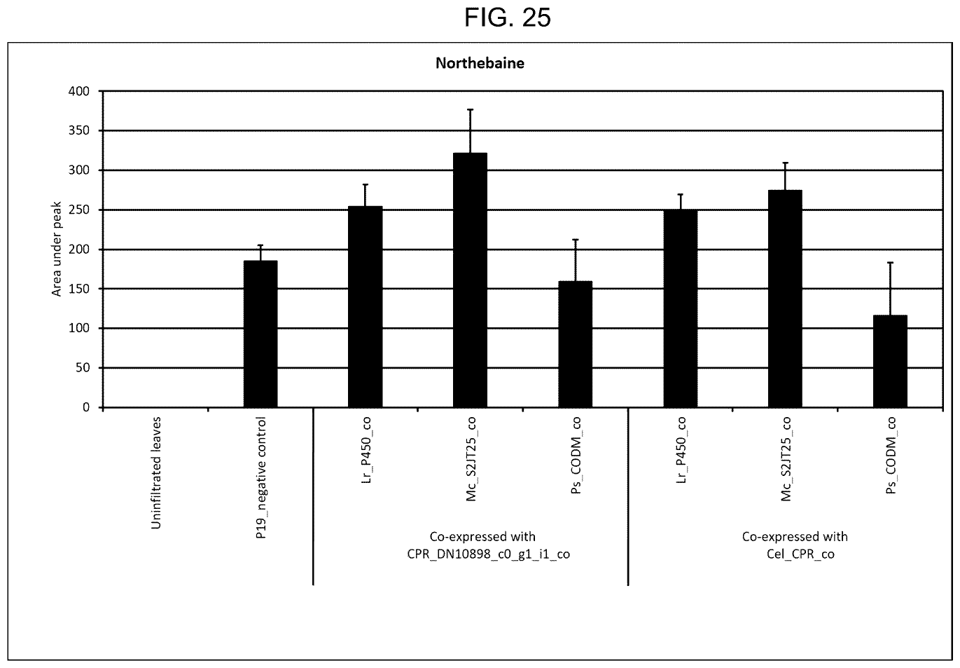

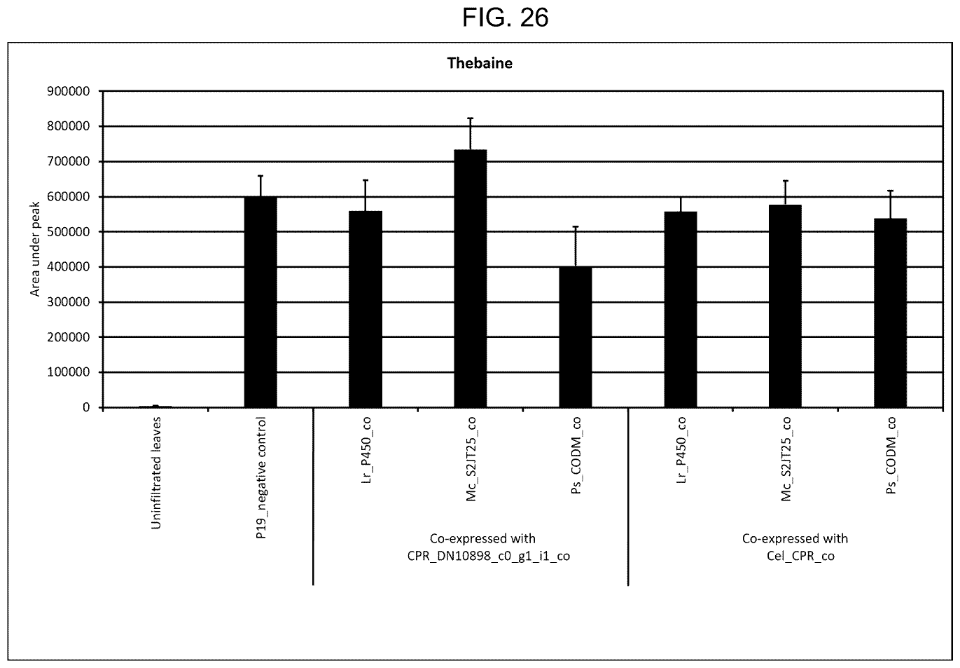

As seen in FIG. 25, a clear increase in northebaine is achieved upon transient co-expression of the N-demethylase genes, Lr_P450_co (SEQ ID NO: 8) or Mc_S2JT25_co (SEQ ID NO: 52) together with either the reductase genes, CPR_DN10898_c0_g1_i1_co (SEQ ID NO: 15) or Cel_CPR_co (SEQ ID NO: 17), in N. benthamiana leaves after thebaine infiltration (FIG. 26). This increase appears to be significantly above the levels detected in the negative P19 control. Such an increase in northebaine levels was however not apparent upon transiently co-expressing the O-demethylase gene, Ps_CODM_co (SEQ ID NO: 132) with either CPR_DN10898_c0_g1_i1_co (SEQ ID NO: 15) or Cel_CPR_co (SEQ ID NO: 17) in N. benthamiana (FIG. 25). The accumulation of small amounts of northebaine in the negative P19 control could point to one or more endogenous N. benthamiana enzymes which are capable of performing some conversion of the thebaine into northebaine.

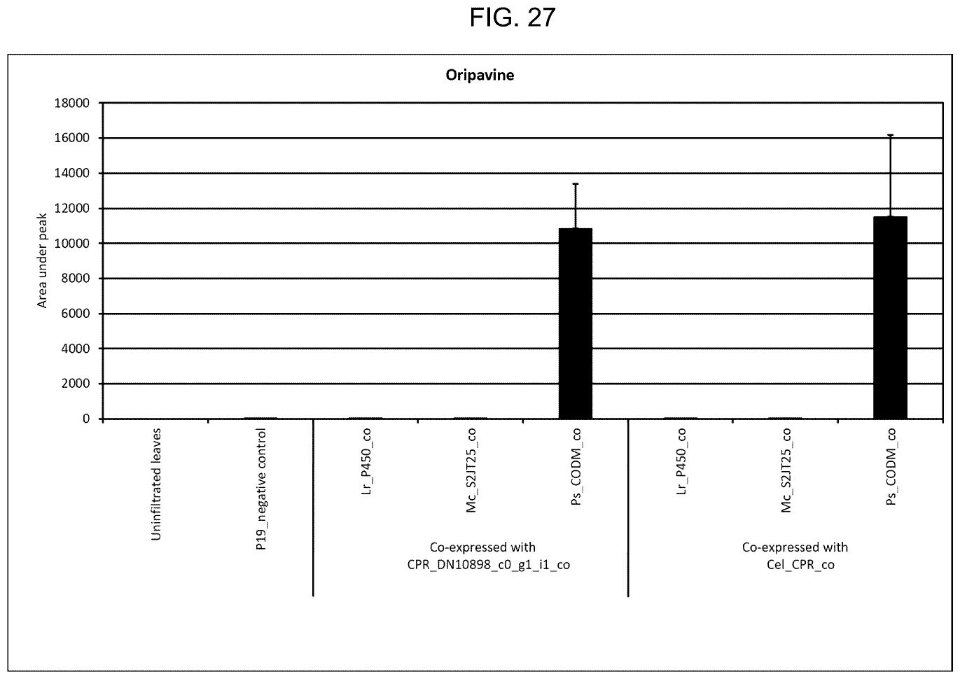

In contrast, but as expected, the Ps_CODM_co (SEQ ID NO: 132) was the only enzyme of the tested demethylases that was capable of converting thebaine into oripavine when compared to the negative control (FIG. 27).

### REFERENCES

1: Nour-Eldin H. H., Hansen B. G., Norholm M. H., Jensen J. K., Halkier B. A. (2006). Advancing uracil-excision based cloning towards an ideal technique for cloning PCR fragments. Nucleic Acids Res. 34:e122.
2: Baulcombe D. C., Molnar A. (2004). Crystal structure of p19—a universal suppressor of RNA silencing. Trends Biochem Sci. 29: 279-81.
3: Bach, S. S., Bassard, J. E., Andersen-Ranberg, J., Moldrup, M. E., Simonsen, H. T., Hamberger, B. (2014). High-Throughput Testing of Terpenoid Biosynthesis Candidate Genes Using Transient Expression in Nicotiana benthamiana. In M Rodriguez Concepcion, ed, Plant Isoprenoids, Methods in Molecular Biology, Vol. 1153. Humana Press, New York.

### Example 62. Production of northebaine and oripavine from thebaine by heterologous Expression of Genes Encoding demethylases in Aspergillus nidulans

A selection of cytochrome P450 enzymes and the P. somiferum CODM (SEQ ID NO: 132) were tested in Aspergillus nidulans strain NID1 (argB2, pyrG89, veA1, nkuAA) (Nielsen et al 2008), in order to evaluate their demethylation capacity of thebaine to either northebaine (N-demethylation) or oripavine (O-demethylation).

A combination of N-demethylases (Lr_P450_co (SEQ ID NO: 8, Mc_S2JT25_co (SEQ ID NO: 52), P450_DN12791_c0_g1_i1_co (SEQ ID NO: 4)) and cytochrome P450 reductase enzymes were tested for demethylation of thebaine to northebaine. The O-demethylase enzyme Ps_CODM_co (SEQ ID NO: 132), was also tested for the conversion of thebaine to oripavine.

All tested gene sequences were codon optimized for Saccharomyces cerevisiae expression and PCR amplified with standard deoxyuracil(dU)-containing primers. The PCR amplified fragments were cloned using the Uracil-Specific Excision Reagent (USER) cloning system (Nour-Eldin et al., 2006) and introduced into a vector system designed for expression and genomic integration in A. nidulans integration site 1 (IS1) (Hansen et al. 2011). The vector used in this study was pU1111-1, together with the gpdA promoter and trpC terminator as described by Hansen et al. 2011.

Transformants were selected using the auxotrophic argB marker in the pU1111-1 plasmid. Correct genomic insertion of the expression cassettes were verified by PCR on fungal colonies, as described by Hansen et al. 2011. Five colonies from each transformation were inoculated in Minimal Medium (MM) containing uridine and uracil at pH 7 and 0.5 mM thebaine which was prepared from a 110 mM thebaine stock dissolved in DMSO. The cultures were incubated at 37° C. with 130 rpm agitation for 84 hours.

Metabolite Extraction and LC-MS-MS Analysis

Metabolites were extracted from 0.5 ml of culture supernantant with 0.5 ml of extraction buffer (80% (v/v) ethanol, 0.1% (v/v) formic acid), equilibrated to 50° C. by incubation for 1 hour at 50° C. with agitation at 600 rpm. The supernatant was isolated and passed through a MultiscreenHTS HV 0.45 μm filter plate (Merck Milipore) before analysis by LC-MS-MS as described in Example 64.

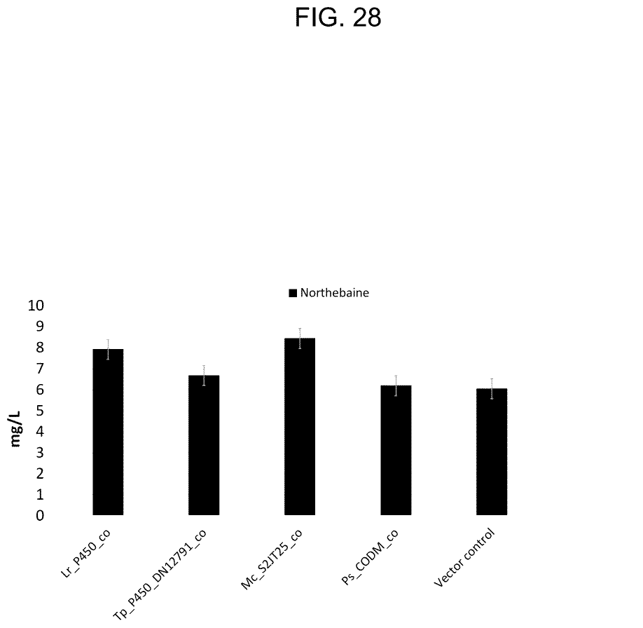

The production of northebaine was achieved upon heterologous expression of the N-demethylase genes Mc_S2JT25_co (SEQ ID NO: 52) and Tp_P450_DN12791_c0_g1_i1_co (SEQ ID NO: 5) (FIG. 28). This production appears to be significantly above the levels detected in the vector control (FIG. 28). Such an increase in northebaine levels was however not apparent upon heterologous expression of Lr_P450_co (SEQ ID NO: 8) (FIG. 28). The accumulation of northebaine in the vector control could point to one or more endogenous A. nidulans enzymes, which are capable of performing some conversion of the thebaine into northebaine. The bioconversion rate of thebaine to northebaine could likely be improved by the combination of the N-demethylase enzymes tested in this study with CPR enzymes, as it was done in the examples with Saccharomyces cerevisiae.

### REFERENCES

Nour-Eldin H. H., Hansen B. G., Norholm M. H., Jensen J. K., Halkier B. A. (2006). Advancing uracil-excision based cloning towards an ideal technique for cloning PCR fragments. Nucleic Acids Res. 34:e122.
Hansen B. G. et al (2011). Versatile enzyme expression and characterization system for Aspergillus nidulans, with the Penicillium brevicompactum polyketide synthase gene from the mycophenolic acid gene cluster as a test case. App. and Environmental Microbiology. 77(9):3044-3051
Nielsen, J. B., M. L. Nielsen, and U. H. Mortensen. (2008). Transient disruption of non-homologous end-joining facilitates targeted genome manipulation in the filamentous fungus Aspergillus nidulans. Fungal Genet. Biol. 45:165-170.

### Example 63. N-demethylation Activity of New Cytochrome P450 Fungal and Plant Homologs

Expression of cytochrome P450 fungal and plant homologs in S. cerevisiae Synthetic DNA fragments, codon-optimized for Saccharomyces cerevisiae expression and encoding different cytochrome P450 fungal and plant homologs together with a codon-optimized CPR from Lichtheimia ramosa (POR1) (Tablet) were synthesized by TWIST Bioscience or Integrated DNA technologies. The codon optimized-sequences Cel_CPR_co (SEQ ID NO: 17), CPR_5866_c0_g1_i1_co (SEQ ID NO: 11), CPR_DN10898_c0_g1_i1_co (SEQ ID NO: 12), Lr_P450_co (SEQ ID NO: 8) ( ), Mc_S2JT25_co (SEQ ID NO: 52) and the O-demethylase enzyme Ps_CODM_co (SEQ ID NO: 132) were PCR amplified with standard primer sets, containing a SpeI (in the forward primer) and XhoI (in the reverse primer) restriction site. All cytochrome P450 genes were cloned into the SpeI and XhoI restriction sites of the P415-TEF vector and controlled by the TEF1 promoter (Mumberg et al., 1995)) (Table 6 shown in Example 4) and all cytochrome P450 reductase gene candidates and the Ps_CODM were cloned into the P413-TEF plasmid under the control of the TEF1 promoter (Mumberg, 1995) (Table 2)

Finally, all generated plasmid-construct were verified by DNA sequencing.

All plasmids containing cytochrome P450 homologs were co-expressed in S. cerevisiae together with each of the following CPRs: Cel_CPR_co (SEQ ID NO: 17), Tp_CPR_5866_c0_g1_i1_co (SEQ ID NO: 11), Tp_CPR_DN10898_c0_g1_i1_co (SEQ ID NO: 14) and POR1 (SEQ ID NO: 130). Cells were fed with 0.5 mM thebaine prepared from a 25 mM stock dissolved in DMSO and incubated in Synthetic Complete (SC) or DELFT media containing 0.1 M potassium phosphate buffer pH 7. Cells were grown at 30° C. with shaking at 300 rpm for 72 h.

Metabolite Detection

Metabolites were analyzed by harvesting the media supernatant and detected directly by LC-MS-MS. LC-MS-MS were as Example 64.

TABLE 1
Different tested gene sequences

| Name | Accession number | ORGANISM |
|---|---|---|
| Mc_S2JT25_co (SEQ ID | S2JT25 | Mucor circinelloides |
| NO: 52 and 53) | | |
| LCOR_01865_co (SEQ ID | A0A068RKI7 | Lichtheimia corymbifera |
| NO: 54 and 55) | | |
| LCOR_09548_co(SEQ ID | A0A068S8J3 | Lichtheimia corymbifera |
| NO: 56 and 57) | | |
| Ar_ORZ22410_co(SEQ | A0A1X2IUG5 | Absidia repens |
| ID NO: 58 and 59) | | |
| P450_DN16393_co (SEQ | | Thamnostylum piriforme |
| ID NO: 60 and 61) | | |
| P450_DN5615_c2_g1_i9 | TRINITY_DN5615_c2_g1_i9 | Cunninghamella echinulata |
| (SEQ ID NO: 62 and 63) | | |
| CYPDN4 (SEQ ID NO: 64 | I1CCX6 | Rhizopus delemar |
| and 65) | | |
| CYPDN5 (SEQ ID NO: 66 | A0A1X0RYU8 | Rhizopus microsporus |
| and 67) | | |
| CYPDN6 (SEQ ID NO: 68 | A0A0B7NKC0 | Parasitella parasitica |
| and 69) | | |
| CYPDN7 (SEQ ID NO: 70 | I1CBZ0 | Rhizopus delemar |
| and 71) | | |
| CYPDN8 (SEQ ID NO: 72 | A0A0C7AZL4 | Rhizopus microsporus |
| and 73) | | |
| CYPDN9 (SEQ ID NO: 74 | A0A1X2HQ99 | Syncephalastrum racemosum |
| and 75) | | |
| CYPDN10 (SEQ ID NO: | A0A0B7NB52 | Parasitella parasitica |
| 76 and 77) | | |
| CYPDN11 (SEQ ID NO: | A0A068SB73 | Lichtheimia corymbifera |
| 78 and 79) | | |
| CYPDN12 (SEQ ID NO: | A0A1C7N669 | Choanephora cucurbitarum |
| 80 and 81) | | |
| CYPDN13 (SEQ ID NO: | A0A1X2HZE4 | Absidia repens |
| 82 and 83) | | |
| CYPDN14 (SEQ ID NO: | A0A168T1R5 | Absidia glauca |
| 84 and 85) | | |
| CYPDN15 (SEQ ID NO: | XM_023607540 | Rhizopus microsporus |
| 86 and 87) | | |
| CYPDN16 (SEQ ID NO: | A0A1X2HFU1 | Syncephalastrum racemosum |
| 88 and 89) | | |
| CYPDN17 (SEQ ID NO: | A0A2G4SQ34 | Rhizopus microsporus |
| 90 and 91) | | |
| CYPDN18 (SEQ ID NO: | A0A0C9MNJ6 | Mucor ambiguus |
| 92 and 93) | | |
| CYPDN19 (SEQ ID NO: | A0A163B0W9 | Phycomyces blakesleeanus |
| 94 and 95) | | |
| CYPDN20 (SEQ ID NO: | A0A163A6R1 | Phycomyces blakesleeanus |
| 96 and 97) | | |
| CYPDN21 (SEQ ID NO: | XM_018432503 | Phycomyces blakesleeanus |
| 98 and 99) | | |
| CYPDN22 (SEQ ID NO: | XM_023609077 | Rhizopus microsporus |
| 100 and 101) | | |
| CYPDN23 (SEQ ID NO: | A0A162U6J3 | Phycomyces blakesleeanus |
| 102 and 103) | | |
| CYPDN24 (SEQ ID NO: | A0A077WFB8 | Lichtheimia ramosa |
| 104 and 105) | | |
| CYPDN25 (SEQ ID NO: | A0A1X2GSN6 | Hesseltinella vesiculosa |
| 106 and 107) | | |
| CYPDN26 (SEQ ID NO: | A0A162N972 | Phycomyces blakesleeanus |
| 108 and 109) | | |
| CYPDN27 (SEQ ID NO: | A0A077WLY1 | Lichtheimia ramosa |
| 110 and 111) | | |
| CYPDN28 (SEQ ID NO: | A0A1X2HYB6 | Absidia repens |
| 112 and 113) | | |
| CYPDN29 (SEQ ID NO: | A0A068SBU5 | Lichtheimia corymbifera |
| 114 and 115) | | |
| CYPDN30 (SEQ ID NO: | A0A1X2H4N2 | Syncephalastrum racemosum |
| 116 and 117) | | |
| CYPDN31 (SEQ ID NO: | A0A291C3B2 | Absidia caerulea |
| 118 and 119) | | |
| CYPDN32 (SEQ ID NO: | A0A173GQ95 | Absidia caerulea |
| 120 and 121) | | |
| CYPDN33 (SEQ ID NO: | A0A162UUM5 | Phycomyces blakesleeanus |
| 122 and 123) | | |
| CYPDN34 (SEQ ID NO: | A0A068SBP8 | Lichtheimia corymbifera |
| 124 and 125) | | |
| CYPDN35 (SEQ ID NO: | A0A1X2GSL0 | Hesseltinella vesiculosa |
| 126 and 127) | | |
| CYPDN36 (SEQ ID NO: | E5KY66 | Nicotiana sylvestris |
| 128 and 129) | | |
| PORI (SEQ ID NO: 130 | A0A077WBH1 | Lichtheimia ramosa |
| and 131) | | |
| Tp_CPR_DN5866_co | | Thamnostylum piriforme |
| (SEQ ID NO: 11 and 9) | | |
| Tp_CPR_DN10898_co | | Thamnostylum piriforme |
| (SEQ ID NO: 14 and 12) | | |
| Ps_CODM (SEQ ID NO: | D4N502 | Papaver somniferum |
| 132 and 133) | | |

TABLE 2
Description of plasmids containing codon-optimized CPR genes.

| Vector | | | |
|---|---|---|---|
| name | Backbone | Promoter-Gene-Terminator | Description |
| pOD11 | P413-TEF | pTEF1-Tp_CPR_DN 10898 -tCYC1 | CPR_DN10898_c0_g1_i1 (co) from T. |
| | | | piriforme |
| pOD12 | P413-TEF | pTEF1- Tp_CPR_DN5866-tCYC1 | CPR_DN5866_c0_gl_i1 (co) from T. |
| | | | piriforme |
| pOD13 | P413-TEF | pTEF1- Cel_CPR_CO-tCYC1 | Cel_CPR (co) from Cunninghamella |
| | | | elegans |
| pOD14 | P413-TEF | pTEFl- POR1-tCYC1 | A0A077WBH1 (co) from Lichtheimia |
| | | | ramosa |

Thebaine N-demethylation by New Fungal and Plant Candidates

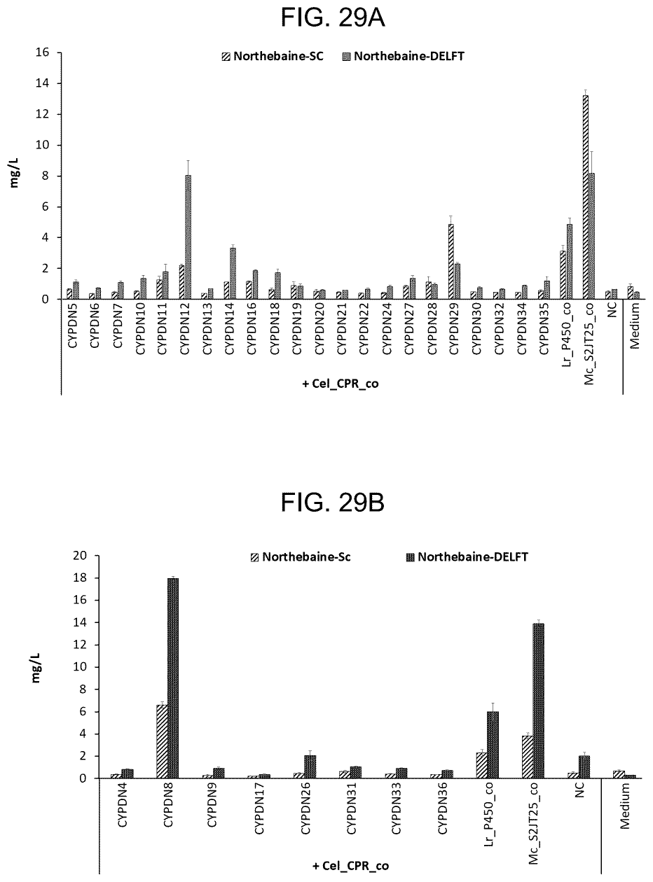

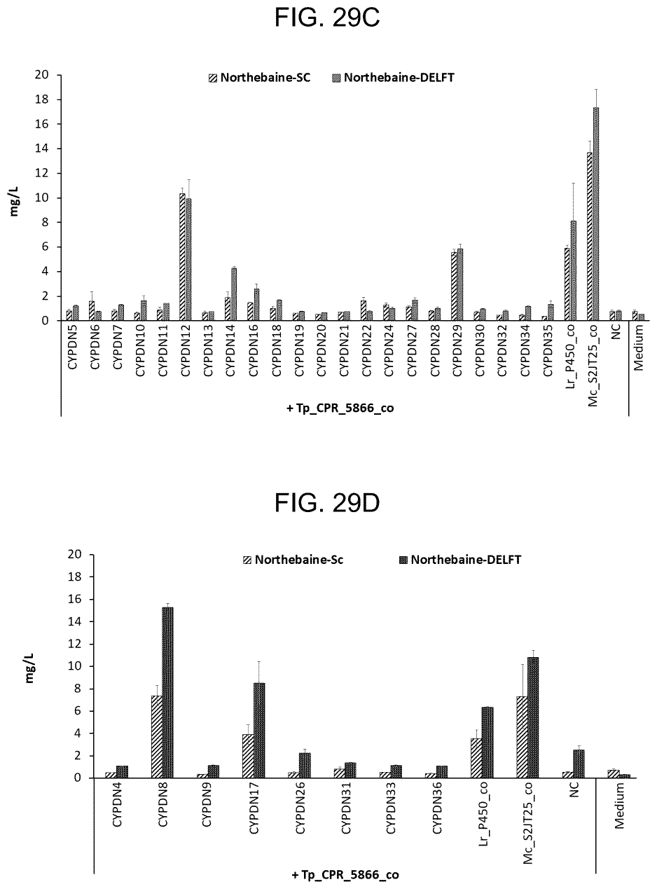

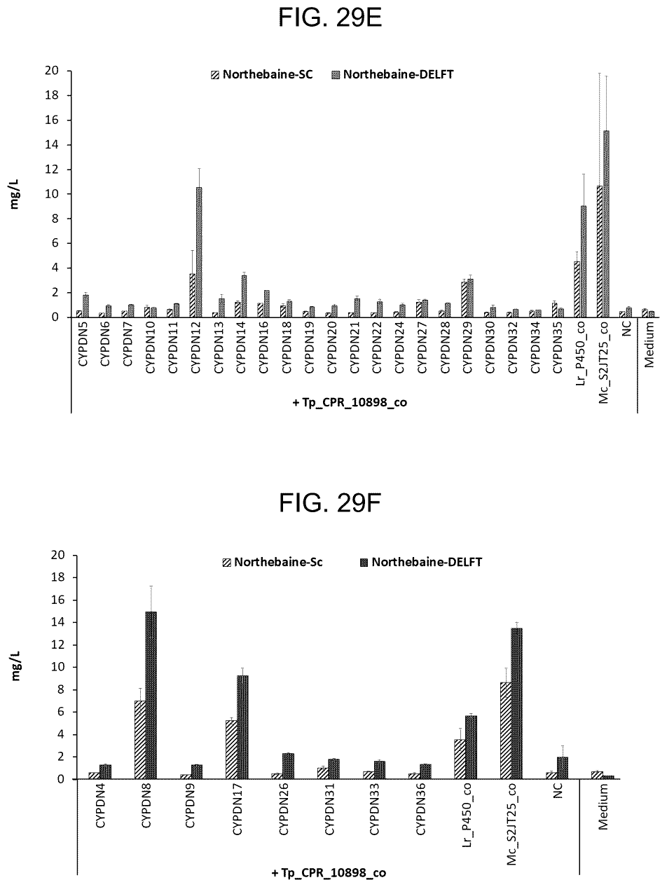

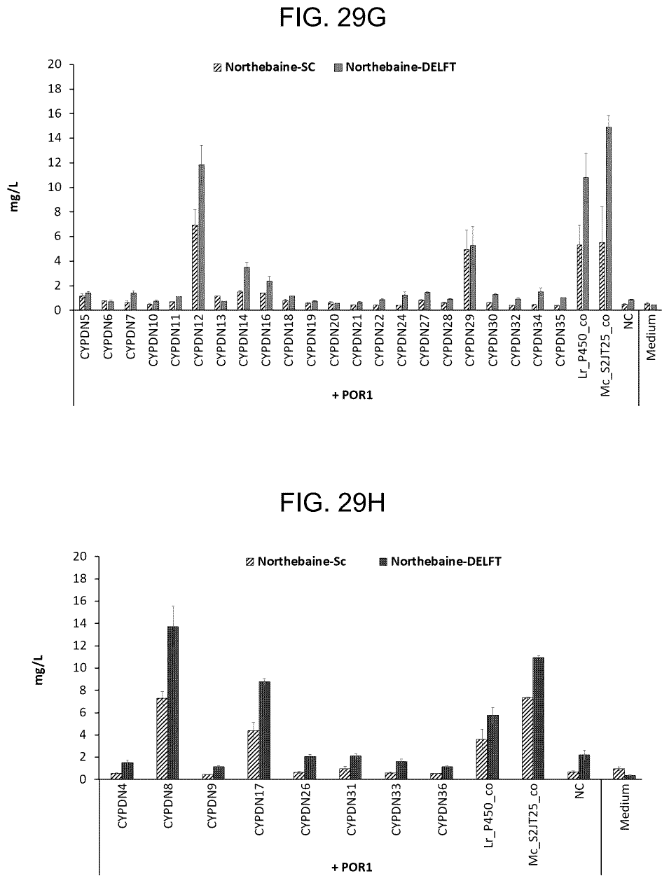

As seen in FIG. 29a-29h, most of the new cytochrome P450 homologs tested show N-demethylation activity towards thebaine when they are expressed together with at least one of the tested CPRs (see Table 3). This activity seemed to markedly increased, in most cases, when the yeast cells were grown in DELFT media when compared to synthetic complete medium.

Thebaine O-demethylation by New Cytochrome P450 Fungal Homologs

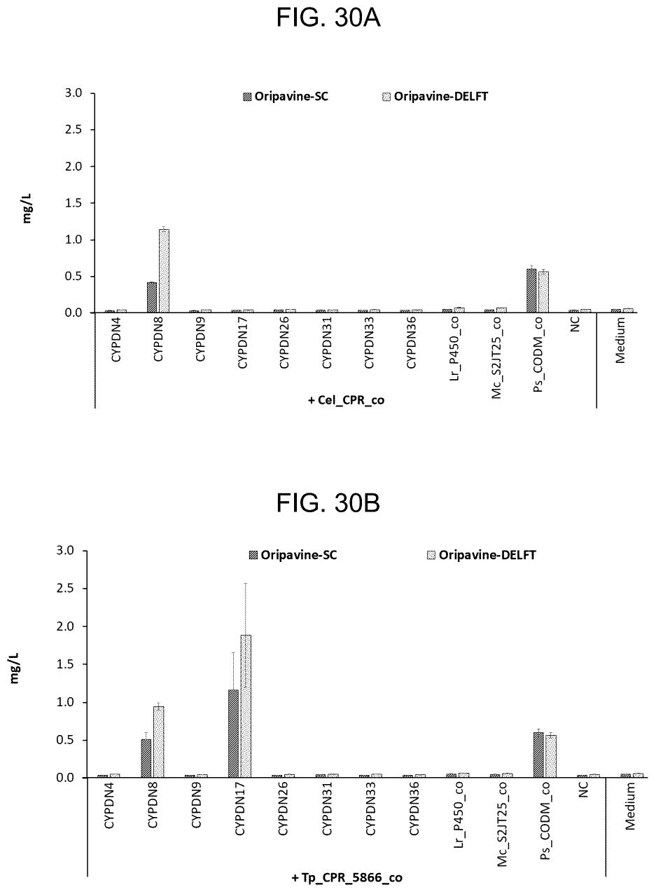

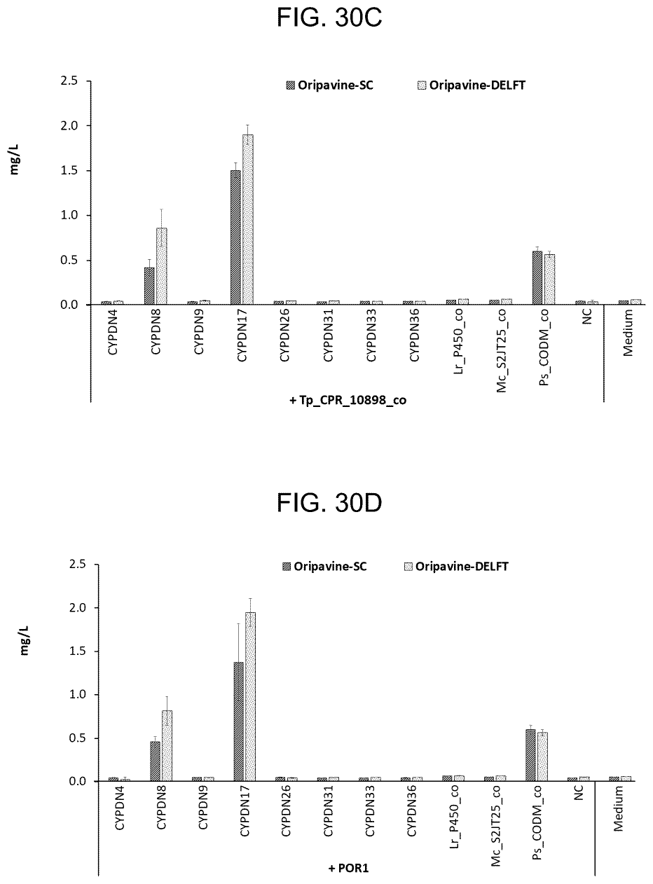

As seen in FIG. 30a-30d, CYPDN8 (SEQ ID NO: 72) and CYPDN17 (SEQ ID NO: 90) homologs display O-demethylation activity and are capable of converting thebaine to oripavine. This O-demethylation activity is apparently substantially higher than the one detected for the positive control Ps_CODM_co. Similarly, we also observed a positive effect on the oripavine production when cells were cultured in DELFT medium compared to synthetic complete medium.

Nororipavine Production by New Cytochrome P450 Fungal Homologs

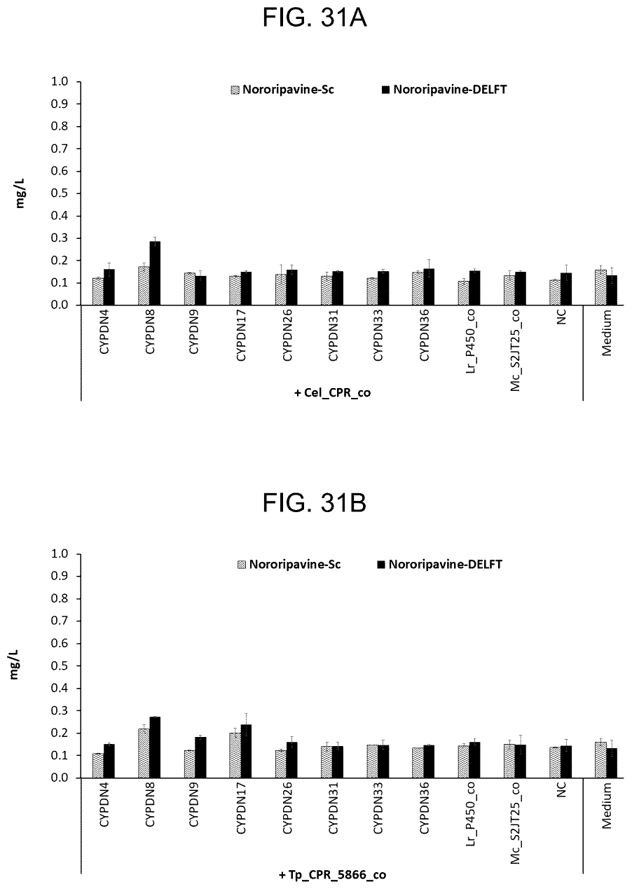

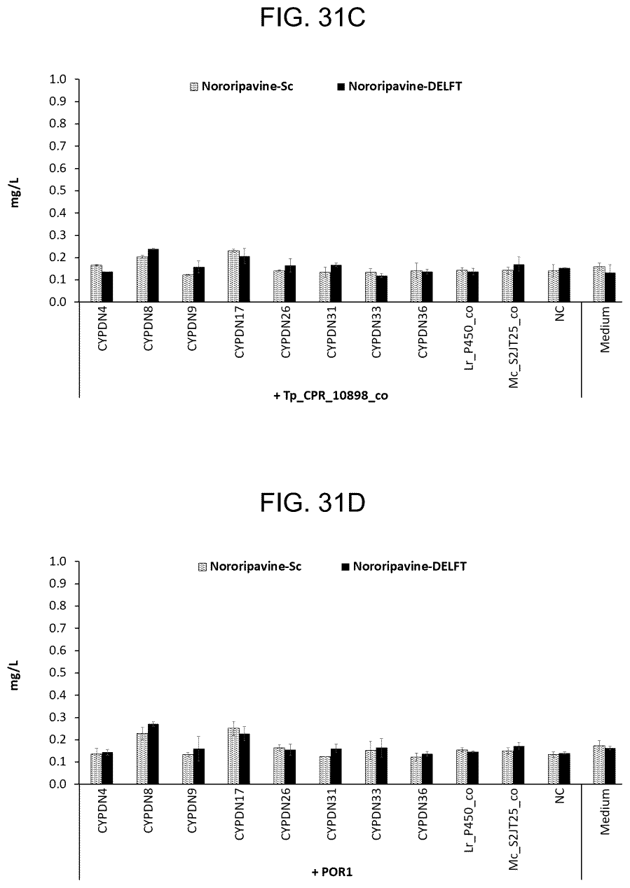

As seen in FIG. 31a-31d, nororipavine production was detected when S. cerevisiae strains expressing cytochrome P450s CYPDN8 (SEQ ID NO: 72) or CYPDN17 (SEQ ID NO: 90) were grown in the presence of thebaine. Based on the previous observations with N- and O-demethylation of thebaine and the detection of nororipavine production, CYPDN8 (SEQ ID NO: 72) or CYPDN17 (SEQ ID NO: 90) appear to possess both N- and O-demethylation activity.

### REFERENCES

Mumberg D., Müller R., Funk M. (1995). Yeast vectors for the controlled expression of heterologous proteins in different genetic backgrounds. Gene. 156(1):119-22.

TABLE 3
Northebaine production by fungal P450 and a N. sylvestris P450.

| | Northebaine |
|---|---|
| Name | production |
| CYPDN4 | − |
| CYPDN5 | + |
| CYPDN6 | + |
| CYPDN7 | + |
| CYPDN8 | + |
| CYPDN9 | − |
| CYPDN10 | + |
| CYPDN11 | + |
| CYPDN12 | + |
| CYPDN13 | + |
| CYPDN14 | + |
| CYPDN16 | + |
| CYPDN17 | + |
| CYPDN18 | + |
| CYPDN19 | − |
| CYPDN20 | + |
| CYPDN21 | + |
| CYPDN22 | + |
| CYPDN24 | + |
| CYPDN26 | − |
| CYPDN27 | + |
| CYPDN28 | + |
| CYPDN29 | + |
| CYPDN30 | + |
| CYPDN31 | + |
| CYPDN32 | − |
| CYPDN33 | − |
| CYPDN34 | + |
| CYPDN35 | + |
| CYPDN36 | − |

−: same or lower northebaine titers than negative control;
+: northebaine titers higher than negative control.

### Example 64. Production of northebaine from thebaine by Heterologous Expression of Genes Encoding N-demethylases in Non-Conventional Yeasts

Example: Cloning of fungal CYP450/CPRs and enzymes in non-conventional yeasts A combination of CYP450/CPRs were tested in a set of non-conventional yeasts in order to evaluate the N-demethylation of thebaine to northebaine. The tested CYP450 candidates were Lr_P450_co (SEQ ID NO: 8) ( ), Mc_S2JT25_co (SEQ ID NO: 52), P450_DN12791_c0_g1_i1_co (SEQ ID NO: 4) and the tested CPR enzymes were CPR_10898_c0_g1_i1_co (SEQ ID NO: 12), Cel_CPR_co (SEQ ID NO: 16) and CPR_5866_c0_g1_i1_co (SEQ ID NO: 9).

All gene sequences were codon-optimized for Saccharomyces cerevisiae expression and cloned in between the S. cerevisiae promoters (TEF1 or PGK1) and terminators (CYC1 or ADH1)) (see Table 1). DNA constructs containing promoter-gene-terminator were PCR amplified from plasmid templates (see Table 1) with standard primer sets containing deoxyuracil (dU). The PCR amplified fragments were cloned using the Uracil-Specific Excision Reagent (USER) based vector system (Jensen et al., 2014; Nour-Eldin et al., 2006) with some modifications. In order to express these genes in various non-conventional yeasts, a self-replicating vector containing a pangenomic optimized yeast replication origin panARS that allows stable plasmid expression in different yeast species, was used (Liachko and Dunham 2014). Nourseothricin/clonNat (natMX) dominant marker was used for selection in the different yeast species. Different combinations of plasmids containing CPRs/P450s were constructed (see Table 2). PanARS plasmids, containing the different promoter-gene-terminator fragments were transformed into different yeast strains (see Table 3) using the lithium acetate method (Gietz and Woods 2007) with some minor modifications. The heat shock step was performed at 40° C. for 1 h and cultures were grown at 30° C. on YPD media for 4 hours before plating to allow cells to acquire antibiotic resistance. The transformed strains expressing the genes of interest were grown in 0.5 ml of YEPD media at pH 7 with 50 mg/L of clonNat and 0.5 mM of thebaine added as a 110 mM stock solution in DMSO. The cultures were incubated at 30° C. with shaking at 300 rpm for 96 hours.

Metabolite Detection

Metabolites were analyzed by harvesting the media supernatant and detected directly by LC-MS-MS. LC-MS-MS were as Example 64.

TABLE 1
Amplified DNA fragments

| promoter-gene-terminator | DNA template |
|---|---|
| pTEF1 - Cel_CPR_co - tADHl | pEV31215 |
| pTEF1 - CPR_5866_c0_g1_i1 _co - tADH1 | pEV32634 |
| pTEF1 - CPR_10898_c0_gl_i1_co - tADH1 | pEV32635 |
| pPGK1 - Lr_P450_co - tCYCl | pEV32228 |
| pPGK1 -P450_DN12791_c0_g1_i1_co - tCYC1 | PEV32227 |
| pPGK1 - Mc_S2JT25_co - tCYC1 | pEV33161 |

TABLE 2
List of plasmids constructs

| | | Plasmid | Bacterial | Selec- |
|---|---|---|---|---|
| | | Back- | Selection | tion |
| Name | DNA constructs | bone | Marker | Marker |
| pOD73 | [pTEF1 - Cel_CPR_co - | pDIV19 | Amp | clonNat |
| | tADHI] + [pPGK1 - | | | |
| | Lr_P450_co - tCYC1] | | | |
| pOD74 | [pTEF1 - | pDIV19 | Amp | clonNat |
| | CPR_5866_c0_g1_i1_co - | | | |
| | tADH1] + [pPGK1 - | | | |
| | P450_DN12791_c0_g1_i1_co - | | | |
| | tCYC1] | | | |
| pOD75 | [pTEF1- | pDIV19 | Amp | clonNat |
| | CPR_10898_c0_g1_i1_co - | | | |
| | tADH1] + | | | |
| | [pPGK1 - Mc_S2JT25_co - | | | |
| | tCYC1] | | | |

TABLE 3
List of yeasts strains tested in this study

| Specie name | Genotype | Collection Ids |
|---|---|---|
| Saccharomyces | WT | CBS 2908 |
| paradoxus | | |
| Kluyveromyces marxianus | WT | IBT 42; CBS 1574 |
| Kluyveromyces marxianus | WT | IBT 82 |
| Kluyveromyces marxianus | WT | IBT 86 |
| Ogataea thermomethanolica | WT | CBS 8099 |

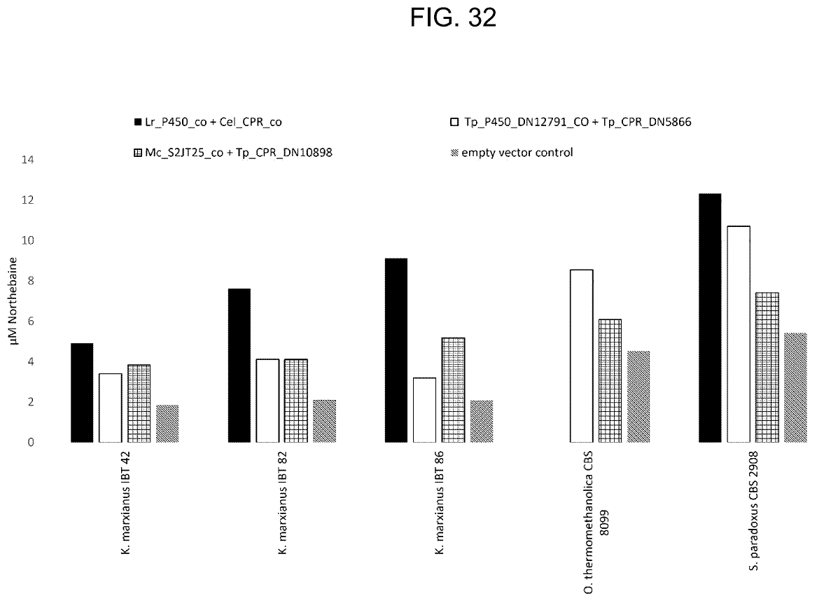

The bioconversion of thebaine to northebaine was achieved upon heterologous co-expression of N-demethylase genes together with cytochrome P450 reductase genes in 3 different strains of K. marxianus and one strain of O. thermomethanolica and one strain of S. paradoxus (FIG. 32). The formation of northebaine is clearly increased above the levels detected in the control strains containing the empty vector. The accumulation of northebaine in the vector control, especially for O. thermomethanolica and S. paradoxus, could point to one or more endogenous enzymes, which are capable of performing some conversion of thebaine into northebaine.

### REFERENCES

Nour-Eldin H. H., Hansen B. G., Norholm M. H., Jensen J. K., Halkier B. A. (2006). Advancing uracil-excision based cloning towards an ideal technique for cloning PCR fragments. Nucleic Acids Res. 34:e122.
Jensen, N. B. et al (2014). EasyClone: Method for iterative chromosomal integration of multiple genes in Saccharomyces cerevisiae. FEMS Yeast Res. 14, 238-248.
Gietz, R. D. & Schiestl, R. H. (2007). High-efficiency yeast transformation using the LiAc/SS carrier DNA/PEG method. Nat Protoc 2(1), 31-4.
Liachko and Dunham (2014). An Autonomously Replicating Sequence for use in a wide range of budding yeasts. FEMS Yeast Res. 14(2): 364-367.

### Tables

TABLE 1
Cytochrome P450 reductases

| Transcript/protein | |
|---|---|
| (Accession Number) | Species/Strain |
| CPR_DN2505_c0_g1_i1 (n.a.) | Thamnostylum piriforme |
| (nucleotide sequence SEQ ID NO: 15) | ATCC 8992 |
| CPR_DN5866_c0_g1_i1 (n.a.) (SEQ ID | Thamnostylum piriforme |
| NO: 9) | ATCC 8992 |
| CPR_DN10898_c0_g1_i1 (n.a.) (SEQ ID | Thamnostylum piriforme |
| NO: 12) | ATCC 8992 |
| NADPH-dependent cytochrome P450 | Cunninghamella elegans |
| oxidoreductase (AAF89958) (SEQ ID NO: | |
| 16) | |
| Cytochrome P450 oxidoreductase | Gibberella fujikuroi |
| (Q7Z8R1) (SEQ ID NO: 18) | |
| NADPH-cytochrome P450 reductase | Saccharomyces cerevisiae |
| (P16603) (SEQ ID NO: 20) | |
| NADPH-cytochrome P450 reductase | Homo sapiens |
| (BAB18572.1) (SEQ ID NO: 50) | |

TABLE 2
Cloning strategy of target genes into yeast expression vectors.

| Parent backbone | Promoter-Gene- | | Restriction | |
|---|---|---|---|---|
| and description | Terminator | Gene Description | sites used | Final construct |
| pEVE2120, single | pPGK1-Tp_P450 (_1 to | P450s from T. piriforme (22 | SfiI/SacII | PEV31493-31564, |
| expression vector | _23)-tADH2, except | ORFs) | | excluding |
| [ARS-CEN/pPGK1- | Tp_P450_6 | | | pEV31508-3150 |
| tADH2/URA3], | pPGK1-Tp_P450_6-tADH2 | P450_DN9560_c0_g1_i1 from | SfiI/BamHI | pEV31508-31510 |
| Amp^{R} | | T. piriforme | | |
| pEVE3307, double | pTEF1-Tp_CPR_1-tADH1 | CPR_DN2505_c0_g1_i1 from | PmeI/PacI | pEV31719-31722 |
| expression vector | | T. piriforme | | |
| [ARS-CEN/pTEF1- | pTEF1-Cel_CPR_co-tADH1 | CPR from Cunninghamella | PmeI/PacI | pEV31215 |
| tADH1/pPGK1- | | elegans (co) | | |
| tCYC1/HIS3], | pTEF1-Sc_CPR-tADH1 | CPR from S. cerevisiae | PmeI/PacI | pEV28686 |
| Amp^{R} | | | | |
| pEVE3308, double | pTEF1-Tp_CPR_2-tADH1 | CPR_DN5866_c0_g1_i1 from | PmeI/PacI | pEV31723-31729 |
| expression vector | | T. piriforme | | |
| [ARS-CEN/PTEF1- | pTEF1-Tp_CPR_3-tADH1 | CPR_DN10898_c0_g1_i1 from | PmeI/PacI | pEV31730-31733 |
| tADH1/pPGK1- | | T. piriforme | | |
| tCYC1/LEU2], | pPGK1-Gf_CPR_co-tCYC1 | CPR from Gibberella fujikuroi | HindIII/SacII | pEV31104 |
| Amp^{R} | | (co) | | |
| pEV31104 | pTEF1-Sc_CPR- | CPR from S. cerevisiae and | PmeI/PacI | pEV31308 |
| | tADH1/pPGK1-Gf_CPR_co- | CPR from Gibberella fujikuroi | | |
| | tCYC1 | (co) | | |
| | pTEF1-Tp_CPR_3- | CPR_DN10898_c0_g1_i1 from | PmeI/PacI | pEV31734-31737 |
| | tADH1/pPGK1-Gf_CPR_co- | T. piriforme and CPR from | | |
| | tCYC1 | Gibberella fujikuroi (co) | | |

Abbreviations:
co, codon optimized for S. cerevisiae.

TABLE 3
Oligonucleotides used for PCR amplification of target
genes from Thamnostylum piriforme.

| | | |
|---|---|---|
| No. | Transcript name | Primer name |

P450 gene candidates

| | | | |
|---|---|---|---|
| 1 | P450_DN2971_c0_g2_i1 | EVPR18235_39D2 | fw |
| | | EVPR18236_39D3 | rev |
| 2 | P450_DN6887_c0_g1_i2_1 | EVPR18237_39D4 | fw |
| | | EVPR18239_39D6 | rev |
| 3 | P450_DN6887_c0_g1_i2_2 | EVPR18238_39D5 | fw |
| | | EVPR18239_39D6 | rev |
| 4 | P450_DN8261_c0_g2_i1 | EVPR18240_39D7 | fw |
| | | EVPR18241_39D8 | rev |
| 5 | P450_DN9169_c0_g2_i1 | EVPR18242_39D9 | fw |
| | | EVPR18243_39E1 | rev |
| 6 | P450_DN9560_c0_g1_i1 | EVPR18244_39E2 | fw |
| | | EVPR18245_39E3 | rev |
| 7 | P450_DN10444_c0_g1_i1 | EVPR18246_39E4 | fw |
| | | EVPR18247_39E5 | rev |
| 8 | P450_DN10880_c0_g1_i3 | EVPR18248_39E6 | fw |
| | | EVPR18249_39E7 | rev |
| 9 | P450_DN12040_c0_g1_i1 | EVPR18250_39E8 | fw |
| | | EVPR18251_39E9 | rev |
| 10 | P450_DN12791_c0_g1_i1 | EVPR18252_39F1 | fw |
| | | EVPR18253_39F2 | rev |
| 11 | P450_DN13606_c0_g1_i3 | EVPR18254_39F3 | fw |
| | | EVPR18255_39F4 | rev |
| 12 | P450_DN13846_c0_g1_i2 | EVPR18256_39F5 | fw |
| | | EVPR18257_39F6 | rev |
| 13 | P450_DN14156_c0_g1_i1 | EVPR18258_39F7 | fw |
| | | EVPR18259_39F8 | rev |
| 14 | P450_DN14346_c0_g2_i2 | EVPR18260_39F9 | fw |
| | | EVPR18261_39G1 | rev |
| 15 | P450_DN14398_c0_g2_i6 | EVPR18262_39G2 | fw |
| | | EVPR18263_39G3 | rev |
| 16 | P450_DN14859_c0_g1_i9 | EVPR18264_39G4 | fw |
| | | EVPR18265_39G5 | rev |
| 17 | P450_DN15259_c0_g1_i7 | EVPR18266_39G6 | fw |
| | | EVPR18267_39G7 | rev |
| 18 | P450_DN15334_c0_g1_i1 | EVPR18268_39G8 | fw |
| | | EVPR18269_39G9 | rev |
| 19 | P450_DN16201_c0_g1_i2_1 | EVPR18270_39H1 | fw |
| | | EVPR18273_39H4 | rev |
| 20 | P450_DN16201_c0_g1_i2_2 | EVPR18271_39H2 | fw |
| | | EVPR18273_39H4 | rev |
| 21 | P450_DN16201_c0_g1_i2_3 | EVPR18272_39H3 | fw |
| | | EVPR18273_39H4 | rev |
| 22 | P450_DN16821_c0_g1_i8 | EVPR18274_39H5 | fw |
| | | EVPR18275_39H6 | rev |
| 23 | P450_DN16821_c0_g1_i12 | EVPR18276_39H7 | fw |
| | | EVPR18277_39H8 | rev |

CPR gene candidates

| | | | |
|---|---|---|---|
| 1 | CPR_DN2505_c0_g1_i1 | EVPR18278_39H9 | fw |
| | | EVPR18279_39I1 | rev |
| 2 | CPR_DN5866_c0_g1_i1 | EVPR18280_39I2 | fw |
| | | EVPR18281_39I3 | rev |
| 3 | CPR_DN10898_c0_g1_i1 | EVPR18282_39I4 | fw |
| | | EVPR18283_39I5 | rev |

Abbreviations:
fw: forward primer;
rev: reverse primer.

TABLE 4
Description of plasmids containing target genes from T. piriforme.
Unless otherwise specified, all clones originated from
cDNA obtained on day 6 after induction.

| Vector name | Backbone | Promoter-Gene-Terminator |
|---|---|---|
| pEV31493 | pEVE2120 | pPGK1-P450_DN2971_c0_g2_i1_A-tADH2 |
| pEV31494 | pEVE2120 | pPGK1-P450_DN2971_c0_g2_i1_B-tADH2 |
| pEV31495 | pEVE2120 | pPGK1-P450_DN2971_c0_g2_i1_C-tADH2 |
| pEV31496 | pEVE2120 | pPGK1-P450_DN6887_c0_g1_i2_1A-tADH2 |
| pEV31497 | pEVE2120 | pPGK1-P450_DN6887_c0_g1_i2_1B-tADH2 |
| pEV31498 | pEVE2120 | pPGK1-P450_DN6887_c0_g1_i2_1C-tADH2 |
| pEV31499 | pEVE2120 | pPGK1-P450_DN6887_c0_g1_i2_2A-tADH2 |
| pEV31500 | pEVE2120 | pPGK1-P450_DN6887_c0_g1_i2_2B-tADH2 |
| pEV31501 | pEVE2120 | pPGK1-P450_DN6887_c0_g1_i2_2C-tADH2 |
| pEV31502 | pEVE2120 | pPGK1-P450_DN8261_c0_g2_i1_A-tADH2 |
| pEV31503 | pEVE2120 | pPGK1-P450_DN8261_c0_g2_i1_B-tADH2 |
| pEV31504 | pEVE2120 | pPGK1-P450_DN8261_c0_g2_i1_C-tADH2 |
| pEV31505 | pEVE2120 | pPGK1-P450_DN9169_c0_g2_i1_A-tADH2 |
| pEV31506 | pEVE2120 | pPGK1-P450_DN9169_c0_g2_i1_B-tADH2 |
| pEV31507 | pEVE2120 | pPGK1-P450_DN9169_c0_g2_i1_C-tADH2 |
| pEV31508 | pEVE2120 | pPGK1-P450_DN9560_c0_g1_i1_A-tADH2 |
| pEV31509 | pEVE2120 | pPGK1-P450_DN9560_c0_g1_i1_B-tADH2 |
| pEV31510 | pEVE2120 | pPGK1-P450_DN9560_c0_g1_i1_C-tADH2 |
| pEV31511 | pEVE2120 | pPGK1-P450_DN10444_c0_g1_i1_A-tADH2 |
| pEV31512 | pEVE2120 | pPGK1-P450_DN10444_c0_g1_i1_B-tADH2 |
| pEV31513 | pEVE2120 | pPGK1-P450_DN10444_c0_g1_i1_C-tADH2 |
| pEV31514 | pEVE2120 | pPGK1-P450_DN10880_c0_g1_i3_A-tADH2 |
| pEV31515 | pEVE2120 | pPGK1-P450_DN10880_c0_g1_i3_B-tADH2 |
| pEV31516 | pEVE2120 | pPGK1-P450_DN10880_c0_g1_i3_C-tADH2 |
| pEV31517 | pEVE2120 | pPGK1-P450_DN12040_c0_g1_i1_A-tADH2 |
| pEV31518 | pEVE2120 | pPGK1-P450_DN12040_c0_g1_i1_B-tADH2 |
| pEV31519 | pEVE2120 | pPGK1-P450_DN12040_c0_g1_i1_C-tADH2 |
| pEV31520 | pEVE2120 | pPGK1-P450_DN12791_c0_g1_i1_A-tADH2 |
| pEV31521 | pEVE2120 | pPGK1-P450_DN12791_c0_g1_i1_B-tADH2 |
| pEV31522 | pEVE2120 | pPGK1-P450_DN12791_c0_g1_i1_C-tADH2 |
| pEV31523 | pEVE2120 | pPGK1-P450_DN13606_c0_g1_i3_A-tADH2 |
| pEV31524 | pEVE2120 | pPGK1-P450_DN13606_c0_g1_i3_B-tADH2 |
| pEV31525 | pEVE2120 | pPGK1-P450_DN13606_c0_g1_i3_C-tADH2 |
| pEV31526 | pEVE2120 | pPGK1-P450_DN13846_c0_g1_i2_A-tADH2 |
| pEV31527 | pEVE2120 | PPGK1-P450_DN13846_c0_g1_i2_B-tADH2 |
| pEV31528 | pEVE2120 | pPGK1-P450_DN13846_c0_g1_i2_C-tADH2 |
| pEV31529 | pEVE2120 | pPGK1-P450_DN14156_c0_g1_i1_A-tADH2 |
| pEV31530 | pEVE2120 | pPGK1-P450_DN14156_c0_g1_i1_B-tADH2 |
| pEV31531 | pEVE2120 | pPGK1-P450_DN14156_c0_g1_i1_C-tADH2 |
| pEV31532 | pEVE2120 | pPGK1-P450_DN14346_c0_g2_i2_A-tADH2 |
| pEV31533 | pEVE2120 | pPGK1-P450_DN14346_c0_g2_i2_B-tADH2 |
| pEV31534 | pEVE2120 | pPGK1-P45O_DN14346_c0_g2_i2_C-tADH2 |
| pEV31535 | pEVE2120 | pPGK1-P450_DN14398_c0_g2_i6_A-tADH2 |
| pEV31536 | pEVE2120 | pPGK1-P450_DN14398_c0_g2_i6_B-tADH2 |
| pEV31537 | pEVE2120 | pPGK1-P450_DN14398_c0_g2_i6_C-tADH2 |
| pEV31538 | pEVE2120 | pPGK1-P450_DN14859_c0_g1_i9_A-tADH2 |
| pEV31539 | pEVE2120 | pPGK1-P450_DN14859_c0_g1_i9_B-tADH2 |
| pEV31540 | pEVE2120 | pPGK1-P450_DN14859_c0_g1_i9_C-tADH2 |
| pEV31541 | pEVE2120 | pPGK1-P450_DN15259_c0_g1_i7_A-tADH2 |
| pEV31542 | pEVE2120 | pPGK1-P450_DN15259_c0_g1_i7_B-tADH2 |
| pEV31543 | pEVE2120 | pPGK1-P450_DN15259_c0_g1_i7_C-tADH2 |
| pEV31544 | pEVE2120 | pPGK1-P450_DN15259_c0_g1_i7_Ad9-tADH2 |
| pEV31545 | pEVE2120 | pPGK1-P450_DN15259_c0_g1_i7_Bd9-tADH2 |
| pEV31546 | pEVE2120 | pPGK1-P450_DN15259_c0_g1_i7_Cd9-tADH2 |
| pEV31547 | pEVE2120 | pPGK1-P450_DN15334_c0_g1_i1_A-tADH2 |
| pEV31548 | pEVE2120 | pPGK1-P450_DN15334_c0_g1_i1_B-tADH2 |
| pEV31549 | pEVE2120 | pPGK1-P450_DN15334_c0_g1_i1_C-tADH2 |
| pEV31550 | pEVE2120 | pPGK1-P450_DN16201_c0_g1_i2_1A-tADH2 |
| pEV31551 | pEVE2120 | pPGK1-P450_DN16201_c0_g1_i2_1B-tADH2 |
| pEV31552 | pEVE2120 | pPGK1-P450_DN16201_c0_g1_i2_1C-tADH2 |
| pEV31553 | pEVE2120 | pPGK1-P450_DN16201_c0_g1_i2_2A-tADH2 |
| pEV31554 | pEVE2120 | pPGK1-P450_DN16201_c0_g1_i2_2B-tADH2 |
| pEV31555 | pEVE2120 | pPGK1-P450_DN16201_c0_g1_i2_2C-tADH2 |
| pEV31556 | pEVE2120 | pPGK1-P450_DN16201_c0_g1_i2_3A-tADH2 |
| pEV31557 | pEVE2120 | pPGK1-P450_DN16201_c0_g1_i2_3B-tADH2 |
| pEV31558 | pEVE2120 | pPGK1-P450_DN16201_c0_g1_i2_3C-tADH2 |
| pEV31559 | pEVE2120 | pPGK1-P450_DN16821_c0_g1_i8_A-tADH2 |
| pEV31560 | pEVE2120 | pPGK1-P450_DN16821_c0_g1_i8_B-tADH2 |
| pEV31561 | pEVE2120 | pPGK1-P450_DN16821_c0_g1_i8_C-tADH2 |
| pEV31562 | pEVE2120 | pPGK1-P450_DN16821_c0_g1_i12_A-tADH2 |
| pEV31563 | pEVE2120 | pPGK1-P450_DN16821_c0_g1_i12_B-tADH2 |
| pEV31564 | pEVE2120 | pPGK1-P450_DN16821_c0_g1_i12_C-tADH2 |
| pEV31719 | pEVE3307 | pTEF1-CPR_DN2505_c0_g1_i1_A-tADH1 |
| pEV31720 | pEVE3307 | pTEF1-CPR_DN2505_c0_g1_i1_B-tADH1 |
| pEV31721 | pEVE3307 | pTEF1-CPR_DN2505_c0_g1_i1_C-tADH1 |
| pEV31722 | pEVE3307 | pTEF1-CPR_DN2505_c0_g1_i1_D-tADH1 |
| pEV31723 | pEVE3308 | pTEF1-CPR_DN5866_c0_g1_i1_Ad9-tADH1 |
| pEV31724 | pEVE3308 | pTEF1-CPR_DN5866_c0_g1_i1_Bd9-tADH1 |
| pEV31725 | pEVE3308 | pTEF1-CPR_DN5866_c0_g1_i1_Cd9-tADH1 |
| pEV31726 | pEVE3308 | pTEF1-CPR_DN5866_c0_g1_i1_A-tADH1 |
| pEV31727 | pEVE3308 | pTEF1-CPR_DN5866_c0_g1_i1_B-tADH1 |
| pEV31728 | pEVE3308 | pTEF1-P450_DN5866_c0_g1_i1_C-tADH1 |
| pEV31729 | pEVE3308 | pTEF1-CPR_DN5866_c0_g1_i1_D-tADH1 |
| pEV31730 | pEVE3308 | pTEF1-CPR_DN10898_c0_g1_1_1A-tADH1 |
| pEV31731 | pEVE3308 | pTEF1-CPR_DN10898_c0_g1_1_1B-tADH1 |
| pEV31732 | pEVE3308 | pTEF1-CPR_DN10898_c0_g1_1_1C-tADH1 |
| pEV31733 | pEVE3308 | pTEF1-CPR_DN10898_c0_g1_1_1D-tADH1 |
| pEV31734 | pEV31104 | pTEF1-CPR_DN10898_c0_g1_1_2A-tADH1/ |
| | | pPGK1-Gf_CPR_coSc-tCYC1 |
| pEV31735 | pEV31104 | pTEF1-CPR_DN10898_c0_g1_1_2B-tADH1/ |
| | | pPGK1-Gf_CPR_coSc-tCYC1 |
| pEV31736 | pEV31104 | pTEF1-CPR_DN10898_c0_g1_1_2C-tADH1/ |
| | | pPGK1-Gf_CPR_coSc-tCYC1 |
| pEV31737 | pEV31104 | pTEF1-CPR_DN10898_c0_g1_i1_2D-tADH1/ |
| | | pPGK1-Gf_CPR_coSc-tCYC1 |

“d9” stands for day 9 after induction.

TABLE 5
Fungal Cytochrome P450 enzymes

| Transcript/protein | Species/Strain |
|---|---|
| DN15259_c0_g1_17 (n.a.) (SEQ ID NO: 1) | Thamnostylum piriforme |
| | ATCC 8992 |
| DN12791_c0_g1_i1 (n.a.) (SEQ ID NO: 4) | Thamnostylum piriforme |
| | ATCC 8992 |
| hypothetical protein LRAMOSA08132 | Lichtheimia ramosa |
| (A0A077WEM0) (SEQ ID NO: 7) | |

TABLE 6
Description of plasmids containing codon-optimized P450 genes.

| Vector | | | |
|---|---|---|---|
| name | Backbone | Promoter-Gene-Terminator | Description |
| pEV32226 | pEVE3306 | pPGK1-P450_DN15259_c0_g1_i7_co- | P450_DN_15259 (co) from T. piriforme |
| | | tADH2 | |
| pEV32227 | pEVE3306 | pPGK1-P450_DN12791_c0_g1_i1_co- | P450_DN_12791 (co) from T. piriforme |
| | | tADH2 | |
| pEV32228 | pEVE3306 | pPGK1-Lr_P450_co-tADH2 | P450 from Lichtheimia ramosa (co) |
| pEV32640 | pEVE3306 | pPGK1-P450_DN_16393_co-tCYC1 | P450_DN16393 (co) from T. piriforme |
| pEV32641 | pEVE3306 | pPGK1-LCOR_01865_co-tCYC1 | LCOR_01865 (co) from Lichtheimia |
| | | | corymbifera |
| pEV32642 | pEVE3306 | pPGK1-LCOR_09548_co-tCYC1 | LCOR_09548 (co) from Lichtheimia |
| | | | corymbifera |
| pEV32643 | pEVE3306 | pPGK1-Ar_ORZ22410_co-tCYC1 | ORZ22410 (co) from Absidia repens |
| pEV33161 | pEVE3306 | pPGK1-Mc_S2JT25_co-tCYC1 | S2JT25 (co) from Mucor circinelloides |
| pOD8 | P415-TEF | pTEF1-Lr_P450_co-tCYC1 | P450 from Lichtheimia ramosa (co) |
| pOD10 | P415-TEF | pTEF1-Mc_S2JT25_co-tCYC1 | S2JT25 (co) from Mucor circinelloides |
| pOD36 | P415-TEF | pTEF1-CYPDN5-tCYC1 | A0A1X0RYU8 (co) from Rhizopus |
| | | | microsporus |
| pOD37 | P415-TEF | pTEF1-CYPDN6-tCYC1 | A0A0B7NKC0 (co) from Parasitella |
| | | | parasitica |
| pOD38 | P415-TEF | pTEF1-CYPDN7-tCYC1 | HCBZ0 (co) from Rhizopus delemar |
| pOD39 | P415-TEF | pTEF1-CYPDN10-tCYC1 | A0A0B7NB52 (co) from Parasitella |
| | | | parasitica |
| pOD40 | P415-TEF | pTEF1-CYPDN11-tCYC1 | A0A068SB73 (co) from Lichtheimia |
| | | | corymbifera |
| pOD41 | P415-TEF | pTEF1-CYPDN12-tCYC1 | A0A1C7N669 (co) from Choanephora |
| | | | cucurbitarum |
| pOD42 | P415-TEF | pTEF1-CYPDN13-tCYC1 | A0A1X2HZE4 (co) from Absidia repens |
| pOD43 | P415-TEF | pTEF1-CYPDN14-tCYC1 | A0A168T1R5 (co) from Absidia glauca |
| pOD44 | P415-TEF | pTEF1-CYPDN16-tCYC1 | A0A1X2HFU1 (co) from |
| | | | Syncephalastrum racemosum |
| pOD45 | P415-TEF | pTEF1-CYPDN18-tCYC1 | A0A0C9MNJ6 (co) from Mucor ambiguus |
| pOD46 | P415-TEF | pTEF1-CYPDN19-tCYC1 | A0A163B0W9 (co) from Phycomyces |
| | | | blakesleeanus |
| pOD47 | P415-TEF | pTEF1-CYPDN20-tCYC1 | A0A163A6R1 (co) from Phycomyces |
| | | | blakesleeanus |
| pOD48 | P415-TEF | pTEF1-CYPDN21-tCYC1 | XM_018432503 (co) from Phycomyces |
| | | | blakesleeanus |
| pOD49 | P415-TEF | pTEF1-CYPDN22-tCYC1 | XM_023609077 (co) from Rhizopus |
| | | | microsporus |
| pOD50 | P415-TEF | pTEF1-CYPDN24-tCYC1 | A0A077WFB8 (co) from Lichtheimia |
| | | | ramosa |
| pOD51 | P415-TEF | pTEF1-CYPDN27-tCYC1 | A0A077WLY1 (co) from Lichtheimia |
| | | | ramosa |
| pOD52 | P415-TEF | pTEF1-CYPDN28-tCYC1 | A0A1X2HYB6 (co) from Absidia repens |
| pOD53 | P415-TEF | pTEF1-CYPDN29-tCYC1 | A0A068SBU5 (co) from Lichtheimia |
| | | | corymbifera |
| pOD54 | P415-TEF | pTEF1-CYPDN30-tCYC1 | A0A1X2H4N2 (co) from |
| | | | Syncephalastrum racemosum |
| pOD55 | P415-TEF | pTEF1-CYPDN32-tCYC1 | A0A173GQ95 (co) from Absidia caerulea |
| pOD56 | P415-TEF | pTEF1-CYPDN34-tCYC1 | A0A068SBP8 (co) from Lichtheimia |
| | | | corymbifera |
| pOD57 | P415-TEF | pTEF1-CYPDN35-tCYC1 | A0A1X2GSL0 (co) from Hesseltinella |
| | | | vesiculosa |
| pOD75 | P415-TEF | pTEF1-CYPDN8-tCYC1 | A0A0C7AZL4 (co) from Rhizopus |
| | | | microsporus |
| pOD79 | P415-TEF | pTEF1-CYPDN4-tCYC1 | I1CCX6 (co) from Rhizopus delemar |
| pOD80 | P415-TEF | pTEF1-CYPDN9-tCYC1 | A0A1X2HQ99 (co) from |
| | | | Syncephalastrum racemosum |
| pOD82 | P415-TEF | pTEF1-CYPDN17-tCYC1 | A0A2G4SQ34 (co) from Rhizopus |
| | | | microsporus |
| pOD85 | P415-TEF | pTEF1-CYPDN26-tCYC1 | A0A162N972 (co) from Phycomyces |
| | | | blakesleeanus |
| pOD86 | P415-TEF | pTEF1-CYPDN31-tCYC1 | A0A291C3B2 (co) from Absidia caerulea |
| pOD87 | P415-TEF | pTEF1-CYPDN33-tCYC1 | A0A162UUM5 (co) from Phycomyces |
| | | | blakesleeanus |
| pOD88 | P415-TEF | pTEF1-CYPDN36-tCYC1 | E5KY66 (co) from Nicotiana sylvestris |
| pEV33313 | pEVE3306 | pPGK1- | P450_DN5328_c5_g3_i1 (co) from |
| | | P450_DN5328_c5_g3_i1_coSc-tCYC1 | Cunninghamella echinulata |
| pEV33314 | pEVE3306 | pPGK1- | P450_DN5457_c2_g2_i2 (co) from |
| | | P450_DN5457_c2_g2_i2_coSc-tCYC1 | Cunninghamella echinulata |
| pEV33315 | pEVE3306 | pPGK1- | P450_DN5457_c2_g2_i3 (co) from |
| | | P450_DN5457_c2_g2_i3_coSc-tCYC1 | Cunninghamella echinulata |
| pEV33316 | pEVE3306 | pPGK1- | P450_DN5457_c2_g8_i1 (co) from |
| | | P450_DN5457_c2_g8_i1_coSc-tCYC1 | Cunninghamella echinulata |
| pEV33317 | pEVE3306 | pPGK1- | P450_DN5458_c5_g4_i1 (co) from |
| | | P450_DN5458_c5_g4_i1_coSc-tCYC1 | Cunninghamella echinulata |
| pEV33318 | pEVE3306 | pPGK1- | P450_DN5584_c2_g2_i2 (co) from |
| | | P450_DN5584_c2_g2_i2_coSc-tCYC1 | Cunninghamella echinulata |
| pEV33319 | pEVE3306 | pPGK1- | P450_DN5615_c2_g1_i9 (co) from |
| | | P450_DN5615_c2_g1_i9_coSc-tCYC1 | Cunninghamella echinulata |

Abbreviations:
co, codon optimized for S. cerevisiae.

TABLE 7
Transformation of EVST25898 with cytochrome P450 P450_DN15259_c0_g1_i7_A and CPR genes

| Genes in EVST25898 | HIS | LEU | URA |
|---|---|---|---|
| P450_DN15259_c0_g1_i7_A/Cel_CPR_co/Sc_CPR/Gf_CPR_co | pEV31215 | pEV31308 | pEV31541 |
| P450_DN15259_c0_g1_i7_A/Cel_CPR_co | pEV31215 | pEVE3308 | pEV31541 |
| P450_DN15259_c0_g1_i7_A/Gf_CPR_co | pEVE3307 | pEV31104 | pEV31541 |
| P450_DN15259_c0_g1_i7_A/Sc_CPR | pEV28686 | pEVE3308 | pEV31541 |
| P450_DN15259_c0_g1_i7_A/CPR_DN2505_c0_g1_i1_A | pEV31719 | pEVE3308 | pEV31541 |
| P450_DN15259_c0_g1_i7_A/CPR_DN2505_c0_g1_i1_D | pEV31722 | pEVE3308 | pEV31541 |
| P450_DN15259_c0_g1_i7_A/CPR_DN5866_c0_g1_i1_Ad9 | pEVE3307 | pEV31723 | pEV31541 |
| P450_DN15259_c0_g1_i7_A/CPR_DN5866_c0_g1_i1_Cd9 | pEVE3307 | pEV31725 | pEV31541 |
| P450_DN15259_c0_g1_i7_A/CPR_DN5866_c0_g1_i1_B | pEVE3307 | pEV31727 | pEV31541 |
| P450_DN15259_c0_g1_i7_A/CPR_DN5866_c0_g1_i1_Cd9 | pEVE3307 | pEV31728 | pEV31541 |
| P450_DN15259_c0_g1_i7_A/CPR_DN10898_c0_g1_i_1A | pEVE3307 | pEV31730 | pEV31541 |
| P450_DN15259_c0_g1_i7_A/CPR_DN10898_c0_g1_i_2D | pEVE3307 | pEV31737 | pEV31541 |

TABLE 8
Mammalian Cytochrome P450 3A4

| Transcript/protein | |
|---|---|
| (Accession Number) | Species/Strain |
| Cytochrome P450 3A4 (P08684) (SEQ ID | Homo sapiens |
| NO: 22) | |
| Uncharacterized protein (H2PLK4) (SEQ | Pongo abelii |
| ID NO: 24) | |
| Uncharacterized protein (A0A096NZ89) | Papio anubis |
| (SEQ ID NO: 26) | |
| Uncharacterized protein (G3SB46) (SEQ | Gorilla gorilla gorilla |
| ID NO: 28) | |
| Uncharacterized protein (F1PDL2) (SEQ | Canis lupus familiaris |
| ID NO: 30) | |

TABLE 9
Mammalian Cytochrome P450 3A5

| Transcript/protein | |
|---|---|
| (Accession Number) | Species/Strain |
| Cytochrome P450 3A5 (P20815) (SEQ | Homo sapiens |
| ID NO: 40) | |
| Cytochrome P450 3A5 (A4ZZ70) (SEQ | Pan troglodytes |
| ID NO: 42) | |
| Cytochrome P450 3A5 (A8CBR0) (SEQ | Macaca fascicularis |
| ID NO: 44) | |
| Cytochrome P450 3A5 (U3ECK3) (SEQ | Callithrix jacchus |
| ID NO: 46) | |

TABLE 11
Cytochrome b5

| Transcript/protein | |
|---|---|
| (Accession Number) | Species/Strain |
| Cytochrome b5 isoform 1 (NP_683725) | Homo sapiens |
| (SEQ ID NO: 48) | |

TABLE 10
Mammalian Cytochrome P450 2C8

| Transcript/protein | |
|---|---|
| (Accession Number) | Species/Strain |
| Cytochrome P450 2C8 (P10632) (SEQ ID | Homo sapiens |
| NO: 32) | |
| Uncharacterized protein (H2Q2B) (SEQ | Pan troglodytes |
| ID NO: 34) | |
| Uncharacterized protein (H2NB34) (SEQ | Pongo abelii |
| ID NO: 36) | |
| Cytochrome P450 2C8 (Q4U0S8) (SEQ | Chlorocebus aethiops |
| ID NO: 38) | |

TABLE 13
Gradient for chiral separation

| Time (min) | % B |
|---|---|
| 0 | 2 |
| 5 | 35 |
| 6 | 100 |
| 7 | 100 |
| 7.1 | 1 |
| 8 | 1 |

TABLE 2
LC-MS conditions

| Parameter | Value |
|---|---|
| Injection volume | 2 μl |
| Column Temperature | 30° C. ± 4° C. |
| Injection method | Partial loop |
| Flow | 0.4 ml/min |
| Auto sampler temperature | 10° C. ± 2° C. |
| Weak wash | 800 μl water/acetonitrile 8:2 |
| Strong wash | 300 μl MeOH |
| Seal wash | 5 min with water/acetonitrile 9:1 |

Table 14: LC-MS conditions

TABLE 3
Mass spectrometer source and detector parameters (TQD)

| Source Parameter | Value |
|---|---|
| Ion Source | Electrospray Positive Mode (ESI+) |
| Capillary Voltage | 3.5 kV |
| Cone Voltage | 16 V |
| Extractor | 3 V |
| RF lens | 0.1 V |
| Source Temperature | 150° C. |
| Desolvation temperature | 350° C. |
| LM resolution 1 | 14 |
| HM resolution 1 | 14 |
| Ion Energy | 0.5 eV |
| Mode | MS/MS |
| Entrance | 50 eV |
| Collision | See Table 4 |
| Exit | 50 eV |
| LM resolution2 | 14 |
| HM resolution2 | 14 |
| Ion Energy2 | 0.5 |
| Desolvation gas | 500 L/hour |
| Cone gas | 50 L/hour |

Table 15: Mass spectrometer source and detector parameters (TQD)

TABLE 4
Multiple reaction monitoring targets and conditions (ESI+)

| | Retention | Parent | Daughter | Dwell | Cone | Collision |
|---|---|---|---|---|---|---|
| Target | time | ion | ion | time | voltage | energy |
| compound | (min) | (m/z) | (m/z) | (s) | (V) | (V) |
| Caffeine | 2.85 | 195 | 138 | 0.222 | 30 | 24 |
| Normorphine | 1.54 | 272 | 152 | 0.222 | 50 | 60 |
| Norcodeine | 2.36 | 286 | 152 | 0.222 | 44 | 56 |
| Nororipavine | 2.54 | 284 | 218 | 0.222 | 20 | 20 |
| Northebaine | 3.79 | 298 | 251 | 0.222 | 18 | 28 |
| Norsalutaridine | 3.16 | 314 | 165 | 0.222 | 34 | 52 |
| Norsalutaridinol | 2.57 | 316 | 178 | 0.222 | 30 | 18 |

Table 16. Multiple reaction monitoring targets and conditions (ESI+)

Items

Exemplary cells, methods and other embodiments of the invention are set out in the following items:
1. A recombinant host cell that expresses one or more genes encoding a cytochrome P450 enzyme capable of N-demethylating and/or O-demethylating reticuline and/or derivatives thereof,
wherein at least one of the genes is a recombinant gene.
2. The recombinant host cell according to item 1, wherein reticuline and/or derivatives thereof are selected from the group consisting of (S)-reticuline, 1,2 dehydroreticuline, (R)-reticuline, salutaridine, salutaridinol, thebaine, oripavine, 7-O-acetyl-salutaridinol, neopinone, codeinone, codeine, morphinone, morphine, hydrocodone, 14-hydroxycodeinone and oxycodone.
3. The recombinant host cell according to any one of items 1-2, wherein the reticuline derivative is thebaine.
4. The recombinant host cell according to any one of items 1-3, wherein the reticuline derivative is oripavine.
5. The recombinant host cell according to any one of items 1-4, wherein the cytochrome P450 enzyme capable of N-demethylating reticuline and derivatives thereof is selected from the group consisting of fungal cytochrome P450 enzymes, mammalian P450 3A4 enzymes, mammalian P450 3A5 enzymes, and mammalian P450 2C8 enzymes.
6. The recombinant host cell according to any one of items 1-5, wherein the cytochrome P450 enzyme capable of N-demethylating and/or O-demethylating reticuline and derivatives thereof has at least 20% sequence identity with a protein sequence selected from the group consisting of A0A077WEM0 (SEQ ID NO: 7), P450_DN15259_c0_g1_i7 (SEQ ID NO: 1), and DN12791_c0_g1_i1 (n.a.) (SEQ ID NO: 4)
such as 30% sequence identity, such as 40% sequence identity, such as 50% sequence identity, such as 60% sequence identity, such as 70% sequence identity,
such as 75% sequence identity, such as 80% sequence identity, such as 85% sequence identity, such as 90% sequence identity, such as 95% sequence identity, such as 97% sequence identity, such as 98% sequence identity, such as 99% sequence identity.
7. The recombinant host cell according to any one of items 1-6, wherein the cytochrome P450 enzyme capable of N-demethylating reticuline and derivatives thereof is selected from the group consisting of P450_DN15259_c0_g1_i7 (SEQ ID NO: 1), P450_DN12791_c0_g1_i1 (SEQ ID NO: 4), A0A077WEM0 (SEQ ID NO: 7), P08684 (SEQ ID NO: 22), H2PLK4 (SEQ ID NO: 24), A0A096NZ89 (SEQ ID NO: 26), G3SB46 (SEQ ID NO: 28), F1PDL2 (SEQ ID NO: 30), P20815 (SEQ ID NO: 40), A4ZZ70 (SEQ ID NO: 42), A8CBR0 (SEQ ID NO: 44), U3ECK3 (SEQ ID NO: 46), P10632 (SEQ ID NO: 32), H2Q2B (SEQ ID NO: 34), H2NB34 (SEQ ID NO: 36), Q4U0S8 (SEQ ID NO: 38), i) CYPDN8 (SEQ ID NO: 72),
ii) Mc_S2JT25 (SEQ ID NO: 52),
iii) CYPDN17 (SEQ ID NO: 90),
iv) CYPDN12 (SEQ ID NO: 80),
v) Lr_P450 (SEQ ID NO: 8),
vi) CYPDN29 (SEQ ID NO: 114),
vii) CYPDN14 (SEQ ID NO: 84),
vii) P450_DN15259_c0_g1_i7 (SEQ ID NO: 3),
ix) LCOR_01865 (SEQ ID NO: 54),
x) P450_DN5615_c2_g1_i9 (SEQ ID NO: 62),
xi) P450_DN12791_c0_g1_i1 (SEQ ID NO: 5),
xii) CYPDN16 (SEQ ID NO: 88),
xiii) CYPDN18 (SEQ ID NO: 92),
xiv) CYPDN27 (SEQ ID NO: 110),
xv) CYPDN35 (SEQ ID NO: 126),
xvi) CYPDN5 (SEQ ID NO: 66),
xvii) CYPDN6 (SEQ ID NO: 68),
xviii) CYPDN7 (SEQ ID NO: 70),
xix) CYPDN10 (SEQ ID NO: 76),
xx) CYPDN11 (SEQ ID NO: 78),
xxi) CYPDN24 (SEQ ID NO: 104),
xxii) CYPDN28 (SEQ ID NO: 112),
xxiii) CYPDN13 (SEQ ID NO: 82),
xxiv) CYPDN31 (SEQ ID NO: 118),
xxv) CYPDN34 (SEQ ID NO: 124),
xxvi) CYPDN22 (SEQ ID NO: 100),
xxvii) CYPDN21 (SEQ ID NO: 98),
xxviii) CYPDN30 (SEQ ID NO: 116),
xxix) Ar_ORZ22410 (SEQ ID NO: 58),
xxx) CYPDN20 (SEQ ID NO: 96),
xxxi) CYPDN17 (SEQ ID NO: 90), and
xxxii) CYPDN8 (SEQ ID NO: 72).
8. The recombinant host cell according to any one of items 1-7, wherein the cytochrome P450 enzyme capable of N-demethylating reticuline and derivatives thereof is P450_DN15259_c0_g1_i7 (SEQ ID NO: 3).
9. The recombinant host cell according to any one of items 1-6, wherein the cytochrome P450 enzyme capable of N-demethylating reticuline and derivatives thereof is P450_DN12791_c0_g1_i1 (SEQ ID NO: 6).
10. The recombinant host cell according to any one of items 1-7, wherein the cytochrome P450 enzyme capable of N-demethylating reticuline and derivatives thereof thereof is P450_DN15259_c0_g1_i7 (SEQ ID NO: 3).
11. The recombinant host cell according to any one of items 1-6 wherein the cytochrome P450 enzyme capable of N-demethylating reticuline and derivatives thereof thereof is P450_DN12791_c0_g1_i1 (SEQ ID NO: 6).
12. The recombinant host cell according to any one of items 1-7, wherein the cytochrome P450 enzyme capable of N-demethylating reticuline and derivatives thereof thereof is P450_DN15259_c0_g1_i7 (SEQ ID NO: 3).
13. The recombinant host cell according to any one of items 1-6, wherein the cytochrome P450 enzyme capable of N-demethylating reticuline and derivatives thereof thereof is P450_DN12791_c0_g1_i1 (SEQ ID NO: 6).
14. The recombinant host cell according to any one of items 1-13, wherein the cytochrome P450 enzyme capable of N-demethylating reticuline and derivatives thereof originates from a fungal organism.
15. The recombinant host cell according to any one of items 1-14, wherein the cytochrome P450 enzyme capable of N-demethylating reticuline and derivatives thereof originates from a fungal organism selected from the group consisting of Thamnostylum piriforme and Lichtheimia ramosa.
16. The recombinant host cell according to any one of items 1-7, wherein the cytochrome P450 enzyme capable of N-demethylating reticuline and derivatives thereof originates from a mammalian organism.
17. The recombinant host cell according to claim 16, items the cytochrome P450 enzyme capable of N-demethylating reticuline and derivatives thereof originates mammalian organism selected from the group consisting of Homo sapiens, Pongo abelii, Papio anubis, Gorilla gorilla gorilla, Canis lupus familiaris, Pan troglodytes, Callithrix jacchus, and Chlorocebus aethiops.
18. The recombinant host cell according to any one of items 1-7, further expressing one or more cytochrome P450 reductase(s) (CPR(s)).
19. The recombinant host cell according to item 18, wherein the cytochrome P450 reductase originates from a fungal or mammalian organism.
20. The recombinant host cell according to any one of items 18-19, wherein the cytochrome P450 reductase originates from an organism selected from the group consisting of Thamnostylum piriforme, Cunninghamella elegans, Gibberella fujikuroi, Saccharomyces cerevisiae, and Homo sapiens.
21. The recombinant host cell according to any one of items 18-20, wherein the cytochrome P450 reductase originates from a fungal organism.
22. The recombinant host cell according to any one of items 1-21, wherein the cytochrome P450 reductase originates from a fungal organism selected from the group consisting of Thamnostylum piriforme, Cunninghamella elegans, Gibberella fujikuroi, and Saccharomyces cerevisiae.
23. The recombinant host cell according to any one of items 18-20, wherein the cytochrome P450 reductase originates from a mammalian organism.
24. The recombinant host cell according to item 23, wherein the cytochrome P450 reductase originates from a mammalian organism which is Homo sapiens.
25. The recombinant host cell according to any one of items 18-22, wherein the cytochrome P450 reductase is selected from the group consisting of cytochrome P450 reductase is selected from the group consisting of DN5866_c0_g1_i1 (SEQ ID NO: 11), DN10898_c0_g1_i1 (SEQ ID NO: 14), AAF89958 (SEQ ID NO: 17), Q7Z8R1 (SEQ ID NO: 19), P16603 (SEQ ID NO: 21), POR1 (SEQ ID NO: 131), and BAB18572.1 (SEQ ID NO: 50).
26. The recombinant host cell according to any one of items 18-21, and 25 wherein the cytochrome P450 reductase is cytochrome P450 reductase is AAF89958 (SEQ ID NO: 17).
27. The recombinant host cell according to any one of items 18-21, and 25, wherein the cytochrome P450 reductase is cytochrome P450 reductase is P16603 (SEQ ID NO: 21).
28. The recombinant host cell according to any one of items 18-21, and 25, wherein the cytochrome P450 reductase is cytochrome P450 reductase is Q7Z8R1 (SEQ ID NO: 19).
29. The recombinant host cell according to any one of items 18-21, and 25, wherein the cytochrome P450 reductase is cytochrome P450 reductase is DN5866_c0_g1_i1 (SEQ ID NO: 11) or POR1 (SEQ ID NO: 131).
30. The recombinant host cell according to any one of items 18-21, and 25, wherein the cytochrome P450 reductase is cytochrome P450 reductase is DN10898_c0_g1_i1 (SEQ ID NO: 14).
31. The recombinant host cell according to any one of items 18-21, and 25, wherein the cytochrome P450 reductase is cytochrome P450 reductase is BAB18572.1 (SEQ ID NO: 50).
32. The recombinant host cell according to any one of items 1-15 and 18-21, wherein the cytochrome P450 enzyme capable of N-demethylating reticuline and derivatives thereof is P450_DN15259_c0_g1_i7 (SEQ ID NO: 3) and the cytochrome P450 reductase is selected from one or more from the list consisting of AAF89958 (SEQ ID NO: 17), Q7Z8R1 (SEQ ID NO: 19) and P16603 (SEQ ID NO: 21).
33. The recombinant host cell according to any one of items 1-15, 18-21, 23-25 and 32, wherein the cytochrome P450 enzyme capable of N-demethylating reticuline and derivatives thereof is P450_DN15259_c0_g1_i7 (SEQ ID NO: 3) and the cytochrome P450 reductase is AAF89958 (SEQ ID NO: 16).
34. The recombinant host cell according to any one of items 1-15 and 18-21, wherein the cytochrome P450 reductase is DN5866_c0_g1_i1 (SEQ ID NO: 11), POR1 (SEQ ID NO.: 131 and/or DN10898_c0_g1_i1 (SEQ ID NO: 14).
35. The recombinant host cell according to any one of items 1-15 and 18-21, wherein the P450 enzyme capable of N-demethylating reticuline and derivatives thereof is P450_DN15259_c0_g1_i7 (SEQ ID NO: 3) and the cytochrome P450 reductase is DN10898_c0_g1_i1 (SEQ ID NO: 14) and/or Q7Z8R1 (SEQ ID NO: 19).
36. The recombinant host cell according to any one of items 1-15 and 18-21, 25 and 28, wherein the cytochrome P450 enzyme capable of N-demethylating reticuline and derivatives thereof is P450_DN15259_c0_g1_i7 (SEQ ID NO: 3) and the cytochrome P450 reductase is Q7Z8R1 (SEQ ID NO: 19).
37. The recombinant host cell according to any one of items 1-15 and 18-21, and 25, wherein the cytochrome P450 enzyme capable of N-demethylating reticuline and derivatives thereof is P450_DN15259_c0_g1_i7 (SEQ ID NO: 3) and the cytochrome P450 reductase is DN5866_c0_g1_i1 (SEQ ID NO: 11) and/or DN10898_c0_g1_i1 (SEQ ID NO: 14).
38. The recombinant host cell according to any one of items 1-25, wherein the derivative is salutaridine and the cytochrome P450 enzyme is a P450 2C8 enzyme.
39. The recombinant host cell according to any one of items 1-25 and 38, wherein the derivative is salutaridine and the cytochrome P450 enzyme is selected from one or more from the group consisting of P10632 (SEQ ID NO: 23), H2Q2B (SEQ ID NO: 25), H2NB34 (SEQ ID NO: 27), Q4U0S8 (SEQ ID NO: 39).
40. The recombinant host cell according to any one of items 1-25, wherein the derivative is salutaridinol and the cytochrome P450 enzyme is a P450 2C8 enzyme.
41. The recombinant host cell according to any one of items 1-25 and 40, wherein the derivative is salutaridinol and the cytochrome P450 enzyme is selected from one or more from the group consisting of P10632 (SEQ ID NO: 33), H2Q2B (SEQ ID NO: 35), H2NB34 (SEQ ID NO: 37), and Q4U0S8 (SEQ ID NO: 39).
42. The recombinant host cell according to any one of items 1-25, wherein the compound is thebaine and the cytochrome P450 enzyme is a P450 34A enzyme.
43. The recombinant host cell according to any one of items 1-25 and 41, wherein the compound is thebaine and the cytochrome P450 enzyme is A4ZZ70 (SEQ ID NO: 43).
44. The recombinant host cell according to any one of items 1-25, wherein the derivative is oripavine and the cytochrome P450 enzyme is a P450 2C8 enzyme.
45. The recombinant host cell according to any one of items 1-25 and 44, wherein the derivative is oripavine and the cytochrome P450 enzyme is Q4U0S8 (SEQ ID NO: 39).
46. The recombinant host cell according to any one of items 1-25, wherein the derivative is morphine and the cytochrome P450 enzyme is a P450 2C8 enzyme or a P450 3A4 enzyme.
47. The recombinant host cell according to any one of items 1-25 and 46, wherein the derivative is morphine and the cytochrome P450 enzyme is selected from one or more from the group consisting of P20815 (SEQ ID NO: 41), A4ZZ70 (SEQ ID NO: 43), P10632 (SEQ ID NO: 33), H2Q2B (SEQ ID NO: 35), H2NB34 (SEQ ID NO: 37), and Q4U0S8 (SEQ ID NO: 39).
48. The recombinant host cell according to any one of items 1-25, wherein the compound is thebaine and the cytochrome P450 enzyme is a P450 3A4 enzyme.
49. The recombinant host cell according to any one of items 1-25, wherein the compound is thebaine and the cytochrome P450 enzyme is A0A096NZ89 (SEQ ID NO: 27).
50. The recombinant host cell according to any one of items 1-46, wherein the cell is a yeast cell, a plant cell, a mammalian cell, an insect cell, a fungal cell, a bacterial cell, an algal cell, or a cyanobacterial cell.
51. The recombinant host cell according to any one of items 1-50, wherein the cell is that originates from a fungal or mammalian organism.
52. The recombinant host cell according to any one of items 1-51, wherein the cytochrome P450 reductase originates from an organism selected from the group consisting of Thamnostylum piriforme, Cunninghamella elegans, Gibberella fujikuroi, Saccharomyces cerevisiae, Mucor piriformis, Aspergillus sp., and Homo sapiens.
53. The recombinant host cell according to any one of items 1-51, wherein the cell is a yeast cell selected from the group consisting of Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., or Rhodospiridium sp.
54. The recombinant host cell according to any one of items 1-53, wherein the host cell is a Saccharomycete.
55. The recombinant host cell according to any one of items 1-54, wherein the host cell is a yeast cell that is a Saccharomyces cerevisiae cell.
56. An in vitro method for N-demethylating a reticuline or a derivative thereof, comprising contacting reticuline or a derivative thereof with a recombinant P450 enzyme capable of N-demethylating reticuline or a derivative thereof.
57. The method according to item 56, further comprising cultivating a recombinant host cell of any one of items 1-55, in a culture medium in presence of reticuline or a derivative thereof, under conditions in which the one or more genes encoding the cytochrome P450 enzymes is/are expressed.
58. The method according to any one of items 56-57, further comprising cultivating the recombinant host of any one of items 1-55 in a culture medium under conditions in which the one or more genes encoding the cytochrome P450 reductase is/are expressed.
59. A method of producing a N-demethylated reticuline (norreticuline) and derivatives thereof, comprising cultivating the recombinant host of any one of items 1-55 in a culture medium under conditions in which the one or more genes encoding the cytochrome P450 enzymes is/are expressed in presence of reticuline or derivatives thereof.
60. The method according to any one of items 56-60, wherein reticuline and derivatives thereof are selected from the group consisting of (S)-reticuline, 1,2 dehydroreticuline, (R)-reticuline, salutaridine, salutaridinol, thebaine, oripavine, neopinone, 7-O-acetyl-salutaridinol, codeinone, codeine, morphinone, morphine, hydrocodone, 14-hydroxycodeinone and oxycodone.
61. The method according to any one of items 56-60, further comprising cultivating the recombinant host of any one of claims 1-55 in a culture medium under conditions in which the one or more genes encoding the cytochrome P450 reductase is/are expressed.
62. A composition comprising a compound selected from the group consisting of N-demethylated reticuline and derivatives thereof obtainable from the methods according to any one of items 56-61, and further comprising elements from a fungal fermentation broth and/or at least one fungal specific metabolite.
63. A composition comprising a N-demethylated reticuline or a derivative thereof, and a recombinant P450 enzyme capable of N-demethylating reticuline or a derivative thereof.
64. A DNA molecule comprising a nucleic acid encoding one or more of the recombinant genes according to any one of items 1-55.
65. An expression vector comprising the DNA molecule according to item 60 and a promoter suitable for expression of the DNA molecule in a cell.
66. A host cell comprising the expression vector of item 65.
67. A method of preparing buprenorphine, or a salt thereof, from Compound HO—I—H (Nororipavine), or a salt thereof:

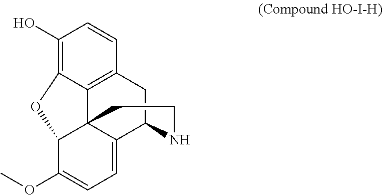 [SMILES: COC1=CC=C2[C@H]3Cc4ccc(O)c5c4[C@@]2(CCN3)[C@H]1O5]

comprising:
(i)(A1) reacting Compound HO—I—H with cyclopropane carboxaldehyde followed by a hydride source; or
(i)(A2) reacting Compound HO—I—H with cyclopropanecarboxylic acid halide followed by a reducing agent; or
(i)(A3) reacting Compound HO—I—H with cyclopropylmethyl halide or activated cyclopropane methanol;
   to provide Compound HO—I-MCP:

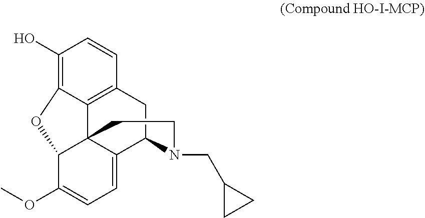 [SMILES: COC1=CC=C2[C@H]3Cc4ccc(O)c5c4[C@@]2(CCN3CC2CC2)[C@H]1O5]

(ii)(B) reacting Compound HO—I-MCP with methyl vinyl ketone to provide Compound HO—II-MCP:

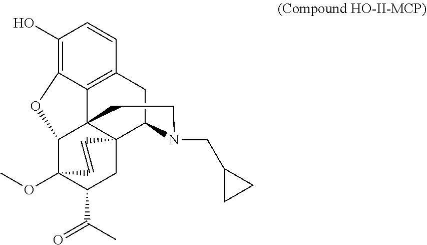 [SMILES: CO[C@]12C=C[C@@]3(C[C@@H]1C(C)=O)[C@H]1Cc4ccc(O)c5c4[C@@]3(CCN1CC1CC1)[C@H]2O5]

(iii)(C) reacting Compound HO—II-MCP with H₂ in the presence of a hydrogenation catalyst to provide Compound HO—IIIB-MCP:

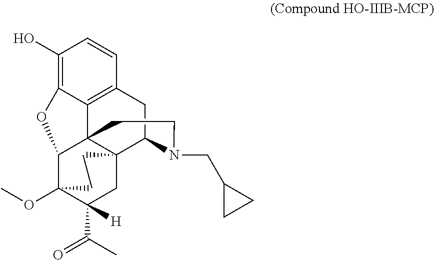 [SMILES: CO[C@]12CC[C@@]3(C[C@@H]1C(C)=O)[C@H]1Cc4ccc(O)c5c4[C@@]3(CCN1CC1CC1)[C@H]2O5]

(iv)(D) reacting Compound HO—IIIB-MCP with tert-butylmagnesium halide to provide buprenorphine.

Another aspect of the disclosure relates to a method of preparing buprenorphine, or a salt thereof, from Compound HO—II-MCP
(iii)(D) reacting Compound HO—II-MCP with tert-butylmagnesium halide to provide Compound HO—IIIA-MCP:

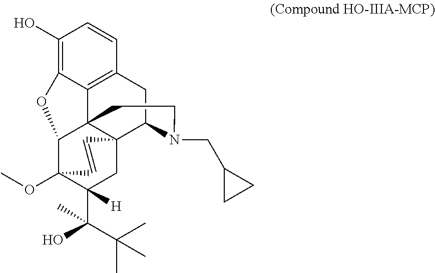 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]

(iv)(C) reacting Compound HO—IIIA-MCP with H2 in the presence of a hydrogenation catalyst to provide buprenorphine.

Another aspect of the disclosure relates to a method of preparing buprenorphine, or a salt thereof, from Compound HO—II-MCP
(iii)(F) reacting Compound HO—II-MCP with benzyl halide, benzyl sulfonate, or activated benzyl alcohol to provide Compound BnO—II-MCP:

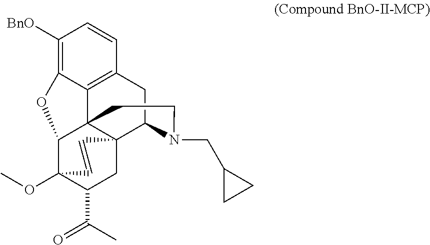 [SMILES: CO[C@]12C=C[C@@]3(C[C@@H]1C(C)=O)[C@H]1Cc4ccc(OCc5ccccc5)c5c4[C@@]3(CCN1CC1CC1)[C@H]2O5]

(iv)(D) reacting Compound BnO—II-MCP with tert-butylmagnesium halide to provide Compound BnO—IIIA-MCP:

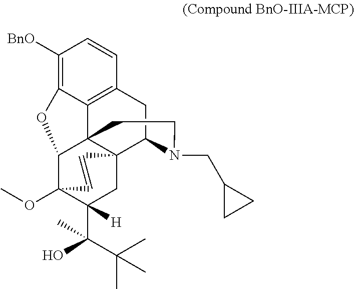 [SMILES: CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5]

(v)(C) reacting Compound BnO—IIIA-MCP with H₂ in the presence of a hydrogenation catalyst to provide buprenorphine

Another aspect of the disclosure relates to a method of preparing buprenorphine, or a salt thereof, from Compound HO—I-MCP
(ii)(F) reacting Compound HO—I-MCP with benzyl halide, benzyl sulfonate, or activated benzyl alcohol to provide Compound BnO-I-MCP:

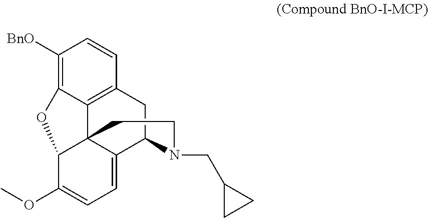 [SMILES: COC1=CC=C2[C@H]3Cc4ccc(OCc5ccccc5)c5c4[C@@]2(CCN3CC2CC2)[C@H]1O5]

(iii)(B) reacting Compound BnO-I-MCP with methyl vinyl ketone to provide Compound BnO—II-MCP:

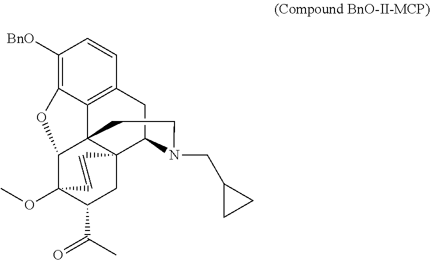 [SMILES: CO[C@]12C=C[C@@]3(C[C@@H]1C(C)=O)[C@H]1Cc4ccc(OCc5ccccc5)c5c4[C@@]3(CCN1CC1CC1)[C@H]2O5]

(iv)(D) reacting Compound BnO—II-MCP with tert-butylmagnesium halide to provide Compound BnO—IIIA-MCP:

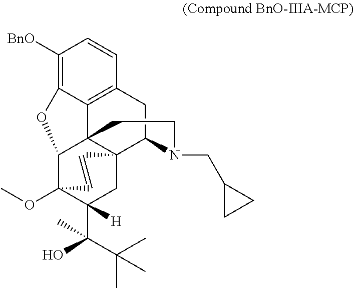 [SMILES: CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5]

(v)(C) reacting Compound BnO—IIIA-MCP with H₂ in the presence of a hydrogenation catalyst to provide buprenorphine.

Another aspect of the disclosure relates to a method of preparing buprenorphine, or a salt thereof, from Compound MeO—I—H (Northebaine), or a salt thereof:

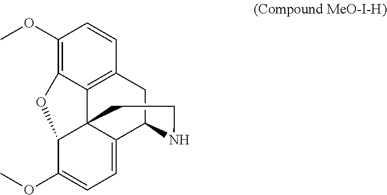 [SMILES: COC1=CC=C2[C@H]3Cc4ccc(OC)c5c4[C@@]2(CCN3)[C@H]1O5]

comprising:
(i)(A1) reacting Compound MeO—I—H with cyclopropane carboxaldehyde followed by a hydride source; or
(i)(A2) reacting Compound MeO—I—H with cyclopropanecarboxylic acid halide followed by a reducing agent; or
(i)(A3) reacting Compound MeO—I—H with cyclopropylmethyl halide or activated cyclopropane methanol;
   to provide Compound MeO—I-MCP:

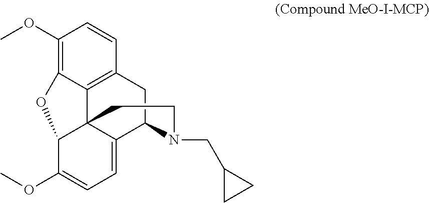 [SMILES: COC1=CC=C2[C@H]3Cc4ccc(OC)c5c4[C@@]2(CCN3CC2CC2)[C@H]1O5]

(ii)(B) reacting Compound MeO—I-MCP with methyl vinyl ketone to provide Compound MeO—II-MCP:

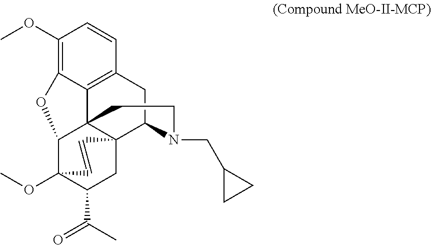 [SMILES: COc1ccc2c3c1O[C@H]1[C@@]4(OC)C=C[C@@]5(C[C@@H]4C(C)=O)[C@@H](C2)N(CC2CC2)CC[C@]315]

(iii)(C) reacting Compound MeO—II-MCP with H2 in the presence of a hydrogenation catalyst to provide Compound MeO—IIIB-MCP:

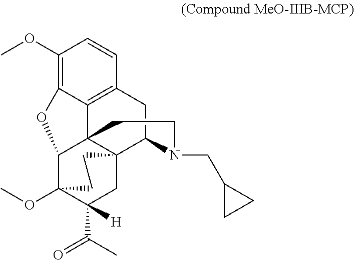 [SMILES: COc1ccc2c3c1O[C@H]1[C@@]4(OC)CC[C@@]5(C[C@@H]4C(C)=O)[C@@H](C2)N(CC2CC2)CC[C@]315]

(iv)(D) reacting Compound MeO—IIIB-MCP with tert-butylmagnesium halide to provide Compound MeO—IV-MCP:

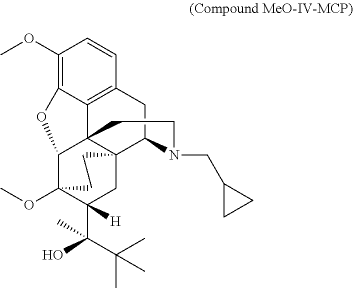 [SMILES: COc1ccc2c3c1O[C@H]1[C@@]4(OC)CC[C@@]5(C[C@@H]4[C@](C)(O)C(C)(C)C)[C@@H](C2)N(CC2CC2)CC[C@]315]

(v)(E) reacting a compound of Compound MeO—IV-MCP with a demethylating agent to provide buprenorphine.

Another aspect of the disclosure relates to a method of preparing buprenorphine, or a salt thereof, from Compound MeO—II-MCP, or a salt thereof
(iii)(D) reacting Compound MeO—II-MCP with tert-butylmagnesium halide to provide Compound MeO—IIIA-MCP:

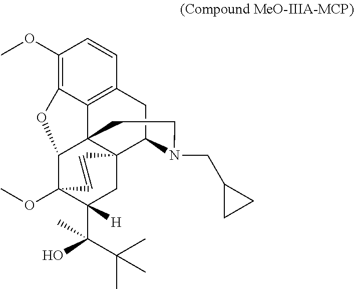 [SMILES: COc1ccc2c3c1O[C@H]1[C@@]4(OC)C=C[C@@]5(C[C@@H]4[C@](C)(O)C(C)(C)C)[C@@H](C2)N(CC2CC2)CC[C@]315]

(iv)(C) reacting Compound MeO—IIIA-MCP with H2 in the presence of a hydrogenation catalyst to provide a compound of Compound MeO—IV-MCP:

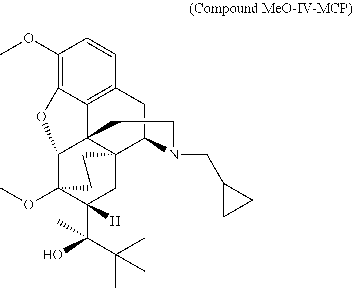 [SMILES: COc1ccc2c3c1O[C@H]1[C@@]4(OC)CC[C@@]5(C[C@@H]4[C@](C)(O)C(C)(C)C)[C@@H](C2)N(CC2CC2)CC[C@]315]

(v)(E) reacting a compound of Compound MeO—IV-MCP with a demethylating agent to provide buprenorphine.

Another aspect of the disclosure relates to a method of preparing buprenorphine, or a salt thereof, from Compound MeO—IIIA-MCP, or a salt thereof
iv)(E) reacting Compound MeO—IIIA-MCP with a demethylating agent to provide Compound HO—IIIA-MCP:

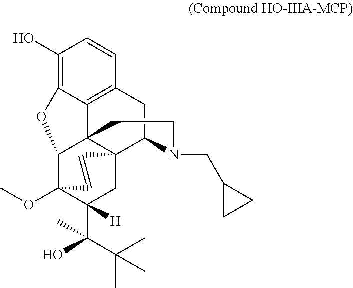 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]

(v)(C) reacting Compound HO—IIIA-MCP with H2 in the presence of a hydrogenation catalyst to provide buprenorphine.
80. A method according to any of items 67-72, wherein the demethylating agent of step (E) is a thiolate.
81. A method according to any of items 67-72, wherein the demethylating agent of step (E) is a dodecane thiolate.
82. A method according to any of items 67-72 and 80-81, wherein step (E) is performed in a solvent comprising a polar aprotic solvent.
83. A method according to any of items 67-72 and 80-81, wherein step (E) is performed in a solvent comprising N-methylpyrrolidone, tetrahydrofuran, ethyl acetate, acetone, dimethylformamide, acetonitrile, dimethylsulfoxide, propylene carbonate, or a mixture thereof.
84. A method according to any of items 67-72 and 80-81, wherein the demethylating agent of step (E) is reacted at a temperature within the range of about 50° C. to about 190° C., for a period of time within the range of about 4 hours to about 2 days.
85. A method according to any of items 73-76, wherein the benzyl halide of step (F) is benzyl chloride or benzyl bromide.
86. A method according to any of items 73-76 and 85, wherein step (F) is performed in the presence of a strong base.
87. A method according to any of items 73-76 and 85, wherein step (F) is performed in the presence of an alkali metal hydride.
88. A method according to any of items 73-76 and 85-87, wherein step (F) is performed in a solvent comprising a polar aprotic solvent.
89. A method according to any of items 73-76 and 85-87, wherein step (F) is performed in a solvent comprising N-methylpyrrolidone, tetrahydrofuran, ethyl acetate, acetone, dimethylformamide, acetonitrile, dimethylsulfoxide, propylene carbonate, or a mixture thereof.
90. A method according to any of items 73-76 and 85-897, wherein the benzyl halide, benzyl sulfonate, or activated benzyl alcohol of step (F) is reacted at one or more temperatures within the range of about of about −20° C. to about 40° C., for a period of time within the range of about 6 hours to about 2 days.
91. A method according to any of items 76-77, wherein step (H) is performed in a solvent comprising N-methylpyrrolidone, tetrahydrofuran, ethyl acetate, acetone, dimethylformamide, acetonitrile, dimethylsulfoxide, propylene carbonate, or a mixture thereof.
92. A method according to any of items 76-77 and 91, wherein the lithium aluminum hydride of step (H) is reacted at a temperature within the range of about 40° C. to about 120° C.
93. A method according to any of items 76-79 and 91-92, wherein step (G) is performed in the presence of a trialkylamine, e.g., triethylamine, diisopropylethylamine, 4-methyl-morpholine, or N-methyl-piperidine.
94. A method according to any of items 76-79 and 91-93, wherein step (G) is performed in a solvent comprising dichloromethane, chloroform, toluene, 1,4-dioxane, diethyl ether, benzene, or a mixture thereof.
95. A method according to any of items 76-79 and 91-94, wherein the acyl halide of step (G) is reacted at one or more temperatures within the range of about of about −20° C. to about 40° C., for a period of time within the range of about about 30 minutes to about 8 hours.
96. A method according to item 78 or 79, wherein step (I) comprises reacting Compound HO—IV—Ac with Schwartz's reagent.
97. A method according to item 96, wherein step (I) is performed in a solvent comprising a polar aprotic solvent, e.g., N-methylpyrrolidone, tetrahydrofuran, ethyl acetate, acetone, dimethylformamide, acetonitrile, dimethylsulfoxide, propylene carbonate, or a mixture thereof.
98. A method according to item 96 or 97, wherein the Schwartz's reagent is reacted at a temperature within the range of about 15° C. to about 40° C., for a period of time within the range of about 5 minutes to about 3 hours.
99. A method according to item 78 or 79, wherein step (I) comprises reacting Compound HO—IV—Ac with base, e.g., KOH.
100. A method according to item 99, wherein step (I) is performed in a solvent comprising a high-boiling-point polar protic or aprotic solvent, e.g., ethylene glycol, diethylene glycol, N-methylpyrrolidone, dimethylformamide, or dimethylsulfoxide.
101. A method according to item 99 or 100, wherein the base is reacted at a temperature within the range of about 50° C. to about 240° C., for a period of time within the range of about 4 hours to about 2 days.
102. A method according to any of items 1-101, comprising step (A1).
103. A method according to item 102, wherein the hydride source of step (A1) is formic acid or sodium cyanoborohydride.
104. A method according to item 102, wherein the hydride source of step (A1) is formic acid.
105. A method according to any of items 102-1049, wherein step (A1) is catalyzed by a ruthenium(II) complex.
106. A method according to any of items 102-104, wherein step (A1) is catalyzed by dichloro(p-cymene)ruthenium(II) dimer.
107. A method according to any of items 102-106, wherein step (A1) is performed in a solvent comprising a polar aprotic solvent.
108. A method according to any of items 102-106, wherein step (A1) is performed in a solvent comprising N-methylpyrrolidone, tetrahydrofuran, ethyl acetate, acetone, dimethylformamide, acetonitrile, dimethylsulfoxide, propylene carbonate, or a mixture thereof.
109. A method according to any of items 102-108, wherein step (A1) is performed in the presence of a trialkylamine.
110. A method according to any of items 102-108, wherein step (A1) is performed in the presence of triethylamine, diisopropylethylamine, 4-methyl-morpholine, or N-methyl-piperidine.
111. A method according to any of items 102-110, wherein the cyclopropane carboxaldehyde of step (A1) is reacted at a temperature within the range of about 30° C. to about 90° C., for a period of time within the range of about 30 minutes to about 5 hours.
112. A method according to any of items 1-101, comprising step (A2).
113. A method according to item 112, wherein the cyclopropanecarboxylic acid halide is cyclopropanecarboxylic acid chloride or cyclopropanecarboxylic acid bromide.
114. A method according to item 112 or 113, wherein the reducing agent is LiAlH₄ or NaBH₄.
115. A method according to any of items 112-114, wherein the reaction with cyclopropanecarboxylic acid halide of step (A2) is performed in a solvent comprising a nonpolar solvent.
116. A method according to any of items 112-114, wherein the reaction with cyclopropanecarboxylic acid halide of step (A2) is performed in a solvent comprising dichloromethane, chloroform, toluene, 1,4-dioxane, diethyl ether, benzene, or a mixture thereof.
117. A method according to any of items 112-116, wherein the reaction with a reducing agent of step (A2) is performed in a solvent comprising a polar aprotic solvent.
118. A method according to any of items 112-116, wherein the reaction with a reducing agent of step (A2) is performed in a solvent comprising N-methylpyrrolidone, tetrahydrofuran, ethyl acetate, acetone, dimethylformamide, acetonitrile, dimethylsulfoxide, propylene carbonate, or a mixture thereof.
119. A method according to any of items 112-118, wherein the cyclopropanecarboxylic acid halide of step (A2) is reacted at one or more temperatures within the range of about −20° C. to about 40° C., for a period of time within the range of about 6 hours to about 2 days.
120. A method according to any of items 112-119, wherein the reducing agent of step (A2) is reacted at a temperature within the range of about 35° C. to about 85° C., for a period of time within the range of about 5 minutes to about 3 hours.
121. A method according to any of items 1-101, comprising step (A3).
122. A method according to item 121, wherein the cyclopropylmethyl halide is cyclopropylmethyl chloride or cyclopropylmethyl bromide.
123. A method according to item 121 or 122, wherein step (A3) is performed in a solvent comprising a polar protic solvent.
124. A method according to item 121 or 122, wherein step (A3) is performed in a solvent comprising n-butanol, isopropanol, ethanol, methanol, water, or a mixture thereof.
125. A method according to any of items 121-124, wherein step (A3) is performed in the presence of a trialkylamine.
126. A method according to any of items 121-124, wherein step (A3) is performed in the presence of triethylamine, diisopropylethylamine, 4-methyl-morpholine, or N-methyl-piperidine.
127. A method according to any of items 121-124, wherein the cyclopropylmethyl halide or activated cyclopropane methanol of step (A3) is reacted a temperature within the range of about 40° C. to about 120° C., for a period of time within the range of about 30 minutes to about 6 hours.
128. A method according to any of items 67-127, wherein step (B) is performed in a solvent comprising a nonpolar solvent.
129. A method according to any of items 67-127, wherein step (B) is performed in a solvent comprising dichloromethane, chloroform, toluene, 1,4-dioxane, diethyl ether, benzene, or a mixture thereof.
130. A method according to any of items 67-129, wherein the methyl vinyl ketone of step (B) is reacted at a temperature within the range of about 40° C. to about 120° C. for a period of time within the range of about 2 hours to about 2 days.
131. A method according to any of items 67-130, wherein the hydrogenation catalyst of step (C) comprises nickel, palladium, platinum, rhodium, or ruthenium.
132. A method according to any of items 67-130, wherein the hydrogenation catalyst of step (C) comprises platinum or palladium supported on carbon.
133. A method according to any of items 67-132, wherein step (C) is performed in a solvent comprising a polar protic or aprotic solvent.
134. A method according to any of items 67-132, wherein step (C) is performed in a solvent comprising n-butanol, isopropanol, ethanol, methanol, N-methylpyrrolidone, tetrahydrofuran, ethyl acetate, acetone, dimethylformamide, acetonitrile, dimethylsulfoxide, propylene carbonate, or a mixture thereof.
135. A method according to any of items 67-134, wherein the hydrogen of step (C) is reacted at a temperature within the range of about 15° C. to about 120° C., for a period of time within the range of about 6 hours to about 3 days.
136. A method according to any of items 67-135, wherein the hydrogen of step (C) is reacted at a pressure within the range of about 1 atm. to about 3 atm.
137. A method according to any of items 67-136, wherein the tert-butylmagnesium halide of step (D) is tert-butylmagnesium chloride or tert-butylmagnesium bromide.
138. A method according to any of items 67-137, wherein step (D) is performed in a solvent comprising a nonpolar solvent.
139. A method according to any of items 67-138, wherein step (D) is performed in a solvent comprising tert-butylmethyl ether, 2-methyl-tetrahydrofuran, diethyl ether, dimethoxymethane, benzene, toluene, or a mixture of thereof.
140. A method according to any of items 1-139, wherein the tert-butylmagnesium halide of step (D) is reacted at a temperature within the range of about 15° C. to about 100° C. for a period of time within the range of about 30 minutes to about 8 hours.
141. A method according to any of items 67-140, further comprising contacting thebaine and/or oripavine with the recombinant host of any of items 1-55 to produce the Compound MeO—I—H or the Compound HO—I—H.
142. A method according to any of items 67-140, wherein the Compound MeO—I—H or the Compound HO—I—H is produced by a method according to any of items 56-61.
143. The recombinant host cell according to any one of items 1-55, wherein the cell is a plant cell, a filamentous fungus, or a yeast cell.
144. The recombinant host cell according to any one of items 1-55 or 143, wherein the cell is a plant cell.
145. The recombinant host cell according to any one of items 1-55 or 143-144, wherein the cell is a plant cell comprising Papaver sp. (e.g. Papaver somniferum or Papaver bracteatum cells), Nicotiana sp. (e.g. Nicotiana benthamiana cells), Arabidopsis sp., Physcomitrella sp., Thalictrum sp. (e.g. Thalictrum flavum), Coptis sp. (e.g Coptis japonica), Lindera sp. (Lindera aggregate), Annona sp. (e.g. Annona squamosa or Annona muricata), Ocotea sp. (e.g. Ocotea fasciculate), Duguetia sp., Sinomenium sp., Berberis sp., Corydalis sp., Ceratocapnos palaestinus, Anomianthus dulcis, Dicentra spectabilis, Glaucium flavum, Eschscholzia californica, Caulophyllum thalicroides, Chelidonium majus, Cocculus laurifolius, Delphinium pentagynum, Cinnamomum camphora, Clematis parviloba, Phylica rogersih, Phellodendron chinensis, Hypecoum lactiflorum, Fumaria officinalis, Croton celtidifolius, Mahonia aquifolium, Illigera parviflora, Aniba canelilla, Cryptocarya odorata, Litsea sp., Machilus thunbergii, Nectandra salicifolia, Neolitsea sp., Phoebe minutiflora, Strychnos holstii, Tinospora cordifolia, or Siparuna tonduziana,
146. The recombinant host cell according to any one of items 1-55 or 143-145, wherein the cell is a Papaver sp. cell.
147. The recombinant host cell according to item 146, wherein the Papaver sp. cell is a Papaver somniferum or a Papaver bracteatum cell.
148. The recombinant host cell according to any one of items 1-55 or 143-146, wherein the cell is a Nicotiana sp. cell.
149. The recombinant host cell according to item 148, wherein the Nicotiana sp. cell is a Nicotiana benthamiana cell.
150. The recombinant host cell according to any one of items 1-55 or 143-146, wherein the cell is an Arabidopsis sp. cell.
151. The recombinant host cell according to any one of items 1-55 or 143-146, wherein the cell is a Physcomitrella sp. cell.
152. The recombinant host cell according to any one of items 1-55 or 143-143, wherein the cell is a a filamentous fungus cell.
153. The recombinant host cell according to item 152, wherein the cell is a filamentous fungus cell comprising Aspergillus nidulans, Aspergillus sydowii, Aspergillus terreus, Aspergillus oryzae, Aspergillus caelatus, Aspergillus chevalieri, Aspergillus longivesica, Aspergillus parvulus, Aspergillus amylovorus, Aspergillus niger, Aspergillus niger, Aspergillus aculeatus, Aspergillus ellipticus, Aspergillus violaceofuscus, Aspergillus brunneoviolaceus, Aspergillus japonicus, Aspergillus brasiliensis, Aspergillus brasiliensis, Aspergillus aculeatinus, Aspergillus thermomutatus, Aspergillus implicatus, Aspergillus acristatus, Penicillium bilaiae, Penicillium rubens, Penicillium chrysogenum, Penicillium expansum, Penicillium antarcticum, Trichoderma reesei, Talaromyces atroroseus, Asteromyces cruciatus, or Neurospora crassa.
154. The recombinant host cell according to any one of items 1-55 or 143-143, wherein the cell is a yeast cell comprising Saccharomyces cerevisiae, Schizosaccharomyces pombe, Yarrowia lipolytica, Candida glabrata, Ashbya gossypii, Cyberlindnera jadinii, Pichia pastoris, Kluyveromyces lactis, Hansenula polymorpha, Candida boidinii, Arxula adeninivorans, Xanthophyllomyces dendrorhous, Candida albicans, Rhodotorula sp., Schwanniomyces occidentalis, Sporidiobolus salmonicolor, Starmerella bacillaris, Sugiyamaella americana, Talaromyces atroroseus, Torulaspora delbrueckii, Trichoderma reesei, Wickerhamia fluorescens, Wickerhamiella sorbophila, Wickerhamiella versatilis, Zygosaccharomyces rouxii, Zygotorulaspora Florentina, Saccharomyces cerevisiae var. ellipsoideus, Saccharomyces paradoxus, Saccharomyces pastorianus, Saccharomyces uvarum, Saccharomycodes ludwigii var. ludwigii, Saitoella complicate, Schizosaccharomyces japonicus, Schizosaccharomyces octosporus, Asteromyces cruciatus, Aureobasidium pullulans, Candida cylindracea, Candida albicans, Cutaneotrichosporon curvatus, Cyberlindnera jadinii, Debaromyces hansenii, Dekkera bruxellensis, Diutina rugosa, Eremothecium gossypii, Galactomyces candidus, Geotrichum candidum, Geotrichum fermentans, Hanseniaspora uvarum, Hanseniaspora vineae, Issatchenkia orientalis, Kazachstania exigua, Kazachstania servazzii, Kluyveromyces lactis, Kluyveromyces marxianus, Komagataella phaffli, Lachancea thermotolerans, Lipomyces starkeyi, Moesziomyces antarcticus, Naumovozyma castellii, Naumovozyma dairenensis, Ogataea polymorpha, Ogataea thermomethanolica, Pachysolen tannophilus, Papiliotrema laurentii, Penicillium arizonense, Pichia fermentans, Rhodotorula mucilaginosa, Saccharomyces bayanus or Rhodospiridium sp.
155. A method of preparing buprenorphine, or a salt thereof, from Compound HO—I—H, or a salt thereof, comprising:
   contacting thebaine and/or oripavine with the recombinant host of any of claims 1-55 to produce the Compound MeO—I—H or the Compound HO—I—H, wherein buprenorphine is produced by a method according to any of items 67-140.
156. The method according to item 155, wherein the Compound MeO—I—H or the Compound HO—I—H produced from the recombinant host undergoes one or more purification steps before buprenorphine is produced by a method according to any of items 67-140.
157. A product obtainable from a method according to any one or more of items 56-61, 67-141 or 155-156.
158. Buprenorphine obtainable from a method according to any one or more of items 67-141 or 155-156.

1. A method of preparing a compound of formula (I): 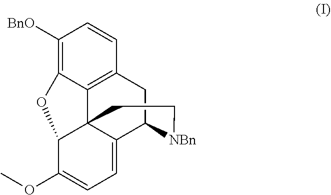 [SMILES: COC1=CC=C2[C@H]3Cc4ccc(OCc5ccccc5)c5c4[C@@]2(CCN3Cc2ccccc2)[C@H]1O5]
wherein the method comprises:
a) contacting thebaine and/or oripavine with a recombinant host cell that expresses one or more genes encoding a cytochrome P450 enzyme capable of N-demethylating and/or O-demethylating the thebaine and/or the oripavine, wherein at least one of the genes is a recombinant gene, to produce a compound of formula (II); 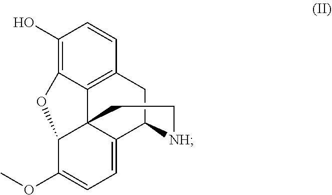 [SMILES: COC1=CC=C2[C@H]3Cc4ccc(O)c5c4[C@@]2(CCN3)[C@H]1O5]
and
b) reacting the compound of formula (II) with benzyl halide, benzyl sulfonate, or activated benzyl alcohol to provide a compound of formula (I); or
c) reacting the compound of formula (II) with an acyl halide to provide a compound of formula (III), 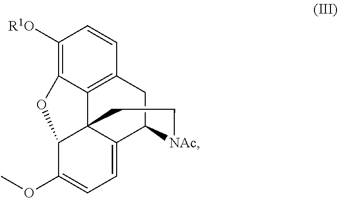
wherein R¹ is H;
and reacting the compound of formula (III) with benzyl halide, benzyl sulfonate, or activated benzyl alcohol to provide a compound BnO—I—Ac (formula III, wherein R¹ is benzyl), and subsequently reacting the compound BnO—I—Ac with lithium aluminum hydride to provide the compound of formula (I).

2. The method according to claim 1, wherein the activated benzyl alcohol is activated with a sulfonate group.

3. The method according to claim 1, wherein sulfonate group is a p-toluene sulfonyl group, a methyl sulfonyl group, or triphenylphosphine.

4. The method according to claim 1, wherein the benzyl halide is benzyl chloride or benzyl bromide.

5. The method according to claim 4, wherein the method is performed in the presence of a strong base.

6. The method according to claim 5, wherein the strong base is an alkali metal hydride.

7. The method according to claim 1, wherein the method is performed in a solvent comprising a polar aprotic solvent.

8. The method according to claim 7, wherein the polar aprotic solvent is N-methylpyrrolidone, tetrahydrofuran, ethyl acetate, acetone, dimethylformamide, acetonitrile, dimethylsulfoxide, propylene carbonate, or a mixture thereof.

9. The method according to claim 1, wherein the benzyl halide, benzyl sulfonate, or activated benzyl alcohol is reacted at a temperature within the range of from −20° C. to 40° C.

10. The method according to claim 1, wherein the benzyl halide, benzyl sulfonate, or activated benzyl alcohol is reacted for a period of time within the range of from 6 hours to 2 days.

11. The method according to claim 1, wherein the method further comprises reacting the compound of formula (I) with methyl vinyl ketone providing a compound of formula II-Bn: 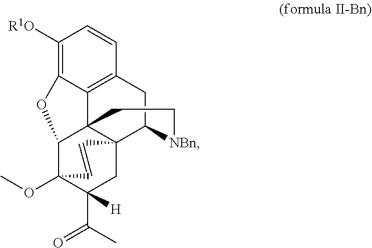
wherein R¹ is benzyl.

12. The method according to claim 11, wherein the method further comprises reacting the compound of formula II-Bn with a tert-butylmagnesium halide providing a compound of formula IIIA-Bn, 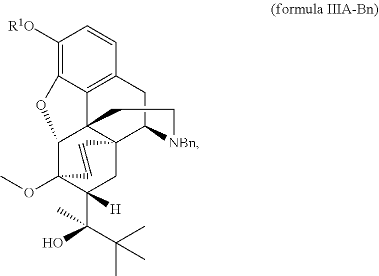
wherein R¹ is benzyl.

13. The method according to claim 12, wherein the method further comprises reacting the compound of formula IIIA-Bn with H2 in the presence of a hydrogenation catalyst providing a compound of formula IV—H, 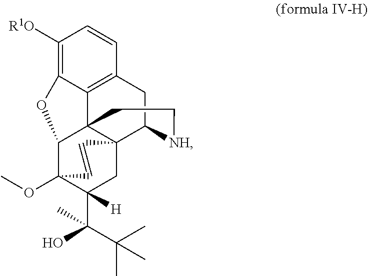
wherein R¹ is H.

14. The method according to claim 13, wherein the method further comprises reacting the compound of formula IV—H, wherein R¹ is H, with cyclopropane carboxaldehyde followed by a hydride source providing buprenorphine.

15. A method of preparing noroxymorphone comprising:
a) contacting thebaine and/or oripavine with a recombinant host cell that expresses one or more genes encoding a cytochrome P450 enzyme capable of N-demethylating and/or O-demethylating the thebaine and/or the oripavine, wherein at least one of the genes is a recombinant gene, to produce a compound of formula (II); 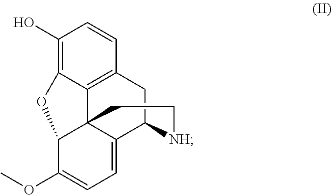 [SMILES: COC1=CC=C2[C@H]3Cc4ccc(O)c5c4[C@@]2(CCN3)[C@H]1O5]
and
b) reacting the compound of formula (II) with benzyl halide, benzyl sulfonate, or activated benzyl alcohol to provide a compound of formula (I): 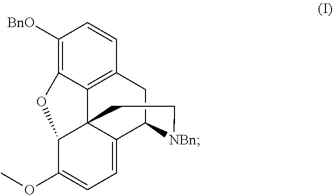 [SMILES: COC1=CC=C2[C@H]3Cc4ccc(OCc5ccccc5)c5c4[C@@]2(CCN3Cc2ccccc2)[C@H]1O5]
and
c) preparing noroxymorphone from the compound of formula (I).

16. A method of preparing naltrexone comprising:
a) contacting thebaine and/or oripavine with a recombinant host cell that expresses one or more genes encoding a cytochrome P450 enzyme capable of N-demethylating and/or O-demethylating the thebaine and/or the oripavine, wherein at least one of the genes is a recombinant gene, to produce a compound of formula (II); 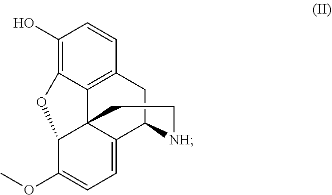 [SMILES: COC1=CC=C2[C@H]3Cc4ccc(O)c5c4[C@@]2(CCN3)[C@H]1O5]
and
b) reacting the compound of formula (II) with benzyl halide, benzyl sulfonate, or activated benzyl alcohol to provide a compound of formula (I): 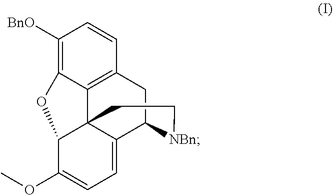 [SMILES: COC1=CC=C2[C@H]3Cc4ccc(OCc5ccccc5)c5c4[C@@]2(CCN3Cc2ccccc2)[C@H]1O5]
and
c) preparing noroxymorphone from the compound of formula (I); and
d) preparing naltrexone from noroxymorphone.

17. A method of preparing naloxone comprising:
a) contacting thebaine and/or oripavine with a recombinant host cell that expresses one or more genes encoding a cytochrome P450 enzyme capable of N-demethylating and/or O-demethylating the thebaine and/or the oripavine, wherein at least one of the genes is a recombinant gene, to produce a compound of formula (II); 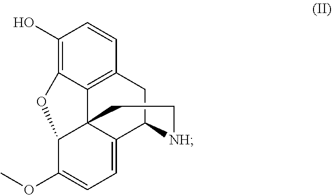 [SMILES: COC1=CC=C2[C@H]3Cc4ccc(O)c5c4[C@@]2(CCN3)[C@H]1O5]
and
b) reacting the compound of formula (II) with benzyl halide, benzyl sulfonate, or activated benzyl alcohol to provide a compound of formula (I): 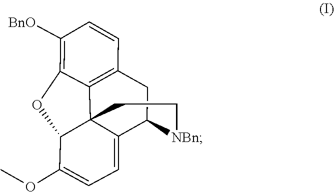 [SMILES: COC1=CC=C2[C@H]3Cc4ccc(OCc5ccccc5)c5c4[C@@]2(CCN3Cc2ccccc2)[C@H]1O5]
and
c) preparing noroxymorphone from the compound of formula (I); and
d) preparing naloxone from noroxymorphone.

18. A method of preparing buprenorphine, or a salt thereof, from a compound of formula (II), 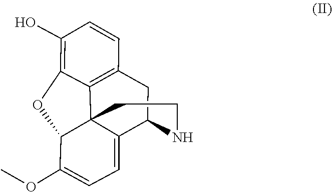 [SMILES: COC1=CC=C2[C@H]3Cc4ccc(O)c5c4[C@@]2(CCN3)[C@H]1O5]
or a salt thereof, comprising:
contacting thebaine and/or oripavine with a recombinant host cell that expresses one or more genes encoding a cytochrome P450 enzyme capable of N-demethylating and/or O-demethylating reticuline and/or derivatives thereof,
wherein at least one of the genes is a recombinant gene to produce compound MeO—I—H or the compound of formula (II), and 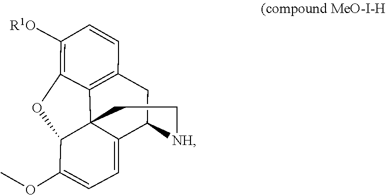
wherein R¹ is methyl)
I) (i)(A1) reacting the compound of formula (II) with cyclopropane carboxaldehyde followed by a hydride source; or
(i)(A2) reacting the compound of formula (II) with cyclopropanecarboxylic acid halide followed by a reducing agent; or
(i)(A3) reacting the compound of formula (II) with cyclopropylmethyl halide or activated cyclopropane methanol;
to provide compound HO—I-MCP: 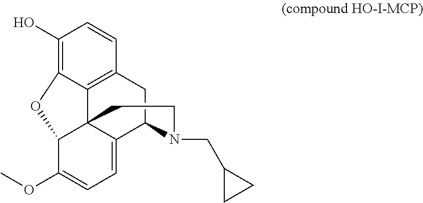 [SMILES: COC1=CC=C2[C@H]3Cc4ccc(O)c5c4[C@@]2(CCN3CC2CC2)[C@H]1O5]
(ii)(B) reacting compound HO—I-MCP with methyl vinyl ketone to provide compound HO—II-MCP: 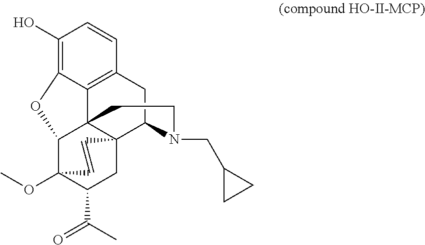 [SMILES: CO[C@]12C=C[C@@]3(C[C@@H]1C(C)=O)[C@H]1Cc4ccc(O)c5c4[C@@]3(CCN1CC1CC1)[C@H]2O5]
and either:
(iii)(C) reacting compound HO—II-MCP with H2 in the presence of a hydrogenation catalyst to provide compound HO—IIIB-MCP: 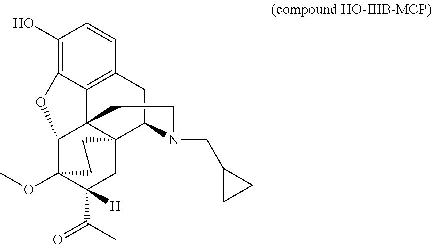 [SMILES: CO[C@]12CC[C@@]3(C[C@@H]1C(C)=O)[C@H]1Cc4ccc(O)c5c4[C@@]3(CCN1CC1CC1)[C@H]2O5]
(iv)(D) reacting compound HO—IIIB-MCP with tert-butylmagnesium halide to provide buprenorphine;
or
(iii)(D) reacting compound HO—II-MCP with tert-butylmagnesium halide to provide compound HO—IIIA-MCP: 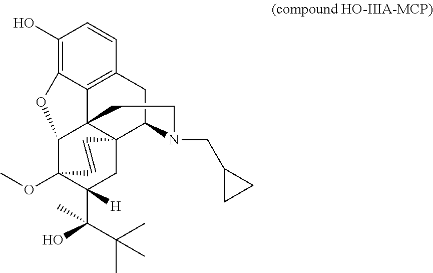 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]
(iv)(C) reacting compound HO—IIIA-MCP with H2 in the presence of a hydrogenation catalyst to provide buprenorphine;
II) (i)(A1) reacting compound MeO—I—H with cyclopropane carboxaldehyde followed by a hydride source; or
(i)(A2) reacting compound MeO—I—H with cyclopropanecarboxylic acid halide followed by a reducing agent; or
(i)(A3) reacting compound MeO—I—H with cyclopropylmethyl halide or activated cyclopropane methanol;
to provide compound MeO—I-MCP: 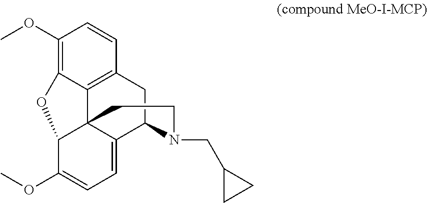 [SMILES: COC1=CC=C2[C@H]3Cc4ccc(OC)c5c4[C@@]2(CCN3CC2CC2)[C@H]1O5]
(ii)(B) reacting compound MeO—I-MCP with methyl vinyl ketone to provide compound MeO—II-MCP: 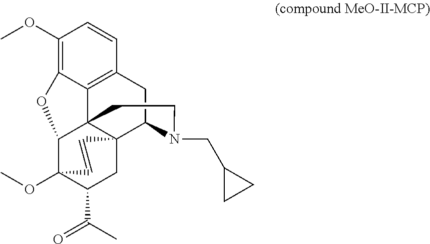 [SMILES: COc1ccc2c3c1O[C@H]1[C@@]4(OC)C=C[C@@]5(C[C@@H]4C(C)=O)[C@@H](C2)N(CC2CC2)CC[C@]315]
and either:
(iii)(C) reacting compound MeO—II-MCP with H2 in the presence of a hydrogenation catalyst to provide compound MeO—IIIB-MCP: 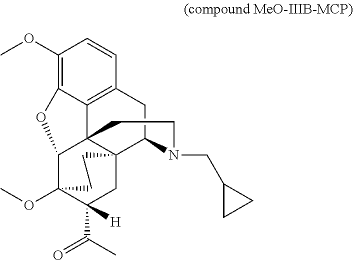 [SMILES: COc1ccc2c3c1O[C@H]1[C@@]4(OC)CC[C@@]5(C[C@@H]4C(C)=O)[C@@H](C2)N(CC2CC2)CC[C@]315]
(iv)(D) reacting compound MeO—IIIB-MCP with tert-butylmagnesium halide to provide compound MeO—IV-MCP: 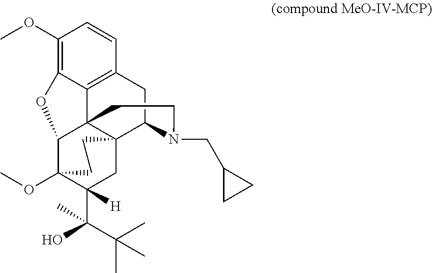 [SMILES: COc1ccc2c3c1O[C@H]1[C@@]4(OC)CC[C@@]5(C[C@@H]4[C@](C)(O)C(C)(C)C)[C@@H](C2)N(CC2CC2)CC[C@]315]
(v)(E) reacting compound MeO—IV-MCP with a demethylating agent to provide buprenorphine;
or
(iii)(D) reacting compound MeO—II-MCP with tert-butylmagnesium halide to provide compound MeO—IIIA-MCP: 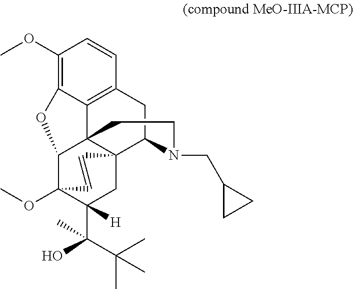 [SMILES: COc1ccc2c3c1O[C@H]1[C@@]4(OC)C=C[C@@]5(C[C@@H]4[C@](C)(O)C(C)(C)C)[C@@H](C2)N(CC2CC2)CC[C@]315]
(iv)(C) reacting compound MeO—IIIA-MCP with H2 in the presence of a hydrogenation catalyst to provide compound MeO—IV-MCP: 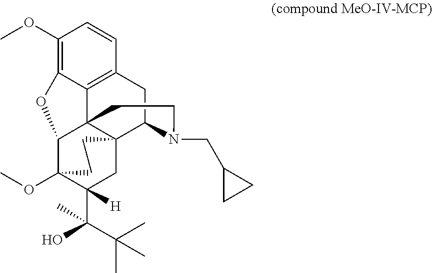 [SMILES: COc1ccc2c3c1O[C@H]1[C@@]4(OC)CC[C@@]5(C[C@@H]4[C@](C)(O)C(C)(C)C)[C@@H](C2)N(CC2CC2)CC[C@]315]
(v)(E) reacting compound MeO—IV-MCP with a demethylating agent to provide buprenorphine;
or
(iii)(D) reacting compound MeO—II-MCP with tert-butylmagnesium halide to provide compound MeO—IIIA-MCP: 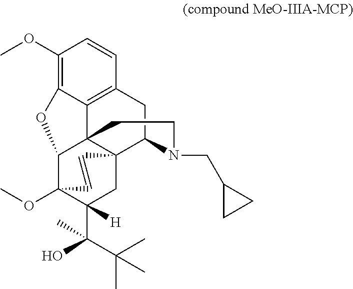 [SMILES: COc1ccc2c3c1O[C@H]1[C@@]4(OC)C=C[C@@]5(C[C@@H]4[C@](C)(O)C(C)(C)C)[C@@H](C2)N(CC2CC2)CC[C@]315]
(iv)(E) reacting compound MeO—IIIA-MCP with a demethylating agent to provide compound HO—IIIA-MCP: 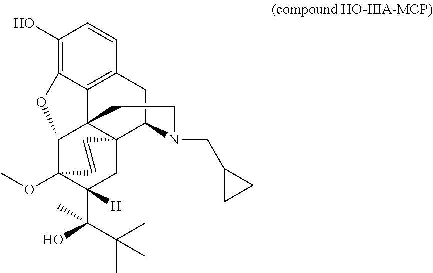 [SMILES: CO[C@]12C=C[C@@]3(C[C@@H]1[C@](C)(O)C(C)(C)C)[C@H]1Cc4ccc(O)c5c4[C@@]3(CCN1CC1CC1)[C@H]2O5]
(v)(C) reacting compound HO—IIIA-MCP with H2 in the presence of a hydrogenation catalyst to provide buprenorphine;
III) (i)(A1) reacting the compound of formula (II) with cyclopropane carboxaldehyde followed by a hydride source; or
(i)(A2) reacting the compound of formula (II) with cyclopropanecarboxylic acid halide followed by a reducing agent; or
(i)(A3) reacting the compound of formula (II) with cyclopropylmethyl halide or activated cyclopropane methanol;
to provide compound HO—I-MCP: 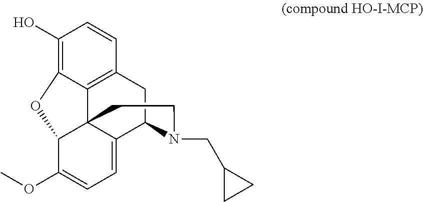 [SMILES: COC1=CC=C2[C@H]3Cc4ccc(O)c5c4[C@@]2(CCN3CC2CC2)[C@H]1O5]
and either:
(ii)(B) reacting compound HO—I-MCP with methyl vinyl ketone to provide compound HO—II-MCP: 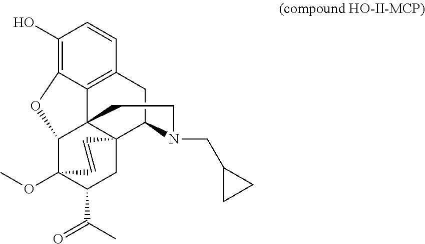 [SMILES: CO[C@]12C=C[C@@]3(C[C@@H]1C(C)=O)[C@H]1Cc4ccc(O)c5c4[C@@]3(CCN1CC1CC1)[C@H]2O5]
(iii)(F) reacting compound HO—II-MCP with benzyl halide, benzyl sulfonate, or activated benzyl alcohol to provide compound BnO—II-MCP: 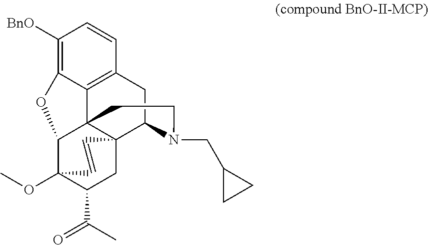 [SMILES: CO[C@]12C=C[C@@]3(C[C@@H]1C(C)=O)[C@H]1Cc4ccc(OCc5ccccc5)c5c4[C@@]3(CCN1CC1CC1)[C@H]2O5]
(iv)(D) reacting compound BnO—II-MCP with tert-butylmagnesium halide to provide compound BnO—IIIA-MCP: 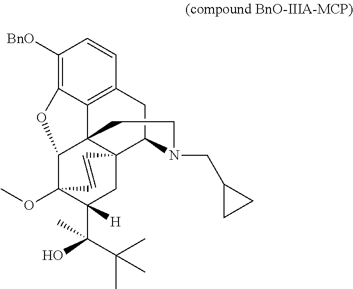 [SMILES: CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5]
(v)(C) reacting compound BnO—IIIA-MCP with H2 in the presence of a hydrogenation catalyst to provide buprenorphine;
or
(ii)(F) reacting compound HO—I-MCP with benzyl halide, benzyl sulfonate, or activated benzyl alcohol to provide compound BnO-I-MCP: 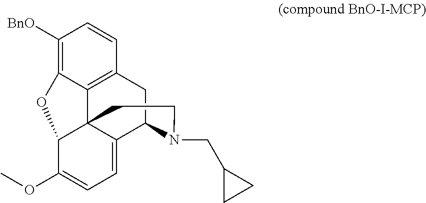 [SMILES: COC1=CC=C2[C@H]3Cc4ccc(OCc5ccccc5)c5c4[C@@]2(CCN3CC2CC2)[C@H]1O5]
(iii)(B) reacting compound BnO-I-MCP with methyl vinyl ketone to provide compound BnO—II-MCP: 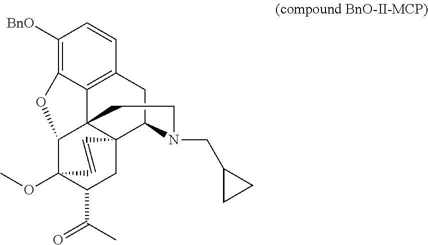 [SMILES: CO[C@]12C=C[C@@]3(C[C@@H]1C(C)=O)[C@H]1Cc4ccc(OCc5ccccc5)c5c4[C@@]3(CCN1CC1CC1)[C@H]2O5]
(iv)(D) reacting compound BnO—II-MCP with tert-butylmagnesium halide to provide compound BnO—IIIA-MCP: 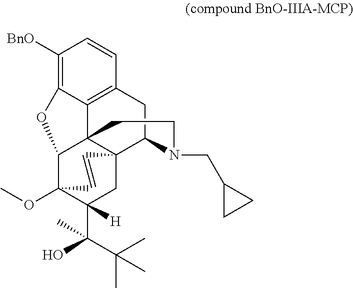 [SMILES: CO[C@]12C=C[C@@]3(C[C@@H]1[C@](C)(O)C(C)(C)C)[C@H]1Cc4ccc(OCc5ccccc5)c5c4[C@@]3(CCN1CC1CC1)[C@H]2O5]
(v)(C) reacting compound BnO—IIIA-MCP with H2 in the presence of a hydrogenation catalyst to provide buprenorphine;
IV) (i)(F) reacting the compound of formula (II) with benzyl halide, benzyl sulfonate, or activated benzyl alcohol to provide the compound of formula (I): 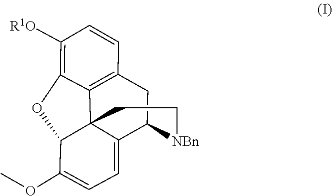
(ii)(B) reacting the compound of formula (I) with methyl vinyl ketone to provide compound BnO—II-Bn: 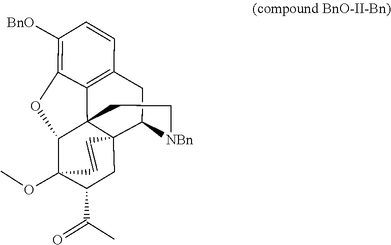 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]
(iii)(D) reacting compound BnO—II-Bn with tert-butylmagnesium halide to provide compound BnO—IIIA-Bn: 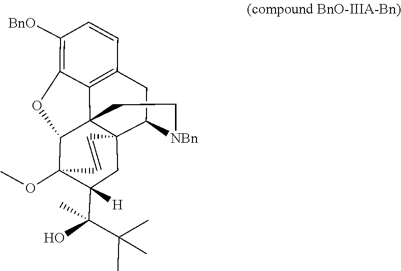 [SMILES: CO[C@]12C=C[C@@]3(C[C@@H]1[C@](C)(O)C(C)(C)C)[C@H]1Cc4ccc(OCc5ccccc5)c5c4[C@@]3(CCN1Cc1ccccc1)[C@H]2O5]
(iv)(C) reacting compound BnO—IIIA-Bn with H2 in the presence of a hydrogenation catalyst to provide a compound of compound HO—IV—H: 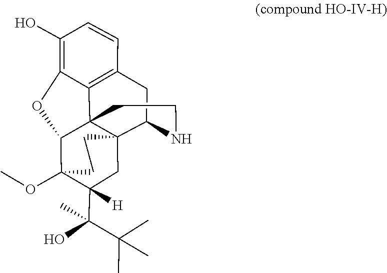 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
(v)(A1) reacting compound HO—IV—H with cyclopropane carboxaldehyde followed by a hydride source; or
(v)(A2) reacting compound HO—IV—H with cyclopropanecarboxylic acid halide followed by a reducing agent; or
(v)(A3) reacting compound HO—IV—H with cyclopropylmethyl halide or activated cyclopropane methanol;
to provide buprenorphine;
V) (i)(G) reacting the compound of formula (II) with optionally substituted benzoyl halide to provide compound HO—I—Ac: 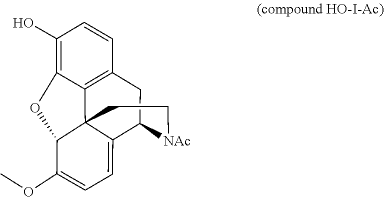
(ii)(F) reacting compound HO—I—Ac with benzyl halide, benzyl sulfonate, or activated benzyl alcohol to provide compound BnO—I—Ac: 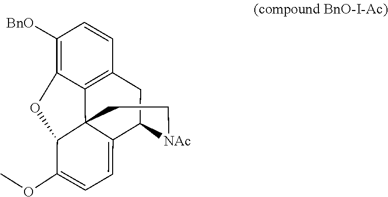
(iii)(H) reacting compound BnO—I—Ac with lithium aluminum hydride to provide the compound of formula (I): 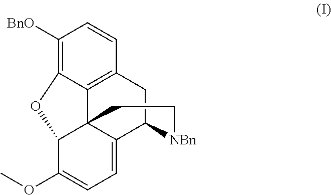 [SMILES: COC1=CC=C2[C@H]3Cc4ccc(OCc5ccccc5)c5c4[C@@]2(CCN3Cc2ccccc2)[C@H]1O5]
(iv)(B) reacting the compound of formula (I) with methyl vinyl ketone to provide compound BnO—II-Bn: 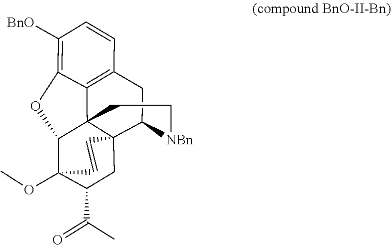 [SMILES: CO[C@]12C=C[C@@]3(C[C@@H]1C(C)=O)[C@H]1Cc4ccc(OCc5ccccc5)c5c4[C@@]3(CCN1Cc1ccccc1)[C@H]2O5]
(v)(D) reacting compound BnO—II-Bn with tert-butylmagnesium halide to provide compound BnO—IIIA-Bn: 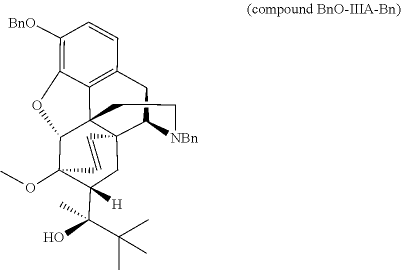
(vi)(C) reacting compound BnO—IIIA-Bn with H2 in the presence of a hydrogenation catalyst to provide compound HO—IV—H: 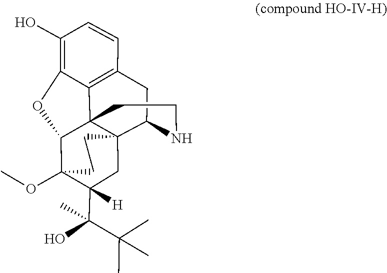 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
(vii)(A1) reacting compound HO—IV—H with cyclopropane carboxaldehyde followed by a hydride source; or
(vii)(A2) reacting compound HO—IV—H with cyclopropanecarboxylic acid halide followed by a reducing agent; or
(vii)(A3) reacting compound HO—IV—H with cyclopropylmethyl halide or activated cyclopropane methanol;
to provide buprenorphine;
VI) (i)(G) reacting the compound of formula (II) with optionally substituted benzoyl halide to provide compound AcO—I—Ac: 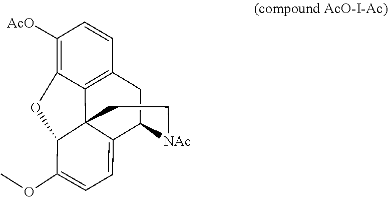
(ii)(B) reacting compound AcO—I—Ac with methyl vinyl ketone to provide compound AcO—II—Ac: 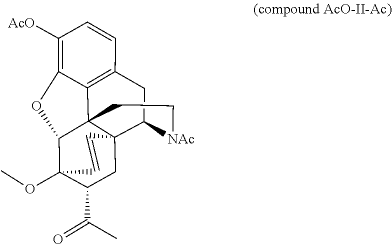
(iii)(D) reacting compound AcO—II—Ac with tert-butylmagnesium halide to provide compound HO—IIIA-Ac: 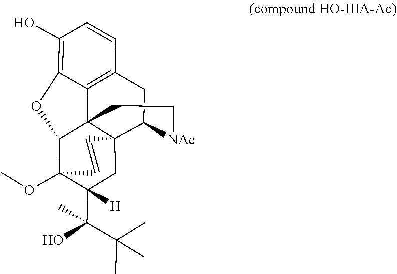
(iv)(H) reacting compound HO—IIIA-Ac with lithium aluminum hydride to provide compound HO—IIIA-Bn: 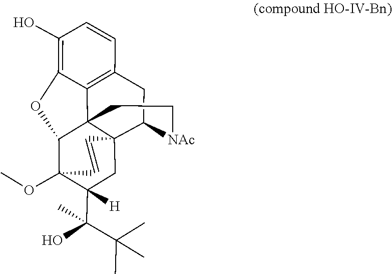
(v)(C) reacting compound HO—IV-Bn with H2 in the presence of a hydrogenation catalyst to provide compound HO—IV—H: 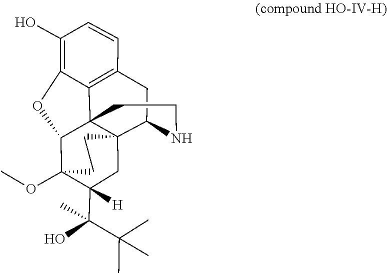 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
(vi)(A1) reacting compound HO—IV—H with cyclopropane carboxaldehyde followed by a hydride source; or
(vi)(A2) reacting compound HO—IV—H with cyclopropanecarboxylic acid halide followed by a reducing agent; or
(vi)(A3) reacting compound HO—IV—H with cyclopropylmethyl halide or activated cyclopropane methanol;
to provide buprenorphine; or
VII) (i)(G) reacting the compound of formula (II) with acyl halide to provide compound AcO—I—Ac: 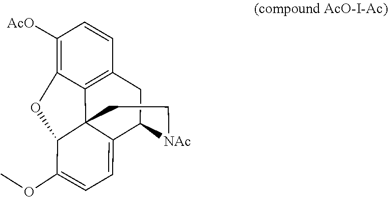
(ii)(B) reacting compound AcO—I—Ac with methyl vinyl ketone to provide compound AcO—II—Ac: 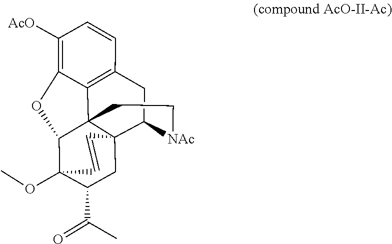
and either:
(iii)(D) reacting compound AcO—II—Ac with tert-butylmagnesium halide to provide compound HO—IIIA-Bn: 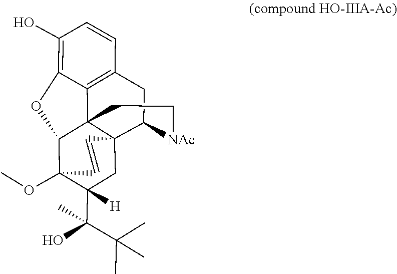
(iv)(C) reacting compound HO—IIIA-Ac with H2 in the presence of a hydrogenation catalyst to provide compound HO—IV—Ac: 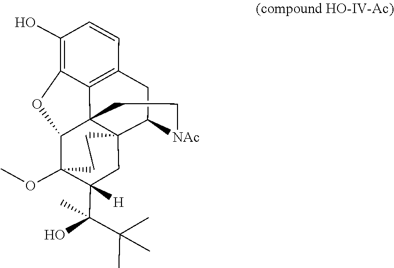
(v)(I) reacting compound HO—IV—Ac with Schwartz's reagent or base to provide compound HO—IV—H: 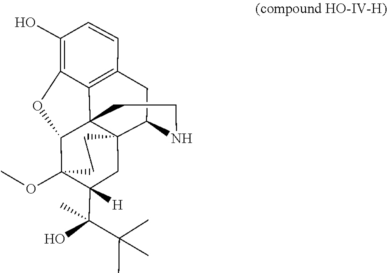 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
(vi)(A1) reacting compound HO—IV—H with cyclopropane carboxaldehyde followed by a hydride source; or
(vi)(A2) reacting compound HO—IV—H with cyclopropanecarboxylic acid halide followed by a reducing agent; or
(vi)(A3) reacting compound HO—IV—H with cyclopropylmethyl halide or activated cyclopropane methanol;
to provide buprenorphine; or
(iii)(C) reacting compound HO—IIIA-Ac with H2 in the presence of a hydrogenation catalyst to provide compound AcO—IIIB—Ac: 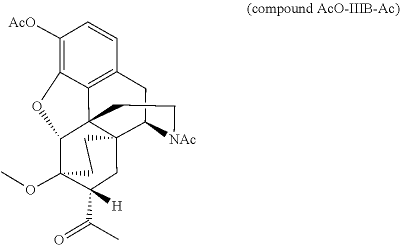
(iv)(D) reacting compound AcO—II—Ac with tert-butylmagnesium halide to provide compound HO—IV—Ac: 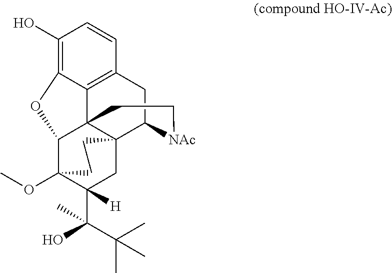
(v)(I) reacting compound HO—IV—Ac with Schwartz's reagent or base to provide compound HO—IV—H: 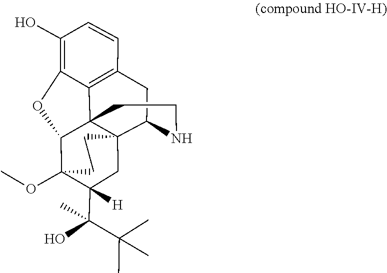 [SMILES: CO[C@]12CC[C@@]3(C[C@@H]1[C@](C)(O)C(C)(C)C)[C@H]1Cc4ccc(O)c5c4[C@@]3(CCN1)[C@H]2O5]
(vi)(A1) reacting compound HO—IV—H with cyclopropane carboxaldehyde followed by a hydride source; or
(vi)(A2) reacting compound HO—IV—H with cyclopropanecarboxylic acid halide followed by a reducing agent; or
(vi)(A3) reacting compound HO—IV—H with cyclopropylmethyl halide or activated cyclopropane methanol;
to provide buprenorphine.

19. The method according to claim 1, wherein the cytochrome P450 enzyme capable of N-demethylating and/or O-demethylating a reticuline derivative has at least 50% sequence identity with a sequence selected from the group consisting of:
i) CYPDN8 (SEQ ID NO: 73),
ii) Mc_S2JT25 (SEQ ID NO: 53),
iii) CYPDN17 (SEQ ID NO: 91),
iv) CYPDN12 (SEQ ID NO: 81),
v) Lr_P450 (SEQ ID NO: 7),
vi) CYPDN29 (SEQ ID NO: 115),
vii) CYPDN14 (SEQ ID NO: 85),
vii) P450_DN15259_c0_g1_i7 (SEQ ID NO: 1),
ix) LCOR_01865 (SEQ ID NO: 55),
x) P450_DN5615_c2_g1_i9 (SEQ ID NO: 63),
xi) P450_DN12791_c0_g1_i1 (SEQ ID NO: 4),
xii) CYPDN16 (SEQ ID NO: 89),
xiii) CYPDN18 (SEQ ID NO: 93),
xiv) CYPDN27 (SEQ ID NO: 111),
xv) CYPDN35 (SEQ ID NO: 127),
xvi) CYPDN5 (SEQ ID NO: 67),
xvii) CYPDN6 (SEQ ID NO: 69),
xviii) CYPDN7 (SEQ ID NO: 71),
xix) CYPDN10 (SEQ ID NO: 77),
xx) CYPDN11 (SEQ ID NO: 79),
xxi) CYPDN24 (SEQ ID NO: 105),
xxii) CYPDN28 (SEQ ID NO: 113),
xxiii) CYPDN13 (SEQ ID NO: 83),
xxiv) CYPDN31 (SEQ ID NO: 119),
xxv) CYPDN34 (SEQ ID NO: 125),
xxvi) CYPDN22 (SEQ ID NO: 101),
xxvii) CYPDN21 (SEQ ID NO: 99),
xxviii) CYPDN30 (SEQ ID NO: 117),
xxix) Ar_ORZ22410 (SEQ ID NO: 59), and
xxx) CYPDN20 (SEQ ID NO: 97).
